(19) United States
(12) Patent Application Publication
(10) Pub. No.: US 2025/0332283 A1
(43) Pub. Date: Oct. 30, 2025

(54) NUCLEOTIDE DELIVERY FROM INJECTABLE HYDROPHOBIC BIOMATERIAL

(71) Applicant: DANMARKS TEKNISKE UNIVERSITET, Kongens Lyngby (DK)

(72) Inventors: Thomas L ANDRESEN, Kongens Lyngby (DK); Jonas R HENRIKSEN, Kongens Lyngby (DK); Anders E HANSEN, Kongens Lyngby (DK); Carl Fredrik MELANDER, Kongens Lyngby (DK); Linda Maria BRUUN, Kongens Lyngby (DK); Anja BRUS, Kongens Lyngby (DK); Gael Francis CLERGEAUD VEIGA, Kongens Lyngby (DK); Kaja Borup LØVSCHALL, Kongens Lyngby (DK); Ditte Elisabeth JÆHGER, Kongens Lyngby (DK); Laura Olivia Marie MARCHARD, Kongens Lyngby (DK); Unnur Jóna BJÖRGVINSDÓTTIR, Kongens Lyngby (DK); Li XIN, Kongens Lyngby (DK)

(21) Appl. No.: 18/868,364

(22) PCT Filed: May 22, 2023
(86) PCT No.: PCT/EP2023/063688

§ 371 Date: Nov. 22, 2024

(30) Foreign Application Priority Data
May 23, 2022 (GB) 2207544.4

(51) Int. Cl. A61K 48/00; A61K 47/69

(52) U.S. Cl. CPC A61K 48/0033; A61K 47/6929; A61K 48/0075

(57) ABSTRACT

The invention provides sustained delivery of nucleic acids from hydrophobic/organic media, formulations and biomaterial depots.

### FIELD OF THE INVENTION

The invention relates to the field of medicine. More particularly, it relates to the field of sustained delivery of nucleic acids from hydrophobic/organic media, formulations and biomaterial depots. Hereby enhanced stability and sustained release of nucleic acids is achieved, with applications in gene transfection, editing, induction and silencing.

### Introduction to the Invention

Gene expression is central for biological processes in living organisms. Through gene expression regulation, the coding-information from a gene is used in the synthesis of a functional gene product (mRNA) that enables it to produce an end product, protein or non-coding RNA, and ultimately affect the cell phenotype and functional behaviour, as the final effect. These products are often proteins, but in non-protein-coding genes such as transfer RNA (tRNA) and small nuclear RNA (snRNA), and other non-coding RNA the product is a functional non-coding RNA.

Altered regulation of gene expression is therefore also central in disease processes and disease mechanisms. This therefore represents a unique therapeutic opportunity for providing cells with the nucleic acid components required for producing therapeutic proteins or regulating biological processes related precisely to a given disease. Their ability to manipulate gene expression or produce therapeutic proteins, make nucleic acid-based therapeutics suitable for pathologies with established genetic targets, including infectious diseases, cancers, immune diseases, tissue engineering and regeneration, hormonal disease, and neurological disorders.

Nucleic acid-based therapeutics hold great promise for improving the treatment of infectious diseases, deficiency diseases, autoimmunity, degenerative disease, storage disorders, hereditary diseases (including both genetic diseases and non-genetic hereditary diseases), and physiological diseases. However, because the nucleic acid therapeutics aim to closely mimic the expression or inhibition of biological and disease processes optimal therapeutic stimulation is challenging. The biological processes are generally continuously active which requires careful continuous stimulation or inhibition. From a therapeutic standpoint this is challenging as the nucleic acid-based therapies are problematic to deliver, have a rapid distribution from e.g., the injection site and a short-lived therapeutic activity, in addition to a poor inherent stability of several nucleic acid classes. Key examples include the induction or inhibition of immune activating signalling molecules for cancer immunotherapy. In the case of immunotherapy, the plastic and reactive components of the anti-cancer immune response are short-lived and rapidly return to a pro-tumorigenic state unless continuously stimulated. This challenge also exists for vaccines where optimal responses are generated through a continuous stimulation and activation of the immune response to generate a durable cellular and or humoral adaptive response.

Nucleic acid-based therapeutics allows for flexible production or inhibition of specific protein expressions. In some cases, production or inhibition of specific proteins may be tolerated systemically or even be aimed as systemic therapeutics. However, in some cases the produced protein or inhibited protein may only be aimed towards a specific tissue e.g., cancerous tissue, inflammatory tissue, specific organs to manipulate a specific biological process at the target site or simple because of poor systemic tolerance. In these cases, direct administration at the site of intended effect may be needed. However, direct intralesional or regional administration may be associated with a rapid distribution of e.g., a poorly tolerated nucleic acid therapeutic. In other cases, administration into the specific tissue of interest may be challenging and require advanced interventional procedures.

Existing nucleic acid-based therapies therefore face a plethora of clinical and pharmacokinetic challenges. Targeted administration of existing nucleic acid-based therapies is therefore hampered by their rapid dispersal throughout the subject following administration. Whilst this is acceptable, or even desirable, for applications which rely upon systemic distribution of therapeutic nucleic acid throughout the subject, it is undesirable for applications which rely upon localised effects e.g. administration to cancerous tissue, inflammatory tissue, specific organs therapies. Existing nucleic acid-based therapies are also associated with rapid clearance of the nucleic acid to the subject, which can be necessitate repeat administration to maintain therapeutic levels within the subject. Thus, there exists a considerable need in the art for compositions and methods which enable localised release of nucleic acid-based therapies to a subject. There is likewise an urgent need for compositions and methods which enable sustained release of therapeutic nucleic acids to a subject.

Existing nucleic acid-based therapies are further hampered by stability issues associated with the aqueous environment within the subject. Such stability issues are particularly problematic when the nucleic acid-based therapy is hydrophobic e.g. when formulated as a lipid nanoparticle (LNP). Thus, there also exists an urgent therapeutic need for compositions and methods which achieve sustained potency of nucleic acid-based therapies within aqueous environments, such as the body of a subject.

The present invention addresses the above needs by providing a composition comprising a nucleic acid component (such as a therapeutic nucleic acid) and a hydrophobic component, wherein the hydrophobic component comprises: (i) a hydrophobic carbohydrate, a lipid, a hydrophobic polymer, or mixture thereof; and (ii) a hydrophobic or amphiphilic molecule that contains at least one primary, secondary, tertiary or quaternary amine, wherein the nucleic acid component and the hydrophobic component are dissolved in an organic solvent, and wherein the composition has a higher viscosity in an aqueous environment as compared to its viscosity in a non-aqueous environment.

Compositions of the invention are fluid when in non-aqueous environments (which may be a substantially pure preparation containing only the composition of the invention), making them ideally suited to administration regimes such as injection. Advantageously, compositions of the invention increase their viscosity when transferred to an aqueous environment (such as when administered to a subject) to provide a depot-like formulation. Advantageously, the increased viscosity considerably reduces dispersion of the composition (including the nucleic acid component) from the site of administration, thereby enhancing the localised therapeutic effect. Moreover, retention of the composition at or close to the site of administration enables accurate targeting to the site of interest, which may be further enhanced e.g. by incorporating an imaging agent (e.g. contrast agent) into the composition. Reduced dispersion enhances localised therapeutic effects and avoids the higher dosage requirements otherwise required to counteract the dilution effect of systemic dispersal. Advantageously, the compositions of the invention can reduce unwanted systemic distribution of therapeutic nucleic acids, thereby reducing nucleic acid-induced systemic toxicity (e.g. ASO-induced systemic toxicity) as compared to administration of free nucleic acids.

Moreover, the hydrophobic environment within the composition helps stabilise nucleic acid components which otherwise degrade more quickly in an aqueous environment. Advantageously, this maintains the potency of therapeutic nucleic acids, thereby enhancing their therapeutic effect over time, and reducing the requirement for repeat administrations. Moreover, nucleic acid components are released from the composition over time, thereby increasing reducing the administration interval required to maintain a therapeutic effect. Advantageously, compositions of the invention may also be tailored to control the release rate of the nucleic acid component to provide an optimal therapeutic effect. Whilst the present invention is particularly well-suited to providing a localised therapeutic effect, compositions of the invention may also be tailored to provide a systemic effect alongside the increased stability and desirable release kinetics described above. The increased stability provided by the present invention acts with sustained and customisable release to provide ‘real-world’ synergistic effects which are directly relevant in the clinic.

In more detail, the present invention allows for controlled and tailored release of nucleic acids, such as nucleic acid-based therapeutics, such as natural and synthetic DNA and RNA classes from hydrophobic media. Specific examples of such formulations contain oils, triglycerides, lipids, hydrophobic polymers, hydrophobic carbohydrates and mixtures thereof. A particular interesting composition of invention comprises carbohydrate-based esters, organic co-solvents and organic solvents.

Advantageously, compositions of the invention can surprisingly be used to formulate and release hydrophilic agents such as nucleic acids that would not normally be soluble in hydrophobic environments, compositions and depots. Compositions of the invention can be used to release nucleic acids such as oligonucleotides, polynucleotides, RNA and DNA in a controlled way over time.

The present invention provides compositions that can solubilize nucleic acids such as oligonucleotides, polynucleotides, RNA and DNA in a hydrophobic environment. The invention furthermore provides compositions from which nucleic acids such as oligonucleotides, polynucleotides, RNA and DNA are released in a controlled way. Compositions of the invention are particularly well-suited to transfection of animal or human cells with the nucleic acid component to achieve an altered biological function.

The present invention is based, in part, on the surprising discovery that a higher concentration of nucleic acid component may be incorporated into the composition when the hydrophobic component comprises a hydrophobic or amphiphilic molecule that contains at least one primary, secondary, tertiary or quaternary amine. The Inventors discovered that incorporating a hydrophobic or amphiphilic amine into the hydrophobic component achieves unexpectedly superior solubilisation or dispersion of nucleic acid components, in turn increasing the therapeutic ‘payload’ potential of the compositions of the invention. This is highly advantageous because it increases the potency potential, reduces the dosage requirements, and enhances the ability to incorporate therapeutic levels of different nucleic acid components within the composition. Without wishing to be bound by theory, the Inventors believe that the hydrophobic or amphiphilic molecule that contains at least one primary, secondary, tertiary or quaternary amine achieves superior complexation with the nucleic acid component, making the nucleic acid component more hydrophobic, as compared to complexation as a salt with e.g. ammonium chloride or other small hydrophilic amines.

The nucleic acid component may further comprise additional molecules that form particles, such as lipid nanoparticles (LNPs), polyplexes, lipoplexes and hydrophobic ion-pairs (HIPs) with the nucleic acid. Formulating the nucleic acid component as such can help improve solubility within the organic solvent. In one embodiment, the nucleic acid component may be complexed either by cationic polymers, cationic lipid mixtures or cationic hydrophobic counterions forming particles, typically with 20-500 nm size or smaller HIP complexes that may or may not form particles. In this process, the native charges of the nucleic acids are screened, which facilitate their transfer to apolar/hydrophobic media such as organic solvents. Increasing the hydrophobicity of the nucleic acid component typically enhances its solubility and dispersal within hydrophobic formulations such as but not limited to oils, triglycerides, lipids, hydrophobic polymers, hydrophobic carbohydrates and mixtures thereof. In some embodiments, the nucleic acid t comprises oligonucleotide(s), polynucleotide(s), RNA or DNA are formulated as an LNP, polyplex, lipoplex or HIP.

Previously, ion pairing has been used to formulate nanoparticles from hydrophobic compounds. These are defined by having logP values greater than 1, 2, 3, 4, or 5 at neutral pH [Pinkerton, N. M., et al, Formation of stable nanocarriers by in situ ion pairing during block-copolymer directed rapid precipitation. Molecular pharmaceutics, 2013. 10 (1): p.319-328]. For example, Song et.al [Song, Y. H., et al, A novel in situ hydrophobic ion pairing (HIP) formulation strategy for clinical product selection of a nanoparticle drug delivery system. Journal of Controlled Release, 2016. 229: p. 106-119.] used ion pairing for AZD281, which had a logP of 2 at a neutral pH of 7. High solubility could mean logP values less than-2 and/or solubilities in aqueous media of over 10 mg/ml at pH=7. Patel and Guadana [Gaudana, R., et al, Design and evaluation of a novel nanoparticulate-based formulation encapsulating a HIP complex of lysozyme. Pharmaceutical development and technology, 2013. 18 (3): p. 752-759; Patel, A., R. Gaudana, and A. K. Mitra, A novel approach for antibody Nanocarriers development through hydrophobic ion-pairing complexation. Journal of Microencapsulation, 2014. 31 (6): p. 542-550] showed that soluble antibody or lysozyme proteins could be complexed with oppositely charged dextran sulfate. Dextran sulfate is a water-soluble polymer. Hydrophobic ion paring has previously been patented for encapsulation and precipitation of APIs in nanoparticles (WO2019090030).

Compositions of the invention are a liquid before administration (e.g. by injection) to the animal or human body. Following administration, compositions of the invention increase their viscosity, typically forming a depot, from which the nucleic acid component is released into the animal or human body. The compositions will upon injection into aqueous media such as tissue typically become a depot. Here from, the nucleic acid component will be released e.g. as free nucleic acid, as an LNP, as a polyplex or lipoplex transfection system, or as HIPs of oligonucleotides, polynucleotides, RNA and DNA. The HIPs may be released as particles or as complexes of single oligonucleotides, polynucleotides, RNA and DNA and may have transfection capabilities on their own, or the HIPs may dissociate and release the native nucleic acid in cell surroundings or within cells.

The invention allows for sustained manipulation of biological processes within cells and tissues for improved therapeutic performance or can be used in vaccination. The present invention provides a number of advantages, such as increased dosing intervals, or reduced toxicity, increased therapeutic effect, reduced fluctuations in protein levels produced from administered gene material, and improved vaccine performance. For vaccines, the present invention can also avoid the requirement for repeated vaccinations (booster).

Depending on the disease of interest, the nucleic acid component (e.g. a therapeutic nucleic acid) may be constructed to serve as a template for production of a specific protein, be a non-protein-coding sequence or a nucleic acid sequence aiming to prevent the expression of a specific gene. Exemplary nucleic acid components comprise DNA, pDNA, ssDNA, dsDNA, antisense DNA, eecDNA, microDNA, spcDNA, episomal DNA, linear DNA, RNA (messenger RNA (mRNA), self-replicating mRNA, transfer RNA (tRNA), and ribosomal RNA (rRNA)), small single or double stranded RNA (dsRNA, RNA interference (RNAi) including microRNA (miRNA), small interfering RNA (siRNA or ASO, with modifications including phosphorotioate (PS), PS morpholino, 2′-O-methyl(2′-OMe), 2′-O-methoxyethyl(2′-MOE), 2′fluoro, 5′methylcystine, G-clamp), splice switching antisense oligonucleotide (SSO), CRISPR-Cas9 sgRNAs, piwi-interacting RNA (piRNA) and/or repeat associated small interfering RNA (rasiRNA). Exemplary nucleic acid components also comprise circular RNA (circRNA), long non-coding RNA (lncRNA), transfer-messenger RNA (tmRNA), ribozymes, aptamers and artificial nucleic acids. In addition to unmodified nucleic acids alternative synthetic nucleic acids analogous include Peptide nucleic acid (PNA), Morpholino and locked nucleic acid (LNA), glycol nucleic acid (GNA), threose nucleic acid (TNA), hexitol nucleic acids (HNA), bridged nucleic acid (BNA) and/or 2′-O-methyl-substituted RNA. Synthetic nucleic acid analogs also include S-constrained ethyl (cEt).

In one embodiment, the nucleic acid component is an ASO.

In one embodiment, the nucleic acid component is an siRNA.

In one embodiment, the nucleic acid component comprises one or more types of nucleic acid, e.g., antigen-encoding nucleic acid. In one embodiment, the nucleic acid component may comprise a chimeric polynucleotide in linear and/or circular form. In another embodiment, the nucleic acid component may comprise a circular polynucleotide and an in vitro transcribing (IVT) polynucleotide. In yet another embodiment, the nucleic acid component may comprise an IVT polynucleotide, a chimeric polynucleotide and a circular polynucleotide.

In one embodiment the nucleic acid component contains nucleic acid encoding proteins selected from categories such as, but not limited to, human proteins, veterinary proteins, bacterial proteins, biological proteins, antibodies, immunogenic proteins, therapeutic peptides and proteins, secreted proteins, plasma membrane proteins, cytoplasmic and cytoskeletal proteins, intracellular membrane bound proteins, nuclear proteins, proteins associated with human disease and/or proteins associated with non-human diseases. In one embodiment, the nucleic acid component contains at least three polynucleotides encoding proteins. In one embodiment, the nucleic acid component contains at least five polynucleotide encoding proteins.

RNA interference (RNAi) as used herein refers to a post-transcriptional, targeted gene-silencing technique in which an interfering RNA degrades messenger RNA (mRNA) that contains the same or a very similar sequence to the interfering RNA. In some embodiments, the therapeutic nucleic acid is an interfering RNA, optionally wherein the interfering RNA is selected from a short interfering RNA (siRNA), micro RNA (miRNA), short hairpin RNA (shRNA), antisense oligonucleotides (ASO), splice switching antisense oligonucleotide (SSO), CRISPR-Cas9 sgRNAs, piwi-interacting RNA (piRNA) and repeat associated small interfering RNA (rasiRNA). Interfering RNAs suppress the expression of a target RNA transcript by annealing to the target RNA transcript to form a nucleic acid duplex and (i) promoting the nuclease-mediated degradation of the RNA transcript; and/or (ii) slowing, inhibiting, or preventing the translation of the RNA transcript, such as by sterically precluding the formation of a functional ribosome-RNA transcript complex or otherwise attenuating formation of a functional protein product from the target RNA transcript, interfering RNA may be provided to a patient in the form of, e.g., a single- or double-stranded oligonucleotide or a transgene encoding the interfering RNA.

In some embodiments, the interfering RNA is operably linked to a promoter that induces expression of the interfering RNA in a muscle cell or neuron. The promoter may be, for example, a desmin promoter, a phosphoglycerate kinase (PGK) promoter, a muscle creatine kinase promoter, a myosin light chain promoter, a myosin heavy chain promoter, a cardiac troponin C promoter, a troponin I promoter, a myoD gene family promoter, an actin alpha promoter, an actin beta promoter, an actin gamma promoter, or a promoter within intron 1 of ocular paired like homeodomain 3 (PITX3).

In some embodiments, the therapeutic nucleic acid is a nucleic acid vaccine. As used herein, a “nucleic acid vaccine” is a vaccine composition which includes a nucleic acid or nucleic acid molecule (e.g., a polynucleotide) encoding an antigen (e.g., an antigenic protein or polypeptide). In some embodiments, the nucleic acid vaccine comprises RNA. In some embodiments, the nucleic acid vaccine comprises messenger RNA (“mRNA”). In some embodiments, the nucleic acid vaccine comprises DNA.

Therapeutic application of the different classes of natural and synthetic nucleic acids can regulate or modulate the gene expression process, including the transcription, RNA splicing, translation, and post-translational modification of a protein. This gives control over the timing, location, and amount of a given gene product (protein or ncRNA) present in a cell and can have a profound effect on both the specific cells function but also induce the production or inhibition of therapeutic proteins that affect cells through autocrine, paracrine, juxtacrine or endocrine signalling.

The flexibility of the gene expression machinery furthermore allows for production of foreign protein that would normally be produced by a pathogen (such as a virus or bacteria) or by a cancer cell. Here, DNA or mRNA may be delivered to produce an immune response, serving as indirect (DNA) or direct (mRNA) blueprint sequences to build the foreign protein of interest. This provides high flexibility for generating platform technologies that can be broadly applied and rapidly modified to accommodate alterations in pathogen of interest e.g., in case of mutations in targeted pathogens or cancer.

In some embodiments, the nucleic acid component is an aptamer. An aptamer may be based on natural or synthetic oligonucleotides. Based on oligonucleotide sequences, they bind to a specific target molecule and can be combined with ribozymes to self-cleave in the presence of their target molecule. Aptamers thereby exhibit affinity for a given target with selectivity and specificity comparable to antibodies. They do, however, possess important advantages as they are engineered completely in vitro, are readily produced by chemical synthesis, possess desirable storage properties, and elicit little or no immunogenicity in therapeutic applications.

Aptamers are nucleic acid molecules that bind a specific target molecule. Aptamers can be engineered in vitro, are readily produced by chemical synthesis, possess desirable storage properties, and elicit little or no immunogenicity in therapeutic applications. These characteristics make aptamers particularly useful in pharmaceutical and therapeutic utilities. As used herein, “aptamer” refers in general to a single or double stranded oligonucleotide or a mixture of such oligonucleotides, wherein the oligonucleotide or mixture is capable of binding specifically to a target. Other aptamers having equivalent binding characteristics can also be used, such as peptide aptamers.

In general, aptamers may comprise oligonucleotides that are at least 5, at least 10 or at least 15 nucleotides in length. Aptamers may comprise sequences that are up to 40, up to 60 or up to 100 or more nucleotides in length. For example, aptamers may be from 5 to 100 nucleotides, from 10 to 40 nucleotides, or from 15 to 40 nucleotides in length. Where possible, aptamers of shorter length are preferred as these will often lead to less interference by other molecules or materials. Aptamers may be generated using routine methods such as the Systematic Evolution of Ligands by Exponential enrichment (SELEX) procedure. SELEX is a method for the in vitro evolution of nucleic acid molecules with highly specific binding to target molecules.

The SELEX method involves the selection of nucleic acid aptamers and in particular single stranded nucleic acids capable of binding to a desired target, from a collection of oligonucleotides. A collection of single-stranded nucleic acids (e.g., DNA, RNA, or variants thereof) is contacted with a target, under conditions favourable for binding, those nucleic acids which are bound to targets in the mixture are separated from those which do not bind, the nucleic acid-target complexes are dissociated, those nucleic acids which had bound to the target are amplified to yield a collection or library which is enriched in nucleic acids having the desired binding activity, and then this series of steps is repeated as necessary to produce a library of nucleic acids (aptamers) having specific binding affinity for the relevant target.

The therapeutic use of nucleic acids is, however, faced by many hurdles The lipid bilayers of cells allow small neutral, slightly hydrophobic molecules <1,000 Daltons (Da) to passively diffuse across them, while preventing large, charged molecules, like DNAs and RNAs, from crossing them. The cells are furthermore protected from invading DNAs and RNAs by nucleases and the innate immune pattern recognition surface and intracellular receptors. All natural nucleic acid therapeutics are large and/or highly charged macromolecules that have no ability to cross lipid bilayers, and range in size from 4-10 kDa for single-stranded siRNAs, to ˜14 kDa for double-stranded siRNAs, to ˜200 kDa for CRISPR-Cas9 sgRNAs to 700-7,000 kDa for self-replicating mRNAs, mRNAs and pDNAs. The therapeutic success of nucleic acid technologies is furthermore not limited to the cell membrane, for RNA delivery escaping the endosomal barrier is central and DNA also requires translocation into the nucleus.

In some embodiments, the nucleic acid component is formulated for transfection applications. For example, non-viral transfection technologies have been extensively evaluated for serving as vehicles for the transfer of nucleic acids across cell membranes and guiding intracellular trafficking. Synthetic transfection technologies have generally been focused on polymers, LNPs, polyplexes, liposomes, lipoplexes, or nanoparticles. In polymer transfection the polycationic polymers such as DEAE-dextran or polyethylenimine (PEI) bind the negatively charged nucleic acids to form a complex that is taken up by the cell via endocytosis. Lipid nanoparticle transfection systems use positively charged lipids (cationic liposomes or mixtures) to form an aggregate with the negatively charged nucleic acids that allows the complex to enter cells. Alternative non-lipid-based particle systems include dendrimers, cell penetrating peptide conjugates and multi-component reagent technologies. Dendrimers are a class of highly branched molecules based on various building blocks and synthesized through a convergent or a divergent method. Dendrimers bind nucleic acids to form dendriplexes that then penetrate the cells. Conjugation of nucleic acids and cell-penetrating peptides (CPPs) may deliver therapeutic nucleic acids to cells by using the intrinsic properties of the CPPs. Methods for cellular delivery of ASOs or siRNAs include conjugation to cell penetrating peptides, targeting ligands, GalNAc, Mannose, carbohydrates, cholesterol conjugation, proteamine-antibody fusion proteins, atelocollagen, stable nucleic acid-lipid particles, or polyethyleneimine-mediated uptake. As an alternative, synthetic nucleic acid modification has been demonstrated to not only increase stability and resistance to nucleases; but also alleviate the need for transfection systems. As an example, phosphorothioate (PS) modified siRNA can stimulate cellular uptake via the caveosomal uptake pathway, which may eliminate the requirement for formulation with transfection system. PS-siRNAs may be subject to intracellular transport and trapping which may be controlled through siRNA-peptide conjugation to optimize therapeutic activity.

### SUMMARY OF THE INVENTION

The invention comprises compositions, methods and material designs for achieving sustained release of nucleic acid from depots, such as NCCells, where the nucleic acid may be released in the form of free species or as transfection systems or transfection particles or combinations hereof.

The invention provides a composition comprising: (a) a nucleic acid component; and (b) a hydrophobic component; wherein: (i) the hydrophobic component comprises a hydrophobic carbohydrate, a lipid, a hydrophobic polymer, or mixture thereof; (ii) the hydrophobic component comprises a hydrophobic or amphiphilic molecule that contains at least one primary, secondary, tertiary or quaternary amine; (iii) the nucleic acid component and the hydrophobic component are dispersed or dissolved in an organic solvent; and (iv) the composition has a higher viscosity in an aqueous environment as compared to its viscosity in a non-aqueous environment.

In one embodiment, the organic solvent diffuses from the composition when the composition is in an aqueous environment. In one embodiment, more than 10% of the organic solvent diffuses from the composition when the composition is in an aqueous environment, e.g. at least 10%, at least 15%, at least 20%, at least 25%, at least 30%, at least 35%, at least 40%, at least 45%, at least 50%, at least 60%, at least 70%, at least 80%, at least 85%, at least 90%, at least 95%, at least 98%, at least 99% or 100% of the organic solvent diffuses from the composition when the composition is in an aqueous environment. In one embodiment, said diffusion occurs during a period of 48 hours following transfer of the composition to an aqueous environment.

In one embodiment, the composition has a viscosity that is at least 10,000 centipoise (cP) higher in an aqueous environment than in its viscosity in a non-aqueous environment.

In one embodiment, the composition is a liquid when in a non-aqueous environment.

In one embodiment, the composition transforms to a gel-like state when transferred from a non-aqueous environment to an aqueous environment.

In one embodiment, the composition transforms to a solid when transferred from a non-aqueous environment to an aqueous environment, optionally wherein the solid comprises a crystalline solid or an amorphous solid.

In one embodiment, the hydrophobic component comprises a hydrophobic carbohydrate.

In one embodiment, the hydrophobic carbohydrate is a carbohydrate ester.

In one embodiment, the hydrophobic component comprises a lipid.

In one embodiment, the hydrophobic component comprises a hydrophobic polymer.

In one embodiment, the hydrophobic component comprises a mixture comprising two or more of a hydrophobic carbohydrate, a lipid and a hydrophobic polymer e.g. a hydrophobic carbohydrate and a lipid; a hydrophobic carbohydrate and a hydrophobic polymer; a lipid and a hydrophobic polymer; or a hydrophobic carbohydrate, a lipid and a hydrophobic polymer.

In one embodiment, the organic solvent is selected from DMSO, benzyl alcohol, benzyl benzoate, propylene carbonate, NMP, and polyethylene glycol. In one embodiment, the organic solvent is selected from anisole, 1-propanol, 1-buthanol, ethanol, NMP or DMSO.

In one embodiment, the organic solvent is a polyhydric alcohol such as but not limited to glycerin, diglycerin, polyglycerin, propylene glycol, polypropylene glycol, ethylene glycol, diethylene glycol, triethylene glycol, polyethylene glycol, polyethylene glycol (PEG), benzyl benzoate, triglycerides, acetone, benzyl alcohol (BnOH), ethanol (EtOH), ethyl lactate, propylene carbonate (PC) and Dimethyl Sulfoxide (DMSO), 1-methyl-2-pyrrolidon (NMP), 1-butanol, 2-butanol, Tert-butylmethyl ether, Ethyl ether, Ethyl formate, Heptane, 3-Methyl,-1-butanol, Methylisobutyletone, 2-Methylisobutylketone, 2-Methyl-I-propanol, Pentane, 1-Pentanol, 1-Propanol, 2-Propanol or combinations thereof.

In one embodiment, the composition comprises a co-solvent selected from glycerol trivalerate, glycerol trihexanoate (GTH), glycerol trioctanoate (GTO), glycerol tridecanoate (GTD), ethyl octanoate, ethyl hexanoate, ethyl decanoate, Ethyl myristate, ethyl laurate, ethyl oleate, ethyl palmitate, corn oil, peanut oil, coconut oil, sesame oil, cinnamon oil, soybean oil, poppyseed oil, Lipiodol and aliphatic alkyl acyl esters and the like.

In one embodiment, the aqueous environment is within the body of a subject. In one embodiment, the aqueous environment is within a tissue of the subject, such as a muscle, cancer tissue or lymph node.

In one embodiment, the carbohydrate, lipid, polymer or mixture thereof contains the at least one primary, secondary, tertiary or quaternary amine. In one embodiment, the carbohydrate, lipid, polymer or mixture thereof does not contain a primary, secondary, tertiary or quaternary amine.

In one embodiment, the nucleic acid component comprises a free nucleic acid.

In one embodiment, wherein the nucleic acid component comprises a hydrophobic ion-pairing (HIP) complex.

In one embodiment, the nucleic acid component comprises a nanoparticle. In one embodiment, the nanoparticle is a lipid nanoparticle or a polymer nanoparticle. In one embodiment, the nanoparticle is 20-500 nm in size.

In one embodiment, the hydrophobic carbohydrate comprises a hydrophobic derivative of a disaccharide or a trisaccharide or a mixture thereof. In one embodiment, the hydrophobic carbohydrate comprises a derivative of sucrose, lactose, maltose, trehalose or raffinose. In one embodiment, the hydrophobic carbohydrate comprises a derivative of lactulose.

In one embodiment, the composition comprises at least 30% (w/w) of hydrophobic carbohydrate.

In one embodiment, the composition further comprises a co-solvent. In one embodiment, the co-solvent is a lipid, a phospholipid, a pegylated lipid, a monoglyceride, a diglyceride or a triglyceride.

In one embodiment, the composition forms a depot when in an aqueous environment.

In one embodiment, the composition forms an NCCell when in an aqueous environment.

In one embodiment, the nucleic acid component comprises a targeting ligand that targets a receptor on a cell surface.

In one embodiment, the nucleic acid component comprises an oligonucleotide and/or a polynucleotide.

In one embodiment, the nucleic acid component comprises DNA or RNA.

In one embodiment, the nucleic acid component comprises a therapeutic nucleic acid. In one embodiment, the therapeutic nucleic acid is selected from siRNA, ASO, mRNA, and DNA.

In one embodiment, the composition is formulated as an injectable.

In one embodiment, the composition is for use in transfection.

In one embodiment, the composition further comprises an imaging agent.

The invention also provides a composition of the invention for use in medicine.

The invention also provides a composition of the invention for use in therapy.

The invention also provides a composition of the invention for use as a controlled release system for a nucleic acid-based component in a subject.

The invention also provides a composition of the invention for use in treating a disease treatable by gene engineering.

The invention also provides a composition of the invention for use in treating a disease treatable by a nucleic acid-based therapy. In one embodiment, the nucleic acid-based therapy is selected from DNA, pDNA, ssDNA, dsDNA, antisense DNA, eecDNA, microDNA, spcDNA, episomal DNA, linear DNA, RNA (messenger RNA (mRNA), self-replicating mRNA, transfer RNA (tRNA), ribosomal RNA (rRNA)), small single or double stranded RNA (dsRNA, RNA interference (RNAi) including microRNA (miRNA), small interfering RNA (siRNA or ASO, with modifications including phosphorotioate (PS), PS morpholino, 2′-O-methyl, 2′-O-methoxyethyl, 2′fluoro, 5′methylcystine, G-clamp), splice switching antisense oligonucleotide (SSO), CRISPR-Cas9 sgRNAs, piwi-interacting RNA (piRNA) and/or repeat associated small interfering RNA (rasiRNA). In one embodiment, the therapeutic nucleic acid is selected from circular RNA (circRNA), long non-coding RNA (lncRNA), transfer-messenger RNA (tmRNA), ribozymes, aptamers and artificial nucleic acids.

In one embodiment, the composition is for use in treating cancer, an inflammatory disease, an immune system disorder, a genetic disease, a regenerative disorder, a non-healing tissue disorder, myelodysplastic syndrome, an autoimmune disorder, rheumatoid disease, a deficiency disease, a hereditary disease, a storage disease, a degenerative disorder, anaemia, an endocrine disorder, a hormone imbalances, hormone inactivation or a psychological disorder.

In one embodiment, the composition is administered by injection or catheterization.

The invention also provides a method of treating a subject by nucleic acid-based therapy, wherein the method comprises administering to the subject a composition of the invention. In one embodiment, the nucleic acid therapy comprises administration of DNA, pDNA, ssDNA, dsDNA, antisense DNA, eecDNA, microDNA, spcDNA, episomal DNA, linear DNA, RNA (messenger RNA (mRNA), self-replicating mRNA, transfer RNA (tRNA), ribosomal RNA (rRNA)), small single or double stranded RNA (dsRNA, RNA interference (RNAi) including microRNA (miRNA), small interfering RNA (siRNA or ASO, with modifications including phosphorotioate (PS), PS morpholino, 2′-O-methyl, 2′-O-methoxyethyl, 2′fluoro, 5′methylcystine, G-clamp), splice switching antisense oligonucleotide (SSO), CRISPR-Cas9 sgRNAs, piwi-interacting RNA (piRNA) and/or repeat associated small interfering RNA (rasiRNA). In one embodiment, the therapeutic nucleic acid is selected from circular RNA (circRNA), long non-coding RNA (lncRNA), transfer-messenger RNA (tmRNA), ribozymes, aptamers and artificial nucleic acids.

In one embodiment, the method comprises treating a disease selected from cancer, an inflammatory disease, an immune system disorder, a genetic disease, a regenerative disorder, a non-healing tissue disorder, myelodysplastic syndrome, an autoimmune disorder, rheumatoid disease, a deficiency diseases, a hereditary disease, a storage disease, a degenerative disorder, anaemia, an endocrine disorder, a hormone imbalances, hormone inactivation or a psychological disorder.

In one embodiment, the method comprises administration by injection or catheterization.

The invention also provides use of a composition of the invention in an in vitro method for transfecting one or more cells.

The invention also provides a method of producing a composition of the invention, wherein the method comprises dissolving or dispersing in an organic solvent, (a) a nucleic acid component; (b) a hydrophobic carbohydrate, a lipid, a hydrophobic polymer, or mixture thereof; and (c) a hydrophobic component, wherein the hydrophobic or amphiphilic molecule contains at least one primary, secondary, tertiary or quaternary amine.

### Definitions

“Nucleic acid component” comprises a nucleic acid sequence that is at least 8 nucleotides in length, and is typically an oligonucleotide or polynucleotide. The invention is not limited to any particular type of nucleic acid sequence, and so the nucleic acid component may comprise e.g. DNA, pDNA, RNA, mRNA, tRNA, siRNA, PS-oligos, Antisense oligonucleotides (ASO), splice switching antisense oligonucleotide (SSO), LNA, PNA and aptamers.

“Oligos” or “Oligonucleotides” refers to short a polymer consisting of a small number of nucleotides. Oligonucleotides are characterized by the sequence of nucleotide residues that make up the entire molecule. The length of the oligonucleotide is usually denoted by “-mer”. For example, an oligonucleotide of eight nucleotides (nt) is a octamer, while one of 25 nt would usually be called a “25-mer”. Oligos may comprise chemically modified nucleotides with enhanced stability.

“pDNA” or “plasmid DNA” or “plasmid” refers to a small, extrachromosomal DNA molecule within a cell that is physically separated from chromosomal DNA and can replicate independently. They are most commonly found as small circular, double-stranded DNA molecules in bacteria; however, plasmids are sometimes present in archaea and eukaryotic organisms.

The term “DNA” refers to pDNA, ssDNA, dsDNA, antisense DNA, eecDNA, microDNA, spcDNA, linear DNA, episomal DNA.

The term “RNA” further encompasses any type of single stranded (ssRNA) or double stranded RNA (dsRNA) molecule known in the art, such as viral RNA, retroviral RNA and replicon RNA, small interfering RNA (siRNA), antisense RNA (asRNA), circular RNA (circRNA), ribozymes, aptamers, riboswitches, immunostimulating/immunostimulatory RNA, transfer RNA (tRNA), ribosomal RNA (rRNA), small nuclear RNA (snRNA), small nucleolar RNA (snoRNA), microRNA (miRNA), and Piwi-interacting RNA (piRNA). The term “RNA” may also refer to long non-coding RNA (lncRNA) and transfer-messenger RNA (tmRNA).

The term “ribozyme” refers to an RNA or fragment thereof that has one or more catalytic activities similar to a protein enzyme, e.g. a ribozyme may be a catalytic RNA molecule that cleaves RNA in a sequence specific manner.

The term “riboswitch” refers to a regulatory segment of a messenger RNA molecule that binds a small molecule, resulting in a change in production of the proteins encoded by the mRNA.

“mRNA” or “messenger RNA” refers to the form of RNA in which genetic information transcribed from DNA as a sequence of bases is transferred to a ribosome.

“tRNA” or “transfer RNA” refers to a small RNA molecule that participates in protein synthesis. Each tRNA molecule has two important areas: a trinucleotide region called the anticodon and a region for attaching a specific amino acid.

“SiRNA” or “silencing RNA” or “small interfering RNA” refers to a class of double-stranded RNA at first non-coding RNA molecules, typically 20-24 (normally 21) base pairs in length, similar to microRNA, and operating within the RNA interference (RNAi) pathway. It interferes with the expression of specific genes with complementary nucleotide sequences by degrading mRNA after transcription, preventing translation.

“Antisense” refers to an oligonucleotide having a sequence that hybridizes to a target sequence in an RNA by Watson-Crick base pairing, to form a heteroduplex with the target sequence. The antisense oligonucleotide may have exact sequence complementarity to the target sequence or near complementarity. These antisense oligonucleotides may block or inhibit translation of the mRNA, and/or modify the processing of an mRNA to produce a splice variant of the mRNA. Antisense oligonucleotides are typically between about 8 to about 100 nucleotides in length, more typically, between about 8 and about 50 nucleotides in length, and even more typically between about 10 nucleotides and about 30 nucleotides in length. Chemical modifications of antisense oligonucleotides include phosphorotioate (PS), PS morpholino, 2′-O-methyl, 2′-O-methoxyethyl, 2′fluoro, 5′methylcystine, G-clamp.

“Splice switching antisense oligonucleotide (SSO)” short, synthetic, antisense, modified nucleic acids that base-pair with a pre-mRNA and disrupt the normal splicing repertoire of the transcript by blocking the RNA-RNA base-pairing or protein-RNA binding interactions that occur between components of the splicing machinery and the pre-mRNA.

“LNA” or “Locked Nucleic Acid” herein refers to a novel type of nucleic acid analog that contains a 2′-0, 4′-C methylene bridge. This bridge-locked in the 3′-endo conformation-restricts the flexibility of the ribofuranose ring and locks the structure into a rigid bicyclic formation. This confers enhanced assay performance and an increased breadth of applications.

“PNA” or “peptide nucleic acid” herein refers to synthetic peptide nucleic acid oligomers that can have higher binding strength.

“PS Oligos” or “Phosphorothioate Oligonucleotides” herein refers to oligos or nucleic acids coupled with phosphodiester and phosphorothioate internucleotide linkages.

“Transfection system” herein refers to an artificial assembly capable of introducing nucleic acids (DNA or RNA) into cells, utilizing means other than viral infection.

“Transfection particle” herein refers to a particle system comprising oligonucleotides, DNA or RNA and transfection agents that together form particles of 30-5000 nm in size. The transfection agents are often cationic and can be polymer or lipid based.

“Coacervates” herein refers to an aqueous phase rich in macromolecules such as synthetic polymers and nucleic acids. It forms through liquid-liquid phase separation (LLPS), leading to a dense phase in thermodynamic equilibrium with a dilute phase. The dispersed droplets of dense phase are also called coacervates, micro-coacervates or coacervate droplets.

“Polyplex” herein refers to a coacervate or complex of nucleic acids and cationic polymers.

“Lipoplex” herein refers to a coacervate or complex of nucleic acids and cationic lipids or lipid mixtures of cationic lipids and helper lipids. Lipoplexes and lipid nanoparticles (LNPs) are in the context of the present disclosure considered the same.

“Lipopolyplex” herein refers to a coacervate or complex of nucleic acids and mixtures of cationic lipids or lipids with cationic polymers or polymers

“Depot” or “Hydrophobic depot” herein refers to a hydrophobic biomaterial or material from which the nucleic acid component (e.g. transfection systems, transfection particles or oligos, polynucleotides and the like) can be release at a controlled rate. These may comprise hydrophobic excipients such as but not limited to organic solvents, organic co-solvents, carbohydrate esters, oils, NCCells and the like.

“Non-water soluble carbohydrates” or “hydrophobic carbohydrates” refers to carbohydrates that are insoluble in water, which is defined as carbohydrates that precipitate when the concentration exceeds 0.1 M at 25 degrees Celsius.

In the context of the present invention, a “gel” is defined as a carrier matrix composition in which the nucleic acid component is dispersed and/or dissolved within.

The term “gel” as used in the present invention includes systems such as gels or amorphous glass matrices, crystalline solids, amorphous solids, which upon injection into a human or an animal increases viscosity where the composition changes from being liquid like to gel-like in its appearance.

The term “NCCell” is used for gel compositions comprising hydrophobic carbohydrate esters that have features of a gel after administration into a human or animal body.

With the term “hydrophobicity” or “hydrophobic” we refer to the effect that a molecule is seemingly repelled from water, that is a molecule that has a logP >0.

With the term “gel-like” compound or material, as used herein, we refer to any compound comprising some of the properties of a gel i.e. a material that exhibits limited flow when in the steady-state. By weight, gels are mostly liquid, yet they behave like solids due to a three-dimensional interactions within the liquid. It is the interactions within the fluid that gives a gel its structure (hardness) and contributes to the adhesive stick. In this way gels are a dispersion of molecules of a liquid within a solid in which the solid is the continuous phase and the liquid is the discontinuous phase providing a gel-like material with a higher viscosity than for that of a liquid.

The term “hydrophobic ion pair” (HIP) refers to the process and product of forming ionic interactions between a charged hydrophilic molecule with an oppositely charged counterion. The counterion contains at least one hydrophobic domain such as an alkyl tail or aromatic ring.

The term “counterion”, “co-ion”, ‘ion pair (ing agent) (IP),’ or ‘salt former, refers to an ion with opposite charge of the hydrophilic molecule.

The term “hydrophobic counterion” is a counterion containing hydrophobic domains.

The terms “low dielectric” media refers to a media with low dielectric constant, such as organic solvents, oils and the like.

As used herein, “drug”, “therapeutic”, “active agent” or “bioactive agent” refers to any compound or mixture of compounds which produces a physiological result, e.g. a nucleic acid component comprising a therapeutic nucleic acid. The physiological result may be e.g., a beneficial or useful result, upon contact with a living organism, e.g., a mammal, such as a human. Active agents are distinguishable from other components of the delivery compositions, such as carriers, diluents, binders, colorants, etc. The active agent may be any molecule, as well as binding portion or fragment thereof, that can modulate a biological process in a living subject. In certain embodiments, the active agent may be a substance used in the diagnosis, treatment, or prevention of a disease or as a component of a medication. In some embodiments, an active agent may refer to a compound that facilitates obtaining diagnostic information about a targeted site in a body of a living organism, such as a mammal or in a human.

The term “treatment”, “treating” or “therapeutic intervention” relates to the management and care of an individual for the purpose of combating a condition such as a disease or disorder. The term is intended to include the full spectrum of treatments for a given condition from which the individual is suffering, such as administration of the therapeutically effective compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of an individual for the purpose of combating the disease, condition or disorder and includes the administration of the active compounds to prevent the onset of the symptoms or complications. The individual to be treated is an animal, preferably a mammal, in particular a human being.

“Intratumoral administration/application” refers to the direct delivery of a composition into or adjacent to a tumor or cancer and/or immediate vicinity of a tumor or cancer. In the context of the present invention the term “intratumoral administration/application” thus typically also refers to locoregional or peritumoral application/administration. Multiple injections into separate regions of the tumor or cancer are also included. Furthermore, intratumoral administration/application includes delivery of a composition into one or more metastases. The composition can be injected directly into the tumor or cancer (tissue) with great precision by imaging-guided injection, preferably using an imaging technique, such as computer tomograpy, ultrasound, gamma camera imaging, positron emission tomography, or magnetic resonance tumor imaging. Further procedures are selected from the group including, but not limited to, direct intratumoral injection by endoscopy, bronchoscopy, cystoscopy, colonoscopy, laparoscope and catheterization. In addition the composition can be injected locoregionally or peritumorally by the same methods. Tumor or cancer tissue includes metastases of the primary tumor, e.g. to lymph nodes, skin, soft tissues, bone, visceral organs or other organs of the body. Thus, in some embodiments, the composition of the invention is administered to tumor or cancer tissue e.g. metastases of the primary tumor. In some embodiments, the composition of the invention is administered to e.g. lymph nodes, skin, soft tissues, bone, visceral organs or other organs of the body.

The term “variant” of a protein or peptide encoded by the delivered therapeutic nucleic acid means that the variant has at least 50%, 70%, 75%, 80%, 85%, 90%, 95%, 98% or 99% amino acid identity over a stretch of 10, 20, 30, 50, 75 or 100 amino acids to the natural protein or peptide. Alternatively, More preferably, an encoded “variant” as used herein is at least 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to the peptide or protein.

“N/P ratio” herein refers to the nitrogen to phosphate ratio, or the ratio of positive to negative charges.

“C12-C18” or “C₁₂-C₁₈” herein refers to the length range for an acyl chain. C12-C18 refers to an acyl chain with 12 to 18 carbons. Cₓ-C_{Y} thus refers to an acyl chain with X-Y carbons in length.

“Polynucleotide” herein refers to the classes of nucleotides includes DNA, pDNA, RNA (messenger RNA (mRNA), self-replicating mRNA, splice switching antisense oligonucleotide (SSO), transfer RNA (tRNA), and ribosomal RNA (rRNA)), small single or double stranded RNA (dsRNA, RNA interference (RNAi) including microRNA (miRNA), small interfering RNA (siRNA or ASO, including chemical modifications; phosphorotioate (PS), PS morpholino, 2′-O-methyl, 2′-O-methoxyethyl, 2′fluoro, 5′methylcystine, G-clamp), CRISPR-Cas9 sgRNAs, piwi-interacting RNA (piRNA) and repeat associated small interfering RNA (rasiRNA). In addition to unmodified nucleic acids alternative synthetic nucleotides analogous include Peptide nucleic acid (PNA), Morpholino and locked nucleic acid (LNA), glycol nucleic acid (GNA), threose nucleic acid (TNA), hexitol nucleic acids (HNA), bridged nucleic acid (BNA) and 2′ fluoro-substituted, 2′-O-methyl-substituted nucleotides.

“Nucleic acid complexes” herein refers to the joint class of LNPs, lipoplexes, polyplexes and hydrophobic ion pairs of nucleic acids.

“Viscosity” herein refers to the state of being thick, sticky, and semi-fluid in consistency, due to internal friction. Viscosity is a quantity expressing the magnitude of internal friction in a fluid, as measured by the force per unit area resisting uniform flow. Viscosity can be quantified in the unit centipoise (cP) and can be measured using an EMS Viscometer (EMS-1000S). Viscosity can be quantified in the unit centipoise (cP) and can be measured using an EMS Viscometer (EMS-1000).

### BRIEF DESCRIPTION OF THE FIGURES

FIG. 1. Size and polydispersity index analyzed by DLS of mCherry pDNA formulated in different transfection systems with varying composition, NP ratios and Ignite flow conditions. Lipid-based LNPs at NP5 and L-PEI25 kDa at different NP ratios were prepared using the Ignite at a flow rate of 9 mL/min, except for the last L-PEI25 kDa sample (12 mL/min), diluted in ultrapure water to 10 μg/mL, and measured on the DLS using automatic mode.

FIG. 2. NTA data examples. NTA based size distribution plots of different mCherry mRNA lipid- and polymer-based transfection particles.

FIG. 3: Example data from Picogreen assay. Percentage of mCherry pDNA encapsulation within various lipid-based transfection particles prepared with the Ignite. The encapsulation (%) is calculated by the ratio of Picogreen signal obtained for intact particles and particles dissociated with 0.5% hydrogenated Triton 100-X.

FIG. 4. RNase protection assay data example. RNase protection assay showing the percentage of mRNA recovery after digestion with RNases. Transfection particles with Luc or mCherry mRNA at 50-100 μg/mL were mixed with 1 μL RNase cocktail, incubated for 1 h at 37° C., and analyzed on HPLC. The results are the ratio between the AUC of the mRNA peaks in the samples with/without RNases. As control, mCherry mRNA free in solution was also used to show the potency of the RNases to degrade naked mRNA when not complexed.

FIG. 5. Agarose gel data example. Agarose gel showing migration of free pDNA, free pDNA-Cy5 labelled, SLNP D-LIN-KC2: Chol: DSPC 50:40:10 pDNA complexed at NP5 and SLNP DLin-KC2: Chol: DSPC 50:40:10 pDNA-Cy5-labeled complexed at NP5

FIG. 6. PS Oligos in water and DMSO. Example of PS silencing particles and free PS oligos transferred from water into DMSO.

FIG. 7. Size of PS oligo complexes. Example of size of Cy5 labelled PS oligos complexed as silencing particles in H₂O and DMSO. Sizes were determined using NTA according to example 6.

FIG. 8. Vacuum oven transfer data. Reduction of water from LPEI25K-pDNA in water mixed with (A) DMSO, (B) PC or (C) BnOH upon vacuum oven transfer. Reduction is given in percent and presented as function of time in the vacuum oven.

FIG. 9. Transfection system size in water and upon transfer to DMSO. The particles in DMSO were resuspended in water in advance of measuring their size using NTA.

FIG. 10. NTA data of particles in water and DMSO. Size histograms of transfection particles in water and upon transfer to DMSO measured by NTA.

FIG. 11. HIP complexes. Pictures of HIP complexes with 0.4 μg/μL mCherry pDNA at NP1 or NP2 transferred from water to DMSO showing no precipitation upon transferring the HIP complexes into DMSO.

FIG. 12. Size of HIP complexes. Size of hydrophobic ion-pair complexes with mCherry mRNA in water and DMSO. Solutions of the HIPs: mRNA complexes at NP2, either in water or transferred to DMSO, were diluted in ultrapure water to a mRNA concentration of 50-100 ng/ml and the size was determined using Nanoparticle Tracking Analysis (NTA). As controls, mRNA complexed with L-PEI25k at NP25 and Free mRNA were included. * indicates no particles detected by NTA.

FIG. 13. Encapsulation of HIP complexes. Encapsulation of mCherry mRNA when complexed with different HIP in water at 0.1 μg/μL mRNA NP2. The encapsulation percentage is calculated from the accessibility of Ribogreen dye to bind mRNA molecules when the complexes are in water or digested using 0.5% TritonX.

FIG. 14. Transfection with HIP complexes. HEK cell viability and mCherry transfection after 24 h treatment with 400 ng mRNA complexed with DOTAP (NP1 and NP2) or DC-Chol (NP1) HIPs in water and DMSO.

FIG. 15. Size of siRNA HIP complexes. Size of ion pairing complexes with Luciferase siRNA in water and DMSO. Solutions of the HIP: siRNA complexes at NP2 either in water or transferred to DMSO were diluted in ultrapure water to a siRNA concentration of 50-100 ng/ml and size was measured using Nanoparticle Tracking Analysis (NTA). As controls, Free siRNA were included. * indicates no particles detected by NTA.

FIG. 16. Encapsulation of siRNA HIP complexes. Encapsulation of Luciferase siRNA when complexed with different HIP counterions in water at 0.1 μg/μL siRNA at NP2. The encapsulation percentage is calculated from the accessibility of Ribogreen dye to bind siRNA molecules when the complexes are in water or digested for 30 minutes at 60° C. with 0.5% TritonX.

FIG. 17. Uptake of PS-Oligos. Fluorescence microscopy uptake in CT26, luc cells. Cells were treated with 0.4 μg KC2: Chol: DSPC, L-PEI40k, DOTAP-HIP, or Free PS-Oligos for 5 hrs. White arrows point out particles inside the cells.

FIG. 18. mRNA/pDNA NCCell compositions. Images of transparent NCCell compositions SuBen: GTH: EtOH: DMSO-55:20:5:10 (vial 1 and 4), 55:20:5:20 (vial 2 and 5), 55:20:10:10 (vial 3 and 6) embedding transfection systems L-PEI25K-pDNA, vial 1-3 and L-PEI25K-mRNA NP25, vial 4-6. All particle concentration were 40 μg pDNA/mRNA per gram NCCell.

FIG. 19. mRNA/pDNA NCCell compositions. Images of transparent NCCell compositions SuBen: GTH: EtOH: DMSO-55:20:5:10 (vial 1 and 4), 55:20:5:20 (vial 2 and 5), 55:20:10:10 (vial 3 and 6) embedding transfection systems DC-Chol-pDNA NP5, vial 1-3 and DC-Chol-mRNA NP5, vial 4-6. Transfection complexes were dissolved at 40 μg pDNA/mRNA per gram NCCell.

FIG. 20. mCherry pDNA NCCell compositions. Picture of homogeneous solutions of NCCells with composition SuBen: GTH: EtOH: DMSO (55:20:5:20) embedded with different transfection systems carrying mCherry pDNA. Transfection particles were dissolved at 20 μg pDNA per gram NCCell.

FIG. 21. NCCell depots. Picture of 100 μL NCCells with composition SuBen: GTH: EtOH: DMSO (55:20:5:20) containing different transfection systems and injected into 2 mL PBS.

FIG. 22. HIP: mRNA NCCell compositions. Picture of homogeneous solutions of NCCells with composition SuBen: GTH: EtOH: DMSO ((55:20:5:20)+0.25% cholesterol) embedded with HIP: mRNA transfection complexes prepared with different cationic counterions. Final concentration was 10 μg mCherry mRNA per gram of NCCell.

FIG. 23. Size of particles released from NCCell compositions. Particle size of lipoplexes and polyplexes of different compositions determined using NTA (KC2: Chol: DSPC (50:40:10) with/without 1.5% DMG-PEG2k at NP5; LPEI25k at NP25. Sizes are given upon in vitro release from NCCells after 24 h and 1 week.

FIG. 24. Particle concentration released from NCCell compositions. Concentration of transfection lipoplexes and polyplexes of different compositions KC2: Chol: DSPC (50:40:10) with/without 1.5% DMG-PEG2k, or LPEI25k with mCherry mRNA or pDNA released from NCCell 1 (SuBen: GTH: EtOH: DMSO 55:20:5:20) or NCCell 2 (SuBen: GTH: EtOH: DMSO 55:20:5:20+0.25% Cholesterol) after 24 h or 1 week determined using NTA.

FIG. 25. Cryo-TEM of polyplexes. Cryo-TEM images of L-PEI25K: L-PEI25K-PEG550 (50:50) pDNA NP25 particles in water (a) or DMSO (b).

FIG. 26. Cryo-TEM of DOTAP based lipoplexes. Cryo-TEM images of DOTAP: Cholesterol (50:50) pDNA NP5 particles in water (a) or DMSO (b).

FIG. 27. Cryo-TEM of DLin-KC2 lipoplexes. Cryo-TEM images of DLin-KC2: Cholesterol: DSPC (50:40:10) pDNA NP5 particles in water (a) or DMSO (b).

FIG. 28. Cryo-TEM of transfection particles released from NCCell compositions. Cryo-TEM images of L-PEI25K pDNA NP25 particles in water (a) or in release medium (b).

FIG. 29. Cryo-SEM of transfection particles in NCCell compositions. Cryo-SEM images of NCCells with embedded L-PEI25K: pDNA NP25 particles. Scale bar 20 μm (a), 10 μm (b) and 5 μm (c).

FIG. 30. Cumulative release of PEI-pDNA transfection particles from NCCells. (A) Cumulative release of L-PEI25k-pDNA NP25 particles from LOIB and SuBen based NCCells. (B) Cumulative release of L-PEI25k-pDNA NP25 particles from SuBen based NCCells with lipid additives. (C) Cumulative release of PEGylated L-PEI25k-pDNA NP25 particles from LOIB based NCCells. Release was quantified using fluorescence following Cy5 labelling of pDNA.

FIG. 31. Cumulative release of lipid based pDNA transfection particles from NCCells. (A) Cumulative release of DC-chol: Chol: DOPE and KC2: Chol: DSPC based lipid transfection systems at NP5 from NCCell. (B) Cumulative release of DC-Chol: Chol: DOPE particles at NP1 and NP5. (C) Cumulative release of DC-Chol: Chol particles at NP1 and NP5. Release was quantified using fluorescence following Cy5 labelling of pDNA.

FIG. 32. Cumulative release of PS transfection particles from NCCells. Cumulative release of PS transfection particles from NCCells with the composition SuBen: GTH: EtOH: DMSO (55:20:5:20:0.25% Cholesterol). Release was quantified using fluorescence following Cy5 labelling of PS.

FIG. 33. Cumulative release of mRNA transfection HIP complexes from NCCells. Cumulative release of mRNA from NCCell with composition SuBen: GTH: EtOH: DMSO ((55:20:5:20)+0.25% Chol) containing HIP: mRNA complexes using large molecule HIPs including DC-Chol, DOTAP, DSTAP and DDAB. The mRNA released at each timepoint was quantified using Ribogreen quantification kit.

FIG. 34. Transfection efficiency of polymer particles with 0.4 μg mCherry pDNA in water and DMSO. Transfection was obtained in HEK cells at 24 hrs post treatment.

FIG. 35. Transfection efficiency of liposomes with mCherry 0.4 μg pDNA and mRNA in water and DMSO. Transfection was obtained in HEK cells at 24 hrs post treatment. A: Percentage mCherry of cells transfected with liposomes complexed mCherry pDNA. B: Percentage mCherry of cells transfected liposomes complexed mCherry mRNA.

FIG. 36. Transfection efficiency of Solid Lipid Nano Particles (SLNPs) with 0.4 μg mCherry pDNA and mRNA in water and DMSO. Transfection was obtained in HEK cells at 24 hrs post treatment. A: Percentage mCherry of cells transfected with SLNPs complexed mCherry pDNA. B: Percentage mCherry of cells transfected with SLNPS complexed mCherry mRNA.

FIG. 37. Silencing of luc cells. Silencing of MDA-MB-231-luc cells with 0.2 μg luc siRNA or scramble siRNA (Ctrl) in transfection particles in water and DMSO. Silencing is measured 48 hrs post treatment.

FIG. 38. Flow cytometric scatterplots of NCCell mCherry transfection. Flow cytometric scatterplots of NCCell mCherry transfection (particles DC-Chol: Chol: DOPE, mRNA, NP5) from three different NCCell formulations with or without addition of 0.25% Cholesterol for the first 48 hours after injection into a 24-well transwell system (A) and again at 120 h after transfer of transwell insert to a newly seeded 24-well cell culture plate (B).

FIG. 39. Sustained transfection of HEK cells. Cumulative IL-12 production from HEK cells in transwell sustained NCCell transfection system. Plots represents NCCell with or without addition of POPC formulated with L-PEI/pDNA (IL-12) NP 25 transfection particles.

FIG. 40. Sustained activation of T-cells. NCCell sustained release IL-12 pDNA induce IL-12 protein production from HEK cell capable of activating murine T cells in vitro. The same NCCells formulation in a transwell insert was repeatedly transferred to new cell culture wells with HEK cells and supernatant collected after removal at indicated time points (A). Activation of T cells by IL-12 containing supernatant (B and C).

FIG. 41. Induction of effector T cell infiltration in tumors by NCCell. Tumor concentration of cytotoxic (CD8+) T cells after injection of free transfection particles, NCCell sustained transfection release system or empty NCCell formulation. A: CD8+ T cell concentration day 2, B: CD8+ T cell concentration day 6 and C: CD8+ T cell to regulatory T cell (CD4+CD25+Foxp3+) ratios at day 2 and day 6 analyses.

FIG. 42. Increased tumor T cell infiltration and activation by NCCell. Flow cytometric analyses of tumor T cell infiltration and activation in MC38 tumors injected with free IL-12 pDNA transfection particles, NCCell IL-12 pDNA transfection technology (SuBen: GTH: EtOH: DMSO (55:20:5:20)), and untreated controls. A: CD8+ T cells, B: IFN-γ+CD8+ T cells, and C: CD4+ T cells concentration at day six after intratumoral injection of respective transfection particles or untreated controls.

FIG. 43. Intratumoral NCCell reduce tumor growth rate. Tumor growth curves of MC38 tumors injected twice a week with 50 μl NCCell. Formulations: L-PEI25K NP25/IL-12 pDNA 80 μg/ml in NCCell composition (SuBen: GTH: EtOH: DMSO 55:20:5:20+0.25% Chol) (4 μg pDNA/50 μl NCCell) or DC-Chol: Chol: DOPE (30:65:5) NP5/IL-12 pDNA 160 μg/ml in NCCell composition (SuBen: GTH: EtOH: DMSO 55:20:5:20+0.25% Chol) (8 μg pDNA/50 μl NCCell) and untreated controls.

FIG. 44. NCCell reduce tumor growth rate and increase median survival time. A: Tumor growth curves of CT26 tumors injected twice with 50 μl NCCell formulation. B: Kaplan-Meier estimates of overall survival. Formulation: DC-Chol: Chol: DOPE (30:65:5) NP5/IL-12 mRNA 80 μg/g in NCCell composition (SuBen: GTH: EtOH: DMSO 55:20:5:20+0.25% Chol).

FIG. 45. Stability of transfection particles in DMSO. Transfection capacity of SLNPs upon 1 week storage in water at 4° C. or DMSO at room temperature compared to freshly made transfection particles. The results are quantified as % mCherry positive HEK cells of live cells, and the result is reported as the ratio of % mCherry positive HEK for old and fresh particles.

FIG. 46. Relative Luc bioluminescence signal reduction by a Luc silencing NCCell composition. Figure illustrates relative difference between transwell Luc signal between cells exposed to Luc or Scrambled siRNA formulated in KC2: Chol: DSPC LNPs formulated in a NCCell composition (SuBen: GTH: EtOH: DMSO (55:20:5:20)).

FIG. 47. NCCell compositions can be prepared using various class 3 solvents. NCCell compositions were prepared with (A) DMSO, (B) 1-propanol, (C) 1-butanol, (D) anisole, (E) propylene carbonate as solvent.

FIG. 48. PEI-Mannose transfection particles carrying mCherry mRNA successfully transfect DC2.4 cells at 0.2 μg and 0.4 μg mRNA doses. DC2.4 cells were incubated with mCherry mRNA polyplexes formed from JetPEI-Mannose, 1 mol % PEI-Mannose, 5 mol % PEI-Mannose and 10 mol % PEI-Mannose for 24 hours and mCherry transcription was evaluated by flow cytometry. All polyplexes successfully transfect DC2.4 cells with 1 mol % PEI-Mannose particles giving the highest percentage of mCherry positive cells.

FIG. 49. NCCell with 1 mol % PEI-Mannose particles of mCherry mRNA successfully transfects DC2.4 cells at a dose of 4 μg mRNA/well. DC2.4 cells were incubated in a transwell setup with 100 μL of NCCell loaded with mCherry mRNA carrying 1 mol % PEI-Mannose particles at N/P ratio 20 and extra embedded PEI25K at N/P ratio 20 for 48 hours, and mCherry transcription was evaluated by flow cytometry.

FIG. 50. Stability of mCherry mRNA encoding PEI polyplexes investigated by flow cytometry. Transfection of polyplexes stored for 35 days at 4° C. were compared to freshly made particles by flow cytometry. (A) percentage of mCherry positive HEK cells, and (B) mCherry MFI after treatment with freshly prepared or stored mRNA L-PEI 40 kDa transfection particles.

FIG. 51. Stability of mCherry pDNA encoding PEI polyplexes investigated by flow cytometry. Transfection of polyplexes stored for 35 days at rt, or at 4° C., were compared to freshly made particles by flow cytometry. (A) percentage of mCherry positive HEK cells, and (B) mCherry MFI after treatment with freshly prepared or stored pDNA L-PEI 40 kDa transfection particles.

FIG. 52. Images of pDNA transfection particles displaying high solubility in DMSO and NCCell. (A) L-PEI 40 kDa polyplex of pDNA dissolved in DMSO at a concentration of 5 mg/mL, (B) DOTAP HIP of pDNA dissolved in DMSO at a concentration of 5 mg/mL, (C) L-PEI 40 kDa polyplex of pDNA dissolved in NCCell at a concentration of 0.5 mg/mL, and (D) DOTAP HIP of pDNA dissolved in NCCell at a concentration of 0.5 mg/mL. All particles demonstrate good solubility in both DMSO and NCCell after concentrating the particles by freeze-drying.

FIG. 53. Transfection efficiency of NCCell delivering dual plasmid pDNA. Transfection particles (L-PEI40K), carrying a dual plasmid pDNA encoding for IL-12 and OX40L, formulated in NCCell (Suben: GTH: EtOH: DMSO 55:20:2:15 (+0.5% POPC) provided sustained transcription of IL-12 for up to 30 days at a dose of 100 μg/mL and 200 μg/mL pDNA. HEK293 cells were seeded in a 24 transwell plate and gels continuously moved to new cells every 2-3 days to assess sustained transfection efficiency as indicated on the X-axis. IL-12 transcription at each timepoint was assessed by ELISA.

FIG. 54. Transfection efficiency of NCCell delivering dual plasmid pDNA. Transfection particles (L-PEI40K), carrying a dual plasmid pDNA encoding for IL-12 and OX40L, formulated in NCCell (Suben: GTH: EtOH: DMSO 55:20:2:15 (+0.5% POPC) provide sustained transcription of OX-40L for up to 23 days at a dose of 100 μg/mL and 200 μg/mL pDNA. HEK293 cells were seeded in a 24 transwell plate and gels continuously moved to new cells every 2-3 days to assess sustained transfection efficiency. OX-40L expression at each timepoint was assessed by flow cytometry and shown as scatter plots.

FIG. 55. Sustained in vitro release of siRNA lipoplexes and polyplexes from NCCell. A) In vitro sustained release of siRNA lipoplexes (DOTAP: DC-Chol: Chol: DOPE 25:25:25:25) for 21 days demonstrating that the release rate can be controlled by GTH content of the NCCell. B) Sustained in vitro release of siRNA polyplexes (L-PEI 40 kDa) for 9 days, demonstrating that the release rate can be controlled by addition of lipid to the NCCell.

FIG. 56. GFP silencing in HEK-GFP cells treated with siRNA polyplexes and lipoplexes in water and DMSO for 48 hours. GFP silencing of siRNA lipoplexes (DOTAP: DC-Chol: Chol: DOPE (25:25:25:25) NP5), and polyplexes (L-PEI40k NP25) in water and DMSO was assessed by flow cytometry. Both formulations, in water and DMSO, silence GFP expression, compared to untreated HEK-GFP cells, 48 hours after treatment.

FIG. 57. GFP silencing in HEK-GFP cells treated with siRNA polyplexes in water and DMSO for 48 hours. GFP silencing of siRNA polyplexes (L-PEI40k NP25) in water and DMSO was assessed by flow cytometry. Compared to a scrambled control, L-PEI40K polyplexes in water and DMSO silence GFP expression, 48 hours after treatment.

FIG. 58. GFP silencing in HEK-GFP cells treated with siRNA polyplexes and lipoplexes in water and DMSO for 48 hours. GFP silencing of siRNA lipoplexes (DOTAP: DC-Chol: Chol: DOPE (25:25:25:25) NP4), and polyplexes (Mannose-JetPEI at NP8, Mannose-JetPEI at NP8 with extra carrier PEI at NP25, and L-PEI40k NP25) in water and DMSO was assessed by flow cytometry. All formulations, in water and DMSO, silence GFP expression, compared to untreated HEK-GFP cells, 48 hours after treatment.

FIG. 59. KRAS silencing in MIA PaCa-2 cells treated with siRNA polyplexes and lipoplexes in water for 48 hours. KRAS silencing with siRNA-cholesterol polyplexes (PAMAM), siRNA-cholesterol lipoplexes (DOTAP: DC-Chol: Chol: DOPE), and siRNA lipoplexes (DOTAP: DC-Chol: Chol: DOPE) was assessed by qPCR. All formulations strongly silence KRAS expression in MIA PaCa-2 cells 48 hours after treatment, most notable when silencing with siRNA-cholesterol, and siRNA lipoplexes.

FIG. 60: Sustained silencing of HEK293-GFP cells by siRNA polyplexes and lipoplexes loaded in NCCell: Sustained silencing was evaluated using HEK293-GFP cells in transwells treated with NC-Cell transfection system formulations comprising GFP siRNA lipoplexes (DOTAP: DC-Chol: Chol: DOPE (25:25:25:25) N/P ratio 5) and GFP siRNA polyplexes (L-PEI40k N/P ratio 25). GFP expression of treated HEK293-GFP cells normalized to untreated samples was evaluated by GFP MFI quantification on flow cytometry and displayed over time.

FIG. 61. Stability of L-PEI 40 kDa polyplexes of siRNA evaluated by flow cytometry. L-PEI 40 kDa polyplexes of siRNA were stored in DMSO for 35 days either at −20° C. or at 4° C. Afterwards, the silencing capacity of the particles was evaluated by flow cytometry and compared to silencing capacity of fresh particles. The GFP MFI was normalized to untreated cells.

FIG. 62. Particle size of various polyplexes formed with ASOs measured by NTA. Polyplexes were prepared by mixing ASOs with various polymers (L-PEI 40 kDa, PAMAM Gen 0.0, and PAMAM Gen 1.0) and the size measurements were performed by NTA.

FIG. 63. NTA size measurements of HIPs prepared with various cationic counterions. The HIPs of ASOs were prepared by mixing with the four different counterions (DOTAP, Spermine-chol, BTMAC, and TEAB). The results demonstrate that only large and hydrophobic cationic counterions form particles, while small counterions do not form particles (marked with X).

FIG. 64: Formation of ASO HIPs using the Bligh-Dyer phase-separation method. (A) Formation of the Bligh-Dyer monophase at volume ratios of 1:2:1 (water: methanol: chloroform). (B) Formation of the biphasic system with the HIP-ASO complexes in the lower chloroform phase. (C) HIPs prepared with the counterions CTAB and Spermine-cholesterol form particles, which was measured by DLS size measurements.

FIG. 65. Freeze-drying or Bligh-Dyer method can be used to obtain high concentrations of ASOs in NCCell. A) BTMAC-ASO HIP up-concentrated by freeze-drying to 10 mg/mL in DMSO. B) Spermine-cholesterol-ASO HIP up-concentrated by freeze-drying to 5 mg/mL in DMSO. C) BTMAC-ASO HIP in NCCell at 1 mg/mL up-concentrated by freeze-drying. D) Spermine-cholesterol-ASO HIP in NCCell at 0.5 mg/mL up-concentrated by freeze-drying. E) PAMAM-ASO polyplex at 5 mg/mL in NCCell up-concentrated by freeze-drying. F) PAMAM-ASO polyplex at 5 mg/ml in NCCell up-concentrated by Bligh-Dyer method.

FIG. 66. In vitro release of Cy-ASOs complexed to PAMAM (A) and Spermine-Cholesterol (B) from NCCells with varying amounts of solvents. The Cy5-ASO signal present in release media was measured as a function of time and are shown as % of cumulative release to input amount of Cy5-ASO.

FIG. 67: NCCell provided sustained in vivo release of ASOs, either prepared as HIP or polyplexes, and the release kinetics was controlled by the composition of the NCCell. A) The percent release of Spermine-Chol HIP of ASO-Cy5 from NCCell with composition SuBen: GTH: EtOH: DMSO 55:20:2:15. B) Day 3 release of PAMAM-ASO polyplex of ASO-Cy5 from NCCell compositions with different levels of GTH (SuBen: GTH: EtOH: DMSO 55:20:2:15 and 55:15:2:10).

FIG. 68. Intratumoral injection of spermine-cholesterol complexed ASOs in NCCell eliminated the systemic spillover observed after intratumoral injection of free ASOs. The concentration of Gd-ASO in liver (A), spleen (B), kidney (C) and lung (D) at 24 and 120 hours after intratumoral injections of Gd-ASO complexed to spermine-cholesterol released from NCCell and free Gd-ASO (determined from ICP-MS of tissues ex vivo). The concentration of Gd-ASO was measured in blood 10 minutes after intratumoral injection (E). After 24 and 120 hours the intratumorally injected NCCell was recovered and the remaining Gd-ASO was measured, and the concentration was compared to the input NCCell Gd-ASO concentration (F).

FIG. 69. ASO-complexes accumulate in immune and cancer cells after intratumoral injection of NCCell. The distribution of Cy5-labeled ASOs across different cancer and immune cell populations in the tumor was analysed by flow cytometry on day 1 and day 3 after treatment. A) Percent of the cell populations that are positive for Cy5-ASO signal. B) The main fluorescent intensity (MFI) of Cy5 in macrophages and neutrophils.

FIG. 70. Silencing of MALAT1 RNA expression in 4T1 cells by PAMAM/ASO polyplexes and Spermine-cholesterol/ASO HIP complexes. Figure illustrates expression levels (RT-qPCR) relative to free ASO and untreated controls 48 hours after treatment.

FIG. 71. Silencing of KRAS RNA expression in MIA PaCa-2 cells by PAMAM/ASO polyplexes and Spermine-cholesterol/ASO HIP complexes. Figure illustrates expression (RT-qPCR) levels relative to free ASO and untreated controls, 48 hours after treatment with HIP/ASO complexes at different concentrations normalized to untreated samples in MIA PaCa-2 cells.

FIG. 72. MALAT1 silencing capabilities are maintained for HIP complexes stored as freeze dried (FD) or suspended in DMSO for 14 days, 6 weeks and 104 days. Figure illustrates MALAT1 expression normalized to untreated samples in 4T1 cells treated with indicated HIP complexes that were, before evaluating silencing capability, stored for 14 days (A), 6 weeks (B), and 104 days (C), as freeze dried (FD) or dissolved in DMSO at either 4° C. or 40° C.

FIG. 73. Sustained silencing of MALAT1 expression in 4T1 cells using NCCell with PAMAM polyplexes, releasing MALAT1 ASOs. Sustained silencing was evaluated using a transwell setup where transwells containing NCCell were transferred to fresh cells every 2-3 cells, at the timepoints indicated on the X-axis. Free MALAT1 ASO (25 nM) was added to fresh cells at identical time points as a control. NCCell with polyplexes carrying MALAT1 ASO successfully silence MALAT1 expression for up to 26 days, to a similar extent as unformulated MALAT1 ASO.

FIG. 74. Tumor growth rate and mass is reduced by NCCell hKRAS ASO. Treatment of MiaPaCa xenograft with NCCell releasing hKRAS ASO reduces tumor growth (FDJ-2A) and significantly (unpaired T-test) reduces tumor mass at the end of the study (day 65 analysis), tumor mass was obtained by weighing dissected tumors without inclusion of remaining NCCell material (B). Timepoints for NCCell treatments are illustrated by T #1-5 and arrows (A). NCCell: PAMAM/hKRAS ASO polyplex formulated in NCCell (SuBen: GTH: EtOH: DMSO 55:20:2:15)

FIG. 75. Tumor cell proliferation is reduced at day 1 after final treatment with NCCell releasing KRAS ASO in the MiaPaca2 pancreatic cancer model. Proliferation was addressed by flow cytometry after staining for the nuclear protein Ki67. Significance was determined using unpaired T-test.

### DETAILED DESCRIPTION OF THE INVENTION

Compositions of the invention achieve sustained release of nucleic acid component (e.g. DNA, pDNA, RNA, mRNA, tRNA, siRNA, SP-oligos, LNA, PNA or aptamers) from the hydrophobic component, wherefrom the nucleic acid component may be released in the form of free nucleic acid species or as transfection systems or transfection particles or combinations hereof. Compositions of the invention surprisingly allow for effective incorporation of highly water soluble, hydrophilic and anionically charged nucleic acids in organic media, oils, carbohydrate ester gels and or NCCells, which may be optimised by effectively screening the charges on the phosphate backbone of the nucleic acids.

In some embodiments, the nucleic acids are adapted to organic solvents, oils, depots (e.g. NCCells) and the like by formation of polyplexes or lipoplexes, or by hydrophobic ion pairing or combinations hereof.

The hydrophobic component comprises a hydrophobic or amphiphilic molecule that contains at least one primary, secondary, tertiary or quaternary amine.

In one embodiment, the primary, secondary, tertiary or quaternary amine is an ionizable lipid. As used herein, the term “ionizable lipid” has its ordinary meaning in the art and may refer to a lipid comprising one or more charged moieties. In some embodiments, an ionizable lipid may be positively charged or negatively charged. An ionizable lipid may be positively charged, in which case it can be referred to as “cationic lipid”. In certain embodiments, an ionizable lipid molecule may comprise an amine group, and can be referred to as an ionizable amino lipid. As used herein, a “charged moiety” is a chemical moiety that carries a formal electronic charge, e.g., monovalent (+1, or −1), divalent (+2, or −2), trivalent (+3, or −3), etc. The charged moiety may be anionic (i.e., negatively charged) or cationic (i.e., positively charged). Examples of positively-charged moieties include amine groups (e.g., primary, secondary, and/or tertiary amines), ammonium groups, pyridinium group, guanidine groups, and imidizolium groups. In some embodiments, the charged moieties comprise amine groups. Examples of negatively-charged groups or precursors thereof, include carboxylate groups, sulfonate groups, sulfate groups, phosphonate groups, phosphate groups, hydroxyl groups, and the like. The charge of the charged moiety may vary, in some cases, with the environmental conditions, for example, changes in pH may alter the charge of the moiety, and/or cause the moiety to become charged or uncharged. In general, the charge density of the molecule may be selected as desired.

In one embodiment, the primary, secondary, tertiary or quaternary amine is a cationic lipid containing an amine group. In one embodiment, the cationic lipid containing an amine group is selected from DOBAQ, DC-Chol, DOTAP, DODMA, C12-200, Dlin-KC2-DMA, Dlin-KC3-DMA, 1,2-dioleoyl-3-dimethylammonium-propane (DODAP), N1-[2-((1S)-1-[(3-aminopropyl)amino]-4-[di(3-amino-propyl)amino]butylcarboxamido)ethyl]-3,4-di [oleyloxy]-benzamide (MVL5), N4-Cholesteryl-Spermine, and 1,2-dioleoyl-sn-glycero-3-phospho (ethyl) ethanolamine.

In one embodiment, the primary, secondary, tertiary or quaternary amine is an amine polymer. In one embodiment, the amine polymer is selected from polyethyleneimine (PEI), polylysine (PLL), chitosans, poly(2-ethyl-2-oxazoline) (ULTROXA), diethylaminoethyl-dextran (DEAE-dextran), dendritic polyamidoamine (PAMAM), poly-beta-amino-esters (PBAE), and PDMAEMA [poly(N,N-dimethylaminoethyl methacrylate].

In one embodiment, the primary, secondary, tertiary or quaternary amine is a HIP. In one embodiment, the HIP is selected from benethamine (N-benzyl-2-phenylethanamine), dodecylamine (laurylamine), hexadecyl trimethylammonium (cetrimonium) bromide (CTAB), maprotiline, Na-Deoxycholyl-L-lysyl-methylester, N,N′-Dibenzyl ethylenediamine (benzathine), N, N-Dimethyl dodecylamine (DDA), N, N-Dimethyl hexylamine, N,N-Dimethyl octadecylamine (dimethyl stearamine) and N4-Cholesteryl-Spermine. In one embodiment, the hydrophobic or amphiphilic molecule that contains at least one primary, secondary, tertiary or quaternary amine has the ability to complex nucleic acid components into nanoparticles that can be dispersed in compositions or depots (e.g. NCCells) of the invention.

In one embodiment, the hydrophobic or amphiphilic molecule that contains at least one primary, secondary, tertiary or quaternary amine allows for control of the release rate of the nucleic acid components from a depot (e.g. NCCells) of the invention.

In one embodiment, the at least one primary, secondary, tertiary or quaternary amine has the ability to complex nucleic acid components into nanoparticles that can be released from a depot (e.g. NCCells) of the invention.

The invention allows for controlled release of nucleic acids as free species or formulated as transfection particles or transfection systems. Once injected into tissue or aqueous media, the organic media, oils, carbohydrate ester gels, or NCCells, further comprising nucleic acid component, may provide sustained release of nucleic acid component (e.g. particles) for hours, weeks or months depending on the tailored release rate. Following, the nucleic acid component (e.g. particles) may be taken up by cells locally of distribute systemically depending on particle design. Organic media, oils, carbohydrate ester gels or NCCells containing hydrophobic ion pairs of nucleic acids may likewise provide sustained release of nucleic acid HIP complexes for hours, weeks or months depending on the tailored release rate.

In one embodiment, at least 30% of the nucleic acid component is released from the depot within 1 hour to 60 days following transfer of the composition of the invention to an aqueous environment, such as within 4 hours, such as within 24 hours, such as within 4 days, such as within 7 days, such as within 14 days, such as within 21 days, such as within 30 days, such as within 60 days.

In one embodiment, at least 40% of the nucleic acid component is released from the depot within 1 hour to 60 days following transfer of the composition of the invention to an aqueous environment, such as within 4 hours, such as within 24 hours, such as within 4 days, such as within 7 days, such as within 14 days, such as within 21 days, such as within 30 days, such as within 60 days.

In one embodiment, at least 50% of the nucleic acid component is released from the depot within 1 hour to 60 days following transfer of the composition of the invention to an aqueous environment, such as within 4 hours, such as within 24 hours, such as within 4 days, such as within 7 days, such as within 14 days, such as within 21 days, such as within 30 days, such as within 60 days.

In one embodiment, at least 60% of the nucleic acid component is released from the depot within 1 hour to 60 days following transfer of the composition of the invention to an aqueous environment, such as within 4 hours, such as within 24 hours, such as within 4 days, such as within 7 days, such as within 14 days, such as within 21 days, such as within 30 days, such as within 60 days.

In one embodiment, at least 70% of the nucleic acid component is released from the depot within 1 hour to 60 days following transfer of the composition of the invention to an aqueous environment, such as within 4 hours, such as within 24 hours, such as within 4 days, such as within 7 days, such as within 14 days, such as within 21 days, such as within 30 days, such as within 60 days.

In one embodiment, at least 80% of the nucleic acid component is released from the depot within 1 hour to 60 days following transfer of the composition of the invention to an aqueous environment, such as within 4 hours, such as within 24 hours, such as within 4 days, such as within 7 days, such as within 14 days, such as within 21 days, such as within 30 days, such as within 60 days.

In one embodiment, at least 90% of the nucleic acid component is released from the depot within 1 hour to 60 days following transfer of the composition of the invention to an aqueous environment, such as within 4 hours, such as within 24 hours, such as within 4 days, such as within 7 days, such as within 14 days, such as within 21 days, such as within 30 days, such as within 60 days.

Following release of a nucleic acid ion pair, it may dissociate and exert its function locally or distribute systemically, or the co-ions may be fully or partially associated with the nucleic acid and aid its transfection capabilities locally or systemically.

As noted above, the nucleic acid component may comprise e.g. DNA, pDNA, RNA, mRNA, self-replicating mRNA, SSO, tRNA, rRNA, ssRNA, dsRNA, RNAi, miRNA, siRNA, ASO, CRISPR-Cas9 sgRNAs, piRNA, rasiRNA, PNA, Morpholino and locked nucleic acid (LNA), GNA, TNA, HNA, BNA, 2′ fluoro-substituted RNAs, 2′-O-methyl-substituted RNA.

Polyplexes: The nucleic acid component may comprise polyplexes of charged nucleic acids for solubilization in hydrophobic solvents, oils or depots (e.g. NCCells). Polyplexes are polymer-based systems containing condensed and/or complexed coacervates of nucleic acids through electrostatic interactions between cationic groups of the polymer and the negatively charged nucleic acids. The polyplex structure protects nucleic acids from enzymatic degradation and enable cargo release to tumor sites. Polymer classes used for polyplex formation include, but are not limited to polyethyleneimine (PEI), polylysine (PLL), polyarginine (PAA), Chitosans, Poly(2-ethyl-2-oxazoline) (ULTROXA), Diethylaminoethyl-dextran (DEAE-dextran), dendritic polyamidoamine (PAMAM), Poly-beta-amino-esters (PBAE), and PDMAEMA [poly(N,N-dimethylaminoethyl methacrylate]. These polymers are utilized as linear, branched, star-shaped and the like forms with varying length, charge density and molecular weight. Co-polymers of cationic and other functional polymers such as but not limited to polyethylene glycol (PEG), polylactic acid (PLA), poly(lactic-co-glycolic acid) (PLGA) and Poly(N-isopropylacrylamide) (PNIPAM) are also used for formation of transfection systems. The latter polymers are also used in mixtures with cationic polymers. Functionalization of the transfection polymers with acyl-chains such as C4-C18, or lipidation with phospholipids or sterols can also be used for modulating the hydrophobicity of the transfection particles. The ratio of polymer to nucleic acid is given by the N/P ratio and spans ranges from 1 to 200. The transfection particles can equally be comprised of a core of cationic polymers complexed with nucleic acids at a desired N/P ratio, and coated with lipids, such as but not limited to, phospholipids, cholesterol, DSPC, DSPE, DSPE-PEG2000, DPPC, DPPE, DPPE-PEG2000, DMPC, DMPE, DMPE-PEG2000.

Polyplexes may be formed as described in example 2 and 4, by mixing of polymers and nucleic acids in water to achieve a desired effective charge of both polymer and nucleic acid before strong charge-charge interactions can be established between the two. Here pH and salt concentration (ionic strength) are important factors, as pH impacts the protonation stage of the cationic polymer, and salt may screen the interactions between the two oppositely charged polymers. The formed polyplexes have typical sizes in the 25-1000 nm range and zeta-potential in the range-10 mV-+50 mV. The highly charged backbone of the nucleic acid has thus screened by opposing charges of the polymers and may be transferred to non-aqueous media. Polyplexes may be functionalized with polymers such as, but not limited to, PEG for enhanced stability and reduced cytotoxicity. Using cationic polymers grafted with e.g. PEG polymers may change the interaction with the nucleic acid and change the size distribution. PEGylation further screens surface charges on the polyplexes and reduces the interaction with serum proteins, which overall impacts the biodistribution of the PEGylated polyplexes but also changes the uptake into different cell types.

Polyplexes can furthermore be coated with coated with lipids, such as but not limited to, phospholipids, cholesterol, DSPC, DSPE, DSPE-PEG2000, DPPC, DPPE, DPPE-PEG2000, DMPC, DMPE, DMPE-PEG2000.

Polyplexes are generally formed in water, buffers, or mixtures with organic solvents. The cationic polymer such as PEI may be dissolved in water or organic solvents such as EtOH, DMSO and the like or mixtures thereof to a concentration of 0.5-5 mg/ml. Following the pH may lowered to pH 2-3 using and acid such as HCl, to protonate and charge the polymer. When the polymer is completely dissolved, the pH may be reset to pH 7-8 using a base such as NaOH. Water, organic solvents or mixtures thereof may be added to adjust the concentration to 1 mg/ml. The dissolved polymer may be sterile filtered and aliquoted into Eppendorf tubes and stored at −20° C. and are ready to use. Polymers can be dissolved to higher concentrations such as 2 and 5 mg/ml using the same protocol. Nucleic acids may be solubilized in ultrapure water at concentration of 0.1-10 μg/μL. Following, the solutions of polymer and nucleic acids are mixed at NP ratios ranging from 1-200 by simple mixing such as by using pipettes and stirring or controlled microfluidic mixing. In this process, complexes of anionic nucleic acid and the cationic polymer are spontaneously formed by a self-organising process driven by electrostatic interactions. Exchange of water, buffer or solvents may following be achieved by tangential flow filtration, spin filtration, dialysis and the like. The formed polyplexes may following be modified by postinsertion of lipids that can partition into the polyplexes or addition of polymers that may associate with the polyplexes through charge-charge interactions. Addition of polymers or lipids to the preformed polyplexes offers methods for attaching targeting ligands to the polyplexes. Greater detail of polyplex preparations and calculation of NP ratios are given in example 2 and 4.

Lipoplexes and lipid nanoparticles (LNPs): The nucleic acid component may comprise lipoplexes of charged nucleic acids for solubilization in hydrophobic solvents, oils, or depots (e.g. NCCells). Lipoplexes are lipid-based systems containing condensed and/or complexed coacervates of nucleic acids through electrostatic interactions between cationic groups of lipids and the negatively charged nucleic acids. The lipid composition of lipoplexes comprises either fixed or ionizable cationic lipids, neutral helper lipids and pegylated lipids. The lipids have difference intrinsic curvature with varying degree of unsaturation in their acyl tail region. The cationic lipids are typically highly unsaturated but may also be saturated and carry a permanent or ionizable cationic charge for countering the negative charge on the nucleic acid. Cationic lipids used include, but are not limited to DOBAQ, DOTAP, DC-Chol, DODMA, C12-200, Dlin-KC2-DMA and Dlin-KC3-DMA. The helper lipids are electrostatically neutral or anionic with a neutral to slightly negative intrinsic curvature and cover the complex of cationic lipids and nucleic acid. The helper lipids also assist in forming a monolayer constituting the interface between the interior of the transfection particle and the aqueous phase, which can be further functionalized with PEGylated lipids or lipids carrying targeting moieties/ligands. Helper lipids include, but are not limited to phosphatidylcholine and phosphorylethanolamine lipids with saturated and or unsaturated C12-C20 acyl chains or sterols. PEGylated lipids of varying PEG length (350, 550, 750, 1000, 2000, 3000, 5000 Da) anchored to C14-C18 acylated, saturated or unsaturated, phosphorylethanolamine lipids, sterols or and the like has been used for steric stabilization and size control of lipid-based transfection particles, and for reducing blood clearance upon systemic administration. Lipoplexes may be formed, as described in example 3 and 4, by mixing of nucleic acids dissolved in water with either lipid mixtures dissolved in organic solvents such as, but not limited to, EtOH, tert-butanol, DMSO, alcohols or preformed extruded liposomes prepared in water or buffer. The complex formation is typically performed in aqueous media or dilute aqueous conditions to achieve full charging of the nucleic acid and thereby optimal charge-charge interaction between the nucleic acids and lipids. Here pH and salt concentration (ionic strength) are important factors, as pH impacts the protonation stage of the cationic lipids, and salt may screen the interactions between the two oppositely charged nucleic acids and lipid matrix. The formed lipoplexes typically have sizes in the 25-1000 nm range and zeta-potential in the range-10 mV-+50 mV. The highly charged backbone of the nucleic acid has thus screened by opposing charges of the lipids and may be transferred to non-aqueous media. The lipoplex structure protects nucleic acids from enzymatic degradation and enables cargo release to tumor sites.

Lipoplexes are generally formed in ultrapure water, buffers, or mixtures with organic solvents. Nucleic acids may be solubilized in ultrapure water at concentration of 0.1-10 μg/μL. The lipid mixture comprising cationic, helper and PEGylated lipids can be dissolved in organic solvent such as EtOH, DMSO and the like in concentration of 10-50 mM. Alternatively, liposomes comprising mixtures of cationic, helper and PEGylated lipids are formed in water or buffer by 1) freeze-drying of lipids dissolved in tert-buthanol: water (9:1), 2) rehydration of lipid powder/cakes in hydration buffer such as HEPES, TRIS or PBS or water, 3) sizing of liposomes by extrusion, sonication or homogenization. Liposomes are typically prepared at concentrations of 5-50 mM. Following, the solutions of lipids and nucleic acids are mixed at NP ratios ranging from 1-200 by simple mixing such as by using pipettes and stirring or controlled microfluidic mixing. In this process, complexes of anionic nucleic acid and the cationic lipid/liposomes are spontaneously formed by a self-organising process driven by electrostatic interactions. Exchange of water, buffer or solvents may following be achieved by tangential flow filtration, spin filtration dialysis and the like. The formed lipoplexes and SLNPs may following be modified by postinsertion of lipids that can partition into the lipoplexes or addition of polymers that may associate with the lipoplexes through charge-charge interactions. Addition of polymers or lipids to the preformed lipoplexes offers methods for attaching targeting ligands to the polyplexes. Greater detail of lipoplex and SLNP preparations and calculation of NP ratios are given in example 3 and 4.

In some embodiments, the lipid nanoparticle is a carbohydrate nanoparticle comprising a carbohydrate carrier and a therapeutic nucleic acid. As a non-limiting example, the carbohydrate carrier may include, but is not limited to, an anhydride-modified phytoglycogen or glycogen-type material, phtoglycogen octenyl succinate, phytoglycogen beta-dextrin, or anhydride-modified phytoglycogen beta-dextrin.

In some embodiments, the therapeutic nucleic acid is formulated in a liposome. Liposomes are artificially-prepared vesicles which may primarily be composed of a lipid bilayer and may be used as a delivery vehicle for the administration of pharmaceutical formulations. The therapeutic nucleic acid may be encapsulated by the liposome and/or it may be contained in an aqueous core which may then be encapsulated by the liposome.

Liposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter. Liposomes may be formulated for targeted delivery. Liposome design may include, but is not limited to, opsonins or ligands in order to improve the attachment of liposomes to unhealthy tissue or to activate events such as, but not limited to, endocytosis. Liposomes may contain a low or a high pH in order to improve the delivery of the composition.

In one embodiment, compositions of the invention comprise liposomes selected from 1,2-dioleyloxy-N,N-dimethylaminopropane (DODMA) liposomes, 1,2-dilinoleyloxy-3-dimethylaminopropane (Dlin-DMA) liposomes, and 2,2-dilinoleyl-4-(2-dimethylaminoethyl)-[1,3]-dioxolane (Dlin-KC2-DMA) liposomes. In one embodiment, compositions of the invention comprise liposomes formed from the synthesis of stabilized plasmid-lipid particles (SPLP) or stabilized nucleic acid lipid particle (SNALP).

In one embodiment, the therapeutic nucleic acid is formulated in a liposome having crosslinks between functionalized lipid bilayers. In one embodiment, the therapeutic nucleic acid is formulated in a liposome comprising a cationic lipid. In one embodiment, liposome formulations comprise from about 35 to about 45% cationic lipid, from about 40% to about 50% cationic lipid, from about 50% to about 60% cationic lipid and/or from about 55% to about 65% cationic lipid. In one embodiment, the ratio of lipid to therapeutic nucleic acid in liposomes is from about 5:1 to about 20:1, from about 10:1 to about 25:1, from about 15:1 to about 30:1 and/or at least 30:1.

In some embodiments, the therapeutic nucleic acid is formulated in a lipid nanoparticle. Lipid nanoparticles are self-assembling cationic lipid based systems which typically comprise a neutral lipid (the liposome base); a cationic lipid (for nucleotide loading); a sterol, e.g. cholesterol (for stabilizing the liposomes); and a molecule capable of reducing particle aggregation, for example a PEG or PEG-modified lipid (for stabilizing the composition, charge shielding and extended circulation in the bloodstream).

The lipid nanoparticles may comprise an ionizable cationic lipid selected from e.g. 2,2-dilinoleyl-4-dimethylaminoethyl-[1,3]-dioxolane (Dlin-KC2-DMA), dilinoleyl-methyl-4-dimethylaminobutyrate (Dlin-MC3-DMA), and di((Z)-non-2-en-1-yl) 9-((4-(dimethylamino) butanoyl)oxy) heptadecanedioate (L319). In one embodiment, the lipid nanoparticle formulation comprises: (i) at least one lipid selected from the group consisting of 2,2-dilinoleyl-4-dimethylaminoethyl-[1,3]-dioxolane (Dlin-KC2-DMA), dilinoleyl-methyl-4-dimethylaminobutyrate (Dlin-MC3-DMA), and di((Z)-non-2-en-1-yl) 9-((4-(dimethylamino) butanoyl)oxy) heptadecanedioate (L319); (ii) a neutral lipid selected from DSPC, DPPC, POPC, DOPE and SM; (iii) a sterol, e.g., cholesterol; and (iv) a PEG-lipid, e.g., PEG-DMG or PEG-CDMA, optionally in a molar ratio of about 20-60% cationic lipid: 5-25% neutral lipid: 25-55% sterol; 0.5-15% PEG-lipid.

In one embodiment, the PEG or PEG modified lipid comprises a PEG molecule of an average molecular weight of 2,000 Da. In other embodiments, the PEG or PEG modified lipid comprises a PEG molecule of an average molecular weight of less than 2,000 Da, for example around 1,500 Da, around 1,000 Da, or around 500 Da. Exemplary PEG-modified lipids include, but are not limited to, PEG-distearoyl glycerol (PEG-DMG) (also referred to as PEG-C14 or C14-PEG), and PEG-cDMA.

In one embodiment, the lipid nanoparticle formulations comprise a cationic lipid, a PEG lipid and a structural lipid and optionally comprise a non-cationic lipid. As a non-limiting example, the lipid nanoparticle may comprise about 40-60% of cationic lipid, about 5-15% of a non-cationic lipid, about 1-2% of a PEG lipid and about 30-50% of a structural lipid. In one embodiment, the cationic lipid is selected from Dlin-KC2-DMA, Dlin-MC3-DMA and L319.

The nucleic acid component may comprise a phosphate conjugate for increasing in vivo circulation times and/or increasing the targeted delivery of the lipid nanoparticle. The nucleic acid component may comprise a polymer conjugate e.g. a water soluble conjugate. The nucleic acid component may comprise a conjugate to enhance the delivery of lipid nanoparticles in a subject and/or to inhibit phagocytic clearance of the lipid nanoparticles in a subject.

The lipid nanoparticle may be a carbohydrate nanoparticle comprising a carbohydrate carrier and a therapeutic nucleotide. As a non-limiting example, the carbohydrate carrier may include, but is not limited to, an anhydride-modified phytoglycogen or glycogen-type material, phtoglycogen octenyl succinate, phytoglycogen beta-dextrin, or anhydride-modified phytoglycogen beta-dextrin.

In one embodiment, compositions are configured to be passively or actively directed to different cell types in vivo, including but not limited to hepatocytes, immune cells, tumor cells, endothelial cells, antigen presenting cells, and leukocytes. An example of passive targeting of formulations to liver cells includes the Dlin-DMA, Dlin-KC2-DMA and Dlin-MC3-DMA-based lipid nanoparticle formulations which bind to apolipoprotein E and promote binding and uptake of these formulations into hepatocytes in vivo. Compositions can also be selectively targeted through expression of different ligands on their surface as exemplified by, but not limited by, folate, transferrin, N-acetylgalactosamine (GalNAc), and antibody targeted approaches.

In one embodiment, the therapeutic nucleic acid is formulated as a solid lipid nanoparticle. A solid lipid nanoparticle (SLN) may be spherical with an average diameter between 10 to 1000 nm. SLNs possess a solid lipid core matrix that can solubilize lipophilic molecules and may be stabilized with surfactants and/or emulsifiers. In a further embodiment, the lipid nanoparticle may be a self-assembly lipid-polymer nanoparticle.

In one embodiment, the therapeutic nucleic acid is formulated as self-assembled nanoparticles. In one embodiment, the self-assembled nanoparticles comprise a core of the therapeutic nucleic acid and a polymer shell.

### Targeting of the Nucleic Acid Component

Targeting of the nucleic acid component may be achieved through the coupling of ligands or moieties to the nucleic acid component (e.g. to nucleic acid, lipids or polymers within the nucleic acid component). Targeting properties may increase interactions with cells including both cell unspecific and cell specific uptake/targeting and regulate intracellular trafficking and function. Coupling to targeting ligands or cell penetrating peptides may also allow for direct cellular uptake without the need for complexation or particle formulation. Co-valent or non co-valent attachment of the targeting ligand allows recognition of specific antigens or receptors on specific cells or enhances cellular uptake or trafficking. Targeting may be performed using Fabs, nanobodies, sd-domain Abs, bi-specific Abs, peptides and proteins, vitamins and, aptamers (RNA aptamers, peptide aptamers and DNA aptamers). Alternatively, the nucleic acid component may be modified for increased cellular uptake by cell penetrating peptides (e.g., TAT, polyArginine, penetratin) which may allow for increased cellular uptake both unspecific and with a selectivity towards specific cells (e.g., B cells with penetratin, folate, mannose, and galactose for cancer cells and macrophages). Targeting ligands include, but are not limited to; RGD, Transferrin, Folate, Vitamin D, a signal peptide or signal sequence, a localization signal or sequence, a nuclear localization signal or sequence (NLS), an antibody, a cell penetrating peptide (CPP), (e.g. TAT, KALA), a ligand of a receptor (e.g. cytokines, hormones, growth factors etc), small molecules (e.g. carbohydrates like mannose, N-Acetylgalactosamine (GalNAc) or galactose or synthetic ligands), small molecule agonists, inhibitors or antagonists of receptors (e.g. RGD peptidomimetic analogues) or any such molecule. Particularly preferred are cell penetrating peptides (CPPs), which include, without being limited thereto protamine, nucleoline, spermine or spermidine, poly-L-lysine (PLL), basic polypeptides, poly-arginine, chimeric CPPs, such as Transportan, or MPG peptides, HIV-binding peptides, HIV-1 Tat, Tat-derived peptides, oligoarginines, members of the penetratin family, e.g. Penetratin, Antennapedia-derived peptides (particularly from Drosophila antennapedia), pAntp, plsl, etc., antimicrobial-derived CPPs e.g. Buforin-2, Bac715-24, SynB, SynB (1), pVEC, hCT-derived peptides, SAP, MAP, PpTG20, proline-rich peptides, Loligomers, arginine-rich peptides, Calcitonin-peptides, FGF, Lactoferrin, poly-L-lysine, poly-arginine, histones, VP22 derived or analog peptides, Pestivirus Erns, HSV, VP22 (Herpes simplex), MAP, KALA or protein transduction domains (PTDs, PpT620, proline-rich peptides, arginine-rich peptides, lysine-rich peptides, Pep-1, L-oligomers, Calcitonin peptide(s). Alternatives may include specific cell targeting using CD19, CD22, CD30, CD33, CD44, CD74, CD276, EGFR, Nectin4, AXL, ALK, PTK7, TM4SF1, LRP1, Somatostatin, RGD, Tenascin3, Nucleolin, Mucin-1, Fibronectin, Tenascin C, MT1-MMP, Glucose receptor, Mannose receptor, Galactose receptor, HER2, Transferrin, Folic acid receptor, Hyaluronan, and PSMA. Targeting may furthermore be directed towards antigen presenting cells are dendritic cells using DEC205, XCR1, CD197, CD80, CD86, CD123, CD209, CD273, CD283, CD289, CD184, CD85h, CD85j, CD85k, CD85d, CD85g, CD85a, CD141, CD1lc, CD83, TSLP receptor, Clec9a or Cdla marker. In one embodiment, the dendritic cells are targeted using the CD141, FLT3L, trombin, DEC205 ligand FSSVRY, or the XCR1 ligand XCL1.

The therapeutic nucleic acid may be formulated with peptides and/or proteins in order to increase transfection of cells by the nucleic acid. In one embodiment, the therapeutic nucleic acid may be formulated with a cell penetrating peptide or protein, or a peptide that enables intracellular delivery. Cell penetrating peptides which may be used with the composition of the invention include, but are not limited to, a cell-penetrating peptide sequence attached to polycations that facilitates delivery to the intracellular space, e.g., HIV-derived TAT peptide, penetratins, transportans, or hCT derived cell-penetrating peptides.

In one embodiment, the cell-penetrating peptide may comprise a first domain and a second domain. The first domain may comprise a supercharged polypeptide. The second domain may comprise a protein-binding partner. As used herein, “protein-binding partner” includes, but is not limited to, antibodies and functional fragments thereof, scaffold proteins, or peptides. The cell-penetrating peptide may further comprise an intracellular binding partner for the protein-binding partner. The cell-penetrating peptide may be capable of being secreted from a cell where the nucleic acid may be introduced.

The therapeutic nucleic acid of the invention include conjugates, such as a nucleic acid covalently linked to a carrier or targeting group, or including two encoding regions that together produce a fusion protein (e.g., bearing a targeting group and therapeutic protein or peptide).

The conjugates include, but are not limited to, naturally occurring substances, such as proteins (e.g., human serum albumin (HSA), low-density lipoprotein (LDL), high-density lipoprotein (HDL), or globulin); carbohydrates (e.g., a dextran, pullulan, chitin, chitosan, inulin, cyclodextrin or hyaluronic acid); or lipids. The conjugates may also comprise a recombinant or synthetic molecule, such as a synthetic polymer, e.g., a synthetic polyamino acid, or an oligonucleotide (e.g. an aptamer).

The conjugates can also include targeting groups, e.g., a cell or tissue targeting agent, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, Mucin carbohydrate, multivalent lactose, multivalent galactose, N-acetyl-galactosamine, N-acetyl-gulucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, an RGD peptide, an RGD peptide mimetic or an aptamer. A targeting group can be a multivalent lactulose.

Targeting groups can be proteins, e.g., glycoproteins, or peptides, e.g., molecules having a specific affinity for a co-ligand, or antibodies e.g., an antibody, that binds to a specified cell type such as a cancer cell, endothelial cell, or bone cell. Targeting groups may also include hormones and hormone receptors. They can also include non-peptidic species, such as lipids, lectins, carbohydrates, vitamins, cofactors, multivalent lactose, multivalent galactose, N-acetyl-galactosamine, N-acetyl-gulucosamine multivalent mannose, multivalent fucose, or aptamers. They can also include multivalent lactulose. The ligand can be, for example, a lipopolysaccharide, or an activator of p38 MAP kinase.

The targeting group can be any ligand that is capable of targeting a specific receptor. Examples include, without limitation, folate, GalNAc, galactose, mannose, mannose-6P, apatamers, integrin receptor ligands, chemokine receptor ligands, transferrin, biotin, serotonin receptor ligands, PSMA, endothelin, GCPII, somatostatin, LDL, and HDL ligands. In particular embodiments, the targeting group is an aptamer. The aptamer can be unmodified or have any combination of modifications disclosed herein.

Hydrophobic Ion-pairs of nucleic acids (HIPs): The nucleic acid component may comprise hydrophobic ion pairs (HIPs) of charged nucleic acids to enhance solubilization in hydrophobic solvents, oils or depots (e.g. NCCells). For this purpose, charged nucleic acids are complexed by hydrophobic counterions forming hydrophobic ion pairs that can be solubilized in hydrophobic media such as but not limited to organic solvents, oils, solid lipid nanoparticles, micelles, liposomes, polymersomes, polymeric nanoparticles and NCCells.

Engineering of nucleic acids' water solubilities can enable new compositions and processing methods by formulation in hydrophobic drug delivery systems, such as but not limited to oils, hydrophobic polymers systems, hydrophobic carbohydrate ester gels and NCCells. This can be performed by reversible covalent conjugation of nucleic acids with hydrophobic or amphiphilic molecules, which results in the production of a nucleic acid complex that can be mixed with hydrophobic compositions such as but not limited to oils, hydrophobic polymers systems, hydrophobic carbohydrate ester gels and NCCells. Nucleic acid salt form engineering is an alternative route to enhance and control solubility in systems that the nucleic acid would otherwise not mix with. Charged functional groups, often phosphates, phosphonates thiophosphates, amines, sulphates, sulfonatates, carboxylates, and the like, on hydrophobic or amphiplic molecules is ion-paired with the nucleic acid to produce a nucleic acid with transiently altered solubilities. The use of hydrophobic ion-pairs to create hydrophobic salt forms can increase nucleic acid hydrophobicity.

Controlling nucleic acid release kinetics and being able to maintain a release of such entities that are active locally, locoregionally or systemically, has important therapeutic indications across several diseases and pathological conditions. The therapeutic indications for the combination of hydrophobic ion-pairing in combination with oils, depots (e.g. NCCells), or other hydrophobic compositions have extensive applications across a broad selection of disease in human or animal.

Hydrophobic ion pairing (“HIP”) of nucleic acids is the process of forming ionic interactions between a charged nucleic acid and a hydrophobic counterion. The complexation increases hydrophobicity by two main mechanisms: First, the nucleic acid's natural charge is masked, reducing solubility in polar solvents such as water. Second, the hydrophobic groups on the counterion, typically nonpolar aliphatic tails or aromatic groups, help to coat the nucleic acid's surface area with hydrophobic domains that exclude water. HIP thus converts nucleic acids into hydrophobic ion pairs that can be solubilized and released from hydrophobic drug delivery vehicles such as but not limited to oils and depots (e.g. NCCells). The HIPs thus remain hydrophobic while solubilized in the oils or NCCell. After release to aqueous media or tissue, the HIPs may dissociate as the water dipole screening of charges lower the charge-charge interaction energy, thereby releasing the nucleic acid upon exposure to aqueous media.

Nucleic acids eligible for HIP include deoxyribonucleic acids (DNAs), threose nucleic acids (TNAs), antisense DNA, glycol nucleic acids (GNAs), peptide nucleic acids (PNAs), hexitol nucleic acids (HNA), Morpholino and locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization) or hybrids thereof and single or double stranded ribonucleic acids (RNAs), including RNA (messenger RNA (mRNA), self-replicating mRNA, transfer RNA (tRNA), and ribosomal RNA (rRNA)), small single or double stranded RNA (dsRNA, RNA interference (RNAi) including microRNA (miRNA), small interfering RNA (siRNA or ASO, including chemical modifications phosphorotioate (PS), PS morpholino, 2′-O-methyl, 2′-O-methoxyethyl, 2′fluoro, 5′methylcystine, G-clamp), splice switching antisense oligonucleotide (SSO), CRISPR-Cas9 sgRNAs, piwi-interacting RNA (piRNA) and repeat associated small interfering RNA (rasiRNA). Nucleic acids eligible for HIP also transfer mRNA (tmRNA). In addition to alternative synthetic nucleotides analogous including 2′-O-methyl-substituted and Phosphorothioate RNA (psRNA) and Aptamers, or the like that has one or more ionizable groups or moieties. The ionizable group may be amines (primary, secondary, tertiary or quaternary), alcohols, carboxylic acids, thiols, sulphonic, sulphenic or sulfinic acids, tetraalkyl phosphonium, phosphonic, phosphenic, phosphinic acids, or phosphorothioates, conjugated systems with delocalized charges, and the like. Compounds eligible for HIP may contain one or multiple ionizable groups of the same or opposite charge. Depending on the acidity (pKa) of the molecules ionizable groups, varying charge of the molecule may be obtained as function of pH. Control of pH during preparation of HIP is important as charge regulation on both the hydrophobic co-ion and the active molecule with ionizable groups is crucial for a successful HIP formation.

Hydrophobic counterions: The counterions, also referred to as co-ions, used for hydrophobic ion pairing should contain at least one charged group and at least one hydrophobic domain. The counterions may be either anionic or cationic and typically contain either one, two or multiple charged groups. The ionizable group of the hydrophobic counterions may be amines (primary, secondary, tertiary or quaternary), alcohols, carboxylic acids, thiols, sulphonic, sulphenic or sulfinic acids, tetraalkyl phosphonium, phosphonic, phosphenic, phosphinic acids, or phosphorothioates, conjugated systems with delocalized charges, and the like.

The counterion can have a logP value of 0 or greater at a pH of 7, and preferably larger than 2 at pH 7. The counterion can have a logP value of greater than 5. The counterion can be an anionic counterion that has a pKa value of from −2 to 5. The counterion can have a pKb value of greater than 3. The counterion can be a quaternized cationic species, for example, a quaternized cationic species that is permanently cationic. The counterion can have an ionic site selected from the group consisting of amines (primary, secondary, tertiary or quaternary), alcohols, carboxylic acids, thiols, sulphonic, sulphenic or sulfinic acids, tetraalkyl phosphonium, phosphonic, phosphenic, or phosphinic acids, conjugated systems with delocalized charges, and the like.

Some counterions may exert pharmacological or diagnostic activity beyond their actions as hydrophobic counterion.

Examples of counterions suitable for HIP complexation with nucleic acids with at least one charge and at least one hydrophobic domain includes, but not limited to, the cationic lipids, DC-chol, DMTAP, DPTAP, DSTAP, DOTAP, DOTMA, DOSPA, DDAB, DMDAP, DPDAP, DSDAP, DODAP, DODMA, DOBAQ, DLin-DMA, DLin-KC2-DMA, DLin-MC3-DMA, C12-200, A6, OF-02, A18-Iso5-2DC18, YSK05, 7C1, GO-C14, L319, OF-Deg-Lin, 306-012B, 3060110, FTT5, 9A1P9, 98N12-5, 304013, cKK-E12, Spermine-chol (also referred to herein as “spermine-cholesterol” or “cholesterol-spermine”), and MVL5, such as the carboxylic acids, acetic acid, propanoic acid, butanoic acid, hexanoic acid, octanoic acid, benzoic acid, Myristic acid, Myristoleic acid, Palmitic acid, Palmitoleic acid, Stearic acid, Oleic acid, Elaidic acid, Linoleic acid, Linolenic acid, Linolelaidic acid, and bilesalts such as Cholic Acid, such as Benethamine (N-benzyl-2-phenylethanamine), Dodecylamine (laurylamine), Hexadecyl trimethylammonium (cetrimonium) bromide (CTAB), Maprotiline, Na-Deoxycholyl-L-lysyl-methylester, N,N′-Dibenzyl ethylenediamine (benzathine), N,N-Dimethyl dodecylamine (DDA), N,N-Dimethyl hexylamine, N,N-Dimethyl octadecylamine (dimethyl stearamine), Tetrabutyl ammonium bromide (TBAB), Tetraheptyl ammonium bromide (THA), Tetrahexyl ammonium bromide, Tetraoctyl ammonium bromide (TOAB), Tetrapentyl ammonium bromide (TPA), Triethylamine (TEA), such as but not limited to the multivalent cationic lipids N1-[2-((1S)-1-[(3-aminopropyl)amino]-4-[di(3-amino-propyl)amino]butylcarboxamido)ethyl]-3,4-di [oleyloxy]-benzamide or N4-Cholesteryl-Spermine, such as counterions belonging to the group of phosphatidic acid (PA), cyclic PA, lysophosphatidic acid (LPA), cyclic LPA, phosphatidylglycerol (PG), lysophosphatidylglycerol (LPG), phosphoinositides (PI), lysophosphatidylinositol (LPI), phosphatidylserine (PS), or Lysophosphatidylserine (LPS)phospholipids and or ether lipids, or sphingolipids (SP) or sphingolysolipids (LSP), all with mixed or non-mixed, saturated and or unsaturated, aliphatic and or aromatic, C6-C22 hydrocarbon chains. Preferred examples of lipid chains include, but are not limited to propionyl, butyryl, hexanoyl, octanoyl, decanoyl, lauroyl, myristoyl, palmitoyl, stearoyl, arachidoyl, behenoyl, lignoceroyl and the like. Other preferred examples of lipid chains are vaccenoyl, (8Z) octadecenoyl, myristoleoyl, myristelaidoyl, palmitoleoyl, palmitelaidoyl, oleoyl, dielaidoyl and the like.

### HIP Complexes of Nucleic Acids

During HIP formation, a nucleic acid forms a hydrophobic ion pair with hydrophobic counterions. Depending on the physicochemical properties of the nucleic acid or hydrophobic counterion different approaches may be applied. If all species of the complex are individually soluble in water, the organic solvent-free method may be employed.

For the organic solvent-free method, both nucleic acid and the hydrophobic counterion are dissolved in water or buffer. The pH of the aqueous buffer may be adjusted so that the charge of the nucleic acid and the hydrophobic counterion is non-zero and have opposing charge. Hereafter the nucleic acid, and the hydrophobic counterion is mixed and agitated whereafter the HIP forms and may precipitate with time. Hereafter the HIP can be spun down by centrifugation, washed, and resuspended in compositions of the invention, organic solvent, oils, depots (e.g. NCCells) and the like. If no precipitation occurs, the HIP complexes may be transferred to organic media by extraction, vacuum distillation transfer, and the like. Advantages of the organic solvent-free method is that potentially toxic solvents are avoided.

For counterions that are insoluble or weakly soluble in water, one of the two following approaches may be used.

In the first approach, the nucleic acid is dissolved in water, and the counterion in methanol. Next, the nucleic acid in water (aq) and the counterion in methanol (MeOH) is mixed with chloroform in the ratio nucleic acid (aq): counterion (MeOH): chloroform (1:2:1). The mixture forms a monophase which is shaken or stirred for >30 min wherein the HIP form. Next, a biphasic system is formed by addition of chloroform and water until reaching the ratio water: MeOH: Chloroform (1:1:1) whereafter the HIP partition into the organic phase. The HIP may be isolated from the organic phase by evaporation of the solvent whereafter it can be resuspended or solubilized in compositions of the invention, organic solvents, oils or depots (e.g. NCCells).

Alternatively, the nucleic acid is dissolved in water and the hydrophobic counterion in chloroform. Equal volumes of the nucleic acid and the counterion is mixed forming a biphasic system. The solution is agitated or stirred for 2-4 hours whereafter the solutions is centrifuged promoting phase separation. Following phase separation, the HIP partitions into the organic phase from where it can be isolated.

Alternatively, the nucleic acid is dissolved in water and the hydrophobic counterion in an organic solvent which is mixable with water. The water and organic phase are mixed, and the solution is agitated or stirred. Following, the aqueous fraction of the mixture may be removed by vacuum distillation at low temperature if the organic solvent has a higher boiling point than water and does not form an azeotrope with water. After distillation, the remaining phase is comprised of the organic solvent and the HIP complex. Relevant solvents for vacuum transfer include, but are not limited to DMSO, PC, EtOH, iso-propanol and BnOH.

Depending on solvent sensitivity of the nucleic acid, variations of the previous described methods using alternative solvent may be applied to minimize denaturation or deterioration of nucleic acid and the like.

Transfer of Nucleic Acid Component from Aqueous Media to the Hydrophobic Component.

Nucleic acid component, optionally comprising transfection particles such as LNPs, polyplexes and lipoplexes, may be formed in aqueous media and then transferred into solvent or co-solvents compatible with the hydrophobic component to aid their incorporation into the hydrophobic component. Exchange of solvents can be achieved by dialysis, tangential flow filtration, spin filtration and the like. Additionally, nucleic acid component prepared in water or buffer can be mixed with organic solvent for vacuum transfer (low temperature distillation). In this process, water containing nucleic acid component and the organic phase is mixed, and the solution is agitated or stirred. Following, the aqueous fraction of the mixture may be removed by vacuum distillation at low temperature if the organic solvent has a higher boiling point than water and does not form an azeotrope with water as described in example 8. After distillation, the remaining phase is comprised of the organic solvent and the nucleic acid component. Relevant solvents for vacuum transfer (vacuum distillation) include, but are not limited to DMSO, PC and BnOH, PEGs, PEG200, PEG400, PEG800, benzyl-benzoate, and propylene glycol. Vacuum distillation can be conducted at temperatures below the boiling point and above the freezing point of the water-organic solvent mixture, such as 50-100° C., or such as 30-70° C. or such as 20-50° C. or such as 0-30° C. or such at room temperature. It can be advantageous to conduct the vacuum distillation at low temperatures to preserve the integrity of the nucleic acid component.

Controlled Release of Nucleic Acid Component from Compositions of the Invention

Compositions of the invention provide controlled release of nucleic acid component. As noted above, in some embodiments, the nucleic acid component comprises a non-complexed polynucleotide, a HIP complexed nucleic acid or a nanoparticle such as lipid nanoparticle, polyplex, lipoplex or lipopolyplex. Preferably, compositions of the invention form a depot within an aqueous environment (such as animal or human tissue) from which the nucleic acid component is released. In one embodiment, the composition contains an organic solvent such as but not limited to DMSO, Ethanol, benzyl alcohol, propylene carbonate (PC), or short polyethylene glycols, or lipid oils. In one preferred composition of the invention the nucleic acids or complexed nucleic acids are injected into human or animal body where the composition forms a depot for controlled release of nucleic acids or complexed nucleic acids, and where the composition comprises nucleic acids or complexed nucleic acids, an organic solvent, and a hydrophobic component, optionally wherein the hydrophobic component comprises a carbohydrate ether, as carbohydrate ester, a lipid, a triglyceride, a diglyceride, a phospholipid, a cholesterol, and the like.

In one embodiment, the depot is an NCCell. After depot formation, an NCCell comprises a hydrophobic matrix made from gel-forming carbohydrate esters, and typically a co-solvent. Overall, the NCCell is hydrophobic, but the carbohydrate backbone of the carbohydrate esters provides options for hydrogen bonding via oxygen. The nucleic acid component may be dispersed in an NCCell as powder, crystals, nanoparticles and the like. The nucleic acid component may be fully solubilized in the NCCell.

The nucleic acid component may therefore interact with the hydrophobic component (e.g. of an NCCell) via hydrogen bonding, dipole or van der Wahls interactions. Enhancing the interaction between the nucleic acid component and hydrophobic component (e.g. of an NCCell) typically reduces release rate of nucleic acid component from the hydrophobic component (e.g. of an NCCell), whereas reducing the interaction between the nucleic acid component and the hydrophobic component (e.g. of an NCCell) typically increases the release rate of the nucleic acid component.

As noted herein, the nucleic acid component may comprise a polyplex, a lipoplex, an LNP, a lipopolyplex, a hydrophobic HIP and the like. Interaction with the hydrophobic component (e.g. of an NCCell) may be regulated via steric, hydrophilic and or hydrophobic interactions. Wherein the nucleic acid component comprises particles, the particle size (e.g. of the a LNP, polyplex, lipoplex or lipopolyplex) and the viscosity of the hydrophobic component (e.g. of an NCCell) determines the shear forces inside the hydrophobic component and hence the speed of diffusion, which impacts the particle release rate. Larger particles are released at a slower rate compared to smaller particles. Functionalization by polymers or charged groups e.g. by addition of hydrophobic (PNIMAP, PLA, PLGA) or hydrophilic (PEG, PEI, PLL, PAA) surface functionalities may impact the release of the nucleic acid complexes. Here changes in nucleic acid complex size, as well as change in their interaction/solubility in aqueous media and in the hydrophobic component both affects the release rate. Larger nucleic acid complexes are usually released at a slower rate, whereas more hydrophobic nucleic acid complexes may have a slower release due to their lower affinity for water and higher affinity for the hydrophobic component. Engineering of nucleic acid complex size and balance of hydrophobic/hydrophilic character are thus important parameters for tuning of their release rates from the hydrophobic component of depots (e.g. NCCells).

Nucleic acid complex sizes may be affected by e.g. i) change in the N/P ratio, ii) use of salt (ionic strength) or change in pH during preparation of the complex, and or iii) changing the lipid or polymer composition. Using more hydrophobic polymers, lipids or co-ions with a larger logP may enhance the interaction of the nucleic acid component (e.g. LNP, lipoplex, polyplex or nucleic acid HIP) with the hydrophobic component (e.g. of an NCCell) via enhanced van der Wahls interaction. Using bulkier co-ions may increase the size of a HIP complex, which increases the steric interaction of the HIP and hydrophobic component (e.g. of an NCCell). Both effects of using more hydrophobic or bulkier co-ions may enhance the interaction of the HIP and hydrophobic component (e.g. of an NCCell) and may lead to altered release rate of the HIP.

### Depot Formation

Composition components: Upon transfer to an aqueous environment (e.g. upon injection into human or animal tissues or other aqueous media), compositions of the invention form a gel or gel-like depot within the tissue or aqueous media. In one embodiment, the composition is an NCCell solution comprising organic solvent(s), oil(s) (co-solvent), gel-forming carbohydrates ester and a nucleic acid component, and is a fluid with viscosities typically, but not limited to, in the range 50-5000 cP. Upon administration of the composition into tissues, the solution is in contact with aqueous fluids which causes phase separation to occur. In this process, the organic solvent diffuses into the aqueous phase (interstitial fluids). The remaining features of the hydrophobic component (e.g. gel-forming carbohydrate (optionally carbohydrate ester), lipid polymer or mixture thereof; and optionally oil (co-solvent)) forms a high viscosity fluid, solid or precipitate or a combination thereof forming a depot at the site of injection. The depot is typically hydrophobic and provides controlled release of the nucleic acid component over time, from hours, days, weeks or months. The viscous fluid, solid, precipitate or combinations thereof is referred to as a gel, gel depot or depot, and may be an NCCell.

Solvents of the composition: Hydrophobic substances such as the gel-forming carbohydrate (optionally carbohydrate ester), lipid polymer or mixture thereof; and optionally the oil (co-solvent), as well as in hydrophilic substances such as water; are soluble or mixable in solvents of the composition. This partial hydrophilic/hydrophobic property of the solvents drives the phase separation since the solvents of the composition readily diffuse out when exposed to an aqueous environment.

The chemical composition of the solvent (dispersion medium) should not be particularly limited. Examples include biocompatible organic solvents such as ethanol, ethyl lactate, propylene carbonate, glycofurol, N-methylpyrrolidone, 2-pyrrolidone, propylene glycol, acetone, methyl acetate, ethyl acetate, methyl ethyl ketone, benzyl alcohol, triacetin, dimethylformamide, dimethylsulfoxide, tetrahydrofuran, caprolactam, decylmethylsulfoxide, such as but not limited to N-methyl-2-pyrrolidone, glycofurol, polyethylene glycol (PEG), benzyl benzoate, triglycerides, acetone, benzyl alcohol, V-(betahydromethyl) lactamide, butylene glycol, caprolactam, caprolactone, corn oil, decylmethylsulfoxide, dimethyl ether, dimethyl sulfoxide, 1-dodecylazacycloheptan-2-one, ethanol, ethyl acetate, ethyl lactate, ethyl oleate, glycerol, glycofurol (tetraglycol), isopropyl myristate, methyl acetate, methyl ethyl ketone, esters of caprylic and/or capric acids with glycerol or alkylene glycols, oleic acid, peanut oil, polyethylene glycol, propylene carbonate, 2-pyrrolidone, sesame oil, [+]-2,2-dimethyl-1,3-dioxolane-4-methanol, tetrahydrofuran, diethylene glycol monoethyl ether, carbitol, triacetin, triethyl citrate, and combinations thereof; or desirably from trichlorofluoromethane, dichlorofluoromethane, tetrafluoroethane, dimethyl ether, propane, butane, and combinations thereof; or specifically from caprylic/capric triglyceride, oleic acid, 1-dodecylazacycloheptan-2-one and the like. Examples of organic solvents also include anisole, 1-propanol, 1-buthanol, ethanol, NMP or DMSO.

Although the gel formulation can be stably dispersed in these solvents (dispersion media), the solvents may be further added with a saccharide derivatives of for example, triglycerides such as tri-pentanoyl glycerol, tri-octanoyl glycerol, tri-dodecanoyl glycerol, a monosaccharide such as glucose, galactose, mannose, fructose, inositol, ribose and xylose, disaccharide such as lactose, sucrose, cellobiose, trehalose and maltose, trisaccharide such as raffinose and melezitose, and polysaccharide such as α-, β-, or γ-cyclodextrin, sugar alcohol such as erythritol, xylitol, sorbitol, mannitol, and maltitol, or a polyhydric alcohol such as glycerin, diglycerin, polyglycerin, propylene glycol, polypropylene glycol, ethylene glycol, diethylene glycol, triethylene glycol, polyethylene glycol, ethylene glycol mono-alkyl ether, diethylene glycol mono-alkyl ether and 1,3-butylene glycol. Solvents may also be added with a saccharide derivative of for example lactulose.

Examples of more preferable solvents are polyhydric alcohol such as glycerin, diglycerin, polyglycerin, propylene glycol, polypropylene glycol, ethylene glycol, diethylene glycol, triethylene glycol, polyethylene glycol, polyethylene glycol (PEG), benzyl benzoate, triglycerides, acetone, benzyl alcohol, ethanol, ethyl lactate, propylene carbonate and Dimethyl Sulfoxide, 1-butanol, 2-butanol, Tert-butylmethyl ether, Ethyl ether, Ethyl formate, Heptane, 3-Methyl-1-butanol, Methylisobutylketone, 2-Methylisobutylketone, 2-Methyl-I-propanol, Pentane, 1-Pentanol, 1-Propanol, 2-Propanol.

Examples of solvents include, but are not limited to Ethanol (EtOH), Dimethyl sulphoxide (DMSO), Dimethyl formamide (DMF), N-methyl pyrrolidone (NMP), Propylene carbonate (PC), Benzyl alcohol (BnOH) or Butyl acetate (BuAc), glycerin, diglycerin, polyglycerin, propylene glycol, polypropylene glycol, ethylene glycol, diethylene glycol, triethylene glycol, polyethylene glycol, polyethylene glycol (PEG), benzyl benzoate, triglycerides, acetone, ethyl lactate. Examples of solvents also include anisole, 1-propanol, 1-buthanol, ethanol, NMP or DMSO.

Solvents described herein may be used alone or in combination.

Oils (also referred to as co-solvent): In some embodiments, the composition comprises an oil (co-solvent). Oils are hydrophobic substances that mix poorly with aqueous media. Upon injection of a composition of the invention into an aqueous environment (e.g. tissue or other aqueous media), the carbohydrate (optionally carbohydrate ester), lipid polymer or mixture thereof, and optional oil (co-solvent) are separated from the solvent due to Non-solvent Induced Phase Separation (“NIPS”). During this phase separation, the carbohydrate (optionally carbohydrate ester), lipid polymer or mixture thereof, and optional oil (co-solvent) forms a gel, gel depot or NCCell with properties governed by the carbohydrate (optionally carbohydrate ester), lipid polymer or mixture thereof, and optional oil (co-solvent).

Examples of oils (co-solvents) include, but are not limited to glycerol trivalerate, glycerol trihexanoate (GTH), glycerol trioctanoate (GTO), glycerol tridecanoate (GTD), ethyl octanoate, ethyl hexanoate, ethyl decanoate, Ethyl myristate, ethyl laurate, ethyl oleate, ethyl palmitate, ethyl myristate, corn oil, peanut oil, coconut oil, sesame oil, cinnamon oil, soybean oil, and poppyseed oil, or Lipiodol and aliphatic alkyl acyl esters.

Co-solvents described herein may be used alone or in combination.

Carbohydrate esters: Upon solvent efflux caused by phase separation, carbohydrate esters alone form viscous fluid depots, amorphous solid depots, crystal solid depots or mixtures thereof.

Examples of carbohydrate esters are Sucrose acetate isobutyrate (SAIB), Sucrose octapropionate (SOP), Sucrose octaisobutyrate (SOIB) Sucrose octabenzoate (SuBen), Lactose octapropionate (LOP), lactose octaisobutyrate (LOIB), Lactose octabenzoate (LacBen), Rafinose undecaisobutyrate (RUIB), Rafinose undecabenzoate (RaBen), MeLOIB (Methyl hepta-O-isobutyryl-α, β-lactoside), and the like. Examples of carbohydrate esters also include lactulose octapropionate, lactulose octaisobutyrate, or lactulose octabenzoate,

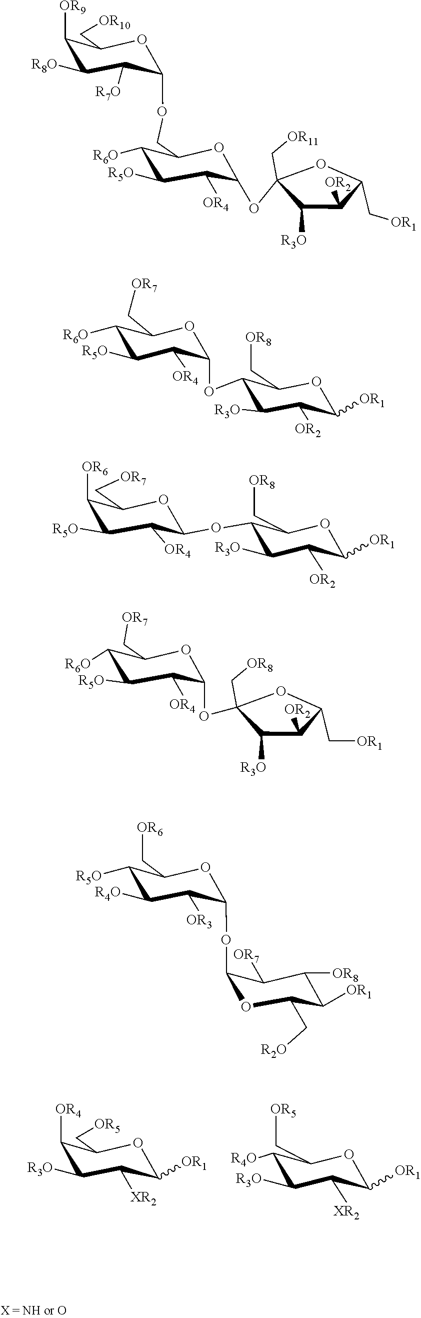

Schematic of chemical structures of exemplary carbohydrate esters, wherein R₁, R₂, R₃, R₄, R₅, R₆, R₇, R₈, R₉, R₁₀, and R₁₁ are selected collectively from the group consisting of hydrogen, methyl, alkanoyl, hydroxyl-substituted alkanoyl, and acyloxy-substituted alkanoyl, alkanyl, hydroxy-substituted alkanyl, acyloxy substituted alkanyl, benzoyl, and substituted benzoyl; or wherein R₁, R₂, R₃, R₄, R₅, R₆, R₇, R₈, R₉, R₁₀, and R₁₁ are independently selected from the group consisting of hydrogen methyl alkanoyl, hydroxyl-substituted alkanoyl, acyloxy-substituted alkanoyl, alkanyl, hydroxysubstituted alkanyl, acyloxy substituted alkanyl, benzoyl and substituted benzoyl; or wherein all groups of R₁, R₂, R₃, R₄, R₅, R₆, R₇, R₈, R₉, R₁₀, and R₁₁ are selected collectively from the group consisting of methyl, acetyl, isobutyryl, propionyl or benzoyl; or wherein R₁, R₂, R₃, R₄, R₅, R₆, R₇, R₈, R₉, R₁₀, and R₁₁ are independently selected from the group consisting methyl, acetyl, isobutyryl, propionyl or benzoyl; and wherein both pure anomers and mixtures of α- and β-anomers of the above mentioned structural variations are claimed.

According to yet another embodiment, the composition comprises at least one gel-forming carbohydrate ester, preferably acetate, propionate, butyrate, iso-butyrate or benzoate esters of Sucrose, Lactose, Trehalose, Raffinose, or Maltose, such as Sucrose octaacetate, Sucrose octapropionate (SOP), Sucrose acetate isobutyrate (SAIB), Sucrose octaisobutyrate (SOIB), Sucrose octabenzoate (SuBen), Lactose octapropionate (LOP), lactose octaisobutyrate (LOIB), Lactose octabenzoate (LacBen), Raffinose undecapropionate (RUP), Raffinose undecaisobutyrate (RUIB), Raffinose undecabenzoate (RaBen), Trehalose octapropionate (TOP), Trehalose octaisobutyrate (TOIB), Trehalose octabenzoate (TreBen), Maltose octapropionate (MOP), Maltose octaisobutyrate (MOIB), Maltose octabenzoate (MaBen), MeLOIB (Methyl hepta-O-isobutyryl-α,β-lactoside), more preferably Lactose octapropionate (LOP), lactose octaisobutyrate (LOIB), Lactose octabenzoate (LacBen), methoxy-LOIB (meLOIB), Raffinose undecabenzoat (RaBen), Raffinose undecaisobutyrate (ROIB), Sucrose octaisobutyrate (SOIB), Trehalose octaisobutyrate (TOIB) or Sucrose octabenzoate (SuBen).

Gel-forming carbohydrate esters described herein may be used alone or in combination. Gel-forming carbohydrate esters described herein may be partly or fully acylated or comprise mixtures of the same. The composition of the invention may comprise mixtures of partial and fully esterified analogues. Some preferred carbohydrate esters include but are not limited to Lactose propionate, Lactose isobutyrate, Lactose benzoate, Raffinose benzoate, Raffinose isobutyrate, Sucrose isobutyrate, Trehalose isobutyrate and Sucrose benzoate, where each compound can be partially or fully functionalized/esterified or a mixture thereof. Moreover, some possible combinations are carbohydrate esters of Lactose isobutyrate, Lactose benzoate, Sucrose isobutyrate, or Sucrose benzoate in mixtures of partially or fully functionalized/esterified analogues, such as the mono, di, tri, quadro, penta, hexa, hepta and octa form, such as the quadro, penta, hexa, hepta and octa form such as the penta, hexa, hepta and octa form, such as but not limited to the penta, hexa, and hepta form. Furthermore, in some embodiments of the present invention, Sucrose benzoate comprising mixtures of the mono, di, tri, quadro, penta, hexa, hepta and octa form, such as the quadro, penta, hexa, hepta and octa form such as the penta, hexa, hepta and octa form, such as but not limited to the penta, hexa, and hepta form may replace SuBen in a depot (e.g. NCCell) composition and result in equal drug release rate, depot (e.g. NCCell) stability, injectability and general performance.

In some embodiments of the present invention, lactose octaisobutyrate comprising mixtures of the mono, di, tri, quadro, penta, hexa, hepta and octa form, such as the quadro, penta, hexa, hepta and octa form such as the penta, hexa, hepta and octa form, such as but not limited to the penta, hexa, and hepta form may replace LOIB in a depot (e.g. NCCell) composition and result in equal drug release rate, depot (e.g. NCCell) stability, injectability and general performance.

Furthermore, in some embodiments of the present invention, Sucrose octaisobutyrate comprising mixtures of the mono, di, tri, quadro, penta, hexa, hepta and octa form, such as the quadro, penta, hexa, hepta and octa form such as the penta, hexa, hepta and octa form, such as but not limited to the penta, hexa, and hepta form may replace SOIB in a depot (e.g. NCCell) composition and result in equal drug release rate, depot (e.g. NCCell) stability, injectability and general performance.

Similarly, carbohydrates esters and derivatives described herein may comprise mixtures of the mono, di, tri, quadro, penta, hexa, hepta and octa form, such as the quadro, penta, hexa, hepta and octa form such as the penta, hexa, hepta and octa form, such as but not limited to the penta, hexa, and hepta form.

Octa-substituted carbohydrates (e.g. SuBen, LacBen or lactulose octabenzoate) typically comprise 8 substitutions. However, it will be appreciated that synthesis of octa-substituted carbohydrates often yields both fully substituted and partly substituted carbohydrates (e.g. carbohydrates having 4, 5, 6, 7 or 8 substitutions, more typically 5, 6, 7 or 8 substitutions). Preparations of octa-substituted carbohydrates thus often contain a mixture of fully substituted and partly substituted carbohydrates, e.g. a mixture comprising carbohydrates having 4, 5, 6, 7 and 8 substitutions; a mixture comprising carbohydrates having 5, 6, 7 or 8 substitutions; a mixture comprising carbohydrates having 6, 7 or 8 substitutions; or a mixture comprising carbohydrates having 7 or 8 substitutions.

Similarly, undeca-substituted carbohydrates typically comprise 11 substitutions. However, it will be appreciated that synthesis of undeca-substituted carbohydrates also often yields both fully substituted and partly substituted carbohydrates (e.g. carbohydrates having 5, 6, 7, 8, 9, 10 or 11 substitutions, more typically 8, 9, 10 or 11 substitutions). Preparations of undeca-substituted carbohydrates thus often contain a mixture of fully substituted and partly substituted carbohydrates, e.g. a mixture comprising carbohydrates having 5, 6, 7, 8, 9, 10 or 11 substitutions; a mixture comprising carbohydrates having 6, 7, 8, 9, 10 or 11 substitutions; a mixture comprising carbohydrates having 7, 8, 9, 10 or 11 substitutions; a mixture comprising carbohydrates having 8, 9, 10 or 11 substitutions; a mixture comprising carbohydrates having 9, 10 or 11 substitutions; or a mixture comprising carbohydrates having 10 or 11 substitutions.

Carbohydrate esters described herein may be used alone or in combination.

### Inherent and Optional Imaging-Radiographic and Computed Tomography (CT) Contrast Agents and Fluorescence

Compositions of the invention may containing lipiodol as a CT contrast oil (co-solvent) or CLA-8 (α,β Lactose octa para-iodobenzoate) or xSAIB (6,6′-(2,4,6-triiodophenoxy) acetoxy-isobutyric-sucrose), which can serve as radiographic contrast agents. This is particularly advantageous because they may be used to guide placement of the composition in tissue, validate of correct placement, and/or act as fiducial markers for guiding therapeutic interventions, including, but not limited to, external beam radiotherapy or surgical procedures. Radiographic contrast may generally be used for CT, radiography or fluoroscopy and guide injection, installation, administration, and smearing of the composition or depot (e.g. NCCell). Alternative CT contrast agent are lactose or sucrose octa para or meta iodobenzoate. In addition to the optional radiographic contrast the composition or depot (e.g. NCCell) has inherent magnetic resonance contrast and can also be appreciated during ultrasound imaging following the absence of water in the composition. Inclusion of fluorescent molecules or nucleic acids encoding the transcription of peptide-based fluorophores can furthermore allow for identification of the composition or depot (e.g. NCCell) position and transcriptional activity by fluorescence imaging e.g., near infrared imaging (NIR) which is becoming increasingly applied for diagnostic and therapeutic interventions in clinical procedures.

In one embodiment, the nucleic acid component (e.g. mRNA, siRNA, pDNA or ASOs) is functionalized with one or more fluorophores such as Cy5, Cy7, Cy7.5 and the like and used for quantification of the biodistribution using for example optical imaging or spectroscopy.

In one embodiment, the nucleic acid component (e.g. mRNA, siRNA, pDNA or ASOs) is functionalized with one or more metal chelators such as DOTA, EDTA and DTPA and the like and used for quantification of the biodistribution using for example ICP-MS or ICP-AES/OES. A preferred embodiment utilises an ASO functionalized with DOTA loaded with gadolinium Gd.

Polymers: Compositions of the invention may comprise polymers which are hydrophobic substances that mix poorly or only partially with aqueous media. Depending on the hydrophobicity, the polymer may predominantly reside inside the depot or be presented at the surface of the depot. Block co-polymers comprising both a hydrophobic and a hydrophilic domain may also be presented at the surface of the depot. Polymers may further be functionalized with cell targeting or simulating ligand that can be presented at the interface of the depot. The polymers may stabilize the depot and provide mechanical stability of internal structures, such as water channels and voids.

Examples of polymers include but are not limited to polymers from the class polyethyleneimine (PEI), polylysine (PLL), polyarginine (PAA), Chitosans, Cellulose, Poly(2-ethyl-2-oxazoline) (ULTROXA), Diethylaminoethyl-dextran (DEAE-dextran), dendritic polyamidoamine (PAMAM) and PDMAEMA [poly(N,N-dimethylaminoethyl methacrylate], glycol (PEG), polylactic acid (PLA), poly(lactic-co-glycolic acid) (PLGA) and Poly(N-isopropylacrylamide) (PNIPAM) and the like.

Examples of polymers include but are not limited to Poly D,L lactide-co-glycolide, PLGA-PEG-PLGA, Poly lactic acid (PLA), poly(lactic-co-glycolic acid) (PLGA), PEG, Polycaprolactone, Polycaprolactone diol, Poly(methyl methacrylate) (PMMA), PVP (polyvinyl pyrrolidone), Poly-allylamine, bPEI (branched polyethylene imine), I-PEI (linear polyethylene imine), Poly(L-lactide) amine terminated, Trimethyl chitosan, Chitin (poly-N-acetyl glucosamine), Potassium hyaluronate (HA), Alpha tocopherol, Poly(propylene glycol) monobutyl ether, PCL-PEG-PCL, Polycaprolactone diol, PEg-OMe, Carrageenan, 2-Diethylamino-etyl cellulose (DEAE cellulose), Cellulose acetate (CA), Ethyl Cellulose (EC), Methyl Cellulose (ME), Cyanoethyl cellulose, Cellulose acetate propionate (CAP), Cellulose acetate phthalate (CaPh), Cellulose acetate butyrate (CAB), Cellulose propionate (CP), Cellulose acetate propionate (CAP), 2-Hydroxyethyl cellulose, Hydroxypropyl cellulose, (Hydroxypropyl)methyl cellulose, Sodium carboxymethyl cellulose and the like.

Examples of polymers include but are not limited to Poly D,L lactide-co-glycolide 75:25, 75-115 KDa, Ester terminated (PLGA ester). Poly D,L lactide-co-glycolide 75:25, 4-15 KDa, acid terminated (PLGA-COOH short). Poly D,L lactide-co-glycolide 75:25, 10-18 KDa, acid terminated (PLGA-COOH medium). Poly-L-lactide (PLA), 10-18 KDa, ester terminated (PLA ester medium). PLA, 18-28 KDa, ether terminated (PLA ether medium). PLA, 2 KDa. PLA, 50 KDa. Alpha-tocopherol PEG-1000 succinate (acid terminated) (VitE-PEG1K). Poly(propylene glycol) monobutyl ether (PPG), 2.5 KDa. PCL-PEG-PCL (1:1:1 KDa). Polycaprolactone diol (PCL Diol), 2 KDa. Polyethylene glycol methyl ether (PEg-OMe), 1KDa. PEG, 1.5 KDa. PEG (2000)-C18, 2 KDa. Polyethylene imine, branched (bPEI), 600 Da. bPEI, 1.2KDa. Cellulose acetate butyrate (CAB), 12KDa. CAB, 30 KDa. CAB, 70 KDa. Cellulose propionate (CP), 70 KDa. Celluose, acetate propionate (CAP), 75 KDa.

Preferred polymers are classes include but are not limited to PEI, CAB, PEG, PLA, PLGA and mixtures or block copolymers thereof.

Examples of polymers include but are not limited to bPEI (branched polyethylene imine), I-PEI (linear polyethylene imine), Ethyl Cellulose (EC), Cyanoethyl cellulose, Cellulose acetate propionate (CAP), Cellulose acetate phthalate (CaPh), Cellulose acetate butyrate (CAB), Cellulose propionate (CP), Cellulose acetate propionate (CAP), 2-Hydroxyethyl cellulose, Hydroxypropyl cellulose, (Hydroxypropyl)methyl cellulose, Sodium carboxymethyl cellulose and the like.

### Other Constituents of Compositions of the Invention

Additional constituents of compositions of the invention include lipids, detergents, surfactants and polymers and the like that may be included for additional benefits. Such constituents may i) provide altered and improved interaction of the composition or depot interface with tissues, ii) impact the surface chemistry of the depot, iii) protect or stabilize the nucleic acid component inside the composition or depot, iv) be released and work as enhancers of transfection or v) modulate the composition or depot materials and thereby act as tuning agents for tailoring release of nucleic acid component e.g. transfection systems.

Interaction with tissues may be modulated by inclusion of amphipathic polymers or lipids that enable presentation of polymers like PEG, PEI, PLL, PAA, Chitosans and the like on the depot (e.g. NCCell) interface. Alternatively, inclusion of charged lipids such as, but not limited to phosphatidylglycerol (PG), phosphatidylserine (PS)phospholipids, phosphatidic acid (PA), charged sterols such as CHEMS and DC-Chol, lysolipids and the like may further modify the depot (e.g. NCCell) interface from being hydrophobic in nature to hydrophilic and compatible with tissues. Such amphipathic molecules will be presented on depots (e.g. NCCells) interface with water or tissues as this interface resemble an oil-water interface where they are known to accumulate. Cell signal molecules such as RGD, cRGD, cyclic peptidomimetic compounds, Integrin binding peptides, and the like may be presented on the depot (e.g. NCCell) interface via coupling to PEGylated or non-pegylated lipids such as DSPE-PEG2k-RGD, DSPE-RGD and the like or via attachment to amphipathic polymers that are presented on the surface of depot (e.g. NCCell).

Inclusion of surfactant, lipids and surface active polymers, that upon injection of depot (e.g. NCCell) into aqueous media or tissues are presented on its surface, will reduce the interfacial tension of depot (e.g. NCCell) and thereby change its wetting behaviour upon application. As the interfacial tension is diminished, the restoring force for coalescence of depot (e.g. NCCell) is likewise reduced, which may lead to easier spreading and covering of tissues with depot (e.g. NCCell).

Tuning of the depot (e.g. NCCell) surface properties by lipids, surfactant and or polymers may also impact the release properties of nucleic acid complexes such as LNPs, lipoplexes, polyplexes and HIP polyplexes from depot (e.g. NCCell). Addition of lipids such as POPC, Cholesterol, DOTAP and the like have effect on the release rate as demonstrated in examples 21 and 37.

Lipids and polymers may further attribute to the stability of the nucleic acid component, e.g. incorporated transfection particles or nucleic acid HIP complexes. Inclusion of additional cationic, helper or PEGylated lipids before incorporation of a lipoplex may prevent solubilization and stabilize lipoplexes composed of these lipids in the depot (e.g. NCCell) formulation. Inclusion of additional cationic polymer such as but not limited to PEI will likewise prevent solubilization and stabilize polyplexes based on PEI-polymers.

Transfection enhancers such as PEI may additionally be included for co-release and stimulation of enhanced transfection.

Compositions of the invention have a lower viscosity in a hydrophobic environment than in an aqueous environment. The lower viscosity makes compositions of the invention ideally suited to administration e.g. by injection or catheterization. Upon transfer to an aqueous environment (e.g. following injection into the body of a human or animal), the composition becomes more viscous, i.e. it goes through a sol-gel transition (liquid to gel) transition, due to the presence of the hydrophobic component. In some embodiments, the viscosity increases by at least 50%, such as at least 80%, such as at least 100%, or at least 150%, or at least 200%, or at least 300%, or at least 500%, or at least 750%, or at least 1000%, or at least 10,000%, or the formulation becomes essentially solid (non-viscous). The composition is preferably adapted for injection via a thin needle used for injection into a body or surgical related procedures, such as but not limited to biopsy. The viscosity of the composition before injection can be any suitable viscosity such that the formulation can be parenterally administered to a patient. Exemplary compositions include, but are not limited to, those having a viscosity (prior to administration/injection) lower than 10,000 centipoise (cP), e.g. lower than 2,000 cP, such as 10 to 2,000 cP, such as 20 to 1,000 cP, such as 150 to 350 cP, such as 400 to 600 cP, such as 600 to 1,200 cP or such as 1,000 to 2,000 cP, or 10 to 600 cP, or 20 to 350 cP, at 20° C. Alternative compositions include, but are not limited to, those having a viscosity (prior to administration/injection) lower than 10,000 centipoise (cP), e.g. lower than 2,000 cP, such as 10 to 2,000 cP, such as 20 to 1,000 cP, such as 150 to 350 cP, such as 400 to 600 cP, such as 600 to 1,200 cP or such as 1,000 to 2,000 cP, or 10 to 600 cP, or 20 to 350 cP, at 5° C. When referred to herein, the (dynamic) viscosity is measured at the specified temperature in accordance with the method described in ASTM D7483.

### Therapeutic Nucleic Acids

The present invention can generate biological therapeutics with specific end products that are dictated by the nucleic acid sequence or sequences delivered therefrom. This provides possibilities to generate biologically optimal therapeutic polypeptides which in combination with the complete sequenced genome of human and most animal species is a very potent therapeutic tool. The technology is not restricted to biological therapeutics with e.g., signal transducing function, agonist or antagonists for various pathways, cell surface receptors, but also disease associated antigens induced or expressed by e.g., virus and cancer cells. The technology can also provide therapeutic polypeptides that replace diseases associated proteins produced in connection with chromosomal and genetic diseases.

The present invention can also deliver nucleic acids that regulate gene expression through gene silencing. Gene silencing is the regulation of gene expression in a cell to prevent the expression of one certain or multiple genes. Gene silencing is being increasingly used to produce therapeutics to combat cancer and other diseases, such as infectious diseases and neurodegenerative disorders. During gene silencing the antisense nucleic acid product, classically a double stranded pairs with a complementary sequence in a mRNA molecule and induces cleavage and degradation. Alternatives to double stranded siRNAs of regulation of gene expression includes antisense ASO, miRNA, but also synthetic Chimeric oligonucleotides containing phosphodiester and phosphorothioate linkages.

To achieve the clinical potential of nucleic acid-based therapeutics these must be efficiently transported to the cells of target tissues. However, there are various barriers that must be fixed before it can be used clinically. For example, “naked” nucleic acids are susceptible to several obstacles that reduce their therapeutic efficacy. The most effective methods to transport nucleic acids into cell are though nanoparticulate systems, including liposomes, polymer nanoparticles, polyplexes, SLNP, lipoplexes, lipopolyplexes. Alternative strategies includes the use of cell penetrating peptides, targeting cell internalizing receptors, and to use of synthetic nucleic acids (e.g., PS-oligos) that can enter cells without carrier technology.

Nucleic acid delivery according to the invention therefore provides a novel method to secure sustained and biologically adapted therapy capable of inducing gene transcription and silencing. The present invention provides complete flexibility towards nucleic acid combinations that can be included and released. This allows for multigene transcription and silencing and combinations hereof. Importantly, the flexible delivery can also provide sustained delivery of aptamers. Aptamers are single-stranded oligonucleotides (DNA or RNA) that fold into defined architectures and bind to targets such as proteins. In binding proteins, they often inhibit protein-protein interactions and thereby may elicit therapeutic effects such as antagonism. Importantly, most aptamers also demonstrate a cell-internalizing property in native living cells, allowing them to directly enter the cells via endocytosis depending on the target.

Nucleic acid degradation is a central obstacle for sustained delivery technologies for gene-engineering. RNA is inherently less stable than DNA due to its chemical structure, however, degradation of both DNA and RNA must be prevented in the optimal delivery system in order to assure sustained biological activity. Strategies to stabilize gene engineering technologies have been achieved synthetic modification. These have however been associated with challenges in connection to tolerability and melting point, which affects binding affinity. Embedding nucleic acids such as transfection complexes (e.g. LNPs, polyplexes, lipoplexes, HIP complexes and the like) in hydrophobic components and/or depots of the invention reduce their contact with water and thereby the rate of hydrolysis. Transfer to organic media further shields the nucleic acids of the transfection systems from enzymatic degradation such as by nucleases, DNAses and RNAses. The reduction in hydrolytic and enzymatic activity both contribute to enhanced storage stability of the transfection agent as well as increased stability in vivo before the transfection system is released from the depot. The latter is highly important for maintaining sustained transfection without degradation of the transfection system before it has been released.

In addition to providing sustained release of nucleic acid component for cellular delivery the present invention can also release molecular therapeutics including, proteins, peptides, small molecules. This feature is very useful in vaccination where the vaccine encoding nucleic acid sequence may be co-delivered with an optimal adjuvant or adjuvant combination. This may be used to secure not only optimal antigen presentation and immune activation but also maintain an effective and optimal immunological effect e.g., memory. The sustained release properties of antigen encoding nucleic acids and adjuvant may therefore alleviate the need for repeated vaccinations but secure single dose protection. In the cases of vaccination against cancer this is of particular interest as this would allow for the formation of an optimal vaccination against the specific sequences delivered. In case of intratumoral or peritumoral administration the optimal adjuvant combination can also secure reversal of the immunosuppressive tumor microenvironment to provide effective cancer elimination, secure response maturation and epitope spreading of the immunological response.

The combination of nucleic acid-based gene engineering and molecule-based free therapeutics in the delivery system of the invention has indications far beyond cancer, including but not limited to metabolic disorders, non-vaccine based cancer immunotherapy, combination of antibody based and chemotherapy based cancer immunotherapy, tissue regeneration, tissue reengineering, infections and immune modulation (e.g., antibiotics in combination with nucleic acids encoding tissue regenerative factors for chronic wounds, diabetic ulcers, non-healing bone diseases, stem cell therapies).

Gene engineering using the sustained delivery properties of the invention has broad therapeutic indications. The majority of therapies require time to generate the optimal outcome and the continuous release of nucleic acid component helps achieve a biologically optimal therapeutic intervention. Central in this is the controllable release kinetics which reduce dose fluctuation and thereby associated side- and adverse effects. The present invention is therefore biologically optimal for immunotherapy (e.g., cancer and infections, including vaccination), metabolic disorders, enzyme replacement, hormonal disease management (e.g., osteoporosis), genetic diseases (e.g., storage diseases), regenerative disease, autoimmune disorders, tissue engineering, cell therapies (e.g., pro-survival factors and polarization of stem cell therapies, adoptive cell therapies, chimeric cell products), transplantation (autologous, allogenic and xeogenic transplants for securing transplant survival, host-graft interaction, adaption and function).

A wide spectrum of therapeutic effects can be achieved by encoding or silencing genes using the present invention. Non-limiting examples include antineoplastic activity, anti-infective activity, antimicrobials activity, local and systemic anti-allergic activity, anti-anemic activity, beta-adrenergic activity, calcium channel activity, anti-hypertensive activity, glucagon like peptide activity, insulin activity, metabolite activity, anti-depressant activity, angiogenic activity, anti-angiogenic activity, growth factor activity, anticonvulsant activity, anti-bacterial activity, anti-fungal activity, anti-viral activity, anti-rheumatic activity, anthelminithic activity, anti-parasitic agent activity, corticosteroid activity, hormone activity, immunomodulating activity, neurotransmitter activity, anti-diabetic activity, statin activity, lipid-lowering activity, activity that reduce illness and mortality in those who are at high risk of cardiovascular disease (e.g., HMG-CoA reductase inhibitors), anti-epileptic activity, anti-haemorrhagic activity, anti-hypertonic activity, antiglaucoma activity, immunomodulatory cytokine activity, sedative activity, chemokine activity, vitamin activity, narcotic activity, wound repair activity, tissue repair activity, tissue engineering, transplantation medicine and combinations thereof of nucleic acid-based on gene engineering delivered by the present invention. Compositions of the invention may be injected, smeared, administered or installed in both soft tissues and bone, non-limiting examples includes sub-cutaneous, intramuscular, intradermal, intranodal, intraosseous, in organs, intratumoral, in infected tissues, in tissue defects, in scars, in wounds, smearing in surgical sites and surgical beds, in and around athroplastics, in and around implants, peritendinous, peri- and intraarticular, on mucosal and serosal surfaces, in wounds and on wound surfaces, in abscesses, phlegmons and body cavities e.g., intraperitoneal, intrathoracic, intravesical, intrauterine, intranasal, in sinuses, in the inner, middles and outer ear and on the skin and body surface.

The dosage ranges for compositions and depots of the invention (e.g. NCCells) are those suitable to produce the desired therapeutic effect. It will be appreciated that the dosage range required depends on the precise nature of the nucleic acid, the age of the patient, the nature, extent or severity of the patient's condition, contraindications, if any, and the judgement of the attending physician. Variations in dosage levels can be adjusted using standard empirical routines for optimisation.

The composition of the invention may be given in a single dose schedule. Alternatively, the pharmaceutical compositions of the invention may be given in a multiple dose schedule. A multiple dose schedule is one in which a primary course of treatment may be with 1-6 separate doses, followed by other doses given at subsequent time intervals required to maintain and/or reinforce the therapeutic activity.

Although the present invention is described in detail herein, it is to be understood that this invention is not limited to the particular methodologies, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

In one embodiment, a polyplex is formed from a nucleic acid and a cationic polymer, and the polyplex is solubilized or dispersed in a composition or depot of the invention e.g. an NCCell.

In one embodiment, a polyplex is formed from nucleic acid and a cationic polymer from but not limited to the class of polyethyleneimine (PEI), polylysine (PLL), polyarginine (PAA), Chitosans, Poly(2-ethyl-2-oxazoline) (ULTROXA), Diethylaminoethyl-dextran (DEAE-dextran), dendritic polyamidoamine (PAMAM), Poly-beta-amino-esters (PBAE) and PDMAEMA [poly(N,N-dimethylaminoethyl methacrylate] or mixtures thereof, and the polyplex is solubilized or dispersed in a composition or depot of the invention e.g. an NCCell.

In one embodiment, a polyplex is formed from a nucleic acid and a cationic polymer from the class of branched or linear polyethyleneimine (PEI), polylysine (PLL), polyarginine (PAA) with a preferred molecular weight of 2-100 kDa or more preferred molecular weight of 15-50 kDa, and the polyplex is solubilized or dispersed in a composition or depot of the invention e.g. an NCCell.

In one embodiment, a polyplex is formed from a nucleic acid and a branched or linear polyethyleneimine (PEI) with a preferred molecular weight 2-100 kDa and NP ratio 5-100 or more preferred a molecular weight of 15-50 kDa and NP ratio 10-50, and the polyplex is solubilized or dispersed in a composition or depot of the invention e.g. an NCCell.

In a preferred embodiment, a polyplex is formed from a nucleic acid and linear polyethyleneimine (PEI) with 25 kDa or 40 kDa molecular weight and at a NP ratio of 10-25, and the polyplex is solubilized or dispersed in a composition or depot of the invention e.g. an NCCell.

In one embodiment, a polyplex is formed from but not limited to a nucleic acid and a branched or linear polyethyleneimine (PEI), and the polyplex is solubilized or dispersed in a composition or depot of the invention (e.g. an NCCell), comprising transfection enhancing or transfection particle stabilizing agents such as but not limited to polyethyleneimine (PEI).

In one embodiment, a polyplex is formed from but not limited to a nucleic acid and a PAMAM, and the polyplex is solubilized or dispersed in a composition or depot of the invention (e.g. an NCCell), comprising transfection enhancing or transfection particle stabilizing agents such as but not limited to PAMAM. In one embodiment, PAMAM is Gen 0.0. In one embodiment, PAMAM is Gen 1.0.

In one embodiment, a polyplex is formed from a nucleic acid and a cationic polymer, and the polymer is functionalized with the targeting ligands such as but not limited to carbohydrates, Galactose, Mannose, peptides, RGD, cRGD, GalNAc or mixtures thereof, and the polyplex is solubilized or dispersed in a composition or depot of the invention e.g. an NCCell. In one embodiment, a polyplex is formed from a nucleic acid and a cationic polymer, and the polymer is functionalized with the targeting ligand mannose, and the polyplex is solubilized or dispersed in a composition or depot of the invention e.g. an NCCell.

In one embodiment, a polyplex is formed from a cationic polymer and a nucleic acid, and the nucleic acid is functionalized with the targeting ligands such as but not limited to carbohydrates, Galactose, Mannose, peptides, RGD, cRGD, GalNAc or mixtures thereof, and the polyplex is solubilized or dispersed in a composition or depot of the invention e.g. an NCCell.

In one embodiment, a polyplex is formed from a nucleic acid and a branched or linear polyethyleneimine (PEI), and PEI is functionalized with the targeting ligands such as but not limited to carbohydrates, Galactose, Mannose, peptides, RGD, cRGD, GalNAc or mixtures thereof, and the polyplex is solubilized or dispersed in a composition or depot of the invention e.g. an NCCell. In one embodiment, a polyplex is formed from a nucleic acid and a branched or linear PEI, and PEI is functionalized with mannose.

In one embodiment, a polyplex is formed from a nucleic acid and a branched or linear polyethyleneimine (PEI), and PEI is functionalized with polymers such as but not limited to polyethylene glycol (PEG), polylactic acid (PLA), poly(lactic-co-glycolic acid) (PLGA) and Poly(N-isopropylacrylamide) (PNIPAM) or mixtures thereof, and the polyplex is solubilized or dispersed in a composition or depot of the invention e.g. an NCCell.

In one embodiment, a polyplex is formed from a nucleic acid and a branched or linear polyethyleneimine (PEI), and PEI is functionalized with lipids such as but not limited to aliphatic or aromatic C3-C22 acyl or alkyl groups, sterols, esterified carbohydrates, PE phospho- or lyso-phospholipids or mixtures thereof, and the polyplex is solubilized or dispersed in a composition or depot of the invention e.g. an NCCell.

In one embodiment, a polyplex is formed from a nucleic acid and PAMAM. In one embodiment, PAMAM is functionalized with the targeting ligands such as but not limited to carbohydrates, Galactose, Mannose, peptides, RGD, cRGD, GalNAc or mixtures thereof, and the polyplex is solubilized or dispersed in a composition or depot of the invention e.g. an NCCell.

In one embodiment, PAMAM is a PAMAM-Folate Dendrimer. Folic acid or folate ligands can be attached to PAMAM dendrimers to enable targeting of folate receptor-expressing cancer cells. Folate receptors are overexpressed in various cancer types, making this targeting moiety useful for cancer-specific delivery.

In one embodiment, PAMAM is a PAMAM-Transferrin Dendrimer. Transferrin ligands can be conjugated to PAMAM dendrimers to target cancer cells that overexpress transferrin receptors. Transferrin receptors play a role in iron uptake and are often upregulated in cancer cells.

In one embodiment, PAMAM is a PAMAM-RGD Dendrimer. RGD (Arg-Gly-Asp) peptides, known for their affinity to integrin receptors, can be attached to PAMAM dendrimers. Integrins are involved in cell adhesion and are overexpressed in tumor cells, making RGD a valuable targeting ligand for cancer therapy.

In one embodiment, PAMAM is a PAMAM-EGF Dendrimer. Epidermal growth factor (EGF) ligands can be conjugated to PAMAM dendrimers to target cancer cells that overexpress the EGF receptor (EGFR). EGFR is often upregulated in various cancers and plays a role in cell growth and proliferation.

In one embodiment, PAMAM is a PAMAM-Hyaluronic Acid Dendrimer. Hyaluronic acid (HA) can be attached to PAMAM dendrimers for targeting CD44 receptors, which are overexpressed in many cancer cells and involved in tumor progression and metastasis. HA is a natural ligand for CD44 receptors.

In one embodiment, PAMAM is a PAMAM-Arginine-Glycine-Aspartic Acid (RGD) Dendrimer. The RGD peptide sequence, which has affinity for integrin receptors, can be incorporated into PAMAM dendrimers. Integrin receptors are present in angiogenic blood vessels and can be targeted for anti-angiogenic therapies.

In one embodiment, PAMAM is a PAMAM-Transactivating Transcriptional Activator (TAT) Dendrimer. The TAT peptide, derived from the human immunodeficiency virus (HIV), can be attached to PAMAM dendrimers to enable cell-penetrating properties. TAT peptide facilitates efficient cellular uptake and can enhance drug delivery to various cell types.

In one embodiment, PAMAM is a PAMAM-LDL Receptor-Targeting Dendrimer. Low-density lipoprotein (LDL) receptor-targeting ligands, such as ApoB or LDL-like peptides, can be conjugated to PAMAM dendrimers for selective targeting of cells expressing LDL receptors. LDL receptors are involved in cholesterol metabolism and are present in certain tumor cells.

In one embodiment, PAMAM is a PAMAM-Neuropilin-1 Dendrimer. Neuropilin-1 (NRP-1) is a receptor that plays a role in tumor angiogenesis. PAMAM dendrimers can be functionalized with NRP-1 targeting ligands to specifically deliver therapeutics to NRP-1 expressing cells.

In one embodiment, PAMAM is a PAMAM-Mannose Dendrimer. Mannose ligands can be attached to PAMAM dendrimers for targeting cells expressing mannose receptors, such as macrophages or dendritic cells. This targeting moiety is useful for applications in immunotherapy and vaccine delivery.

In one embodiment, one or multiple different polyplexes are solubilized or dispersed in a composition or depot of the invention e.g. an NCCell.

In one embodiment, a lipoplex is formed from a nucleic acid and lipids comprising either single or mixtures of cationic-, helper/structural- and PEGylated lipids, and the lipoplex is solubilized or dispersed in a composition or depot of the invention e.g. an NCCell.

In one embodiment, a LNP is formed from a nucleic acid and lipids, and the LNP is solubilized or dispersed in a composition or depot of the invention e.g. an NCCell. In one embodiment, the LNP comprises a neutral lipid; a cationic lipid; a sterol, e.g. cholesterol; and a PEG or PEG-modified lipid.

In one embodiment, a LNP or lipoplex is formed from a nucleic acid and a lipid mixture comprising 20-60 mol % cationic lipids, 0-60 mol % helper/structural lipid and 0-10 mol % PEGylated lipids, and the LNP or lipoplex is solubilized or dispersed in a composition or depot of the invention e.g. an NCCell.

In one embodiment, a LNP or lipoplex with NP 1-50 or more preferred NP 5-25, or yet more preferred NP 5-15 is formed from a nucleic acid and a lipid mixture comprising 20-60 mol % cationic lipids, 0-60 mol % helper/structural lipid and 0-10 mol % PEGylated lipids, and the LNP or lipoplex is solubilized or dispersed in a composition or depot of the invention e.g. an NCCell.

In one embodiment, a LNP or lipoplex is formed from a nucleic acid and lipids, where single or multiple cationic lipids are selected from the list: DC-chol, DMTAP, DPTAP, DSTAP, DOTAP, DOTMA, DOSPA, DDAB, DMDAP, DPDAP, DSDAP, DODAP, DODMA, DOBAQ, Dlin-DMA, Dlin-KC2-DMA, Dlin-MC3-DMA, C12-200, A6, OF-02, A18-Iso5-2DC18, YSK05, 7C1, GO-C14, L319, OF-Deg-Lin, 306-012B, 3060110, FTT5, 9A1P9, 98N12-5, 304013, cKK-E12, Spermine-chol, and MVL5, and the LNP or lipoplex is solubilized or dispersed in a composition or depot of the invention e.g. an NCcell.

In a preferred embodiment, a LNP or lipoplex is formed from a nucleic acid and lipids, where single or multiple cationic lipids are selected from the list: DC-chol, DOTAP, Dlin-DMA, Dlin-KC2-DMA and Dlin-MC3-DMA, and the LNP or lipoplex is solubilized or dispersed in a composition or depot of the invention e.g. an NCCell.

In one embodiment, a LNP or lipoplex is formed from a nucleic acid and lipids, where helper/structural lipids are selected from the class of sterols such as but not limited to cholesterol, lanosterol, ergosterol and the like, C12-C18 acyl saturated and unsaturated phosphatidylcholine (PC) or Phosphatidylethanolamine (PE) or Phosphatidylglycerol phospholipids such as DLPC, DLPE, DLPG, DMPC, DMPE, DMPG, DPPC, DPPE, DPPG, DSPC, DSPE, DSPG, DOPC, DOPE and DOPG, and the LNP or lipoplex is solubilized or dispersed in a composition or depot of the invention e.g. an NCCell.

In a preferred embodiment, a LNP or lipoplex is formed from a nucleic acid and lipids, where helper/structural lipids are selected from the list of cholesterol, DSPC, DSPE, DSPG, DOPC, DOPE and DOPG and PEGylated lipids, and the LNP or lipoplex is solubilized or dispersed in a composition or depot of the invention e.g. an NCCell.

In one embodiment, a LNP or lipoplex is formed from a nucleic acid and lipids, where PEGylated lipids are selected from the list of DLPE, DMPE, DPPE, DSPE, DOPE phospholipids PEGylated with either PEG350, PEG500, PEG750, PEG1k, PEG2k, PEG3k or PEG5k, sterols such as cholesterol PEGylated with either PEG350, PEG500, PEG600, PEG750, PEG1k, PEG2k, PEG3k or PEG5k or 1,2-Dimyristoyl-rac-glycero-3-methoxypolyethylene glycol-2000 (DMG-PEG2k), Distearoyl-rac-glycerol-PEG2K (DSG-PEG2k), and the LNP or lipoplex is solubilized or dispersed in a composition or depot of the invention e.g. an NCCell.

In a preferred embodiment, a LNP or lipoplex is formed from a nucleic acid and lipids, where PEGylated lipids are selected from list of 1,2-Dimyristoyl-rac-glycero-3-methoxypolyethylene glycol-2000 (DMG-PEG2k), Distearoyl-rac-glycerol-PEG2K (DSG-PEG2k), DMPE-PEG2k, DSPE-PEG2k or mixtures thereof, and the LNP or lipoplex is solubilized or dispersed in a composition or depot of the invention e.g. an NCCell.

In a preferred embodiment, a LNP or lipoplex is formed from a nucleic acid and lipid mixtures comprising DC-Chol, Chol, DOPE or DSPC and DMG-PEG2k or DMPE-PEG2k, and LNP or lipoplex is solubilized or dispersed in a composition or depot of the invention e.g. an NCCell.

In a preferred embodiment, a LNP or lipoplex is formed from a nucleic acid and lipid mixtures comprising 20-60 mol % DC-Chol, 40-80 mol % Chol, 1-10 mol % DOPE and 0-10 mol % DMG-PEG2k, or more preferred 20-40 mol % DC-Chol, 50-70 mol % Chol, 1-5 mol % DOPE and 0-5 mol % DMG-PEG2k, and LNP or lipoplex is solubilized or dispersed in a composition or depot of the invention e.g. an NCCell.

In a preferred embodiment, a LNP or lipoplex is formed from a nucleic acid and lipid mixtures comprising 30 mol % DC-Chol, 65 mol % Chol and 5 mol % DOPE, and LNP or lipoplex is solubilized or dispersed in a composition or depot of the invention e.g. an NCCell.

In a preferred embodiment, a LNP or lipoplex is formed from a nucleic acid and lipid mixtures comprising Dlin-KC2-DMA, Chol and DOPC or DSPC and DMG-PEG2k or DMPE-PEG2k, and LNP or lipoplex is solubilized or dispersed in a composition or depot of the invention e.g. an NCCell.

In a preferred embodiment, a LNP or lipoplex is formed from a nucleic acid and lipid mixtures comprising 30-70 mol % Dlin-KC2-DMA, 20-60 mol % Chol, 2-20 mol % DSPC and 0-10 mol % DMG-PEG2k, or more preferred 40-60 mol % Dlin-KC2-DMA, 30-50 mol % Chol, 5-15 mol % DSPC and 0-5 mol % DMG-PEG2k, and LNP or lipoplex is solubilized or dispersed in a composition or depot of the invention e.g. an NCCell.

In yet a preferred embodiment, a LNP or lipoplex is formed from a nucleic acid and lipid mixtures comprising 50 mol % Dlin-KC2-DMA, 40 mol % Chol and 10 mol % DSPC, and LNP or lipoplex is solubilized or dispersed in a composition or depot of the invention e.g. an NCCell.

In a preferred embodiment, a LNP or lipoplex is formed from a nucleic acid and lipid mixtures comprising DC-Chol, Dlin-KC2-DMA, Chol and DOPC or DSPC and DMG-PEG2k, DMPE-PEG2k and LNP or lipoplex is solubilized or dispersed in a composition or depot of the invention e.g. an NCCell.

In a preferred embodiment, a LNP or lipoplex is formed from a nucleic acid and lipid mixtures comprising 15-35 mol % DC-Chol, 15-35 mol % Dlin-KC2-DMA, 20-60 mol % Chol, 2-20 mol % DSPC and 0-10 mol % DMG-PEG2k, or more preferred 20-30 mol % DC-Chol, 20-30 mol % Dlin-KC2-DMA, 30-50 mol % Chol, 5-15 mol % DSPC and 0-5 mol % DMG-PEG2k, and LNP or lipoplex is solubilized or dispersed in a composition or depot of the invention e.g. an NCCell.

In yet a preferred embodiment, a LNP or lipoplex is formed from a nucleic acid and lipid mixtures comprising 25 mol % DC-Chol, 25 mol % Dlin-KC2-DMA, 40 mol % Chol and 10 mol % DSPC, and LNP or lipoplex is solubilized or dispersed in a composition or depot of the invention e.g. an NCCell.

In a preferred embodiment, a LNP or lipoplex is formed from a nucleic acid and lipid mixtures comprising DOTAP and Chol and LNP or lipoplex is solubilized or dispersed in a composition or depot of the invention e.g. an NCCell.

In a preferred embodiment, a LNP or lipoplex is formed from a nucleic acid and lipid mixtures comprising 25-75 mol % DOTAP and 25-75% Chol, or more preferred 40-60 mol % DOTAP, 40-60 mol % Chol and LNP or lipoplex is solubilized or dispersed in a composition or depot of the invention e.g. an NCCell.

In a preferred embodiment, a LNP or lipoplex is formed from a nucleic acid and lipid mixtures comprising 50 mol % DOTAP, 50 mol % Chol, and LNP or lipoplex is solubilized or dispersed in a composition or depot of the invention e.g. an NCCell.

In one embodiment, a LNP or lipoplex is formed from a nucleic acid and lipids comprising either single cationic-, helper-, PEGylated-lipids and lipids functionalized with cell targeting ligands and the LNP or lipoplex is solubilized or dispersed in a composition or depot of the invention e.g. an NCCell.

In one embodiment, a LNP or lipoplex is formed from lipids comprising either single cationic-, helper-, PEGylated-lipids and nucleic acid functionalized with cell targeting ligands and the LNP or lipoplex is solubilized or dispersed in a composition or depot of the invention e.g. an NCCell.

In one embodiment, one or multiple LNPs or lipoplexes coding for different targets are solubilized or dispersed in a composition or depot of the invention e.g. an NCCell.

In one embodiment, one or multiple polyplexes coding for different targets are solubilized or dispersed in a composition or depot of the invention e.g. an NCCell.

In one embodiment, one or multiple polyplexes and or LNPs or lipoplexes coding for different targets are solubilized or dispersed in a composition or depot of the invention e.g. an NCCell.

In one embodiment, a HIP is formed from a nucleic acid and counterions, and the HIP is solubilized or dispersed in a composition or depot of the invention e.g. an NCCell.

In one embodiment, a HIP is formed from a nucleic acid and counterions containing one or more ionizable groups, and the HIP is formulated said solubilized in a composition or depot of the invention e.g. an NCCell.

In one embodiment, a HIP is formed from a nucleic acid and hydrophobic counterions comprising one or multiple hydrophobic domains, and the HIP is formulated said solubilized in a composition or depot of the invention e.g. an NCCell.

In one embodiment, a HIP is formed from a nucleic acid and one or combination of multiple counterion, and the HIP is formulated said solubilized in a composition or depot of the invention e.g. an NCCell.

In one embodiment, a HIP is formed from a nucleic acid and counterions at different charge ratios, said NP ratios, and the HIP is formulated said solubilized in a composition or depot of the invention e.g. an NCCell.

In one embodiment, a HIP is formed from a nucleic acid and counterions at NP ratio 1-20, or more preferred an NP ratio of 1-10, or yet more preferred and NP ration of 1-5 and the HIP is formulated said solubilized in a composition or depot of the invention e.g. an NCCell.

In one embodiment, HIP complexes comprise hydrophobic counterions selected from the class of ionic detergents, ionic lipids and lysolipids, fatty acids and ionic polymers.

In one embodiment, HIP complexes comprise hydrophobic counterions with one or multiple ionizable groups belonging to the group of amines (primary, secondary, tertiary or quaternary), alcohols, carboxylic acids, thiols, sulphonic, sulphenic or sulfinic acids, tetraalkyl phosphonium, phosphonic, phosphenic, or phosphinic acids, conjugated systems with delocalized charges, and the like.

In one embodiment, HIP complexes comprise hydrophobic counterions with one or multiple ionizable groups belonging to the group of alcohols, carboxylic acids, amines, organophosphorus compounds, or organosulfur compounds functionalized with one or multiple aliphatic or aromatic C4-C22 hydrocarbon chains, or preferable functionalized with one or multiple aliphatic or aromatic C8-C16 hydrocarbon chains, or yet more preferable functionalized with one or multiple aliphatic C8-C12 hydrocarbon chains.

In one embodiment, HIP complexes comprise hydrophobic counterions with one or multiple ionizable groups belonging to the group of alcohols, carboxylic acids, amines, organophosphorus compounds, or organosulfur compounds functionalized with one or multiple aliphatic or aromatic C4-C22 hydrocarbon chains, such as but not limited to methanol, ethanol, propanol, butanol, hexanol, octanol, decanol, dodecanol, phenol, hydroquinone, catechol, resorcinol, and the like. Acid Acetic acid, propanoic acid, butanoic acid, hexanoic acid, octanoic acid, benzoic acid, Myristic acid, Myristoleic acid, Palmitic acid, Palmitoleic acid, Stearic acid, Oleic acid, Elaidic acid, Linoleic acid, Linolenic acid, Linolelaidic acid, Arachidonic acid, Elauricacid acid, Mycolic Acids, α-mycolic acid (C80), α-mycolic acid, methoxy cis, α-mycolic acid, keto cis, Cyclopropyl Lipids, Cis-9,10-methylenehexadecanoic acid, 1-palmitoyl-2-cis-9,10-methylenehexadecanoyl-sn-glycero-3-phosphocholine, 1-palmitoyl-2-cis-9,10-methylenehexadecanoyl-sn-glycero-3-phosphoethanolamine, (s)-12-methyltetradecanoic acid, 13-methyltetradecanoic acid, cis-9-oleicacid, cis-9-octadecenoicacid, trans-9-octadecenoicacid, (2-16)-octadecenoicacid,, 2-tridecenoic acid, 11-tridecenoic acid, 12 tridecenoicacid, 2-dodecenoicacid, 5-dodecenoicacid, 6-dodecenoic acid, 7-dodecenoic acid, 9-dodecenoic, 10-dodecenoic acid, 11-dodecenoic acid, 11-eicosenoic acid, 14-eicosenoic acid, 2-undecenoic acid, 6-undecenoic acid, 9-undecenoic acid, 10 undecenoicacid, 2-decenoicacid, 3-decenoicacid, 8-decenoicacid, 9-decenoicacid, acrylic acid, A-methylacrylic acid, vinyl acetic acid, b-b-dimethylacrylic acid, beta-pentenoic acid, alylacetic acid, angelic acid, tiglic acid, 2-heptadecenoic, 9-hep-tadecenoic acid, 2-hexadecenoic acid, 9-hexadecenoicacid (cis form); 2-tetradecenoicacid, 4-tetradecenoicacid, radecenoicacid, 8-tetradecenoicacid; 9-tetradecenoic acid;2-nonenoicacid, 3-nonenoicacid, 8-nonenoicacid, 2-octenoicacid, 3-octenoicacid, 7-octenoicacid, 2-hep tenoicacid, 3-heptenoicacid;4-heptenoicacid, 5-hep tenoicacid, 6-heptenoicacid, 2-hexenoicacid, 3-hex Enoicacid, 4-hexenoicacid, 5-hexenoicacid, 15-tet-racosenoicacid; 17-hexacosenicacid, 21-triacentenoic acid, 2- to 8-nonynoic acid, Octadecadienoic acid 8:10, oleic acids (i.e., cis-9-oleicacidorcis-9-octadecenoicacid), octadecadienoicacid, (8-trans and 10-transforms), 8:11 acidsiselaidicacid (i.e., trans-9-octadecenoicacid), octadecadienoicacid, (8-cis and 11-cis forms), 9:11 octadecadienoicacid, (9-cis and 11-cis and I-trans forms), 5:12-octadecadienoic acid, (5-cis, 5-trans, 12 trans and 12-cisforms), 9:12-octadecadienoic acid, (9-cis, 9-trans, 12-trans and 12-cis forms), 10:12 octadecadienoic acid, (10-cis, 10-trans, 12-cis and 12 trans forms), 10:13-octadecadienoic acid, (10-cis and 13-cisforms), 11:14-octadecadienoicacid, (11-cis and 14-cisforms), beta-vinylacrylic acid, sorbic acid, geranicacid, tetra-triethenoidfaty acids, triethenoidfaty acid, clupandoic acid, moroctic acid, arachidonic acid, alpha and beta parinaric acids, oleicacid, linoleicacid, licanic acid, eleostearic acid, ricinoleicacid, clupanodonicacid, palmitoleic acid, eleostearic acid, cis-11-methyl-2-dodecenoic acid, trans-2-decanoic acid (T2DA), heptylcyclopropane-1-carboxylic acid (2CP), palmitic acid, Cis-2-decanoic acid (C2DA), palmitoleic acid, Palmitelaidic acid, 7 (Z), 10 (Z)-Hexadecadienoic acid, Linoleic acid, γ-Linolenic acid, Arachidonic acid, chenodeoxycholic acid, deoxycholic acid, formic acid or hexanoic acid, and the like. 1-Hydroxy-2-naphthoic acid (xinafoic acid), 2-Naphthalene sulfonic acid (NSA), Brilliant blue FCF, Carboxy methyl polyethylene glycol (CM-PEG), Cholesteryl hemisuccinate, Cholic acid (sodium cholate), Decanoic acid (sodium decanoate/sodium caprate), Docosahexaenoic acid, Hexadecylphosphate, Linoleic acid, N,N-Dipalmitoyl-L-lysine, Oleic acid (sodium oleate also used), Pamoic (disodium pamoate also used), acetate, cholesteryl sulfate, Sodium decanesulfonate (SDES), deoxycholate, docusate (AOT, dioctyl sulfosuccinic acid, bis-2-ethylhexyl-sulfosuccinate), dodecyl benzenesulfonate (SDBS), dodecyl sulfate (sodium lauryl sulfate), laurate (sodium dodecanoate), n-octadecyl sulfate (sodium stearyl sulfate), stearate (stearic acid also used), stearoyl glutamate (SSG), taurodeoxycholate (STDC), tetradecyl sulfate, tripolyphosphate, Taurocholic acid (sodium taurocholate also used), Vitamin E (a-tocopherol) succinate, Arginine-hexadecanoyl ester (AHE), Arginine-nonyl ester (ANE), Benethamine (N-benzyl-2-phenylethanamine), Dodecylamine (laurylamine), Hexadecyl trimethylammonium (cetrimonium) bromide (CTAB), Maprotiline, Na-Deoxycholyl-L-lysyl-methylester, N,N′-Dibenzyl ethylenediamine (benzathine), N, N-Dimethyl dodecylamine (DDA), N,N-Dimethyl hexylamine, N, N-Dimethyl octadecylamine (dimethyl stearamine), Stearylamine (octadecylamine), Tetrabutyl ammonium bromide (TBAB), Tetraheptyl ammonium bromide (THA), Tetrahexyl ammonium bromide, Tetraoctyl ammonium bromide (TOAB), Tetrapentyl ammonium bromide (TPA), Triethylamine (TEA), Cholesteryl, hemisuccinate, Sulfonated Cholesterol, 25-hydroxycholesterol-3-sulfate, cholesterol 3-sulfate, Dehydroepiandrosterone sulfate and the like.

In one embodiment, HIP complexes comprise hydrophobic counterions with one or multiple ionizable groups belonging to the group of bile-acids, such as but not limited to Cholic Acids, 3α,6α, 7α-trihydroxy-5β-cholanic acid, 3α, 66, 7α, 12α-tetrahydroxy-5β-cholan-24-oic acid, 5β-cholanic acid-3β-ol, 5α-cholanic acid-3β-ol, 5β-cholanic acid-3α, 12α-diol, 5β-cholanic acid-3α,7α-diol, 5β-cholanic acid-3α, 7β-diol, 5β-cholanic acid-3α,7α, 12α-triol, 5α-cholanic acid-3α,7α, 12α-triol, 5β-cholanic acid-3α,6α-diol, 5β-cholanic acid-3,7,12-trione, 5β-cholanic acid-3, 12α-diol-7-one, 5β-cholanic acid-3-one, 5β-cholanic acid-3α-ol, 58-cholanic acid-7α, 12α-diol, 3α,6α,7α, 12α-tetrahydroxy-5β-cholan-24-oic acid, 3α, 78, 12α-trihydroxy-5β-cholan-24-oic acid, 5β-cholanic acid-3α-ol-7, 12-dione, 5β-cholanic acid-3α,6α,7β-triol, 5β-cholanic acid-3α,6β,7α-triol, 53-cholanic acid-3α,6β,73-triol, 58-cholanic acid-3α-ol-6,7-dione, 5β-cholanoic acid-3α,7α-diol-12-one, 5β-cholanoic acid-7α, 12α-diol-3-one, 5β-cholanoic acid-3α-ol-7-one, 53-cholanoic acid-3α-ol-12-one, 23-nor-5β-cholanoic acid-3α, 12α-diol, 8 (14),53-cholenoic acid-3α, 12α-diol, 5β-cholanic acid-12α-ol-3-one, 5β-cholanic acid-7α-ol-3-one, 5β-cholanic acid-3α,6β-diol, 58-chol-14-enoic acid-3α, 12α-diol, Including, but not limited to, cholic acid sulfates, glycocholic acids, taurocholic acids and functionalized bile acids

In one embodiment, HIP complexes comprise hydrophobic counterions with one or multiple ionizable groups belonging to the group of phosphatidic acid (PA), cyclic PA, lysophosphatidic acid (LPA), cyclic LPA, phosphatidylglycerol (PG), lysophosphatidylglycerol (LPG), phosphoinositides (PI), lysophosphatidylinositol (LPI), phosphatidylserine (PS), or Lysophosphatidylserine (LPS)phospholipids and or ether lipids, or sphingolipids (SP) or sphingolysolipids (LSP), all with mixed or non-mixed, saturated and or unsaturated, aliphatic and or aromatic, C6-C22 hydrocarbon chains. Preferred examples of lipid chains include, but arenot limited to propionyl, butyryl, hexanoyl, octanoyl, decanoyl, lauroyl, myristoyl, palmitoyl, stearoyl, arachidoyl, behenoyl, lignoceroyl and the like. Other preferred examples of lipid chains are vaccenoyl, (8Z) octadecenoyl, myristoleoyl, myristelaidoyl, palmitoleoyl, palmitelaidoyl, oleoyl, dielaidoyl and the like.

In one embodiment, HIP complexes comprise hydrophobic counterions with one or multiple ionizable groups belonging to the group of PA, LPA, PG, LPG, PI, LPI, PS, or LPS phospholipids, or SP or LSP all with mixed or non-mixed, saturated or unsaturated, sterol modified, fatty acid modified, or headgroup modified such as but not limited to PEGylated, fluorophore or chelator functionalized. Functionalized here includes but is not limited to deuteration, fluorescence, biotinylation, diphytanoylation, bromination, oxidization, diacetylenation, fluorination, and modifications with bioactive molecules.

In one embodiment, HIP complexes comprise hydrophobic counterions with one or multiple ionizable groups belonging to the group of multivalent cationic lipids such as but not limited to N1-[2-((1S)-1-[(3-aminopropyl)amino]-4-[di(3-amino-propyl)amino]butylcarboxamido)ethyl]-3,4-di [oleyloxy]-benzamide or N4-Cholesteryl-Spermine.

In one embodiment, HIP complexes comprise hydrophobic counterions with one or multiple ionizable groups belonging to the group of ionizable charge cationic lipids such as but not limited to DOBAQ (N-(4-carboxybenzyl)-N, N-dimethyl-2,3-bis(oleoyloxy) propan-1-aminium), 1,2-distearoyl-3-dimethylammonium-propane, 1,2-dipalmitoyl-3-dimethylammonium-propane, 1,2-dimyristoyl-3-dimethylammonium-propane, 1,2-dioleoyl-3-dimethylammonium-propane (DODAP), 1,2-dioleyloxy-3-dimethylaminopropane (DODMA), Dlin-DMA, Dlin-KC2-DMA, Dlin-KC3-DMA, C12-200, A6, OF-02, A18-Iso5-2DC18, YSK05, 7C1, GO-C14, L319, OF-Deg-Lin, 306-012β, 3060110, FTT5, 9A1P9, 98N12-5, 304013, CKK-E12 and the like.

In one embodiment, HIP complexes comprise hydrophobic counterions with one or multiple ionizable groups belonging to the group of fixed charge cationic lipids such as but not limited to 3-[N-(N′, N′-dimethylaminoethane)-carbamoyl]cholesterol (DC-Cholesterol), N-(2-hydroxyethyl)-N,N-dimethyl-2,3-bis(oleoyloxy) propan-1-aminium (DORI), O,O′-ditetradecanoyl-N-(α-trimethylammonioacetyl) diethanolamine, ethylphosphocholines (EPCs) of lauroyl, myristoyl, palmitoyl, stearoyl, and oleoyl. Such as Dimethyldioctadecylammonium, 1,2-dimyristoyl-3-trimethylammonium-propane, 1,2-dipalmitoyl-3-trimethylammonium-propane, 1,2-stearoyl-3-trimethylammonium-propane and 1,2-dioleoyl-3-trimethylammonium-propane. Such as 1,2-di-O-octadecenyl-3-trimethylammonium propane.

In one embodiment, HIP complexes comprise charged polymers such as but not limited to polyethyleneimine (PEI), polylysine (PLL), polyarginine (PAA), Chitosans, Poly(2-ethyl-2-oxazoline) (ULTROXA), Diethylaminoethyl-dextran (DEAE-dextran), dendritic polyamidoamine (PAMAM) and PDMAEMA [poly(N,N-dimethylaminoethyl methacrylate] or mixtures thereof. In one embodiment, HIP complexes comprise PAMAM.

In one embodiment, HIP complexes comprise a polymer functionalized with a targeting ligand such as but not limited to carbohydrates, Galactose, Mannose, peptides, RGD, CRGD, GalNAc or mixtures thereof, and the HIP complexes are solubilized or dispersed in a composition or depot of the invention e.g. an NCCell.

In one embodiment, HIP complexes comprise hydrophobic counterions (e.g. selected from the class of ionic detergents, ionic lipids and lysolipids, fatty acids and ionic polymers) functionalized with a targeting ligand such as but not limited to carbohydrates, Galactose, Mannose, peptides, RGD, cRGD, GalNAc or mixtures thereof.

In one embodiment, a HIP particle is formed from a nucleic acid and counterions, and the HIP is solubilized in a composition or depot of the invention e.g. an NCCell.

In one embodiment, a HIP particle is formed from a nucleic acid and counterions, and the HIP is solubilized in a composition or depot of the invention e.g. an NCCell wherefrom it is released as particles or as non-particle HIP complexes.

In one embodiment, a HIP particle is formed from a nucleic acid and counterions, and the HIP is solubilized in a composition or depot of the invention e.g. an NCCell, wherefrom it is released and the HIP dissociates and releases the native nucleic acid.

In one embodiment, a HIP particle comprises hydrophobic counterions with one or multiple ionizable groups selected from but not limited to DC-Chol, Dioleoyl-3-trimethylammonium-propane (DOTAP), Distearoyl-3-trimethylammonium-propane (DSTAP), Spermin-chol (GL67), Tetraethyl ammonium bromide (TEAB), Benzyl trimethyl ammonium chloride (BTMAC), Didodecyl dimethyl ammonium bromide (DDAB), Tetrahexyl ammonium bromide (THAB), Cetyl trimethyl ammonium bromide (CTAB), Dlin-KC2-DMA (KC2), Dlin-MC3-DMA (MC3), N-(4-carboxybenzyl)-N,N-dimethyl-2,3-bis(oleoyloxy) propan-1-aminium (DOBAQ), N1-[2-((1S)-1-[(3-aminopropyl)amino]-4-[di(3-amino-propyl)amino]butylcarboxamido)ethyl]-3,4-di [oleyloxy]-benzamide (MVL5).

In one embodiment, the LNP composition, polyplex composition, lipoplex composition or HIP complex comprises, but is not limited to: L-PEI25K, L-PEI40K, L-PEI25K, L-PEI25K: L-PEI25K-PEG550 (50:50), L-PEI25K-PEG1000, L-PEI25K-PEG2000, JET-PEI, JET-PEI-Mannose, JET-PEI-Galactose, L-PEI40Max, DOTAP: Cholesterol 50:50, DOTAP: Cholesterol: DOPE 50:25:25, DOTAP: Cholesterol: DOPE 50:40:10, DOTAP: Cholesterol: DOPE 50:45:5, DOTAP: Cholesterol: DOPE 50:30:20, DOTAP: Cholesterol: DOPE 40:30:30, DOTAP: Cholesterol: DOPE 40:40:20, DOTAP: Cholesterol: DOPE 40:50:10, DOTAP: Cholesterol: DOPE 30:30:40, DOTAP: Cholesterol: DOPE 30:40:30, DOTAP: Cholesterol: DOPE 30:50:20, DC-Cholesterol: Cholesterol: DOPE 25:70:5, DC-Cholesterol: Cholesterol: DOPE 25:60:15, DC-Cholesterol: Cholesterol: DOPE 25:50:25, DC-Cholesterol: Cholesterol: DOPE 30:65:5, DC-Cholesterol: Cholesterol: DOPE 30:35:35, DC-Cholesterol: Cholesterol: DOPE 40:30:30, DC-Cholesterol: Cholesterol: DOPE 40:40:20, DC-Cholesterol: Cholesterol: DOPE 40:50:10, DC-Cholesterol: Cholesterol: DOPE 50:45:5, DC-Cholesterol: Cholesterol: DOPE 50:25:25, DC-Cholesterol: Cholesterol 33:67, DC-Cholesterol: Cholesterol 30:70, DC-Cholesterol: Cholesterol 25:75, DC-Cholesterol: Cholesterol 20:80, Dlin-KC2: Chol: DSPC 50:40:10, DC-Chol: Chol: DOPE (30:65:5), DOTAP: Chol: DOPE (50:45:5), KC2: Chol: DSPC (50:40:10), KC2: DC-Chol: Chol: DSPC (25:25:40:10), KC2: Chol: DSPC (50:40:10), KC2: Chol: DSPC (50:40:10), KC2: DC-chol: Chol: DSPC (25:25:40:10), DC-Chol: Chol: DOPE (33:33:33), DC-Chol: Chol: DOPE (30:65:5), DC-Chol: Chol (50:50), KC2: Chol: DSPC (50:40:10), KC2: Chol: DSPC (50:40:10)+1.5% DMG-PEG2k, DOTAP: Cholesterol (50:50 liposomal formulation), DC-chol: Chol (50:50), DC-Chol: Chol: DOPE (33:33:33), DC-Chol: Chol: DOPE (30:65:5), KC2: Chol: DSPC (50:40:10), Lipofectamine, DC-Chol: DOPE (33:67), DC-Chol: DOPE: DMG-PEG2K (33:67:1.5), DC-Chol: Chol: DOPE (33:33:33), DOTAP: DOPE: Chol (50:5:45), DOTAP: DOPE: Chol (50:25:25), KC2-Chol: DSPC (50:40:10)+1.5% PEG, MC3: chol: DSPC (50:40:10), DOTAP, DSTAP, DC-cholesterol, Spermin-cholesterol, Tetraethyl ammonium bromide (TEAB), Benzyl trimethyl ammonium chloride (BTMAC), Didodecyl dimethyl ammonium bromide (DDAB), Tetrahexyl ammonium bromide (THAB), Cetyl trimethyl ammonium bromide (CTAB), KC2, Dlin-KC2-DMA (KC2), Dlin-KC3-DMA (MC3), N-(4-carboxybenzyl)-N,N-dimethyl-2,3-bis(oleoyloxy) propan-1-aminium (DOBAQ), or N1-[2-((1S)-1-[(3-aminopropyl)amino]-4-[di(3-amino-propyl)amino]butylcarboxamido)ethyl]-3,4-di [oleyloxy]-benzamide (MVL5).

In one embodiment, one or more LNPs, HIPs, polyplexes or lipoplexes are formed using PEI 25kDA functionalized with a targeting ligands such as but not limited to carbohydrates, Galactose, Mannose, peptides, RGD, cRGD, GalNAc or mixtures thereof. In one embodiment, one or more LNPs, HIPs, polyplexes or lipoplexes are formed using PEI 25kDA functionalized with 1, 5 or 10 mol % of a targeting ligand such as but not limited to carbohydrates, Galactose, Mannose, peptides, RGD, cRGD, GalNAc or mixtures thereof. In one embodiment, one or more LNPs, HIPs, polyplexes or lipoplexes are formed using PEI 25kDA functionalized with 1, 5 or 10 mol % mannose.

In one embodiment, one or more LNPs, HIPs, polyplexes or lipoplexes are formed using PEI 40 kDa functionalized with a targeting ligands such as but not limited to carbohydrates, Galactose, Mannose, peptides, RGD, cRGD, GalNAc or mixtures thereof. In one embodiment, one or more LNPs, HIPs, polyplexes or lipoplexes are formed using PEI 40 kDa functionalized with 1, 5 or 10 mol % of a targeting ligand such as but not limited to carbohydrates, Galactose, Mannose, peptides, RGD, cRGD, GalNAc or mixtures thereof. In one embodiment, one or more LNPs, HIPs, polyplexes or lipoplexes are formed using PEI 40 kDa functionalized with 1, 5 or 10 mol % mannose.

In one embodiment, the composition comprises, but is not limited to: SuBen: GTH: EtOH: DMSO (55:20:5:10), SuBen: GTH: EtOH: DMSO (55:20:5:20), SuBen: GTH: EtOH: DMSO (55:20:10:10), SuBen: GTH: EtOH: DMSO (55:20:5:20+0.25% w/w Cholesterol), LOIB: GTH: EtOH: DMSO (70:10:5:15), SuBen: GTH: EtOH: DMSO (55:20:2:20), SuBen: GTH: EtOH: DMSO (55:20:2:20+0.5% POPC), LOIB: GTO: EtOH: DMSO (70:10:5:15), or LOIB: GTO: EtOH: DMSO (65:7.5:5:22.5).

In one embodiment, the composition is an NCCell solution comprising: SuBen: GTO: EtOH, SuBen: GTH: EtOH, SuBen: Ethyl-palmitate: EtOH, SuBen: GTO: DMSO, SuBen: GTH: DMSO, SuBen: Ethyl-palmitate: DMSO, SuBen: GTH: EtOH: DMSO, LacBen: GTO: EtOH, LacBen: GTH: EtOH, LacBen: Ethyl-palmitate: EtOH, LacBen: GTO: DMSO, LacBen: GTH: DMSO, LacBen: Ethyl-palmitate: DMSO, LacBen: GTH: EtOH: DMSO, TreBen: GTO: EtOH, TreBen: GTH: EtOH, TreBen: Ethyl-palmitate: EtOH, TreBen: GTO: DMSO, TreBen: GTH: DMSO, TreBen: Ethyl-palmitate: DMSO, TreBen: GTH: EtOH: DMSO, SOIB: GTO: EtOH, SOIB: GTH: EtOH, SOIB: Ethyl-palmitate: EtOH, SOIB: GTO: DMSO, SOIB: GTH: DMSO, SOIB: Ethyl-palmitate: DMSO, SOIB: GTH: EtOH: DMSO, LOIB: GTO: EtOH, LOIB: GTH: EtOH, LOIB: Ethyl-palmitate: EtOH, LOIB: GTO: DMSO, LOIB: GTH: DMSO, LOIB: Ethyl-palmitate: DMSO, or LOIB: GTH: EtOH: DMSO,

In one embodiment, the composition is an NCCell solution comprising: SuBen: GTO: EtOH, SuBen: GTH: EtOH, SuBen: Ethyl-palmitate: EtOH, SuBen: GTO: DMSO, SuBen: GTH: DMSO, SuBen: Ethyl-palmitate: DMSO, SuBen: GTH: EtOH: DMSO, LacBen: GTO: EtOH, LacBen: GTH: EtOH, LacBen: Ethyl-palmitate: EtOH, LacBen: GTO: DMSO, LacBen: GTH: DMSO, LacBen: Ethyl-palmitate: DMSO, LacBen: GTH: EtOH: DMSO, TreBen: GTO: EtOH, TreBen: GTH: EtOH, TreBen: Ethyl-palmitate: EtOH, TreBen: GTO: DMSO, TreBen: GTH: DMSO, TreBen: Ethyl-palmitate: DMSO, TreBen: GTH: EtOH: DMSO, SOIB: GTO: EtOH, SOIB: GTH: EtOH, SOIB: Ethyl-palmitate: EtOH, SOIB: GTO: DMSO, SOIB: GTH: DMSO, SOIB: Ethyl-palmitate: DMSO, SOIB: GTH: EtOH: DMSO, LOIB: GTO: EtOH, LOIB: GTH: EtOH, LOIB: Ethyl-palmitate: EtOH, LOIB: GTO: DMSO, LOIB: GTH: DMSO, LOIB: Ethyl-palmitate: DMSO, or LOIB: GTH: EtOH: DMSO, wherein the solution comprises 40-80% carbohydrate ester, 0-35% co-solvent, and 10-30% solvent. In one embodiment, the composition comprises 30-70%, 30-60%, 30-50%, 30-40%, 40-80%, 50-80%, 60-80%, or 70-80% carbohydrate ester. In one embodiment, the composition comprises 0-30%, 0-25%, 0-20%, 0-15%, 0-10%, 5-30%, 10-30%, 15-30%, 20-30%, or 25-30% co-solvent. In one embodiment, the composition comprises 10-25%, 10-20%, 10-15%, 15-30%, 20-30%, or 25-30% solvent.

In one embodiment, a HIP is formed as a powder from a nucleic acid and counterions, and the HIP powder is solubilized in a composition or depot of the invention e.g. an NCCell.

In one embodiment, a HIP is formed from a nucleic acid and counterions and is isolated from an organic or aqueous phase, and the HIP is solubilized in a composition or depot of the invention e.g. an NCCell.

In one embodiment, a HIP is formed from a nucleic acid and counterions, and the HIP is added from an organic phase to a composition or depot of the invention e.g. an NCCell.

In one embodiment, a composition or depot of the invention (e.g. an NCCell) comprises a HIP formed from a nucleic acid and counterions, where the counterion exerts an additive or synergistic pharmacological effect.

In one embodiment, the HIP formulated in a composition or depot of the invention (e.g. an NCCell) comprises a nucleic acid and counterions that may have, but not limited to, anti-cancer activity, cytotoxic, hormonal effect, anti-microbial activity, tissue regenerative effect (e.g., pro-angiogenic, pro-fibrogenic, pro-osteogenic factors).

In one embodiment, the HIP comprises an imaging agent. In one embodiment, the HIP formulated in a composition or depot of the invention (e.g. an NCCell) comprises a nucleic acid and counterions that are agents with but not limited to radiographic contrast, MRI contrast, NIR or standard fluorescence, chromophores or radiometal chelators.

In one embodiment, a HIP complex is formed from counterions and a nucleic acid, and the nucleic acid is functionalized with one or more targeting ligands such as but not limited to RGD, cRGD, Transferrin, Folate, a signal peptide or signal sequence, a localization signal or sequence, a nuclear localization signal or sequence (NLS), an antibody, a cell penetrating peptide (CPP), (e.g. TAT, KALA), a ligand of a receptor (e.g. cytokines, hormones, growth factors etc), small molecules (e.g. carbohydrates like mannose or galactose or synthetic ligands), small molecule agonists, inhibitors or antagonists of receptors (e.g. RGD peptidomimetic analogues) or any such molecule. Particularly preferred are cell penetrating peptides (CPPs), which include, without being limited thereto protamine, nucleoline, spermine or spermidine, poly-L-lysine (PLL), basic polypeptides, poly-arginine, chimeric CPPs, such as Transportan, or MPG peptides, HIV-binding peptides, Tat, HIV-1 Tat (HIV), Tat-derived peptides, oligoarginines, members of the penetratin family, e.g. Penetratin, Antennapedia-derived peptides (particularly from Drosophila antennapedia), pAntp, plsl, etc., antimicrobial-derived CPPs e.g. Buforin-2, Bac715-24, SynB, SynB (1), pVEC, hCT-derived peptides, SAP, MAP, PpTG20, proline-rich peptides, Loligomers, arginine-rich peptides, Calcitonin-peptides, FGF, Lactoferrin, poly-L-lysine, poly-arginine, histones, VP22 derived or analog peptides, Pestivirus Erns, HSV, VP22 (Herpes simplex), MAP, KALA or protein transduction domains (PTDs, PpT620, proline-rich peptides, arginine-rich peptides, lysine-rich peptides, Pep-1, L-oligomers, Calcitonin peptide(s).

In one embodiment, a HIP complex is formed from counterions and a nucleic acid, and the counterion (e.g. a polymer or lipid) is functionalized with one or more targeting ligands such as but not limited to RGD, cRGD, Transferrin, Folate, a signal peptide or signal sequence, a localization signal or sequence, a nuclear localization signal or sequence (NLS), an antibody, a cell penetrating peptide (CPP), (e.g. TAT, KALA), a ligand of a receptor (e.g. cytokines, hormones, growth factors etc), small molecules (e.g. carbohydrates like mannose or galactose or synthetic ligands), small molecule agonists, inhibitors or antagonists of receptors (e.g. RGD peptidomimetic analogues) or any such molecule. Particularly preferred are cell penetrating peptides (CPPs), which include, without being limited thereto protamine, nucleoline, spermine or spermidine, poly-L-lysine (PLL), basic polypeptides, poly-arginine, chimeric CPPs, such as Transportan, or MPG peptides, HIV-binding peptides, Tat, HIV-1 Tat (HIV), Tat-derived peptides, oligoarginines, members of the penetratin family, e.g. Penetratin, Antennapedia-derived peptides (particularly from Drosophila antennapedia), pAntp, plsl, etc., antimicrobial-derived CPPs e.g. Buforin-2, Bac715-24, SynB, SynB (1), pVEC, hCT-derived peptides, SAP, MAP, PpTG20, proline-rich peptides, Loligomers, arginine-rich peptides, Calcitonin-peptides, FGF, Lactoferrin, poly-L-lysine, poly-arginine, histones, VP22 derived or analog peptides, Pestivirus Erns, HSV, VP22 (Herpes simplex), MAP, KALA or protein transduction domains (PTDs, PpT620, proline-rich peptides, arginine-rich peptides, lysine-rich peptides, Pep-1, L-oligomers, and Calcitonin peptide(s).

In one embodiment, targeting of nucleic acid constructs to specific cells is achieved using CD19, CD22, CD30, CD33, CD44, CD74, CD276, EGFR, Nectin4, AXL, ALK, PTK7, TM4SF1, LRP1, Somatostatin, RGD, Tenascin3, Nucleolin, Mucin-1, Fibronectin, Tenascin C, MT1-MMP, Glucose receptor, Mannose receptor, Galactose receptor, HER2, Transferrin, Folic acid receptor, Hyaluronan, and PSMA.

In another embodiment, the targeted cells are antigen presenting cells or dendritic cells. In one embodiment, the dendritic cells are targeted using DEC205, XCR1, CD197, CD80, CD86, CD123, CD209, CD273, CD283, CD289, CD184, CD85h, CD85j, CD85k, CD85d, CD85g, CD85α, CD141, CD1Ic, CD83, TSLP receptor, Clec9a or Cdla marker. In one embodiment, the dendritic cells are targeted using the CD141, FLT3L, trombin, DEC205 and ligand FSSVRY, or the XCR1 ligand XCL1.

In one embodiment, one or more LNPs, HIPs, polyplexes or lipoplexes are formulated in compositions or depots of the invention (e.g. an NCCell) comprising a carbohydrate ester, a co-solvent and a solvent.

In one embodiment, one or more LNPs, HIPs, polyplexes or lipoplexes are formulated in compositions or depots of the invention (e.g. an NCCell) comprising a carbohydrate ester, a CT contrast agent, a co-solvent and a solvent.

In one embodiment, one or more LNPs, HIPs, polyplexes or lipoplexes are formulated in compositions or depots of the invention (e.g. an NCCell) comprising 30-80% carbohydrate ester, 5-20% CT contrast agent, 5-30% co-solvent and 10-30% solvent.

In one embodiment, one or more LNPs, HIPs, polyplexes or lipoplexes are formulated in compositions or depots of the invention (e.g. an NCCell) comprising 30-70%, 30-60%, 30-50%, 30-40%, 40-80%, 50-80%, 60-80%, or 70-80% carbohydrate ester.

In one embodiment, one or more LNPs, HIPs, polyplexes or lipoplexes are formulated in compositions or depots of the invention (e.g. an NCCell) comprising 5-15%, 5-10%, 10-20%, or 15-20% CT contrast agent.

In one embodiment, one or more LNPs, HIPs, polyplexes or lipoplexes are formulated in compositions or depots of the invention (e.g. an NCCell) comprising 5-25%, 5-20%, 5-15%, 5-10%, 10-30%, 15-30%, 20-30%, or 25-30% co-solvent.

In one embodiment, one or more LNPs, HIPs, polyplexes or lipoplexes are formulated in compositions or depots of the invention (e.g. an NCCell) comprising 10-25%, 10-20%, 10-15%, 15-30%, 20-30%, or 25-30% solvent.

In one embodiment, one or more LNPs, HIPs, polyplexes or lipoplexes are formulated in compositions or depots of the invention (e.g. an NCCell) comprising a carbohydrate ester such SOP, SAIB, SOIB, SuBen, LOP, LOIB, LacBen, MeLOIB and the like, a CT contrast agent such as xSAIB, CLA-8 and the like, a co-solvent such as glycerol trihexanoate (GTH), glycerol trioctanoate (GTO) and glycerol tridecanoate (GTD) or Lipiodol and the like, and a solvent such as EtOH, DMSO and the like.

In one embodiment, one or more LNPs, HIPs, polyplexes or lipoplexes are formulated in compositions or depots of the invention (e.g. an NCCell) comprising a carbohydrate ester such as SuBen, LacBen, TreBen, SOIB, LOIB or combinations thereof, the co-solvent GTO, GTH, GTD, ethyl-palmitate, ethanol or combinations thereof, and the solvent EtOH, DMSO, NMP, BnOH, PC or combinations thereof.

In one embodiment, one or more LNPs, HIPs, polyplexes or lipoplexes are formulated in compositions or depots of the invention (e.g. an NCCell) comprising 30-80% carbohydrate ester such SOP, SAIB, SOIB, SuBen, LOP, LOIB, LacBen, MeLOIB and the like, 5-20% CT contrast agent such as xSAIB, CLA-8 and the line, 5-30% co-solvent such as glycerol trihexanoate (GTH), glycerol trioctanoate (GTO) and glycerol tridecanoate (GTD) or Lipiodol and the like, and 10-30% solvent such as EtOH, DMSO and the like.

In one embodiment, one or more LNPs, HIPs, polyplexes or lipoplexes are formulated in compositions or depots of the invention (e.g. an NCCell) comprising 30-80% carbohydrate ester such as SOP, SAIB, SOIB, SuBen, LOP, LOIB, LacBen, MeLOIB or combinations thereof, 5-20% CT contrast agent such as xSAIB, CLA-8 and the like, 5-30% co-solvent such as glycerol trihexanoate (GTH), glycerol trioctanoate (GTO) and glycerol tridecanoate (GTD) or Lipiodol and the like or combinations thereof, and 10-30% solvent such as EtOH, DMSO and the like or combinations thereof.

It will be understood that references herein to “one or more LNPs, HIPs, polyplexes or lipoplexes” embrace compositions comprising only LNPs, only HIPs, only polyplexes, or only lipoplexes as well as compositions comprising two or more (e.g. three or more, or all four) of LNPs, HIPs, polyplexes and lipoplexes. For example, one or more LNPs, HIPs, polyplexes or lipoplexes embraces compositions comprising LNPs and HIPS; LNPs and polyplexes; LNPs and lipoplexes; HIPs and polyplexes; HIPs and lipoplexes; polyplexes and lipoplexes; LNPs, HIPS and polyplexes; LNPs, HIPs and lipoplexes; HIPs, polyplexes and lipoplexes; LNPs, polyplexes and lipoplexes; and HIPS, LNPs, polyplexes and lipoplexes.

In one embodiment, LNPs, HIPs, polyplexes or lipoplexes are formulated in compositions or depots of the invention (e.g. an NCCell) and their release is reduced by increasing the viscosity by lowering of the co-solvent content.

In one embodiment, either LNPs, HIPs, polyplexes or lipoplexes are formulated in compositions or depots of the invention (e.g. an NCCell) and their rate of release is reduced by increasing the viscosity by lowering of the co-solvent to carbohydrate ester ratio, or by replacing short chain fatty acid carbohydrate esters such as but not limited to LOP and SAIB with longer fatty acid carbohydrate esters such as but not limited to SuBen and LacBen.

In one embodiment, either LNPs, HIPs, polyplexes or lipoplexes are formulated in compositions or depots of the invention (e.g. an NCCell) and their rate of release is reduced by increasing the viscosity by replacing short chain fatty acid carbohydrate esters (LOP, SAIB) with longer fatty acid carbohydrate esters.

In one embodiment, either LNPs, HIPs, polyplexes or lipoplexes are formulated in compositions or depots of the invention (e.g. an NCCell) and their rate of release is modulated by incorporation of lipids from the list DOTAP, DC-Chol, Cholesterol (Chol), POPC, POPG, DSPE-PEG2k, DMG-PEG2k and the like.

In one embodiment, one or more LNPs, HIPs, polyplexes or lipoplexes and combinations thereof are formulated in a composition or depot of the invention e.g. an NCCell.

In one embodiment, the invention provides compositions or depots (e.g. an NCCell) formulated for sustained release of the nucleic acid component (e.g. one or more LNPs, HIPs, polyplexes, or lipoplexes), wherein the composition or depot comprises at most 20% co-solvent, optionally at most 15%, or at most 10%.

In one embodiment, the invention provides compositions or depots (e.g. an NCCell) formulated for delayed release of the nucleic acid component (e.g. one or more LNPs, HIPs, polyplexes, or lipoplexes), wherein the composition or depot comprises at most 20% solvent, optionally at most 15%, or at most 10%. It will be understood that compositions or depots which are formulated for delayed release exhibit reduced initial release of the nucleic acid component upon administration (e.g. within 6 hours, within 12 hours, within 1 day, within 2 days, or within 3 days of administration) as compared to compositions or depots having a higher solvent concentration.

### Exemplary Nucleic Acids

In one embodiment deoxyribonucleic acids (DNAs), threose nucleic acids (TNAs), antisense DNA, glycol nucleic acids (GNAs), peptide nucleic acids (PNAs), hexitol nucleic acids (HNA), Morpholino and locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization) or combinations thereof are delivered from a composition or depot of the invention e.g. an NCCell.

One embodiment relates to the delivery of single or double stranded ribonucleic acids (RNAs), including RNA (messenger RNA (mRNA), self-replicating mRNA, transfer RNA (tRNA), and ribosomal RNA (rRNA)), small single or double stranded RNA (dsRNA, RNA interference (RNAi) including microRNA (miRNA), small interfering RNA (siRNA or ASO), splice switching antisense oligonucleotide (SSO), CRISPR-Cas9 sgRNAs, cyclic mRNA, piwi-interacting RNA (piRNA) and repeat associated small interfering RNA (rasiRNA). In addition to alternative synthetic nucleic acids analogous including phosphorotioates (PS), (including PS morpholino, 2′-O-methyl, 2′-O-methoxyethyl, 2′fluoro, 5′methylcystine and G-clamp) in any combinations thereof from a composition or depot of the invention e.g. an NCCell.

In one embodiment, the nucleic acid component is functionalized with one or more fluorophores such as Cy5, Cy7, Cy7.5 and the like.

In one embodiment, the nucleic acid component is functionalized with one or more metal chelators such as DOTA, EDTA and DTPA and the like.

One embodiment relates to the delivery of single or double stranded ribonucleic acids and deoxyribonucleic acids (DNAs) in any combinations thereof from a composition or depot of the invention e.g. an NCCell.

In one embodiment a treatment of a disease or disorder in a human or animal is provided by delivery one or more nucleic acids that encode therapeutic proteins or peptides or variants hereof. Another embodiment relates to the treatment of a disease or disorder in a human or animal by nucleic acids having a pharmaceutical activity on their own or in a combination of nucleic acid classes. In another embodiment, nucleic acid constructs may encode one or more antibodies or fragments thereof. Antibody includes monoclonal antibodies (including full length antibodies which have an immunoglobulin Fc region), antibody compositions with polyepitopic specificity, multispecific antibodies (e.g., bispecific antibodies, diabodies, and single-chain molecules), as well as antibody fragments.

In one embodiment composition of the invention is injected or administrated into healthy or diseased tissue of a human or animal. In one embodiment, the delivery system is administered into cancerous tissue (e.g., one or more tumors, metastases, organs, and/or lymph nodes), inflamed or infected tissues or lesions (e.g., infections in organs, soft and/or bone tissue, autoimmune diseases, abscesses), or into tissue that is undergoing a reparatory or regenerative process or requires tissue regeneration or revitalization (e.g., non-union bone fractures, vasculature compromised areas, chronic wounds, vascular defects and compromised regions, stroke, haemorrhage, burns, skin and tissue defects). In another embodiment, the delivery system is administered in non-diseased tissue (e.g., subcutaneous, intramuscular, intraperitoneal, intranodal) to stimulate a systemic therapeutic (e.g., hormone replacement, supplementation or antagonism, or vaccination, or enzyme replacement).

In one embodiment, the composition or depot of the invention e.g. an NCCell further comprises one or more pharmaceutically active drugs, compounds, diluents and/or excipients in addition to one or more gene engineering nucleic acids. In one embodiment, the composition or depot of the invention e.g. an NCCell further comprises at least one molecular adjuvant or therapeutic agent.

Excipients include but are not limited to any and all solvents, dispersion media, diluents, or other liquid vehicles, dispersion or suspension aids, surface active agents, isotonic agents, thickening or emulsifying agents, preservatives, solid binders, lubricants, flavoring agents, stabilizers, antioxidants, osmolality adjusting agents, pH adjusting agents and the like. Excipients include, but are not limited to, inert diluents, dispersing and/or granulating agents, surface active agents and/or emulsifiers, disintegrating agents, binding agents, preservatives, buffering agents, lubricating agents, and/or oils. Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

In one embodiment, the composition or depot of the invention e.g. an NCCell induces or enhances an immune response, preferably an immune response against cancer. In another embodiment, the composition or depot of the invention e.g. an NCCell induces or enhances an immune response, preferably an immune response against an infectious disease.

According to the invention, the DNA in a composition or depot of the invention e.g. an NCCell can encode for an RNA sequence that again encodes a therapeutic peptide or protein or variant. For example, DNA encoding and expressing an antigen or an immune activating compound.

In one embodiment, gene silencing is achieved through the delivery of antisense or silencing nucleic acids.

In one embodiment, one or more DNA sequences or DNA classes that are pharmaceutically active on their own are delivered in the composition or depot of the invention e.g. an NCCell, e.g., it has one or more pharmaceutical activities such as those described for pharmaceutically active proteins. For example, the DNA or synthetic DNA analogue may have one or more strands. Such agents include antisense-DNA, dsDNA or synthetic DNA such as PNA, Morpholino and locked nucleic acid (LNA), GNA, TNA, HNA or BNA, targeted to a target transcript, e.g., a transcript of an endogenous disease-related transcript of a subject.

In one embodiment, the RNA delivered is pharmaceutically active or encodes at least one pharmaceutically active peptide or protein such as an immunologically active peptide or protein. In one embodiment, the RNA encodes at least one antigen. In one embodiment, the antigen is a disease-associated antigen or elicits an immune response against a disease-associated antigen or cells expressing a disease-associated antigen. In one embodiment, the RNA encodes at least one cancer associated antigen or neo-antigen or elicits an immune response against cancer associated antigen.

In one embodiment, the RNA in the composition or depot of the invention e.g. an NCCell is pharmaceutically active in its own, e.g., the RNA may be one or more strands for RNA interference (RNAi). Such agents include transfer RNA (tRNA), ribosomal RNA (rRNA)), transferRNA (TRNA, viral RNA (vRNA), small single or double stranded RNA (dsRNA, RNA interference (RNAi) including microRNA (miRNA), small interfering RNA (siRNA or ASO), splice switching antisense oligonucleotide (SSO), CRISPR-Cas9 sgRNAs, piwi-interacting RNA (piRNA) and repeat associated small interfering RNA (rasiRNA), 2′ fluoro-substituted RNAs, and 2′-O-methyl-substituted RNA and psRNA, targeted to a target transcript, e.g., a transcript of an endogenous disease-related transcript of a subject. Such agents also include lncRNA.

In one embodiment, the DNA or RNA delivered is pharmaceutically active or encodes at least one pharmaceutically active peptide or protein in combination with one or more RNA sequences or classes or DNA sequences or DNA classes that are pharmaceutically active on their own, e.g., have silencing activity, in any possible combination delivered in the composition or depot of the invention e.g. an NCCell.

Examples of nucleic acid construct-encoded pharmaceutically active peptides and proteins include, but are not limited to, cytokines and immune system proteins such as immunologically active compounds (e.g., interleukins, colony stimulating factor (CSF), granulocyte colony stimulating factor (G-CSF), granulocyte-macrophage colony stimulating factor (GM-CSF), erythropoietin, tumor necrosis factor (TNF), interferons, integrins, addressins, seletins, homing receptors, T cell receptors, immunoglobulins, soluble major histocompatibility complex antigens, immunologically active antigens such as bacterial, parasitic, or viral antigens, allergens, autoantigens, antibodies), hormones (insulin, thyroid hormone, catecholamines, gonadotrophins, trophic hormones, prolactin, oxytocin, dopamine, bovine somatotropin, leptins and the like), growth hormones (e.g., human grown hormone), growth factors (e.g., epidermal growth factor, nerve growth factor, insulin-like growth factor and the like), growth factor receptors, enzymes (tissue plasminogen activator, streptokinase, cholesterol biosynthestic or degradative, steriodogenic enzymes, kinases, phosphodiesterases, methylases, de-methylases, dehydrogenases, cellulases, proteases, lipases, phospholipases, aromatases, cytochromes, adenylate or guanylaste cyclases, neuramidases and the like), receptors (steroid hormone receptors, peptide receptors), binding proteins (growth hormone or growth factor binding proteins and the like), transcription and translation factors, tumor growth suppressing proteins (e.g., proteins which inhibit angiogenesis), structural proteins (such as collagen, fibroin, fibrinogen, elastin, tubulin, actin, and myosin), blood proteins (thrombin, serum albumin, Factor VII, Factor VIII, insulin, Factor IX, Factor X, tissue plasminogen activator, protein C, von Wilebrand factor, antithrombin III, glucocerebrosidase, erythropoietin granulocyte colony stimulating factor (GCSF) or modified Factor VIII, anticoagulants and the like.

In one embodiment, a cancerous disease associated with malignant neoplasia such as malignant neoplasm of lip, mouth or throat, such as malignant neoplasm of the tongue, the base of tongue, gum, floor of mouth, palate, parotid gland, major salivary glands, tonsil, oropharynx, nasopharynx, piriform sinus, hypopharynx or other parts of lip, mouth or throat or malignant neoplasms of digestive organs such as malignant neoplasms of oesophagus, stomach, small intestine, colon, rectosigmoid junction, rectum, anus and anal canal, liver and intrahepatic bile ducts, gallbladder, other parts of biliary tract, pancreas and spleen, malignant neoplasms of respiratory and intrathoracic organs such as malignant neoplasms of the nasal cavity and middle ear, accessory sinuses, larynx, trachea, bronchus and lung, thymus, heart, mediastinum and pleura, malignant neoplasms of bone and articular cartilage such as malignant neoplasm of bone and articular cartilage of limbs, bone and articular cartilage, malignant melanoma of skin, sebaceous glands and sweat glands, malignant neoplasms of mesothelial and soft tissue such as malignant neoplasm of mesothelioma, Kaposi's sarcoma, malignant neoplasm of peripheral nerves and autonomic nervous system, malignant neoplasm of retroperitoneum and peritoneum, malignant neoplasm of connective and soft tissue such as blood vessels, bursa, cartilage, fascia, fat, ligament, lymphatic vessel, muscle, synovia, tendon, head, face and neck, abdomen, pelvis or overlapping lesions of connective and soft tissue, malignant neoplasm of breast or female genital organs such as malignant neoplasms of vulva, vagina, cervix uteri, corpus uteri, uterus, ovary, Fallopian tube, placenta or malignant neoplasms of male genital organs such as malignant neoplasms of penis, prostate, testis, malignant neoplasms of the urinary tract, such as malignant neoplasms of kidney, renal pelvis, ureter, bladder, urethra or other urinary organs, malignant neoplasms of eye, brain and other parts of central nervous system such as malignant neoplasm of eye and adnexa, meninges, brain, spinal cord, cranial nerves and other parts of central nervous system, malignant neoplasms of thyroid and other endocrine glands such as malignant neoplasm of the thyroid gland, adrenal gland, parathyroid gland, pituitary gland, craniopharyngeal duct, pineal gland, carotid body, aortic body and other paraganglia, malignant neoplasms of head, face and neck, thorax, abdomen and pelvis, secondary malignant neoplasm of lymph nodes, respiratory and digestive organs, kidney and renal pelvis, bladder and other and urinary organs, secondary malignant neoplasms of skin, brain, cerebral meninges, or other parts of nervous system, bone and bone marrow, ovary, adrenal gland, malignant neoplasms of lymphoid, haematopoietic and related tissue such as Hodgkin's disease, follicular non-Hodgkin's lymphoma, diffuse non-Hodgkin's lymphoma, peripheral and cutaneous T-cell lymphomas, non-Hodgkin's lymphoma, lymphosarcoma, malignant immunoproliferative diseases such as Waldenström's macroglobulinaemia, alpha heavy chain disease, gamma heavy chain disease, immunoproliferative small intestinal disease, multiple myeloma and malignant plasma cell neoplasms such as plasma cell leukaemia, plasmacytoma, solitary myeloma, lymphoid leukaemia such as acute lymphoblastic leukaemia, myeloid leukaemia, monocytic leukaemia, blast cell leukaemia, stem cell leukaemia, and other and unspecified malignant neoplasms of lymphoid, haematopoietic and related tissue such as Letterer-Siwe disease, malignant histiocytosis, malignant mast cell tumour, true histiocytic lymphoma or other types of malignant neoplasia is treated using nucleic acid constructs from a composition or depot of the invention e.g. an NCCell.

Accordingly, the invention provides treatment of carcinoma in situ of oral cavity, oesophagus, stomach, digestive organs, middle ear and respiratory system, melanoma in situ, carcinoma in situ of skin, carcinoma in situ of breast, carcinoma in situ of female or male genitals, carcinoma in situ of bladder, urinary organs or eye, thyroid and other endocrine glands, or other types of carcinoma in situ.

In one embodiment, the nucleic acid encodes or silences a cytokine, chemokine, interleukin, or a combination of these involved in and preferably induces or enhances development of an anti-cancer immune reaction. In one embodiment the nucleic acid sequence encodes or silences one or more interleukin, including but not limited to IL-1, IL-2, II-15, IL-15 sushi, IL-6, IL-4, IL-6, IL-7, IL-10, IL-12, single chain IL-12p35-IL-12p40 fusion protein, IL-21, and/or IL-36. In one embodiment the nucleic acid sequence encodes or silences one or more interferons, including but not limited to IFN-alpha, IFN-beta, and IFN-gamma. In one embodiment, the nucleic acid sequence delivered encodes or silences therapeutic targets of immune signalling pathways, including but not limited to, activation or inhibition of TLR, RIG-1, STING, NOD-like, TNF-a, IFN-a, IFN-b, IFN-g, GM-CSF, OX40 (CD124) TNFSFR (e.g., CD40, OX40), SHP-1, SHP-2, SHP1/2 TIGIT, TGF-β1-3, TGF-β1-3 receptor, glycogen synthase kinase 3, STAT, Wnt/β-catenin, PI3Ks, c-KIT, mTOR, C-Myc, MET, BRAF, MEK, IDO1, Arg, A2AR, HIF-1 and 2, VISTACOX) 1 and/or 2, CTLA-4, PDLI, PDL2, PD1, B7-H3, B7-H4, BTLA, HVEM, TIM3, GAL9, LAG3, VISTA, KIR, 2B4, CD160, CGEN-15049, CHK 1, CHK2, A2aR, and B-7 family ligands, and any combination thereof. In one embodiment, the nucleic acid sequence delivered encodes or silences therapeutic targets of immune signalling pathways, including but not limited to, activation or inhibition of STAT1, STAT2, STAT3, STAT4, STAT5A, STAT5β, STAT6, and any combination thereof.

In one embodiment, the nucleic acid encodes or silences a long non-coding RNA including (lncRNA), including, without being limited thereto, MALAT1, HOTAIR, NEAT1, GAS5, ANRIL, and TUG1 and any combination thereof.

In one embodiment the RNA sequence is immunostimulatory, including, without being limited thereto, RNA sequences includes sequences with a short poly U tail or poly A tail and/or sequences encoding ligands of TLRs, preferably selected from human family members TLR1-TLR10 or murine family members TLR1-TLR13, more preferably selected from (human) family members TLR1-TLR10, even more preferably from TLR7 and TLR8, and ligands for intracellular receptors for RNA (such as RIG-I or MDA-5, etc.).

In one embodiment, the nucleic acid sequence delivered encodes a cytokine which is involved in and preferably induces or enhances development, priming, expansion, differentiation and/or survival of T cells, preferably an interleukin such as an interleukin selected from the group consisting of IL-2, IL-7, IL-12, single chain IL-12p35-IL-12p40 fusion protein, IL-15, IL-15 sushi, IL-27, IL-36 and IL-21.

In one embodiment, the nucleic acid component comprises a single nucleic acid construct or multiple constructs encoding, alone or in any therapeutic combination of IL-12, IL-12 single chain (IL-12p35-IL-12p40 fusion protein), IL-15, IL-15 sushi, OX40L, CD40, GM-CSF, IFN-a, IFN-b, and IFN-g. One embodiment describes delivery of single nucleic acid construct or multiple gene silencing constructs (e.g., siRNA and/or ASO), as single targets or in any given combination of IDO1, Arg, A2AR, SHP1, SHP2, SHP1/2, HIF-1 and 2, TGFb1, TGFb2, TGFb3, PD-1, PD-L1, CTLA-4, LAG3, TIM-3.

In one embodiment, the nucleic acid component comprises silencing nucleic acid constructs against immune checkpoints selected from the group consisting of CTLA-4, PDLI, PDL2, PD1, B7-H3, B7-H4, BTLA, HVEM, TIM3, GAL9, LAG3, VISTA, KIR, 2β4, CD160, CGEN-15049, CHK 1, CHK2, A2aR, and B-7 family ligands, and any combination thereof.

In one embodiment, gene silencing is applied for the treatment, inhibition, and/or prevention of cancer, including targeting genes involved in the development, progression, survival, expansion, metastases formation, therapeutic resistance, immune escape and inflammation of cancer. Examples of cancer genes to be targeted include, without limitation (examples of types of cancer, without limitation, to be treated in parentheses): BCL2 (melanoma, lung, prostate cancers or Non-Hodgkin lymphoma), GRM1 (melanoma), PDGF beta (testicular and lung cancers), Erb-B (breast cancer), Src (colon cancer), CRK (colon and lung cancers), GRB2 (squamous cell carcinoma), RAS (pancreatic, colon and lung cancers, and leukemia), MEKK (squamous cell carcinoma, melanoma or leukemia), JNK (pancreatic or breast cancers), RAF (lung cancer or leukemia), Erk1/2 (lung cancer), PCNA (p21) (lung cancer), MYB (colon cancer or chronic myelogenous leukemia), c-MYC (Burkitt's lymphoma or neuroblastoma), JUN (ovarian, prostate or breast cancers), FOS (skin or prostate cancers), Cyclin D (esophageal and colon cancers), VEGF (esophageal and colon cancers), EGFR (breast cancer), Cyclin A (lung and cervical cancers), Cyclin E (lung and breast cancers), WNT-1 (basal cell carcinoma), beta-catenin (adenocarcinoma or hepatocellular carcinoma), c-MET (hepatocellular carcinoma), PKC (breast cancer), NFKB (breast cancer), STATS (prostate cancer), survivin (cervical or pancreatic cancers), Her2/Neu (breast cancer), topoisomerase (ovarian and colon cancers), topoisomerase Il alpha (breast and colon cancers), p73 (colorectal adenocarcinoma), p21 (WAF1/CIP1) (liver cancer), p27 (KIP1) (liver cancer), PPM1D (breast cancer), RAS (breast cancer), caveolin | (esophageal squamous cell carcinoma), MIB | (male breast carcinoma), MTAI (ovarian carcinoma), M68 (adenocarcinomas of the esophagus, stomach, colon, and rectum), mutant p53 (gall bladder, pancreatic and lung cancers), mutant DN-p63 (squamous cell carcinoma), mutant pRb (oral squamous cell carcinoma), mutant APC1 (colon cancer), mutant BRCA1 (breast and ovarian cancers), mutant PTEN (hamartomas, gliomas, and prostate and endometrial cancers), MLL fusions (acute leukemias), BCR/ABL fusion (acute and chronic leukemias), TEL/AML1 fusion (childhood acute leukemia), EWS/FLI1 fusion (Ewing Sarcoma), TLS/FUS 1 fusion (Myxoid liposarcoma), PAX3/FKHR fusion (Myxoid liposarcoma), and AML1/ETO fusion (acute leukemia).

Treatment of cancer represents a field where combination strategies are especially desirable since frequently the combined action of two, three, four or even more cancer drugs/therapies generates synergistic effects which are considerably stronger than the impact of a monotherapeutic approach. In one embodiment cancer treatment is combined using immune- or vaccination-based mechanisms. In one embodiment composition or depot of the invention (e.g. NCCell) may effectively comprise or be combined with various other drugs and/or methods targeting similar or other specific mechanisms, non-limiting examples of anti-cancer drugs and therapies include Chemotherapy, radiation therapy, immunotherapy, surgery, antibodies, cytokines, chemokines, costimulatory molecules, fusion proteins, toll like receptor agonists, kinase inhibitors, vaccination, small molecule targeted therapy drugs, viral vaccines, adoptive cell transfer (e.g., lymphocyte, NK-cell, DC), peptide-based targeted therapies.

In one embodiment nucleic acid constructs are administrated to induce an immune response, in particular a cellular immune response, directed against a disease-associated antigen or cells expressing a disease-associated antigen such as cancer cells. In particular, the invention envisions the use of RNA encoding antigenic proteins or peptides (also termed “antigen” herein) inducing an immune response, in particular a T cell response, against the disease-associated antigen or cells expressing the disease-associated antigen. These antigenic proteins or peptides may comprise a sequence essentially corresponding to or being identical to the sequence of the disease-associated antigen or one or more fragments thereof. In one embodiment, the antigenic protein or peptide comprises the sequence of an MHC presented peptide derived from the disease-associated antigen. Immunisation with RNA encoding intact or substantially intact disease-associated antigen or fragments thereof such as MHC class I and class Il peptides makes it possible to elicit a MHC class I and/or a class Il type response and thus, stimulate T cells such as CD8+ cytotoxic T lymphocytes which are capable of lysing diseased cells and/or CD4+ T cells. Such immunization may also elicit a humoral immune response (B cell response) resulting in the production of antibodies against the antigen. Accordingly, the composition or depot (e.g. NCCell) may be used in vaccination, wherein an immune response is stimulated by introduction into a subject a suitable RNA molecule which codes for an antigenic protein or peptide. The invention may be used as a therapeutic or prophylactic vaccine for the treatment or prevention of a disease such as a disease as disclosed herein. In one embodiment the delivery of vaccine constructs may be targeted through the use ligands that secure specific or increased or primarily uptake in e.g., dendritic cells, macrophages. In one embodiment, a disease-associated antigen is a tumor antigen. In this embodiment, the agents and compositions described herein may be useful in treating cancer or cancer metastasis. Preferably, the diseased organ or tissue is characterized by diseased cells such as cancer cells expressing a disease-associated antigen and preferably presenting the disease-associated antigen in the context of MHC molecules. In one embodiment the delivered nucleic acid sequence or construct includes one or more antigen encoding sequences and an immune activating nucleic acid sequence. In one embodiment the immune encoding sequence of the vaccine construct is a short poly U tail or poly A tail. In another embodiment the immune activating construct of the nucleic acid vaccine construct encodes a pattern recognition receptor stimulating peptide or is by its structure recognized by pattern recognition receptors, examples include but are not limited to TLR7, TLR8, RIG-1, STING, MDA-5, LGP2, TLR4, TLR3, TLR9. In one embodiment the delivered nucleic acid sequence or construct includes one or more antigen encoding sequences and a nucleic acid sequence encoding or silencing an inflammatory gene or anti-inflammatory gene or immune modulating gene, including but not limited to IL-12, IL-2, IL-12 single chain IL-12p35-IL-12p40 fusion protein), IL-15, IL-15 sushi, IL-21, IL-36, IL-27, IFN-a, IFN-b, IFN-g, GM-CSF, OX40L, TGFb1-3, PD-L1, SHP1, SHP2, SHP1/2, arginase-1, and IDO.

In one embodiment, the nucleic acid component comprises a nucleic acid encoding one or more neoantigenic peptides. In one embodiment, the encoded protein is between about 8 to about 100 amino acids in length. In one embodiment, the encoded antigenic peptide also comprises flanking amino acids to the specific neo-antigen and these may not be native flanking amino acids. In one embodiment, the delivered nucleic acids encode for one or more isolated neo-antigenic peptides.

In one embodiment, the nucleic acid component comprises a nucleic acid encoding an antigen that is a cancer antigen, i.e., a constituent of cancer cells such as a protein or peptide expressed in a cancer cell which may be derived from the cytoplasm, the cell surface or the cell nucleus, those which primarily occur intracellularly or as surface antigens on cancer cells. For example, cancer antigens include the carcinoembryonal antigen, alpha-1-fetoprotein, isoferritin, and fetal sulphoglycoprotein, cc2-H-ferroprotein and γ-fetoprotein, p53, ART-4, BAGE, beta-catenin/m, Bcr-abL CAMEL, CAP-1, CASP-8, CDC27/m, CD 4/m, CEA, the cell surface proteins of the claudin family, such as CLAUDIN-6, CLAUDIN-18.2 and CLAUDIN-12, c-MYC, CT, Cyp-B, DAM, ELF2M, ETV6-AML1, G250, GAGE, GnT-V, Gapl OO, HAGE, HER-2/neu, HPV-E7, HPV-E6, HAST-2, hTERT (or hTRT), LAGE, LDLR/FUT, MAGE-A, preferably MAGE-A1, MAGE-A2, MAGE-A3, MAGE-A4, MAGE-A5, MAGE-A6, MAGE-A7, MAGE-A8, MAGE-A9, MAGE-A10, MAGE-A1 1, or MAGE-A12, MAGE-B, MAGE-C, MART-1/Melan-A, MC1 R, Myosin/m, MUC1, MUM-1,-2,-3, NA88-A, NF1, NY-ESO-1, NY-BR-1, pl 90 minor BCR-abL, Pm I/RARa, PRAME, proteinase 3, PSA, PSM, RAGE, Rul or RU2, SAGE, SART-1 or SART-3, SCGB3A2, SCP 1, SCP2, SCP3, SSX, SURVIVIN, TEL/AMLI, TPI/m, TRP-1, TRP-2, TRP-2/1NT2, TPTE and WT, preferably WT-1. Accordingly, a tumor antigen preferably comprises any antigen which is expressed in and optionally characteristic with respect to type and/or expression level for tumors or cancers as well as for tumor or cancer cells.

One embodiment relates to the co-delivery of a nucleic acid encoding for an antigen and an immunomodulator or adjuvant. In one embodiment, the immune modulator, activator or adjuvant is a molecular drug or multiple drugs or one or more nucleic acid sequence encoding for proteins with immune modulating properties or any combination of these. Therapeutic targets includes, but are not limited to Poly(I: C), Poly ICLC, STING agonists, MDA5, RIG-1 agonists, TLR agonists, 1018 ISS, aluminium salts, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, JuvImmune, LipoVac, MF59, monophosphoryl lipid A, Montanide IMS 1312 VG, Montanide ISA 206 VG, Montanide ISA 50 V2, Montanide ISA 51 VG, OK-432, OM-174, OM-197-MP-EC, ISA-TLR2 agonist, ONTAK, PepTel@ vector system, PLG microparticles, resiquimod, SRL172, virosomes and other virus-like particles, YF-17D, VEGF trap, R₈₄₈, beta-glucan, Pam3Cys, Pam3CSK4, acrylic or methacrylic polymers, copolymers of maleic anhydride, and QS21 stimulon. A costimulatory ligand, a TNF ligand, an Ig superfamily ligand, CD28, CD80, CD86, ICOS, CD40L, OX40, CD27, GITR, CD30, DR3, CD69, or 4-1BB. In one embodiment, the modulator or adjuvant is selected from the group of interleukins, including but not limited to IL-1, IL-2, Il-15, IL-6, IL-12 single chain IL-12p35-IL-12p40 fusion protein), IL-15, IL-15 sushi, IL-21, IL-27, IL-36 cytokines and chemokines, including but not limited to CCR7, CCL20, CCR7, CXCL9, CXCL10, CXCL11, FLT3L, OX40L, TNFs and IFNs.

In one embodiment, a single or combination of multiple vaccine epitope encoding nucleic acids are co-delivered with an TLR agonist and a TGFb inhibitor. In one embodiment a single or combination of multiple vaccine epitope encoding nucleic acids are co-delivered with an TLR7/8 agonist and an immune checkpoint inhibitor, including but not limited to inhibitors of PD-1, PD-L1, CTLA-4, LAG3, and TIM-3. In one embodiment a single or combination of multiple vaccine epitope encoding nucleic acids are co-delivered with R848 and RepSox. In one embodiment a single or combination of multiple vaccine epitope encoding nucleic acids are co-delivered with R848 and galunisertib.

Adoptive cell therapies for the treatment of cancer are challenged by poor therapeutic response rates, poor cell engraftment and survival. In one embodiment, the invention relates to nucleic acid delivery to increase performance of adoptive cell therapies by providing a sustained delivery of nucleic acid constructs (e.g., mRNA or pDNA) that encodes the cognate antigen or antigens recognized by the administered cell product to improve adaptive cell engraftment, proliferation, activation and survival and increase epitope spreading to endogenous adaptive immune cells. In one embodiment the combination of adoptive cell therapy and antigen delivery according to the invention relates to the combination of adoptive T cell products, including but not limited to ACTs and CAR-Ts. In one embodiment the invention provides delivery of a nucleic acid (e.g., mRNA or pDNA) encoding an antigen of which the human or animal being treated already has existing immunological activity in connection with previous infection or vaccination. In one embodiment the nucleic acid encoding an antigen against which the subject already has activity is injected into cancerous tissue to generate intralesional inflammation and produce epitope spreading towards cancer associated antigens or neo-antigens expressed by the cancer cells.

In one embodiment the administered antigen encoding construct is combined with a single or multiple molecular drugs or nucleic acids encoding an immune activating adjuvant or molecular adjuvants with immune activating, modulating or polarizing effects and/or cell recruitment activity, including but not limited to TLR7/8 agonist and an immune checkpoint inhibitor, including but not limited to inhibitors of PD-1, PD-L1, CTLA-4, LAG3, and TIM-3, immune modulators TGFb, Arg1, IDO inhibitors, cell recruiting chemokines CCR7, CCL20, CCR7, CXCL9, CXCL10, CXCL11, FLT3L, OX40L, TNFs and IFNs, and interleukins IL-1, IL-2, II-15, IL-6, IL-12, IL12p70, IL-12p40, IL-12p35, IL-12 single chain IL-12p35-IL-12p40 fusion protein), IL-15, IL-15 sushi, IL-21, IL-27, IL-36.

In one embodiment a single or multiple vaccine encoding nucleic acids, including but not limited to mRNA (including self-amplifying) and pDNA, are delivered in a composition or depot (e.g. NCCell) of the invention to serve as a single or multi-epitope vaccine. In one embodiment the delivered antigens encode for virulent bacterial antigenic proteins. One embodiment relates to the delivery of antibody encoding nucleic acids. In one embodiment the encoded antibodies neutralize virulence factor activities and inhibits complement-mediated bacterial lysis. In one embodiment the delivered vaccine nucleic acid construct and adjuvant encode vaccine that can be used to combat antimicrobialresistancy, including the vaccination against bacteria. In one embodiment the delivered antigens encode for Glycosyltransferase, Elastin Binding Protein, and Staphylococcal secretory antigen. Another embodiment relates to the delivery of single epitope or multiple epitopes encoding nucleic acid sequences or constructs that encode for, but not limited to double-mutant of Streptolysin-O (SLOdm), the backbone protein of pilus island 2a (BP-2a) from Group A (GAS, Streptococcus pyogenes) and Group B (GBS, Streptococcus agalactiae), M tuberculosis MPT83, LamB-LSA50, FhuA-LSA250, LPXTGp5, isaA, aurealysin, and protein A.

In one embodiment the delivered vaccine nucleic acid construct and adjuvant encode vaccine that can be used to treat, combat or prevent virus infections. In one embodiment a vaccine nucleic acid (e.g., mRNA) encoding a virus associated antigen is codelivered with a nucleic acid sequence encoding an immune activating adjuvant or a molecular drug with immune activation, modulating or polarizing activity. In one embodiment the encoded vaccine antigen can be, either as a single epitope or a combination of antigen encoding constructs, including but not limited to influenza antigens (viral glycoproteins HA, NA, NP, M1, M2, NS1, NEP), rabies antigens (RABV-G), corona virus antigens encoding spike protein(S), envelope protein (E), membrane protein (M), nucleocapsid protein (N) and hemagglutinin esterase dimer protein (H), RSV virus (prefusion F protein), Human metapneumovirus (HMPV) and parainfluenza virus type 3 (PIV3) (F protein), HCMV (HCMV pentamer complex and gB antigens (UL128, UL 130, UL131, gB, gH, gL), Zika (prME structural protein), EBV (EBV glycoproteins; gp42, gp220, gH, gL), and HIV (HIV-1 TV1 Env gp140, HIV-1 TV1 Env gp140, HIV-1 TV1 Env gp140 protein with MF59, Gag).

In one embodiment, nucleic acid constructs are delivered that direct therapeutic activity or vaccinate against autoimmune disorders and diseases. In one embodiment vaccinations against autoreactivity is achieved through the generation of regulatory T cells that provide bystander immunosuppression in the treatment of disease induced by cognate and noncognate autoantigens. In one embodiment, the delivered nucleic acid construct encodes 1 methylpseudouridine-modified mRNA coding for disease releated antigens including but not limited to myelin oligodendrocyte glycoprotein (MOG35-55).

In one embodiment, the delivered nucleic acids encode polypeptides or silence genes that act as agonist or antagonists for cytokines, chemokines, and growth factors, non-limiting examples of such cytokines are lymphokines, interleukins, monokines, chemokines, leukotrins, growth factors and hormones. Examples include encoding polypeptides or silencing of genes that are active agonists or antagonist for such as but not limited to growth factors including vascular endothelial growth factor (VEGFs), hepatic growth factor; fibroblast growth factor (FGFs); integrin; thrombopoietin (TPO); nerve growth factors such as NGF-β; platelet growth factor; transforming growth factors (TGFs); insulin-like growth factor-l and -II (IGFs); erythropoietin (EPO); osteoinductive factors; interferons, such as interferon-α, -β and -γ; colony stimulating factors (CSFs), such as macrophage-CSF (M-CSF), granulocyte-macrophage-CSF (GM-CSF), granulocyte-CSF (GCSF) and the like; interleukins (IIs); tumor necrosis factors, such as TNF-α or TNF-β; and other polypeptide factors, including LIF and kit ligand (KL). Examples include encoding polypeptides or silencing of genes that are active agonists or antagonist for such as but not limited to growth factors including fibronectin, epidermal growth factor (EGF), Sonic Hedgehog (SHH), Wnt proteins, bone morphogenetic proteins (BMPs), noggin, connective tissue growth factor (CTGF), SOX9, and hypoxia inducible factors. As used herein, the term cytokine includes proteins from natural sources or from recombinant sources (e.g., from T-cell cultures and biologically active equivalents of the native sequence cytokines). In one embodiment single or multiple nucleic acids encoding or silencing genes are delivered in combination with a single or combination of multiple antibiotics for the treatment of diseases, including but not limited to non-healing bone fractures, vascular defects, degenerative diseases, chronic infected wounds, diabetic ulcers.

One embodiment relates to treatment of endocrine disorders by gene engineering, including endocrine disorders in the hypothalamus, pineal body, pituitary gland, thyroid and parathyroid, thymus, adrenal gland, pancreas, ovaries and testicles associated with, but not limited to, Acromegaly, Adrenal Insufficiency & Addison's Disease, Cushing's Syndrome, Cystic Fibrosis, Graves' Disease, Hashimoto's Disease, Human Growth Hormone & Creutzfeldt-Jakob Disease, Hyperthyroidism (Overactive Thyroid), Hypothyroidism (Underactive Thyroid), Multiple Endocrine Neoplasia Type 1, Polycystic Ovary Syndrome (PCOS), Pregnancy & Thyroid Disease, Primary Hyperparathyroidism, Prolactinoma, Thyroid Tests, Turner Syndrome. Treatment of endocrine disorders also includes diabetes insipidus, Syndrome of inappropriate antidiuretic hormone (SIADH), multiple endocrine neoplasia type 2 (MEN2), congenital adrenal hyperplasia (CAH), and hypopituitarism.

In one embodiment, the delivered nucleic acids encode or silence genes of hormones, non-limiting examples of hormones include human growth hormone; parathyroid hormone (PTH); thyroxine; insulin; proinsulin; relaxin; prorelaxin; glycoprotein hormones; prolactin; placental lactogen; tumor necrosis factor-α and -β; mullerian-inhibiting substance; gonadotropin-associated peptide; inhibin; activin; adrenocorticotropic hormone (ACTH), amylin, angiotensin, atrial natriuretic peptide (ANP), calcitonin, cholecystokinin (CCK), gastrin, ghrelin, glucagon, follicle-stimulating hormone (FSH), insulin, Glucagon-like peptide-1 (GLP-1), leptin, luteinizing hormone (LH), melanocyte-stimulating hormone (MSH), oxytocin, parathyroid hormone (PTH), prolactin, renin, somatostatin, thyroid-stimulating hormone (TSH) thyrotropin-releasing hormone (TRH), vasopressin (arginine vasopressin (AVP) or anti-diuretic hormone (ADH)), and vasoactive intestinal peptide (VIP).

One embodiment relates to treatment genetic disease in a human or animal. This includes, but is not limited to, stimulating the production of the functional enzyme, protein or amino acid to help increase gene function in recessive diseases and diminishing the mutant protein formation through gene silencing to improve the phenotype, or increase the healthy wild-type gene to counterbalance the negative effects of the mutant gene in dominant diseases.

In one embodiment the composition or depot of the invention (e.g. NCCell) provides sustained released of gene engineering nucleic acid-constructs that encode or silence genes associated with hepatic disorders. In one embodiment, this is achieved through the direct administration of composition or depot of the invention (e.g. NCCell) in the liver or other organ or tissue of interest for treating. In one embodiment this achieved through injection in another place in the body and therefrom release nucleic acid constructs that have targeting moieties, this includes but is not limited to antibodies, nanobodies, GalNAc ligands for targeting the liver, mannose for targeting phagocytic cells, galactose, lactobionic acid, asialofetuin or folate for targeting of e.g., liver and/or cancer cells. In one embodiment the composition or depot of the invention (e.g. NCCell) release gene silencing constructs that target livers cell by expressing GalNAc. In one embodiment the liver targeting constructs deliver silencing nucleic acids that interfere with the production of an abnormal form of transthyretin. In one embodiment the liver targeting constructs deliver silencing nucleic acids, e.g., small interfering RNA (siRNA) directed towards delta-aminolevulinate synthase 1 (ALAS1).

One embodiment relates to treatment genetic disease in a human or animal. This includes, but is not limited to, stimulating the production of the functional enzyme, protein or amino acid to help increase gene function in recessive diseases and diminishing the mutant protein formation through gene silencing to improve the phenotype, or increase the healthy wild-type gene to counterbalance the negative effects of the mutant gene in dominant diseases.

In one embodiment, composition or depot of the invention (e.g. NCCell) provide sustained released of gene engineering nucleic acid-constructs that encode or silence genes associated with hepatic disorders. In one embodiment this is achieved through the direct administration in the liver or other organ or tissue of interest for treating. In one embodiment this achieved through injection in another place in the body and therefrom release nucleic acid constructs that have targeting moieties, this includes but is not limited to antibodies, nanobodies, GalNAc ligands for targeting the liver, mannose for targeting phagocytic cells, galactose, lactobionic acid, asialofetuin or folate for targeting of e.g., liver and/or cancer cells. This also includes but is not limited to aptamers, peptides, small molecules and glycans, including GalNAc and sialic acid.

In one embodiment, the composition or depot of the invention (e.g. NCCell) NCCell release gene silencing constructs that target liver cells using GalNAc including but not limited to e.g., small interfering RNA (siRNA), 2′-O-MOE-PS, 2′-O-MOE GalNAc, siRNA-GalNAc, GalNAc-ASO, 2′-O-MOE. In one embodiment the liver targeting constructs deliver silencing nucleic acids that interferes with the production of an abnormal form of transthyretin.

In one embodiment the composition or depot of the invention (e.g. NCCell) delivers silencing nucleic acids, e.g., siRNA, PS-oligos, ASO, 2′-O-MOE-PS, 2′-O-MOE, directed towards genes associated with genetic disease, hereditary disease, metabolic, obesity, diabetes, cardiovascular disease, liver disease, dyslipidemias, with silencing targets including but not limited to TGF beta 2, ApoC-III, Angiopoietin-like protein 3, Caspase 2, Alpha-1 antitrypsin, FGFR4, DGAT 2, Pre kallikrein, Glucagon receptor, Clusterin, Hsp27, Lp (a), ANGPTL3, STAT3, C5, TTR, ICAM-1, Anti-thrombin, TTR, p53, DMD pre-mRNA, apo-B-100, ALS1, mutated SOD1, mHTT, PCSK9.

In one embodiment liver targeting constructs, e.g., using GalNAc, deliver silencing nucleic acid constructs, e.g., small interfering RNA (siRNA), 2′-O-MOE-PS, 2′-O-MOE GalNAc, siRNA-GalNAc, GalNAc-ASO, 2′-O-MOE directed towards delta-aminolevulinate synthase 1 (ALAS1), Alpha-1 antitrypsin, Glucagon receptor, apo-B-100, Angiopoietin-like protein 3, ApoC-III, Anti-thrombin. In one embodiment, the liver targeting construct comprises sialic acid.

In one embodiment the composition or depot of the invention (e.g. NCCell) provides an injectable liquid ready for use in the treatment by injection in tissues, including soft tissue, organs, and bones for e.g. as part of a surgical treatment, post-surgical treatment or radiation therapy, in order to treat disorders of supportive tissue in a human or animal by regenerating and healing of bone tissue, soft tissue, soft tissue infections and/or treating bone infections. In one embodiment, the tissue is eye tissue. Such disorders may be, but not limited to bone loss, peritendinous adhesion formation, excessive granulation, chronic wounds, scar tissue, fibrosis, bone fracture, bone non-union, cartilage regeneration, bone trauma, cartilage regeneration, osteoarthritis, musculoskeletal diseases and disorder, soft tissue infections and/or osteomyelitis and encoded polypeptides include, but are not limited to hormones, antibiotic peptides, or growth factors and analogues of these, including but not limited to nucleic acids encoding BMPs, VEGFs (VEGF-A, VEGF-B, VEGF-C, VEGF-D, and placental growth factor (PIGF)), FGFs, TGFb1-3, BMPs, BMP-4, BMP2, BMP7, M-CSF, G-CSF, EGFs, EPO, HGFs, IGFs, OGFs, CGRPs, NRGs, PDGF, KGF, Ils, GDF9, GDNF, Calcitonin gene-related peptides (CGRP), Thrombin Peptide 508 (Chrysalin), and BMPs belonging to the transforming growth factor β (TGFβ) superfamily, including but not limited to TGFβs, activins/inhibins, Mullerian-inhibiting substance (MIS) and glial cell line-derived neurotrophic factor, GFOGER (glycine-phenylalanine-hydroxyproline-glycine-glutamate-arginine), SVVYGLR peptide, amide derivatives of stilbene, Bergenin, P15, RADA16-I, RANKL-binding peptide, core-binding factor A1 (CBFA1) Runt-related transcription factor-2 (RUNX-2), AC-100, mechano growth factor E, Osteoactivin and B2A2-K-NS (B2A) collagen-mimetic peptide HIP complexed and formulated in a composition or depot of the invention (e.g. NCCell).

One embodiment relates to treatment of infectious disease by delivering nucleic acids that encode on or more antimicrobial peptides (AMP) or antiviral peptides (AVP).

In one embodiment the composition or depot of the invention (e.g. NCCell) provides an injectable liquid for use in the treatment of diseases and disorders in the eye by delivering nucleic acids that encode or silence genes associated with, but not limited to Age-related macular degeneration (AMD), Retinitis pigmentosa (RP), Leber congenital amaurosis (LCA), Glaucoma, and Diabetic retinopathy.

In one embodiment the composition or depot of the invention (e.g. NCCell) provides an injectable liquid for use in the treatment of diseases and disorders in the heart by delivering nucleic acids that encode or silence genes associated with, but not limited to Heart failure, Myocardial infarction, Cardiomyopathy, and Arrhythmias

In one embodiment the composition or depot of the invention (e.g. NCCell) is administered (e.g. by injection) using image guidance, including but not limited to CT-, ultrasound-, MR-, PET-, SPECT-, fluoroscopy-, or endoscopy-guided imaging.

In one embodiment the composition or depot of the invention (e.g. NCCell) is administered (e.g. injected or applied) using image guidance, including but not limited to CT-, ultrasound-, MR-, PET-, SPECT-, fluoroscopy-, or endoscopy-guided imaging to secure optimal positioning and coverage of the intended treatment site e.g., infected or damaged or traumatized tissue or autoimmune or peri-infected areas and lesions or cancerous tissue.

In one embodiment the composition or depot of the invention (e.g. NCCell) is administered (e.g. injected) in tissues or organs in relation or connection with tissue defects or tissue trauma or degenerative or autoimmune diseases or disease or infections or infected tissues to provide release of nucleotides.

In one embodiment the composition or depot of the invention (e.g. NCCell) is used in relation to surgical procedures involving bone and joints. In a more preferred embodiment the composition or depot of the invention (e.g. NCCell) is administered (e.g. injected or applied or smeared) during surgical procedures involving trauma or fractures or arthroplasties or device installation.

In one embodiment, the composition or depot of the invention (e.g. NCCell) has a viscosity which allows injection or application and direct smearing into or on bone. In a more preferred embodiment the composition or depot of the invention (e.g. NCCell) is administered (e.g. injected) into trabecular bone or bone defects or surgically debrided bone lesions or bone marrow to provide release of nucleotides. In one embodiment, the composition or depot of the invention (e.g. NCCell) is administered (e.g. injected) into the trabecular bone wherefrom the nucleic acid component is released.

In one embodiment, the composition or depot of the invention (e.g. NCCell) is administered (e.g. injected) into joint spaces and tissues associated with joints. In a more preferred embodiment the composition or depot of the invention (e.g. NCCell) is administered (e.g. injected) in connection with infections in joints or prosthetic joints.

In one embodiment, the composition or depot of the invention (e.g. NCCell) is administered (e.g. injected or installed or applied) in connection with installation of artificial joints or surgical implants to induce tissue healing and regeneration or prevent the generation of prosthetic joint infections or implant associated infections. In a more preferred embodiment the composition or depot of the invention (e.g. NCCell) is administered (e.g. injected or installed or applied) in connection with non-healing or slow healing defect or infections in bone and joints and prosthetic material.

In one embodiment, the composition or depot of the invention (e.g. NCCell) is administered (e.g. injected) into trabecular bone or bone marrow releases nucleotides into the surgical bed or site and into the surgical cavity and into surrounding tissues. In one embodiment, the composition or depot of the invention (e.g. NCCell) is administered (e.g. injected or applied or smeared) into the subcutaneous space or intramuscularly or intraperitoneally.

In one embodiment the composition or depot of the invention (e.g. NCCell) is administered (e.g. injected or applied or smeared) into or onto tissues. In a more preferred embodiment the composition or depot of the invention (e.g. NCCell) is administered (e.g. injected or applied or smeared) into or onto tissue associated with surgical sites to improve healing and regeneration of tissues or stimulate an immune reaction or immune polarization.

In one embodiment the composition or depot of the invention (e.g. NCCell) is administered (e.g. injected or applied or smeared) in or onto allografts, autografts, xenografts, synthetic grafts, alloplastic grafts, composite grafts or amnion grafts.

In one embodiment the composition or depot of the invention (e.g. NCCell) is administered (e.g. injected or applied or smeared) in or onto surgical flaps or tissue grafts or prostheses or implants.

In one embodiment the composition or depot of the invention (e.g. NCCell) is administered (e.g. injected or applied or smeared) in or onto structures in the outer and or inner ear. In a more preferred embodiment the composition or depot of the invention (e.g. NCCell) is administered (e.g. injected or applied) to tissues and structures in the ear and surroundings including the mastoid bone.

In one embodiment the composition or depot of the invention (e.g. NCCell) has a viscosity which allows for injection or application or smearing in or onto teeth or periodontal spaces or maxillary bone or mandibulary bone. In a preferred embodiment the composition or depot of the invention (e.g. NCCell) has a viscosity that allows for injection in the periodontal spaces and release nucleotides for the treatment of periodontal diseases.

In one embodiment the composition or depot of the invention (e.g. NCCell) has a viscosity which allows for injection or application or smearing in the eye and structures in relation to the eye. In a preferred embodiment the composition or depot of the invention (e.g. NCCell) is administered (e.g. injected) into the suprachoroidal space or retrobulbar tissue or subretinal or transscleral or subconjunctival or juxtascleral or intravitreal space or tissues.

In one embodiment the composition or depot of the invention (e.g. NCCell) has a viscosity which allows for injection or application or smearing in the structures or tissues of the urinary system or urinary tract. In a preferred embodiment the composition or depot of the invention (e.g. NCCell) is administered (e.g. injected) in the bladder wall.

In one embodiment the composition or depot of the invention (e.g. NCCell) has a viscosity which allows for injection or application or smearing in gastrointestinal tract. In a preferred embodiment the composition or depot of the invention (e.g. NCCell) is administered (e.g. injected) in the gastric or intestinal wall.

In one embodiment the composition or depot of the invention (e.g. NCCell) is administered (e.g. injected) into the heart. In a more preferred embodiment, the composition or depot of the invention (e.g. NCCell) is injected or applied or smeared into or on the heart musculature.

In one embodiment, the composition or depot of the invention (e.g. NCCell) has a viscosity which allows for spraying or smearing or injection on implants prior to installation. In one embodiment, the composition or depot of the invention (e.g. NCCell) has a viscosity which allow for use in connection with the installation of Cochlear Implants, Deep Brain Stimulation (DBS) Systems, Spinal Cord Stimulators (SCS), Implantable Cardioverter Defibrillators, Ventricular Assist Devices, Neurostimulators, Retinal Implants, Artificial Urinary Sphincter (AUS), Gastric Electrical Stimulators, Intrathecal Drug Delivery Systems.

In one embodiment the injected or administered tissue comprises a cancer lesion e.g., primary malignant or benign tumor, a metastasis, a lymph node, a surgical bed for anti-cancer immune activation in connection with treatment e.g., surgery or radiation therapy.

In one embodiment, solid support material is included in a composition or depot of the invention (e.g. NCCell). Exemplary solid support materials include but are not limited to calcium sulphate, calcium phosphate, hydroxyapatite, silver nanoparticles, silver microparticles, organophosphates, whitlockite (WH), octacalcium phosphate, tetracalcium phosphate, beta-TCP, zirconia phosphate, silver nitrate (AgNO3), chitosan, PLGA, PEG, polycaprolactone (PCL), poly-I-lactic acid, monocalcium phosphate monohydrate, bioglass, polytetrafluoroethylene, polyethylene-oxide-terephtalane, polybuthylene-terephtalate-polymer, titanium, titanium salts, polypropylenefumarate (PPF), poly-methylmethaacrylate (PMMA), tricalcium phosphate, hydroxyapatite.

In one embodiment, the nucleic acid component (e.g. nucleic acid component comprising ASO or siRNA), is formulated as polyplexes, lipoplexes or HIPs using PAMAM generation 0, 1, 2, 3, 4, 5, 6, 7, 8, 9, 10 or 11. Surface modified PAMAM with hydroxyl, succinamic acid, carboxylate or C12 substitutions may also be used. PAMAM generation 0, 1 or 2 is preferred, and generation 0 is most preferred. Preferred NP ratios are NP 1-100, such as 2-50, such as 5-15.

In one embodiment, the nucleic acid component, (e.g. nucleic acid component comprising ASO or siRNA), is formulated as polyplexes, lipoplexes or HIPs using L-PEI, Mannose-JetPEI, such as L-PEI 20 kDa or 40kDA with an NP ratio of 1-100, such as 5-50 such as 10-30 such as 20-25.

In one embodiment, the nucleic acid component (e.g. nucleic acid component comprising ASO or siRNA) is formulated as PAMAM generation 0.0 polyplex (NP10). In one embodiment, the nucleic acid component (e.g. comprising ASO or siRNA) is formulated as L-PEI 40 kDa polyplex (NP25). In one embodiment, the nucleic acid component (e.g. comprising ASO or siRNA) is formulated as Mannose-JetPEI polyplex (NP8). In one embodiment, the nucleic acid component (e.g. comprising ASO or siRNA) is formulated as Mannose-JetPEI (NP8) polyplex including extra L-PEI 40 kDa (NP25) in the NCCell. In one embodiment, the nucleic acid component (e.g. comprising ASO or siRNA) is formulated as DOTAP: Chol: DOPE (50:45:5) NP2-5. In one embodiment, the nucleic acid component (e.g. comprising ASO or siRNA) is formulated as DOTAP: Chol (50:50) NP2-5. In one embodiment, the nucleic acid component (e.g. comprising ASO or siRNA) is formulated as DOTAP: DC-chol: Chol: DOPE 25:25:25:25 NP2-5. In one embodiment, the nucleic acid component (e.g. comprising ASO or siRNA) is formulated as PAMAM gen 0.0 polyplex NP10-20. In one embodiment, the nucleic acid component (e.g. comprising ASO or siRNA) is formulated as Spermine-chol HIP NP1-2. In one embodiment, the nucleic acid component (e.g. comprising ASO or siRNA) is formulated as DC-chol: DOTAP (50:50) dual HIP NP4.

In one embodiment, the nucleic acid component, (e.g. nucleic acid component comprising ASO or siRNA), is formulated as polyplexes, lipoplexes or HIPs and solubilized or dispersed in NCCell at a nucleotide concentration of 0.01-100 mg per gram NCCell, such as 0.1-10 mg per gram NCCell, such as 1-5 mg per gram NCCell such as 2-5 mg per gram NCCell.

In one embodiment, an NCCell comprising either LNPs, HIPs, polyplexes or lipoplexes is co-loaded with extra L-PEI at a concentration corresponding to NP1-100, such as NP 10-50 such as NP 20-30. Addition of extra L-PEI has advantageously been found to enhance transfection.

In one embodiment, one or more LNPs, HIPs, polyplexes or lipoplexes are co-formulated with small molecule APIs, peptides, antibodies and or FaBs to provide sustained release of both the nucleic acid component (e.g. gene transcribing or gene silencing nucleotides) and small molecule APIs, peptides, antibodies and or Fabs with direct therapeutic activity.

In one embodiment, one or more LNPs, HIPs, polyplexes or lipoplexes are co-formulated with small molecule APIs, peptides, proteins, antibodies FaBs, wherein the API is one or more anticancer agents, immunotherapeutic, chemotherapeutics, antimicrobials (antibiotics, antivirals, antifungals, antiprotozoals, and antihelminthics), anti-inflammatory agents, anticoagulant, antidepressant, antiepileptic, antipsychotic, sedatives, antidiabetic, cardiovascular, and/or analgesic agents; wherein the peptide is one or more therapeutic peptides, agonistic and antagonistic receptor ligands, peptides with regulatory or enzymatic activity, hormones, peptides with special targeting activities, vaccines, antigens, therapeutic peptides, diagnostic peptides, and/or steroids; wherein the protein is one or more immunotherapeutic agonists and antagonists, growth factors, cytokines, chemokines, hormones, glycoprotein hormones and antibodies, affibodies, peptide and/or nanobodies.

In one embodiment, one or more LNPs, HIPs, polyplexes or lipoplexes are co-formulated with one or more small molecule APIs, wherein the one or more APIs are selected from anticancer agents, immunotherapeutic agents, chemotherapeutics agents, antimicrobials (antibiotics, antivirals, antifungals, antiprotozoals, and antihelminthics), anti-inflammatory agents, anticoagulant agents, antidepressant agents, antiepileptic agents, antipsychotic agents, sedatives, antidiabetic agents, cardiovascular agents, and/or analgesic agents.

In one embodiment, one or more LNPs, HIPs, polyplexes or lipoplexes are co-formulated with one or more peptides, wherein the one or more peptides are selected from therapeutic peptides, agonistic and antagonistic receptor ligands, peptides with regulatory or enzymatic activity, hormones, peptides with special targeting activities, vaccines, antigens, therapeutic peptides, diagnostic peptides, and/or steroids.

In one embodiment, one or more LNPs, HIPs, polyplexes or lipoplexes are co-formulated with one or more proteins, wherein the one or more proteins are selected from immunotherapeutic agonists and antagonists, growth factors, cytokines, chemokines, hormones, glycoprotein hormones and antibodies, affibodies, peptide and/or nanobodies.

In one preferred embodiment, one or more LNPs, HIPs, polyplexes or lipoplexes are co-formulated with TLR7/8 agonist and TGFb inhibitors such as but not limited to R848 and RepSox.

In one embodiment, the nucleic acid component of the composition is stable for at least 5 days (e.g. at least 10 days, at least 15 days, at least 20 days, at least 25 days, at least 30 days, or at least 35 days) when stored at up to 4° C. It will be understood that a stable nucleic acid component does not demonstrate an appreciable decrease in activity (e.g. transfection activity) following storage as compared to the activity of nucleic acid components in aqueous solutions at room temperature.

The invention also provides a method for improving the stability of a nucleic acid component (e.g. mRNA) the method comprising incorporating the nucleic acid component into a composition of the invention, wherein the nucleic acid component is stable for at least 5 days (e.g. at least 10 days, at least 15 days, at least 20 days, at least 25 days, at least 30 days, or at least 35 days). In one embodiment, the composition is stored at up to 4° C. In one embodiment, the nucleic acid component comprises mRNA and the method comprises improving mRNA transfection efficiency.

The invention also provides a method for improving the stability of a nucleic acid component (e.g. mRNA), the method comprising transferring the nucleic acid component from an aqueous solution to a solvent by extraction, vacuum distillation transfer, and the like, wherein the nucleic acid component is stable for at least 5 days (e.g. at least 10 days, at least 15 days, at least 20 days, at least 25 days, at least 30 days, or at least 35 days). In one embodiment, the composition is stored at up to 4° C. In one embodiment, the nucleic acid component comprises mRNA and the method comprises improving mRNA transfection efficiency.

Methods for determining stability of nucleic acids are well known in the art. For example, the stability of a nucleic acid can be determined by incorporating a fluorophore into the nucleic acid component. Activity of the nucleic acid can be determined by detecting presence of the fluorophore in cells (the level of fluorescence provides a means for quantifying the amount of mRNA that has been transcribed).

Transfection activity may be evaluated by mean fluorescence (MFI) activity on flow cytometry of cells being treated with the particles stored either in composition of the invention or water for a period of time (e.g. 35 days). MFI is a direct measure of how mRNA has been transferred and transcribed in cells and therefore illustrate the transfer effectivity, biological activity, and availability of mRNA (which are each a measure of nucleic acid stability).

### EXAMPLES

Example 1: Preparation of NCCell formulations

Example 2: Polyplex preparation by simple mixing

Example 3: Lipoplex preparation by simple mixing

Examples 4: Preparation of Lipid and Polymeric Nanoparticles by microfluidic mixing

Examples 5: Preparation of hydrophobic ion pairing complexes

Example 6: Characterization of transfection particles

Example 7: Preparation and characterization of PS-oligos particles

Examples 8: Transfer of particles from water to organic media

Example 9: Ion-pairing of pDNA: mRNA and transfer from water to organic media

Example 10: Ion-pairing of silencing siRNA and transfer from water to organic media

Example 11: PS-oligos accumulate intracellularly when formulated as SLNP, HIPS, polyplexes and as free PS-oligos

Example 12: Preparation of NCCells with PEI pDNA/mRNA transfection systems

Example 13: Preparation of NCCells with DC-Chol-based pDNA/mRNA transfection systems

Example 14: Preparation of NCCells with KC2: DC-chol pDNA/mRNA transfection systems

Example 15: Preparation of NCCells with HIP-pDNA/mRNA transfection systems

Example 16: NCCells release particles

Example 17: Cryo-TEM imaging of polyplex particles in water and DMSO

Example 18: Cryo-TEM imaging of lipoplexes in water and DMSO

Example 19: Cryo-TEM imaging of polyplex particles in water released from NCCells

Example 20: Cryo-SEM imaging of NCCells containing polyplex particles

Example 21: Controlled release of transfection particles from NCCells evaluated by fluorescence

Example 22: Release of PS oligos from NCCells evaluated by fluorescence

Example 23: Controlled release of HIP: mRNA complexes from NCCells evaluated by Ribogreen

Example 24: Transfection capabilities of pDNA, mRNA, and siRNA particles are maintained in organic media

Example 25: NCCells provide sustained release of effective transfection particles

Example 26: NCCells can provide sustained release of transfection particles yielding a continuous production of IL-12 at levels capable of activation T lymphocytes

Example 27: NCCells increase T lymphocyte infiltration and cytotoxic T cell to regulatory T cell ratio in syngeneic murine cancer models

Example 28: NCCells sustained IL-12 transfection reduces tumor growth of a murine syngeneic colorectal cancer models

Example 29: Stability of SLNP transfection particles in water and DMSO

Example 30: NCCell display gene silencing capability

Example 31: Preparation of NCCell with various solvents

Example 32: Synthesis of Mannose Functionalized PEI

Example 33: Preparation of mannose-PEI particles and in vitro transfection using free mannose-PEI particles, and particles embedded in NCCell

Example 34: Stability of mRNA and pDNA transfection particles stored for 35 days at various conditions

Example 35: Method for up-concentrating pDNA transfection particles in NCCell

Example 36: In vitro sustained transfection from NCCell with OX40L/IL-12 dual plasmid

Example 37: Sustained in vitro release of siRNA particles from NCCell evaluated by fluorescence

Example 38: In vitro silencing by siRNA-complexes

Example 39: Sustained in vitro silencing by siRNA-complexes released from NCCell

Example 40: Stability of siRNA L-PEI 40 kDa polyplexes stored for 35 days in DMSO

Example 41: Chemical modifications of ASOs

Example 42: Preparation of polyplexes of ASOs

Example 43: Hydrophobic ion pairs of ASOs form particles when using large, hydrophobic counterions

Example 44: Hydrophobic ion-pairs of ASOs prepared by Bligh-Dyer method

Example 45: Methods for loading high concentrations of ASO complexes into NCCell

Example 46: Tailored in vitro release of ASOs from NCCell analyzed by fluorescence

Example 47: NCCell composition controls in vivo release kinetics of ASO HIPs and polyplexes

Example 48: Pharmacokinetics and biodistribution of Gd-DOTA labelled ASO released from NCCell analyzed with ICP-MS

Example 49: In vivo cellular uptake of ASO-complexes released from NCCell

Example 50: In vitro MALAT1 and KRAS silencing from ASO-complexes

Example 51: Stability of HIP complexes of ASO when stored over time

Example 52: Sustained in vitro silencing of MALAT1 by ASO released from NCCell

Example 53: KRAS efficacy in vivo

Examples relating to NCCell formulations are provided as non-limiting examples of compositions of the invention.

### Example 1: Preparation of Compositions of the Invention

Aim: To describe the preparation procedure of NCCell formulations as exemplary compositions of the invention, and formulation of transfection particles/systems herein.

### Method:

NCCell formulations are prepared by mixing carbohydrate esters e.g. LOIB, SuBen, LacBen, RaBen etc. with solvents and co-solvents e.g. GTO, GTH, DMSO, PC, EtOH etc. Lipids such as POPC, DOPE, Chol and the like may further be included in the NCCell. Compositions are given in weight percent (or weight ratio) and the corresponding amount of compound is weighed into a single glass vial. The mixture is placed in an ultrasonication bath at 70-80° C. for 1-2 hours and occasionally vortexed to generate homogenous solutions (NCCell) that are subsequently stored at 4° C. until further use. Transfection particles/systems are incorporated into the NCCell formulation by addition of transfection particles/systems dissolved in organic solvents such as DMSO, PC or EtOH. NCCell is subjected to gentle stirring until the formulation is transparent and homogeneous. The final NCCell formulation is transferred and stored in sealed vials at room temperature or at 4° C. Certain formulations may be stored at −20° C.

### Example 2: Polyplex Preparation by Simple Mixing

Aim: The aim of this example is to describe the preparation of polymer-based RNA/DNA transfections systems, such as systems based on polyethyleneimine (PEI) using simple mixing.

### Principle:

Nucleic acids are complexed to a cationic polymer such as polyethyleneimine (PEI) by charge-charge interactions in water. The complexes in water are mixed with DMSO and subjected to vacuum oven treatment. Water will evaporate much faster than DMSO resulting in transfer of the water-dissolved particles into DMSO. Free PEI is soluble in DMSO whereas nucleic acids are not. The complex is soluble in DMSO.

### Protocol:

Preparation of PEI solutions: PEI is dissolved in water to 1.25 mg/ml and the pH is lowered to pH 2 using 1 M HCl. When the PEI is completely dissolved, the pH is reset to 7 using 1M NaOH. Add water so the final volume is 100 ml and the concentration is 1 mg/ml. The dissolved PEI is sterile filtered and aliquoted into Eppendorf tubes and stored at −20° C. and are ready to use. PEI can be dissolved to higher concentrations such as 2 and 5 mg/ml using the same protocol.

N/P ratios: The N/P ratio is a measure of the charge balance of the DNA-PEI complexes. The positive charge of PEI is originated from the nitrogen of the repeat unit of PEI, NHCH₂CH₂, that has a molecular weight of 43 g/mol. The negative charge in the plasmid DNA backbone comes from the phosphate group of the nucleotides. The average molecular weight of the nucleotides is assumed to be 330 g/mol.

### Example of N/P Ratio Calculation

Complexation of 1 μg of plasmid DNA with PEI at N/P ratio 10, requires 1.3 μL 1 mg/ml (23 mM=23 nmol/μL) of PEI solution.

$$Phosphate molar concentration = plasmid DNA weight / nucleotide molecular$$

$${n}_{P} = 1 \times {10}^{- 6} g / 330 g / mol = 3 nmol .$$

$$At N / P ratio 10 , {n}_{N} = {n}_{p} \times 10 = 3 nmol \times 10 = 30 nmol .$$

$$The amount of PEI needed = 30 nmol / 23 nmol / μL = 1.3 μl .$$

### Examples for Approximate N/P Ratios

NP 8:10 μg of nucleic acids dissolved in 39-89 μl water are coupled to 11 μl of L-PEI25K stock solution (1 mg/ml). The nucleic acids and L-PEI25K are first diluted in water. A final pDNA/mRNA concentration of 0.1-0.2 μg/μl is suitable. The 11 μL of L-PEI25K is added to the 39-89 μL pDNA/mRNA solution and quickly mixed by tapping or vortexing. After 15-30 minutes at room temperature the complexes can be used for transfection or further processed.

NP 25:10 μg of nucleic acids dissolved in 17-67 μL water are coupled to 33 μl of L-PEI25K stock solution (1 mg/ml). The nucleic acids and L-PEI25K are first diluted in water. A final pDNA/mRNA concentration of 0.1-0.2 μg/μl is suitable. The 11 μL of L-PEI25K is added to the 17-67 μL pDNA/mRNA solution and quickly mixed by tapping or vortexing. After 15-30 minutes at room temperature the complexes can be used for transfection or further processed.

### Example 3: Lipoplex Preparation by Simple Mixing

Aim: The aim of this example is to describe the preparation of lipid based RNA/DNA transfections systems using liposomes based on DOTAP, DC-Chol, Dlin-KC2-DMA using simple mixing.

### Principle:

Liposomes comprising cationic lipids such as DOTAP, DC-Chol and Dlin-KC2-DMA are mixed with RNA or DNA by simple mixing in buffered systems leading to formation of lipoplexes.

### Protocol:

Preparation of liposomes: liposomes are prepared from freeze-dried lipid mixtures. 50 mM lipid stocks are prepared in Tert-butanol: H₂O (milliQ water) 90:10. The lipids are mixed according to the desired molar ratio:

Example formulations: DOTAP: Cholesterol 50:50, DOTAP: Cholesterol: DOPE 50:25:25, DOTAP: Cholesterol: DOPE 50:40:10, DOTAP: Cholesterol: DOPE 50:45:5, DOTAP: Cholesterol: DOPE 50:30:20, DOTAP: Cholesterol: DOPE 40:30:30, DOTAP: Cholesterol: DOPE 40:40:20, DOTAP: Cholesterol: DOPE 40:50:10, DOTAP: Cholesterol: DOPE 30:30:40, DOTAP: Cholesterol: DOPE 30:40:30, DOTAP: Cholesterol: DOPE 30:50:20, DC-Cholesterol: Cholesterol: DOPE 25:70:5 DC-Cholesterol: Cholesterol: DOPE 25:60:15, DC-Cholesterol: Cholesterol: DOPE 25:50:25 DC-Cholesterol: Cholesterol: DOPE 30:65:5, DC-Cholesterol: Cholesterol: DOPE 30:35:35 DC-Cholesterol: Cholesterol: DOPE 40:30:30, DC-Cholesterol: Cholesterol: DOPE 40:40:20 DC-Cholesterol: Cholesterol: DOPE 40:50:10, DC-Cholesterol: Cholesterol: DOPE 50:45:5 DC-Cholesterol: Cholesterol: DOPE 50:25:25, DC-Cholesterol: Cholesterol 33:67 DC-Cholesterol: Cholesterol 30:70, DC-Cholesterol: Cholesterol 25:75 DC-Cholesterol: Cholesterol 20:80

The lipids at the chosen ratio and with a stock concentration of 50 mM are pipetted into freeze dry-compatible vials. The lipid mix is snap-frozen in liquid nitrogen and freeze dried overnight.

The lipid powder is hydrated in sterile MQ water for one hour and thereafter subjected to high pressure extrusion. Extrusion proceeds through one round where a 200 and a 100 nm filter is stacked followed by five rounds with two stacked 100 nm filters. If needed the temperature can be increased to 65° C. The final lipid concentration is measured by analyzing the phosphor content using ICP-MS and the size and zeta potential are measured using dynamic light scattering (DLS).

### N/P Ratios

DOTAP has one nitrogen atom that is positively charged and is used as an example of a lipid carrying a single positive charge per molecule. The N/P ratio is a measure of the ionic balance of the DNA/RNA-DOTAP complexes. The negative charge in the plasmid DNA/RNA backbone comes from the phosphate group of the nucleotides. The average molecular weight of the nucleotides is assumed to be 330 g/mol.

### Example of N/P Ratio Calculations

Complexation of 1 μg of plasmid DNA with DOTAP liposomes at N/P ratio 10, requires 2 μL 30 mM DOTAP liposomes containing 50% DOTAP.

Molar phosphate content in 1 μg of plasmid

$${n}_{P} = 1 \times 1 {0}^{- 6} g / 330 g / mol = 3. 03 \times 1 {0}^{- 9} mol = 3 nmol .$$

At N/P ratio 10, the molar content of nitrogens required are nn=npx10=3nmol×10=30 nmol which is contained in 2 μl 30 mM DOTAP liposomes (50 mol % DOTAP).

### Examples for Approximate N/P Ratios Using 10 μg of Nucleic Acids

NP 1. 10 μg of nucleic acids are complexed with 2 μl of 30 mM DOTAP liposome stock solution (50 mol % DOTAP).

NP 5. 10 μg of nucleic acids are complexed with 10 μl of 30 mM DOTAP liposome stock solution (50 mol % DOTAP).

NP 10. 10 μg of nucleic acids are complexed with 20 μl of 30 mM DOTAP liposome stock solution (50 mol % DOTAP).

A final pDNA/RNA concentration of 0.1-0.2 μg/μl is suitable. The nucleic acids and DOTAP liposomes are first diluted in water. The DOTAP liposomes are added to the pDNA/RNA solution and quickly mixed by tapping or vortexing. After 15-30 minutes at room temperature the complexes can be used for transfection or further processed.

DC-Cholesterol has two nitrogen atoms, however the nitrogen next to the carboxyl group is not positively charged. DC-Cholesterol contains only one protonatable nitrogen atom

The N/P ratio is a measure of the ionic balance of the DNA-DC-Chol complexes. The positive charge of DC-Chol is originated from the single protonatable nitrogen. The negative charge in the plasmid DNA backbone comes from the phosphate group of the nucleotides. The average molecular weight of the nucleotides is assumed to be 330 g/mol.

### Example of N/P Ratio Calculations

$$Phosphate molar concentration = plasmid DNA weight / nucleotide molecular weight = 1 \times {10}^{6} / 330 = 3.03 \times {10}^{- 9} M = 3 nmol .$$

$$At N / P ratio 10 , N = P \times 10 = 3 \times 10 = 30 .$$

One microliter of 9 mM DC-Chol liposomes has 9 nmol of nitrogen. The amount of DC-Chol liposomes needed=30/9=3.33 μl.

### Examples for Approximate N/P Ratios Using 10 μg of Nucleic Acids

NP 1. 10 μg of nucleic acids are coupled to 3.33 μl of DC-Chol liposome stock solution (9 mM DC-Chol).

NP 5. 10 μg of nucleic acids are coupled to 16.65 μl of DC-Chol liposome stock solution (9 mM DC-Chol).

NP 10. 10 μg of nucleic acids are coupled to 33.33 μl of DC-Chol liposome stock solution (9 mM DC-Chol).

### Example 4: Preparation of Lipid and Polymeric Nanoparticles by Microfluidic Mixing

Aim: Describe the basic principles of particle formation using microfluidic mixing Principle: Lipids, including cationic and/or ionizable lipids, are dissolved in pure ethanol, vortexed and mixed to the desired molar ratio. In the case of polymeric particles, polymers are dissolved in ultrapure water. In parallel, DNA or RNA is diluted in either Sodium Acetate buffer (25 mM, pH 4) or ultrapure water. Both solutions are briefly vortexed, loaded into syringes of appropriate volume and attached to the channels of a cartridge in the microfluidic mixing system (Nanoassemblr Ignite, Precision Nanosystems). The nanoparticles are formulated by microfluidic mixing of the lipids in organic phase and the DNA or RNA aqueous phase at a volume ratio of 1:3, or 1:1 in the case of polymers, at a flow rate ranging from 9-15 mL/min, aiming for a specific nitrogen: phosphate (NP) molar ratio. After formulation, the solution is vortexed and allowed to rest at room temperature for 15-30 minutes. For the lipid nanoparticles (LNP), the ethanol is exchanged to either Hepes buffer (25 mM, 150 mM, pH7.4) or ultrapure water by spin filtration. The LNP solution is placed in Amicon Centrifugal Filter Units (100 kDa MWCO) and spin down at 500g until sample volume has been reduced to half. Then exchange buffer/water is added to the retained sample to bring it back to original volume and gently mixed by pipetting along the membranes. This process is repeated six times and after the last centrifugation exchange buffer/water is added to bring the LNP concentration to the final target value. Particle solutions are stored at 4° C. until further use.

### Example 5: Preparation of Hydrophobic Ion Pairing Complexes

Aim: Describe the basic preparation of hydrophobic ion pairs (HIPs) of polynucleotides.

Principle: Hydrophobic cationic counterions are dissolved in ultrapure water at 1 mg/mL. The solution containing cations is further diluted to match the desired NP ratio and mixed with solutions of either mRNA, pDNA, siRNA or PS-oligos, in ultrapure water at 0.4 μg/μL concentration, aiming for a final nucleotide concentration of 0.1 μg/μL. After mixing, the HIP complexes are vortexed and then left for 30 minutes at room temperature. The HIP transfection complexes can be stored at 4° C. until further use.

### Example 6: Characterization of Transfection Particles

Aim: Describe the methods/techniques used to characterize the transfection particles. Methods:

Dynamic light scattering (DLS): Size, polydispersity Index (PDI) and surface charge (Zeta-Potential) of the particles are measured by dynamic light scattering (DLS) using a Zetasizer Nano ZS (Malvern). The particle solution is diluted to 10-50 μg/mL DNA/RNA with ultrapure water or filtered buffer and placed in a microcuvette. Size measurements are done by performing three main runs on the DLS in automatic mode. For Zeta-Potential measurements, samples are diluted the same way to final volume of 500-700 μL and measured using a Dip Cell kit performing three main runs in automatic mode.

Nanoparticle Tracking Analysis (NTA): Transfection particles, in water or organic solvent such as DMSO, are diluted in either ultrapure water or filtered buffer to a concentration of 50-100 ng/mL DNA/RNA. Between 0.5-1 mL of the solution is injected using a syringe into the cell of the instrument and the size and concentration of the particles are measured using Nanoparticle Tracking Analysis (NTA, ZetaView, Particle Metrix). The results are the average of 11 measurements made at different locations in the cell with the camera sensitivity set between 60-85 and shutter at 100.

Quantification of DNA/RNA concentration and encapsulation: Concentration of DNA/RNA in the samples and ratio of encapsulated DNA/RNA is determined using Picogreen/Ribogreen assay following the manufacturer's protocol (Invitrogen). Particles are diluted in Assay Buffer to approx. 50-100 ng/ml DNA/RNA to fit within a standard range with 100 ng/ml as maximum. 300 μL of the diluted particles are 2-fold diluted with Triton solution (TX, 0.5% hydrogenated Triton X-100), vortexed and incubated at 60° C. for 30 min under shaking conditions. After incubation, the dissociated particles are left to cool down to room temperature. Then 100 μL of particles diluted in Assay Buffer or dissociated in TX are placed in triplicates in a flat black 96-well plate. Two standard curves are prepared by diluting a DNA/RNA standard in Assay Buffer or TX covering a range from 0-100 ng/ml, and 100 μL of each standard is placed in triplicates in the 96-well plate. Using a multipipette, 100 μL of the Picogreen/Ribogreen dye is added to each of the samples and left in the dark for 5 minutes. Fluorescence (Ex.485 nm/Em. 535 nm, bandwidth 10 nm) is measured on a Spark platereader (Tecan) using optimal settings. Concentration of DNA/RNA is determined by interpolating with the standard curve and encapsulation percentage is calculated dividing concentration measured in Assay Buffer divided by concentration measured in TX and multiplied by 100.

RNase protection assay by HPLC: Particles are diluted to 100 μg/mL Luc or mCherry mRNA in the presence or absence of RNase enzyme cocktail (100×diluted, Invitrogen) and incubated for 1 h at 37° C. The reaction is then terminated by adding 5 μL of proteinase K. Concentration of RNA is determined by HPLC (Shimadzu) using UPLC C18 column and solvent gradient of 1 min at 5% B, 5-20% B in 8 min, 20-50% B 4 min and 50-100% B in 1 min, where (A) is 1% Hexafluoro Isoprop (HFIPA), 0.1% Diisopropiletilamina (DIPEA) in MQ, and (B) is 1% HFIPA, 0.1% DIPEA in CH3CN. The ratio of protected RNA is calculated using the formula:

$$Percentage of protected RNA = 1 - ( AUC of RNA with RNase / AUC of RNA without RNase ) \times 100$$

Gel shift assay: Agarose gel mobility shift assay.

The ability of pDNA to complex to poly- or lipoplexes was analyzed by agarose gel electrophoresis. Uncomplexed nucleic acids migrates rapidly through the gel toward the anode but when complexed in larger structures the nucleic acids will migrate slower or remain in the well.

Poly or lipoplexes at a predetermined N/P ratio mixed with loading dye (2:10 ratio), and 10 μL were loaded on 1% agarose gel in Tris-acetate buffer for 30 minutes at 100 V. The gel was stained with Sybr Safe at a 10 000× dilution. A DNA ladder was included for reference and the DNA bands were visualized with an UV illuminator.

### Data Examples

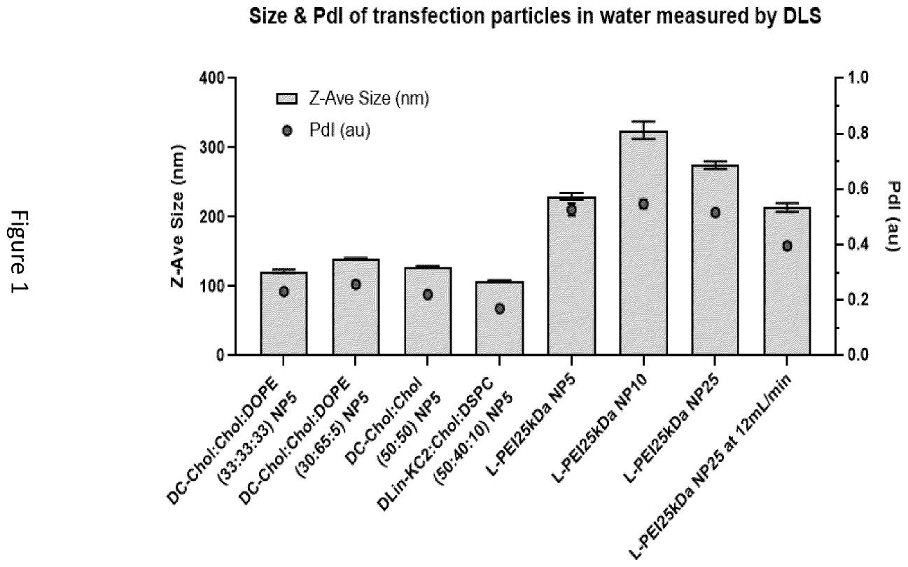

DLS: Example of size and PDI results of transfection particles with mCherry pDNA diluted in ultrapure water to 10 μg/mL and measured by DLS are given in FIG. 1. The results show that lipid-based transfection particles are slightly smaller (100-130 nm) compared to the PEI-based particles (200-300 nm).

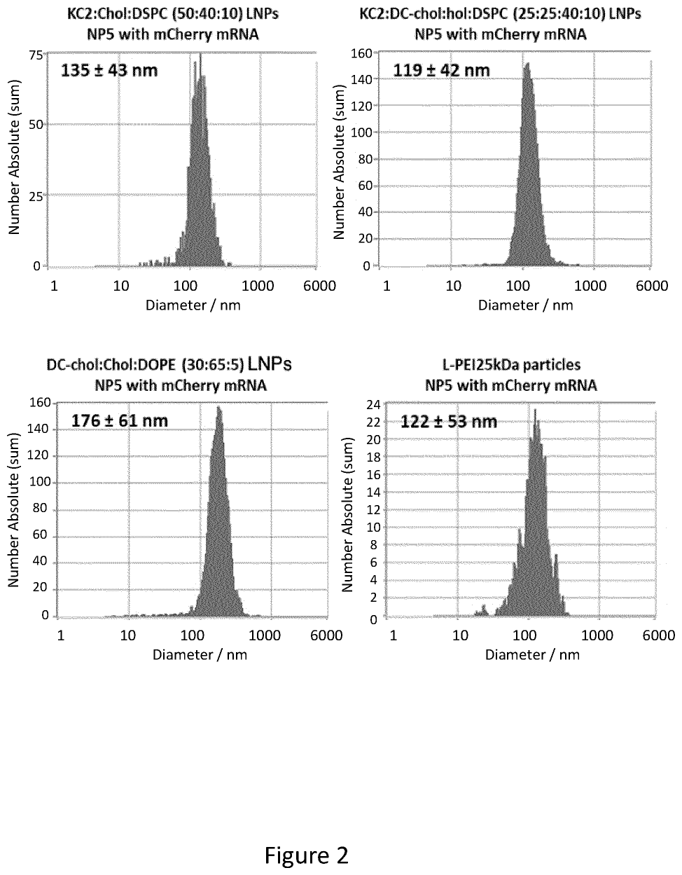

NTA: Example of size distribution histograms of transfection particles with mCherry mRNA at NP5 diluted in ultrapure water to 100 ng/mL and measured using the NTA is presented in FIG. 2. The particles based on Dlin-KC2 (KC2), DC-chol lipids or combination of both, show narrow size distribution profiles with average size between 100-130 nm. The results also show homogeneous L-PEI25k particles of similar sizes.

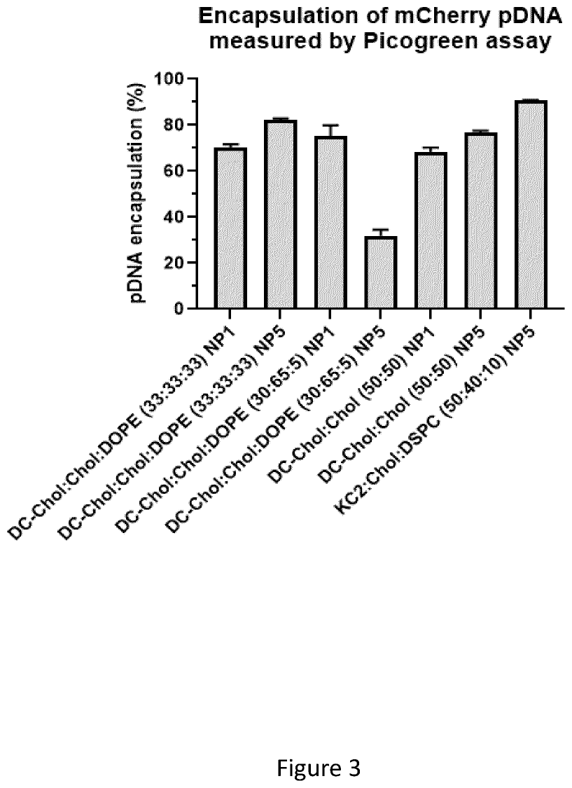

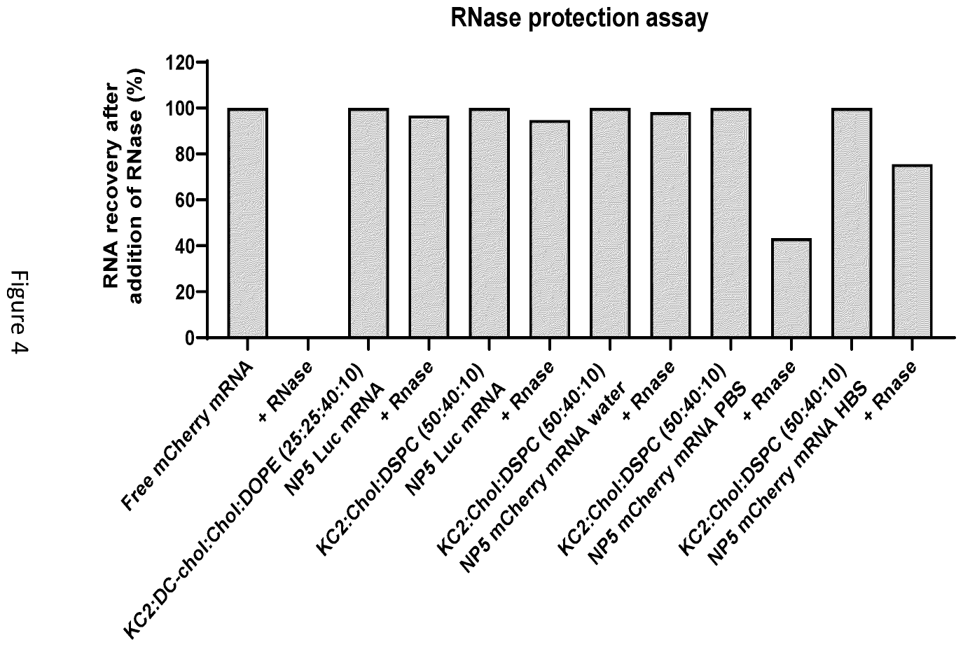

Picogreen: Example of quantification results using the Picogreen assay is presented in FIG. 3, showing the degree of encapsulation of mCherry pDNA in transfection particles made with the Ignite flow system.

Example of RNase protection assay using HPLC is given in FIG. 4. The lipid-based transfection particles prepared with the Ignite were diluted to 50-100 μg/mL mRNA as well as the control free mCherry mRNA in water solution (100 μg/mL). 100 μL of each solution was incubated for 1 h at 37° C. in the presence or absence of 1 μL RNase enzyme cocktail, and then the mRNA content was determined by HPLC as previously described. The results indicate that when mRNA is formulated in the transfection particles it remains protected from RNase degradation.

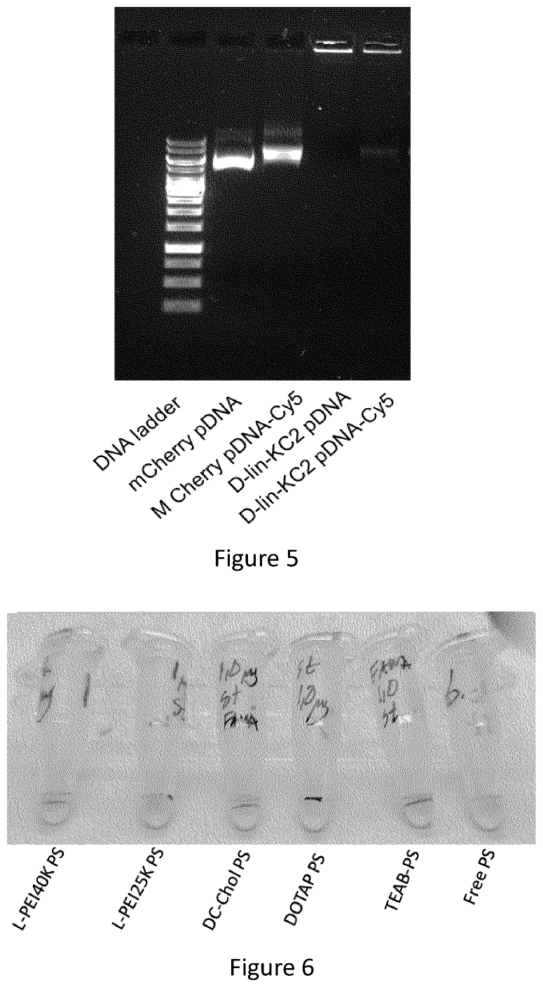

Gel shift assay: free mCherry pDNA, Cy5 labelled mCherry pDNA, and Dlin-KC2: Chol: DSPC 50:40:10 SLNPs of both pDNAs were prepared according to example 4. The samples were loaded onto an agarose gel and the developed according to protocol. Examples of the DNA ladder, complexes and uncomplexed pDNA bands are shown in FIG. 5. Here normal migration of free pDNA is observed whereas pDNA of Dlin-KC2: Chol: DSPC 50:40:10 SLNP are not migrating in the gel.

### Conclusion

This example presents the different techniques/methods used to investigate the main characteristics of the transfection particle systems. These include DLS and NTA for particle size, PDI, surface charge, Ribogreen/Picogreen and gel-shift assay to evaluate the concentration and encapsulation of mRNA/pDNA, HPLC to determine the mRNA concentration in the particles in the presence and absence of Rnases, thus assessing the protection against RNase degradation.

### Example 7: Preparation and Characterization of PS-Oligos Particles

Aim: Demonstrate preparation and characterization of silencing particles made of Cy5-labelled-PS-oligos (PS).

Methods: Here we demonstrate uptake of scramble cy5-labled PS-oligo, that are single-stranded synthetic nucleic acids with a special chemical modification, 29-deoxy-2-fluoro-D-arabinonucleic acid. Cy5-PS-oligos were obtained from AUM Biotech, USA.

PEI-PS particles: Complexes with composition L-PEI40K PS or L-PEI25K PS were prepared according to example 2 at NP5. Polyplexes were prepared at 0.1 PS μg/μL. Liposome-based PS oligos particles: DC-Chol: Chol: DOPE (30:65:5), DOTAP: Chol: DOPE (50:45:5) NP5, were prepared according to example 3. Lipoplexes were prepared at 0.1 PS mg/μL.

SLNP-based PS silencing particles: KC2: Chol: DSPC (50:40:10), KC2: DC-Chol: Chol: DSPC (25:25:40:10) NP5, were prepared according to example 4. SLNPs were prepared at 0.1 PS μg/μL.

HIP-based_PS_silencing particles: TEAB-PS complexes at NP2, were prepared according to example 5. The HIP complexes were prepared at 0.1 PS μg/μL. PS: PS was dissolved in H₂O at 0.1 PS μg/μL.

Silencing complexes in DMSO: 50 μL of polyplexes, lipoplexes or HIP complexes were mixed with 50 L DMSO and were transferred to DMSO using a vacuum oven as described in example 8.

### Results and Discussion

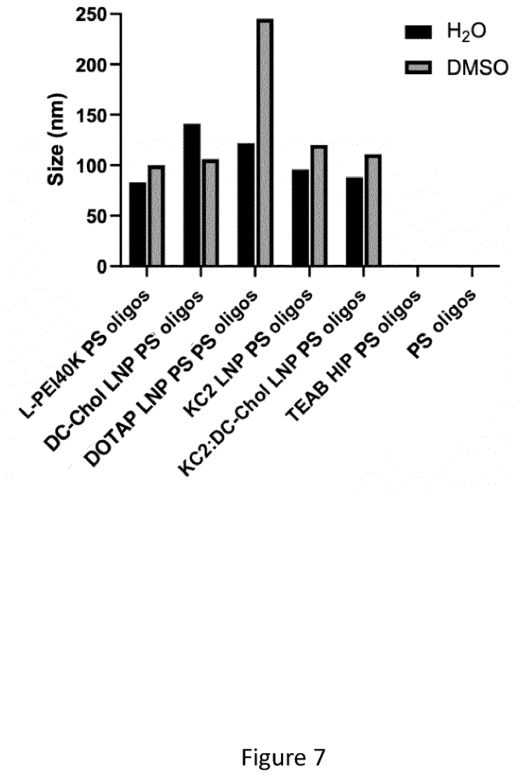

Silencing particles were prepared using Cy5 labelled PS oligos according to the methods section. Silencing particles could be transferred into DMSO without any precipitations. Example complexes are shown in FIG. 6. Particle size was measured in both water and DMSO using NTA. L-PEI40K, DC-Chol liposomes, DOTAP-liposomes, KC2 LNPs and KC2-DC-Chol LNPs all formed particles with PS oligos and the sizes in H₂O and DMSO ranged between 80-250 nm. HIP complexes prepared with TEAB and free PS did not form any measurable particles. FIG. 7.

### Conclusion

Silencing complexes with PS oligos and PEI-polymers, liposomes and LNPs can be prepared in water and transferred into DMSO and maintain similar sizes. The small HIP TEAB and free PS do not form particles but can be transferred from water into DMSO.

### Example 8: Transfer of Particles from Water to Organic Media

Aim: Describe the principles of transferring lipoplexes and polyplexes from aqueous to organic media such as propylene carbonate (PC), Benzyl alcohol (BnOH) and Dimethyl sulfoxide (DMSO).

Principle: The NCCell system is intrinsically hydrophobic, and water-containing transfection systems cannot be readily and homogeneously dissolved. Therefore, polyplexes and lipoplexes are transferred from water phase in which they were prepared to an organic solvent with higher boiling point like PC, BnOH or DMSO before being solubilized in a NCCell. This can be achieved by simply mixing transfection particles in water with organic solvent e.g. DMSO and followed by evaporation of the water using a vacuum oven. In this process, the polyplexes or lipoplexes will be up-concentrated in remaining organic phase. The process can be performed at room temperature.

### Particle Preparation:

L-PEI25k-pDNA (mCherry) transfection particles were prepared according to Examples 2. Briefly, LPEI-25k dissolved in water at 1 mg/ml was injected into a water solution containing mCherry pDNA and rapidly mixed. The final pDNA concentration was between 0.2-0.3 μg/μl and the NP ratio was 25. The samples were incubated for 30 minutes at room temperature before being further processed.

LPEI40k-pDNA (mCherry) transfection particles were prepared according to Examples 2. Briefly, LPEI-40k dissolved in water at 1 mg/ml was injected into a water solution containing mCherry pDNA and rapidly mixed. The final pDNA concentration was between 0.2-0.3 μg/μl and the NP ratio was 25. The samples were incubated for 30 minutes at room temperature before being further processed.

KC2: Chol: DSPC (50:40:10) LNPs with mCherry mRNA or Luc pDNA were prepared according to example 4. Briefly, solutions of lipids in ethanol and mRNA/pDNA in sodium acetate buffer were mixed using the Ignite to form LNPs at a ratio of NP 5. After short incubation at room temperature, the ethanol was exchanged with water/buffer, and particles up-concentrated to 0.4 μg/mL mRNA/pDNA.

Vacuum transfer: Varying volumes (25-150 μL) of LPEI25k polyplexes or KC2 LNPs with pDNA or mRNA prepared in water is mixed with varying volumes (10-100 μL) of either DMSO, PC or BnOH in a clean Eppendorf tube. Two holes in the lid is made with a 18G needle and the sample is subjected to vacuum oven treatment (Vacutherm Thermo Scientific pressurized below 20 mbar at room temperature). The total sample weight is recorded at time Oh, 1 h, 2 h and 3 h using a microbalance and the reduction in water content was determined by weight.

Comparison of particle size in water and upon transfer to DMSO: The transfection systems PEI-based systems (LPEI25k and LPEI40k) with pDNA or mRNA, and KC2-based LNPs with mRNA or pDNA in water were diluted in PBS and their size was determined using NTA according to Example 6. Particles were transferred to DMSO using the vacuum oven by mixing 50 μL sample and 50 μL DMSO. After complete transfer of particles into DMSO, these were diluted in water/PBS and their size were determined using NTA.

### Results and Discussion

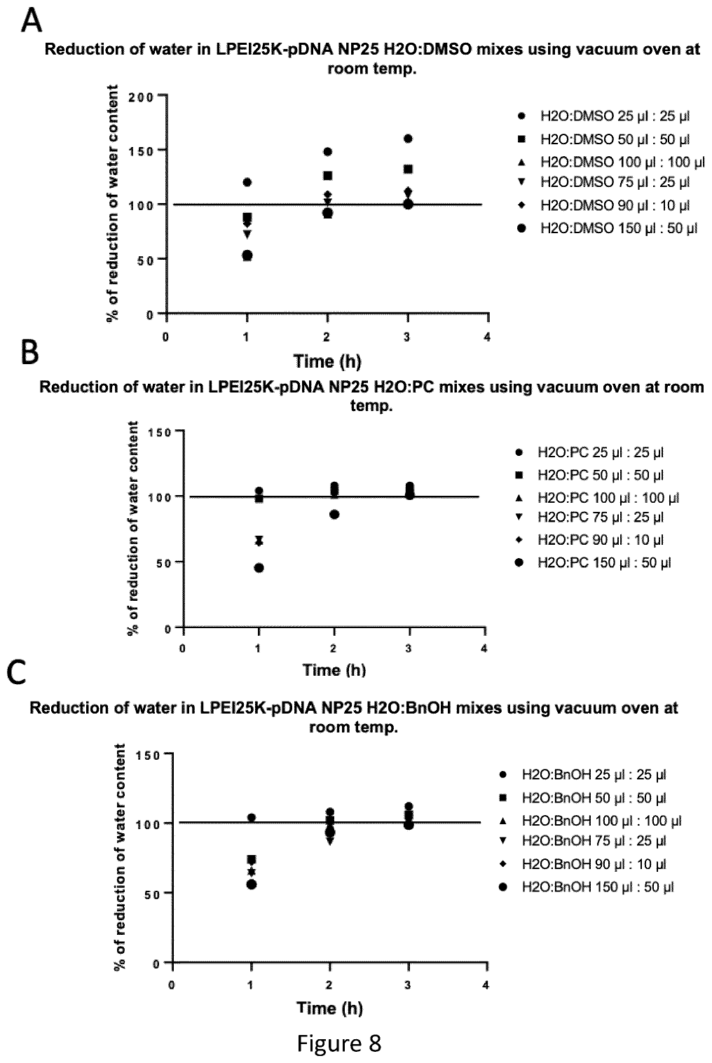

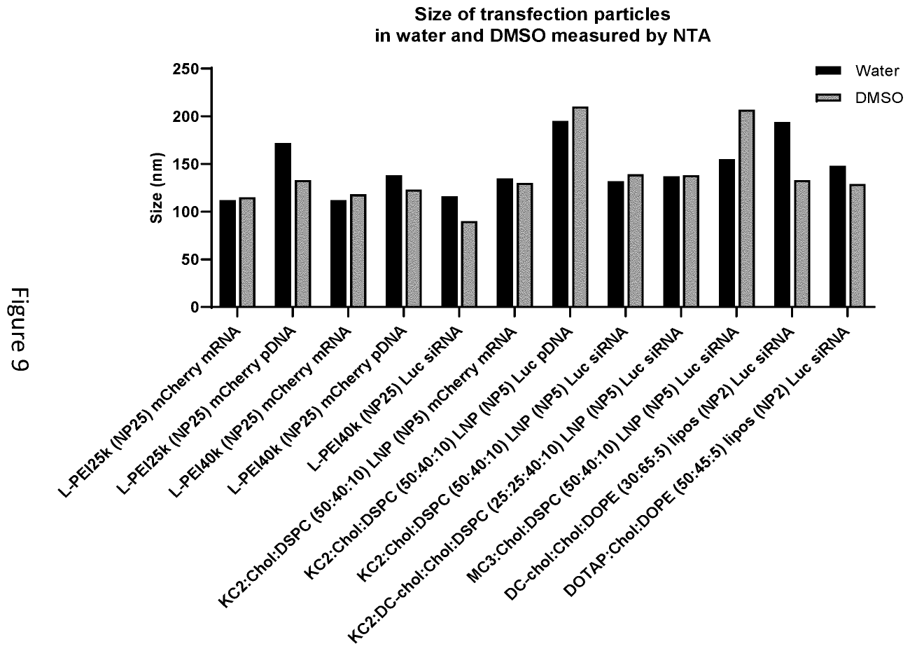

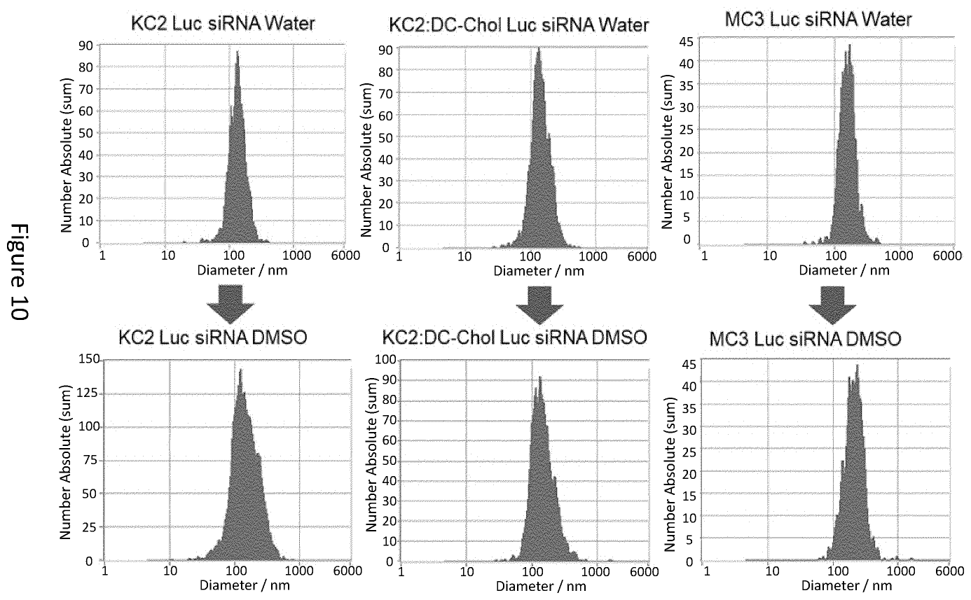

The reduction in water content upon vacuum oven transfers is presented FIG. 8. The data show efficient removal of water from LPEI25k-pDNA particle in water upon mixing with either DMSO, PC or BnOH independently of the water to organic solvent ratio. A gradual removal of water over 3 hours is observed for all samples, where >100% removal indicate loss of organic solvent. The highest loss of organic solvent was obtained for DMSO and only minor losses were obtained upon transfer to PC or BnOH.

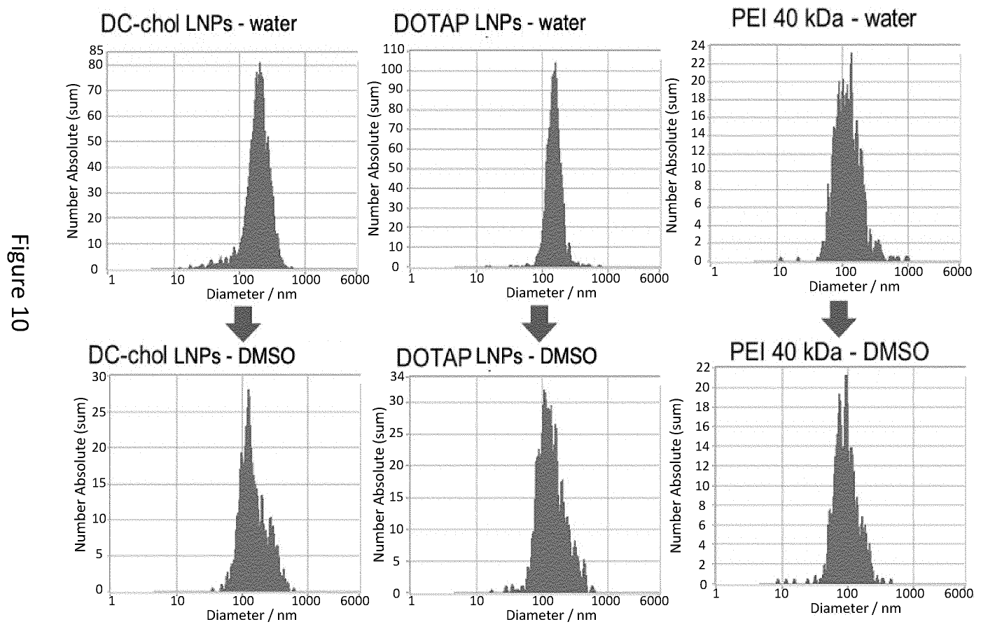

Particle sizes were determined for a range of transfection systems in water and upon transfer to DMSO using NTA and the results are compiled in FIG. 9. These results show that particle size is conserved upon transfer to DMSO followed by resuspension into water/HBS.

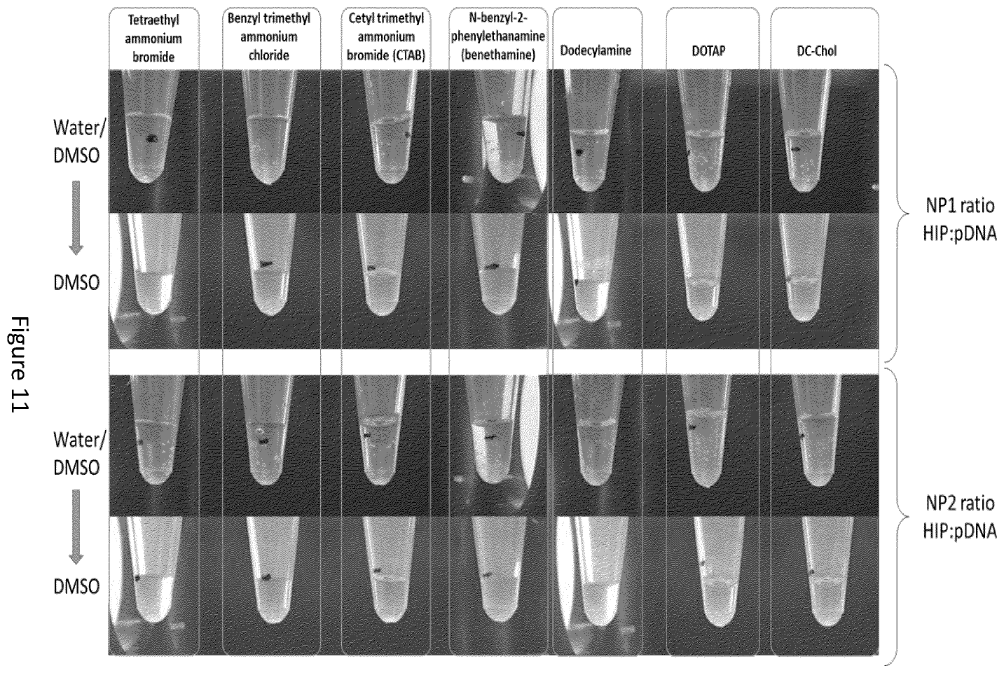

Example of size distribution histograms of the samples presented in FIG. 10 measured by NTA. FIG. 10 shows that particle size distribution is preserved upon transferring the transfection particle into DMSO.

### Conclusion

Transfection systems prepared in aqueous media can effectively be transferred to either DMSO, PC or BnOH, where water is removed by vacuum oven distillation at room temperature. A range of particles were prepared and transferred, and the particle size was preserved upon transfer to DMSO followed by resuspension in water.

### Example 9: Ion-Pairing of pDNA: MRNA and Transfer from Water to Organic Media

Aim: Describe the complexation of hydrophobic cations with DNA or RNA molecules through ionic interactions and formation of hydrophobic ion-pairs (HIPs) to protect the nucleic acids and allow the complexes to be transferred into organic media.

Principle: Complexation of hydrophobic cations with nucleotides involves the ionic interaction between a cationic charged hydrophobic molecule and a negatively charged phosphates on the DNA or RNA molecules. Examples of molecules suitable as cationic HIPs for DNA/RNA complexation are DOTAP, DC-cholesterol, Spermin-cholesterol, Tetraethyl ammonium bromide (TEAB), Benzyl trimethyl ammonium chloride (BTMAC), Didodecyl dimethyl ammonium bromide (DDAB), Tetrahexyl ammonium bromide (THAB), Cetyl trimethyl ammonium bromide (CTAB), Dlin-KC2-DMA (KC2), Dlin-KC3-DMA (MC3), N-(4-carboxybenzyl)-N,N-dimethyl-2,3-bis(oleoyloxy) propan-1-aminium (DOBAQ), and N1-[2-((1S)-1-[(3-aminopropyl)amino]-4-[di(3-amino-propyl)amino]butylcarboxamido)ethyl]-3,4-di [oleyloxy]-benzamide (MVL5)

Method: Solutions of the hydrophobic cations DOTAP, DC-cholesterol, Spermin-cholesterol, Tetraethyl ammonium bromide (TEAB), Benzyl trimethyl ammonium chloride (BTMAC), Didodecyl dimethyl ammonium bromide (DDAB), Tetrahexyl ammonium bromide (THAB), Cetyl trimethyl ammonium bromide (CTAB), Dlin-KC2-DMA (KC2), Dlin-KC3-DMA (MC3), N-(4-carboxybenzyl)-N,N-dimethyl-2,3-bis(oleoyloxy) propan-1-aminium (DOBAQ), and N1-[2-((1S)-1-[(3-aminopropyl)amino]-4-[di(3-amino-propyl)amino]butylcarboxamido)ethyl]-3,4-di [oleyloxy]-benzamide (MVL5), see table 1, were prepared in ultrapure water at 1 mg/mL. In parallel, solutions of pDNA or mRNA were prepared in ultrapure water as well at specific concentration depending on the NP ratio. Both solutions are then quickly mixed and vortexed at selected NP ratios aiming for a pDNA/mRNA final concentration of 200-400 μg/mL. After vortexing thoroughly, the solutions are allowed to rest for 30 minutes at room temperature to allow the complexes to form. The HIP: DNA/RNA complexes can be stored at 4° C. or transferred into DMSO as described in Example 5. The capacity of the complexes to stabilize the nucleotides in DMSO is assessed by inspecting for precipitates and using Ribo/Picogreen assay (Example 6). In addition, some of the complexes form particles, and their size can be measured by NTA both after formation in water and after transferring into DMSO. The toxicity and transfection properties of the complexes was studied in vitro in HEK cells by flow cytometry (Example 27).

### Results and Discussion:

Several hydrophobic cations can be used to complex polynucleotides by forming hydrophobic ion-pairs, which allow them to be soluble in hydrophobic environments such as organic solvents. A list of counterion examples ranging from small cationic molecules to larger cationic amphipathic molecules is presented in table 1:

TABLE 1
List of cationic or ionizable counter-ions for hydrophobic ion pairing of DNA/RNA

| | Compound | LogP | pKa |
|---|---|---|---|
| Compound structure | name | value* | value* |
| 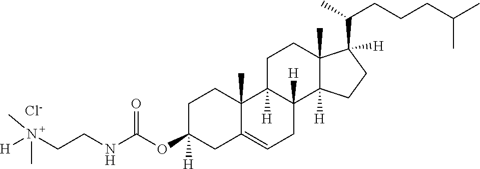 | DC-Chol | 10.53 | 8.9 |
| 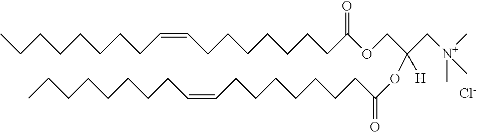 [SMILES: CCCCCCCC/C=C\CCCCCCCC(=O)OCC(C[N+](C)(C)C)OC(=O)CCCCCCC/C=C\CCCCCCCC ; [Cl-]] | Dioleoyl-3- trimethyl ammonium propane (DOTAP) | 13.48 | N/A |
| 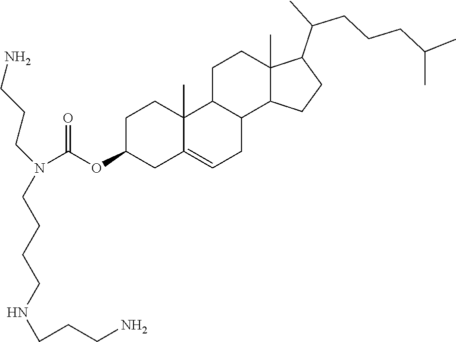 [SMILES: CC(C)CCCC(C)C1CCC2C3CC=C4C[C@@H](OC(=O)N(CCCN)CCCCNCCCN)CCC4(C)C3CCC12C] | Spermin-chol (GL67) | 9.64 | 7.4, 8.5, 10.1, 10.2 |
| 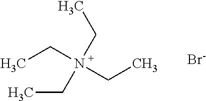 | Tetraethyl ammonium bromide (TEAB) | −2.55 | N/A |
| 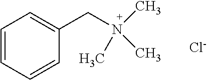 | Benzyl trimethyl ammonium chloride (BTMAC) | −2.25 | N/A |
| 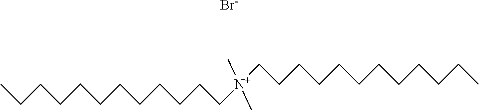 [SMILES: [Br-] ; CCCCCCCCCCCC[N+](C)(C)CCCCCCCCCCCC] | Didodecyl dimethyl ammonium bromide (DDAB) | 12.73 | N/A |
| 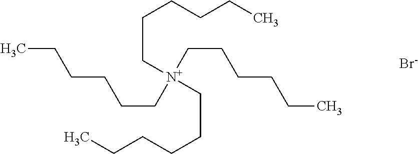 [SMILES: CCCCCC[N+](CCCCCC)(CCCCCC)CCCCCC ; [Br-]] | Tetrahexyl ammonium bromide (THAB) | 5.32 | N/A |
| 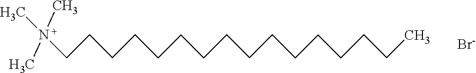 | Cetyl trimethyl ammonium bromide (CTAB) | 2.69 | N/A |
| 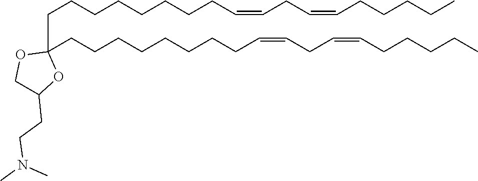 [SMILES: CCCCC/C=C\C/C=C\CCCCCCCCC1(CCCCCCCC/C=C\C/C=C\CCCCC)OCC(CCN(C)C)O1] | Dlin-KC2-DMA (KC2) | 10.16 | 9.3 |
| 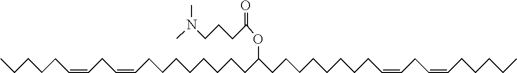 [SMILES: CCCCC/C=C\C/C=C\CCCCCCCCC(CCCCCCCC/C=C\C/C=C\CCCCC)OC(=O)CCCN(C)C] | Dlin-MC3-DMA (MC3) | 17.69 | 9.4 |
| 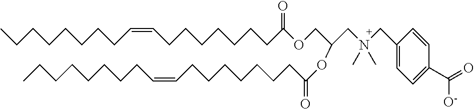 | N-(4-carboxy- benzyl)-N,N- dimethyl-2,3- bis(oleoyloxy)- propan-1- aminium (DOBAQ) | 14.60 | 4.1 |
| 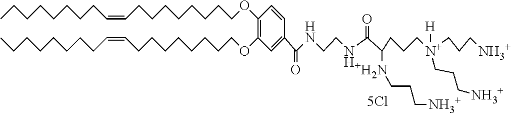 [SMILES: CCCCCCCC/C=C\CCCCCCCCOc1ccc(C(=O)NCCNC(=O)C(CCC[NH+](CCC[NH3+])CCC[NH3+])[NH2+]CCC[NH3+])cc1OCCCCCCCC/C=C\CCCCCCCC] | N1-[2-((1S)-1- [(3-aminoprop- yl)amino]-4- [di(3-amino- propyl)amino] butylcarbox- amido)ethyl]- 3,4-di[oleyloxy] benzamide (MVL5) | 17.60 | 4.9, 6.4, 10.0, 10.1, 10.1 |

*calculated using ChemDraw

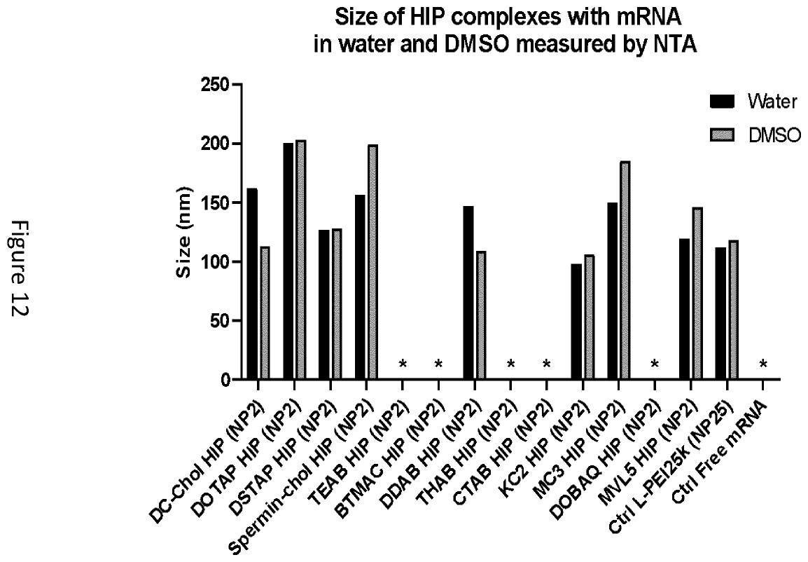

Ion pairing of DNA/RNA with HIPs complexes allows the stability of the DNA/RNA molecules upon transferring into organic solvents. FIG. 11 shows HIP: pDNA complexes at 0.4 μg/μL pDNA concentration remained soluble in water and upon transfer to DMSO where all samples appear transparent without any precipitation.

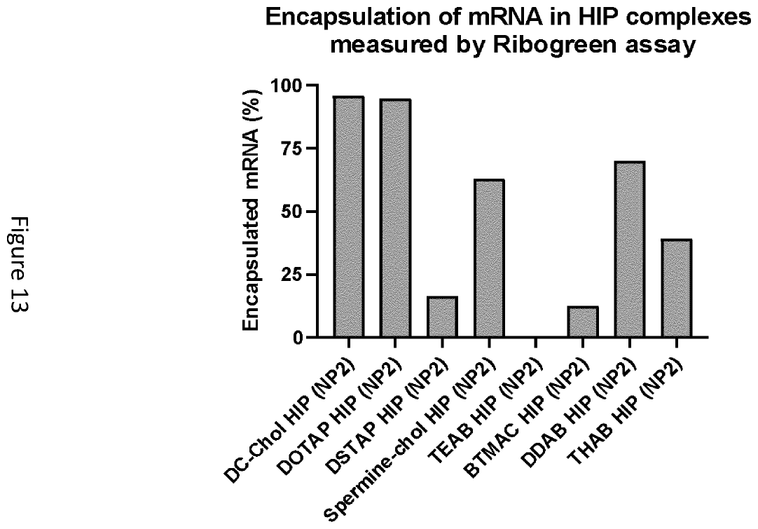

Ion pairing of DNA/RNA with counterions with larger structures can form particles that can be detected using NTA. In FIG. 12 the size of these complexes is presented after complexation in water and also after transferring into DMSO. The results show that the HIPs that form particle complexes maintain their sizes after transferring into DMSO, indicating the particle stability of the complexes in DMSO.

The capacity of the HIP molecules to complex DNA/RNA and encapsulate the DNA/RNA from exposure to molecules in solution is also highly dependent on the nature of the counterion. In FIG. 13 we show how HIP: mRNA complexes using DC-chol, DOTAP, Spermin-chol or DDAB as HIP molecules result in higher encapsulation of mRNA molecules compared to smaller HIP molecules such as TEAB, BTMAC or THAB. The encapsulation of mRNA is measured as the capacity of the Ribogreen dye to bind mRNA in these HIP complexes in water and after digestion (or dissolution) of the complexes using 0.5% TritonX.

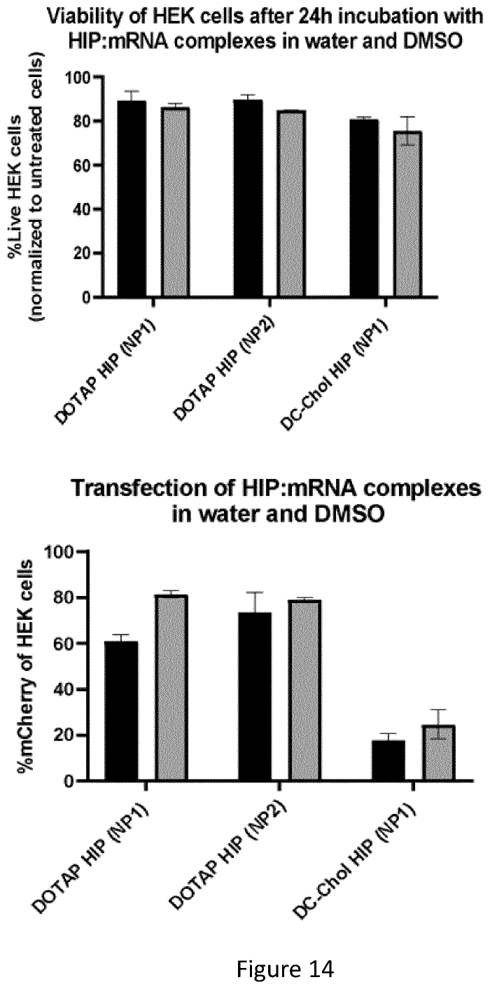

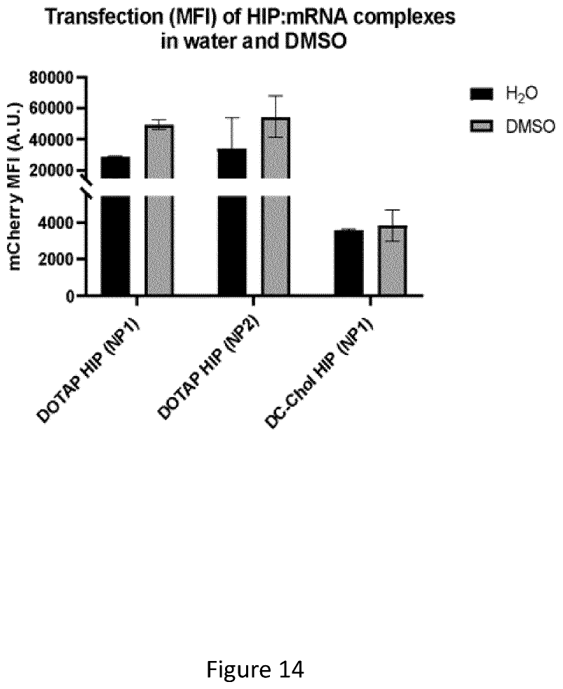

The HIP: mRNA complexes were able to maintain their transfection capacity when transferred into DMSO. FIG. 14 shows the percentage and MFI of HEK cells transfected with mCherry mRNA after 24 h treatment with 400 ng mRNA complexed with DOTAP or DC-Chol HIPs and measured by flow cytometry. The viability of HEK cells was not affected after treatment with the HIPs complexes either in water or DMSO.

### Conclusion

Due to the intrinsic hydrophilic nature of DNA/RNA, complexation with hydrophobic ion pairs (HIPs) allows the transfer of nucleic acids into organic solvents, such as DMSO, a prerequisite for embedding the transfection complexes in the NCCells. In this example we present a range of small cationic HIPs that are able to complex mCherry pDNA and mRNA, and stabilize the nucleic acids upon transfer into DMSO. We show that the degree of mRNA protection after complexation to molecules in solution, such as Ribogreen dye, can be fine-tuned by selecting the optimal HIP counterion. In addition, we show that depending on the structure of the HIP some of complexes form particles that can be measured by NTA, and that the particles remain stable upon transfer into DMSO. Finally, we show the ability of these HIP: mRNA complexes in water and DMSO to transfect HEK cells with mCherry mRNA in vitro without affecting the cell viability.

### Example 10: Ion-Pairing of Silencing siRNA and Transfer from Water to Organic Media

Aim: Describe the complexation of hydrophobic cations with silencing siRNA molecules through ionic interactions to protect the nucleic acids and allow the complexes to be transferred into organic media.

Principle: Complexation of hydrophobic cations with nucleotides and formation of hydrophobic ion-pairs, involves the ionic interaction between cationic charged hydrophobic molecules and negatively charged phosphates on the silencing siRNA molecules. Examples of molecules suitable as cationic HIPs for siRNA complexation are DOTAP, DSTAP, DC-cholesterol, Spermin-cholesterol, Tetraethyl ammonium bromide (TEAB), Benzyl trimethyl ammonium chloride (BTMAC), Didodecyl dimethyl ammonium bromide (DDAB), Tetrahexyl ammonium bromide (THAB) and Cetyl trimethyl ammonium bromide (CTAB).

Method: Solutions of the hydrophobic cations DC-cholesterol, DOTAP, DSTAP, Spermin-cholesterol, Tetraethyl ammonium bromide (TEAB), Benzyl trimethyl ammonium chloride (BTMAC), Didodecyl dimethyl ammonium bromide (DDAB), Tetrahexyl ammonium bromide (THAB) and Cetyl trimethyl ammonium bromide (CTAB), see table 1, were prepared in ultrapure water at 1 mg/mL. In parallel, solutions of Luciferase siRNA were prepared in ultrapure water as well at specific concentration depending on the desired NP ratio (in this case NP2). Solutions of siRNA and cations were quickly mixed and vortexed at selected NP ratios aiming for a siRNA final concentration of 0.1 μg/μL. After vortexing thoroughly, the solutions were allowed to rest for 30 minutes at room temperature to allow the complexes to form. The HIP: siRNA complexes can be stored at 4° C. or transferred into DMSO as described in Example 8. The capacity of the complexes to stabilize the nucleotides in DMSO is assessed by inspecting for precipitates and using Ribogreen assay (Example 6). In addition, some of the complexes form particles, which their size can be measured by NTA both after formation in water and after transferring into DMSO.

### Results and Discussion:

Different cationic hydroponic ion pairs (HIPs) can be used to complex the nucleotides to allow them to be soluble in hydrophobic environments of organic solvents. A list of some HIPs examples ranging from small cationic molecules to larger cationic amphipathic molecules is presented in table 2:

TABLE 2
List of cationic or ionizable counter-ions for hydrophobic ion pairing of DNA/RNA.

| | | LogP | pKa |
|---|---|---|---|
| Compound structure | Compound name | value* | value* |
| 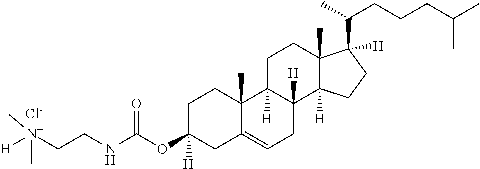 | DC-Chol | 10.53 | 8.9 |
| 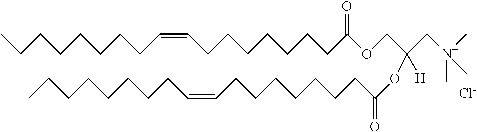 [SMILES: CCCCCCCC/C=C\CCCCCCCC(=O)OCC(C[N+](C)(C)C)OC(=O)CCCCCCC/C=C\CCCCCCCC ; [Cl-]] | Dioleoyl-3-trimethyl- ammonium-propane (DOTAP) | 13.48 | N/A |
| 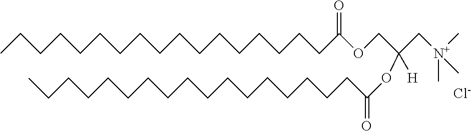 [SMILES: CCCCCCCCCCCCCCCCCC(=O)OCC(C[N+](C)(C)C)OC(=O)CCCCCCCCCCCCCCCCC ; [Cl-]] | Distearoyl-3-trimethyl- ammonium-propane (DSTAP) | 12.52 | N/A |
| 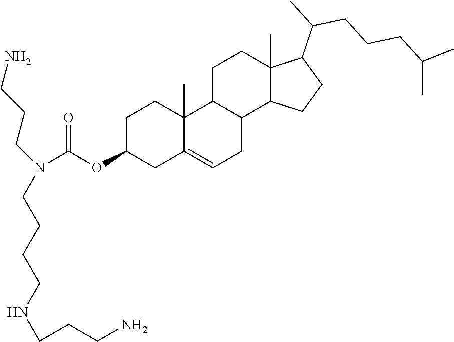 [SMILES: CC(C)CCCC(C)C1CCC2C3CC=C4C[C@@H](OC(=O)N(CCCN)CCCCNCCCN)CCC4(C)C3CCC12C] | Spermin-chol (GL67) | 9.64 | 7.4, 8.5, 10.1, 10.2 |
| 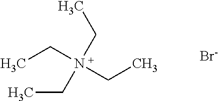 | Tetraethyl ammonium bromide (TEAB) | −2.55 | N/A |
| 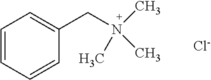 | Benzyl trimethyl- ammonium chloride (BTMAC) | −2.25 | N/A |
| 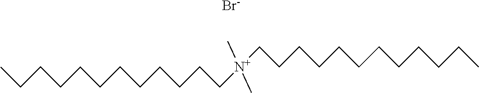 [SMILES: [Br-] ; CCCCCCCCCCCC[N+](C)(C)CCCCCCCCCCCC] | Didodecyl dimethyl ammonium bromide (DDAB) | 12.73 | N/A |
| 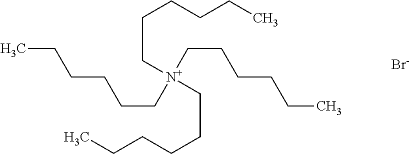 [SMILES: CCCCCC[N+](CCCCCC)(CCCCCC)CCCCCC ; [Br-]] | Tetrahexyl ammonium bromide (THAB) | 5.32 | N/A |
| 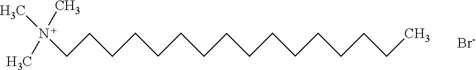 | Cetyl trimethyl ammonium bromide (CTAB) | 2.69 | N/A |

*calculated using ChemDraw

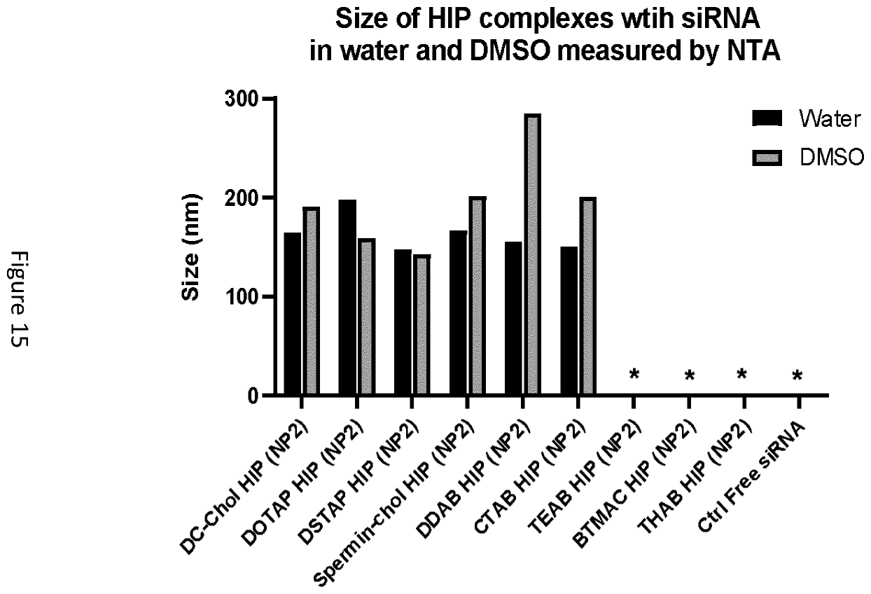

Ion pairing of silencing siRNA with HIPs complexes allows the stability of the siRNA molecules in water and upon transferring into organic solvents. Counterions with larger structures can form particles that can be detected using NTA. In FIG. 15 the size of these complexes is presented after complexation in water and also after transferring into DMSO. The results show that the HIPs that form particle complexes maintain their sizes after transferring into DMSO, indicating the particle stability of the complexes in DMSO.

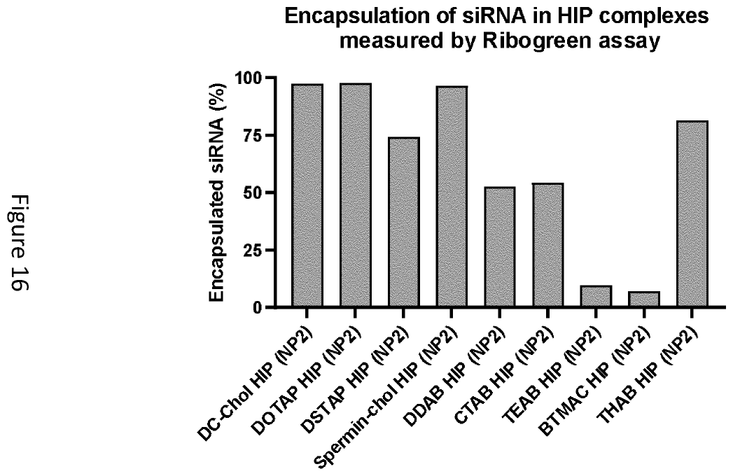

The capacity of the HIP molecules to complex siRNA and encapsulate the siRNA from exposure to molecules in solution is dependent on the nature of the counterion. In FIG. 16 we show how HIP: siRNA complexes using DC-chol, DOTAP, DSTAP, Spermin-chol, DDAB or CTAB as HIP molecules result in higher encapsulation of siRNA molecules compared to smaller HIP molecules such as TEAB or BTMAC. The THAB counterion also showed high siRNA encapsulation even though particles could not be measured by NTA (FIG. 15). The encapsulation of siRNA is measured as the capacity of the Ribogreen dye to bind siRNA in these HIP complexes in water and after digestion the complexes using 0.5% TritonX.

### Conclusion

Complexation hydrophobic cations and nucleic acids allows for transfer of the formed hydrophobic ion-pairs into organic solvents, such as DMSO, a prerequisite for embedding the transfection complexes in the NCCells. In this example we present a range of small cationic HIPs that can complex luciferase siRNA and stabilize the nucleic acids upon transfer into DMSO. We show that the degree of siRNA protection after complexation to molecules in solution, such as Ribogreen dye, can be fine-tuned by selecting the optimal HIP counterion. In addition, we show that depending on the structure of the HIP some of the complexes form particles that can be measured by NTA, and that the particles remain stable upon transfer into DMSO.

### Example 11: Ps-Oligos Accumulate Intracellularly when Formulated as SLNP, HIPs, Polyplexes and as Free Ps-Oligos

Aim: Demonstrate in vitro internalization of PS-oligos from water and DMSO when formulated as SLNP, HIP, polyplexes, and free PS-oligos.

Method: Transfection particles with composition KC2: Chol: DSPC (50:40:10) NP5, L-PEI40k NP25, DOTAP-HIP NP2 with scrambled PS-Oligos-Cy5 were prepared at 0.1 μg/μl in water and 0.2 μg/μl in DMSO according to example 2, 3, 5 & 7.

Transfection complexes in DMSO: 50 μL of lipid particles were mixed with 50 μL DMSO and were transferred to DMSO using a vacuum oven as described in example 8.

25K CT26. Luc cells per well were seeded on u-Ibidi chamber slides and left overnight for attachment. The cells were treated with 0.4 μg particles or free PS-oligo-cy5 in 300 μl transfection volume for 5 hours for proper internalization. Cells were then washed with 1×PBS and then stained the nucleus with Hoechst (1:500) and the membrane with Cellmask green (1:1000) for 30 min at 4° C. After staining, the cells were washed twice with lice cell imaging solution. Images were acquired using an inverted Nikon Ti2 Spinning Disc confocal microscope and processed using Fiji.

### Results:

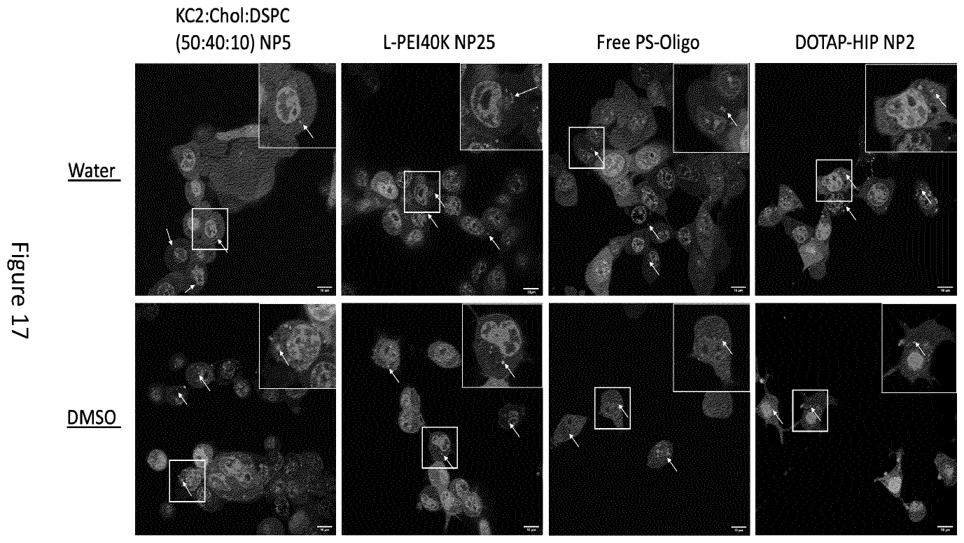

The images indicate that all PS-Oligo formulations, KC2, L-PEI40k, DOTAP-HIP, and free Oligos can get into the cells when prepared in water and DMSO (FIG. 17). There is an indication of more internalization of complexed oligos compared to free oligos.

### Conclusion:

Scrambled PS-Oligos prepared as KC2 or L-PEI40K particles or DOTAP-HIP complexes are internalized by CT26.luc cells in vitro. Free scrambled PS-Oligos are also internalized by the cells.

### Example 12: Preparation of NCCells with PEI pDNA/mRNA Transfection Systems

Aim: Demonstrate solubility of PEI pDNA or PEI mRNA transfection systems in NCCell

### Methods:

NCCell preparation: NCCell formulation with compositions SuBen: GTH: EtOH: DMSO-55:20:5:10, 55:20:5:20, 55:20:10:10 were prepared according to example 1.

PEI-pDNA and PEI-mRNA preparation: transfection complexes with composition: L-PEI25K-pDNA/mRNA NP25 was complexed in water at a concentration of 0.2 μg pDNA/mRNA/μl in 250 μl preparations according to example 2.

Transfection complexes in DMSO: 100 μL transfection complex in water was mixed with 50 μL DMSO and were transferred to DMSO using a vacuum oven as described in example 8.

Embedding transfection complexes in NCCell: 50 μL transfection complex in DMSO and 450 μL NCCell were mixed in an Eppendorf tube and the sample was stirred at room temperature for 5 min. Final concentration of pDNA/mRNA in NCCells was 40 μg/g

### Results and Discussion

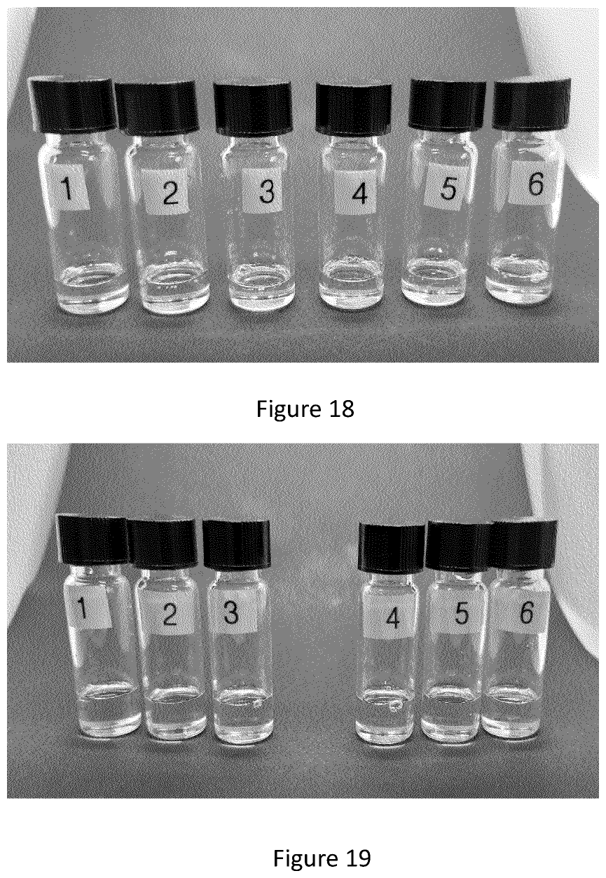

Transfection systems with compositions L-PEI25K-pDNA/mRNA NP25 were prepared in water and following transferred to DMSO. The particles in DMSO were embedded in NCCell compositions and were visually assessed. Examples of NCCells embedding L-PEI25K-pDNA/mRNA NP25 are shown in FIG. 18.

The results presented in FIG. 18 display transparent formulation indicating homogeneous distribution of particles in the samples.

### Conclusion

L-PEI25K-complexed with pDNA or mRNA at NP25 were successfully embedded in NCCell compositions SuBen: GTH: EtOH: DMSO-55:20:5:10, 55:20:5:20, 55:20:10:10.

### Example 13: Preparation of NCCells with DC-Chol-Based pDNA/mRNA Transfection Systems

Aim: Demonstrate solubility of DC-Chol-based pDNA or mRNA transfection systems in NCCells.

### Methods:

NCCell preparation: NCCell formulation with compositions SuBen: GTH: EtOH: DMSO-55:20:5:10, 55:20:5:20, 55:20:10:10 was prepared according to example 1.

DC-Chol-pDNA and DC-Chol-mRNA preparation: DC-Chol-based liposomes (DC-Chol: Chol: DOPE 30:65:5) were prepared as described in example 3. DC-Chol-based liposomes-pDNA/mRNA (mCherry) NP5 was complexed in water at a concentration of 0.2 μg pDNA/mRNA/μl in 250 μl preparations according to example 3.

Transfection complexes in DMSO: 100 μL of particles in water, totally 20 μg pDNA/mRNA were mixed with 50 μL DMSO and directly transferred to DMSO using a vacuum oven as described in example 8.

Embedding transfection complexes in NCCell: 50 μL transfection complex in DMSO and 450 μL NCCell were mixed in an Eppendorf tube, and the sample was stirred at room temperature for 5 min. The final pDNA/mRNA concentration was 40 μg per gram NCCell.

### Results and Discussion

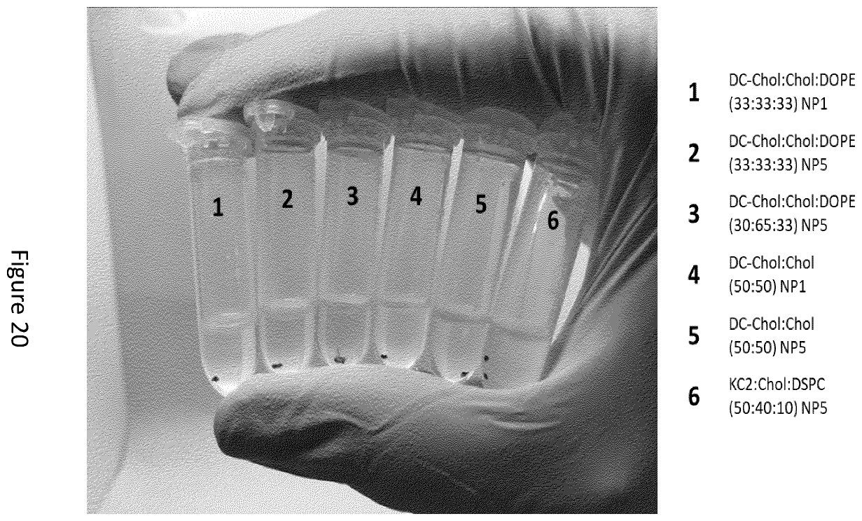

Transfection systems with compositions DC-Chol-pDNA and DC-Chol-mRNA NP5 were prepared in water and following transferred to DMSO. The particles in DMSO were embedded in NCCell compositions and were visually assessed. Examples of NCCells embedding DC-Chol-pDNA and DC-Chol-mRNA NP5 are shown in FIG. 19.

The results presented in FIG. 19 display transparent formulation indicating homogeneous distribution of particles in the samples.

### Conclusion

DC-Chol-based liposomes-complexed with pDNA or mRNA at NP5 were successfully embedded in NCCell compositions SuBen: GTH: EtOH: DMSO-55:20:5:10, 55:20:5:20, 55:20:10:10.

### Example 14: Preparation of NCCells with KC2: DC-Chol pDNA/mRNA Transfection Systems

Aim: Demonstrate solubility of KC2: DC-chol pDNA or mRNA transfection systems in NCCell

### Methods:

NCCell preparation: NCCell formulation with composition SuBen: GTH: EtOH: DMSO (55:20:5:20) was prepared according to example 1.

KC2: DC-chol particles with pDNA/mRNA preparation: transfection complexes with composition KC2: Chol: DSPC (50:40:10), KC2: DC-chol: Chol: DSPC (25:25:40:10), DC-Chol: Chol: DOPE (33:33:33), DC-Chol: Chol: DOPE (30:65:5), DC-Chol: Chol (50:50) were prepared at 0.2 μg/mL pDNA using the ignite flow mixer according to example 4.

Transfection complexes in DMSO: 50 μL of transfection particles was mixed with 50 μL DMSO and were transferred to DMSO using a vacuum oven as described in example 8.

Embedding transfection complexes in NCCell: 50 μL transfection complex in DMSO and 450 μL NCCell with composition SuBen: GTH: EtOH: DMSO (55:20:5:20) were mixed in an eppendorf tube, and the sample was stirred at room temperature for 5 min. The final pDNA concentration was 20 μg per gram NCCell.

### Results and Discussion

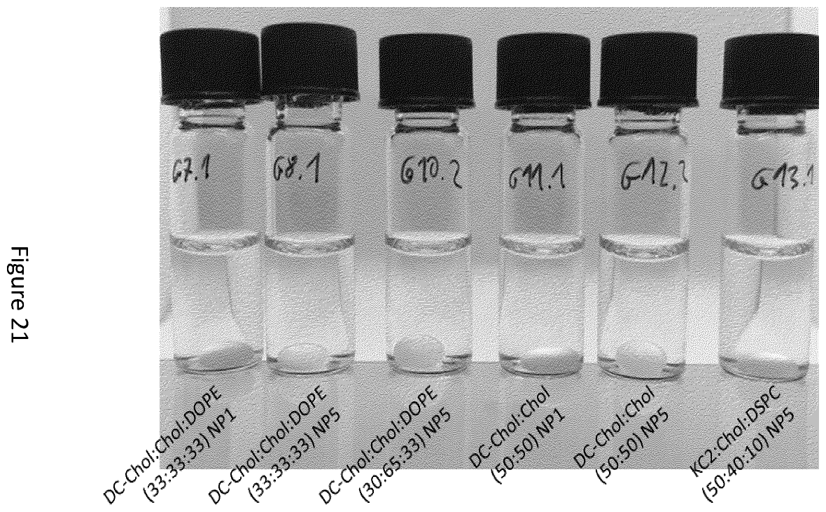

Transfection systems with composition KC2: DC-chol LNP combinations with pDNA at NP1 and NP5 were prepared in water and following transferred to DMSO. The particles in DMSO were embedded in NCCell with composition SuBen: GTH: EtOH: DMSO (55:20:5:20) and were visually assessed. Examples of NCCells embedding different transfection systems are shown in FIG. 20. Similar homogeneous solutions are also obtained with different NCCell compositions.

The results presented in FIG. 20 display transparent formulations indicating homogeneous distribution of particles in the samples.

Upon injection of these NCCells into water phase, the systems become a depot of transfection particles (FIG. 21)

### Conclusion

Combinations of KC2: DC-chol transfection lipid nanoparticles complexed with pDNA or mRNA at NP1 or NP5 were successfully embedded in NCCell composition SuBen: GTH: EtOH: DMSO (55:20:5:20). Identical results are observed when embedding into NCCells of different compositions.

### Example 15: Preparation of NCCells with HIP-pDNA/mRNA Transfection Systems

Aim: Demonstrate solubility of ion paired pDNA or mRNA transfection systems in NCCell

### Methods:

NCCell preparation: NCCell formulation with composition SuBen: GTH: EtOH: DMSO ((55:20:5:20)+0.25% Cholesterol) was prepared according to example 1.

Ion paired pDNA/mRNA complex preparation: Ion paired transfection complexes were prepared with the cationic counterions DOTAP, DSTAP, Spermin-chol, Tetraethyl ammonium bromide (TEAB), Didodecyl dimethyl ammonium bromide (DDAB), Tetrahexyl ammonium bromide (THAB) and Cetyl trimethyl ammonium bromide (CTAB), in water at a NP2 ratio with final 0.1 μg/μL mCherry mRNA according to example 5.

Transfection complexes in DMSO: 50 μL of HIP: mRNA transfection complexes were mixed with 25 μL DMSO, and transferred to DMSO using a vacuum oven as described in example 8. Final mRNA concentration in DMSO was 0.2 μg/μL.

Embedding transfection complexes in NCCell: 25 μL transfection complex in DMSO and 475 μL NCCell with composition SuBen: GTH: EtOH: DMSO ((55:20:5:20)+0.25% Cholesterol) were mixed in an Eppendorf tube, and the sample was stirred at room temperature for 5 min. The final mRNA concentration was 10 μg per gram of NCCell

### Results and Discussion

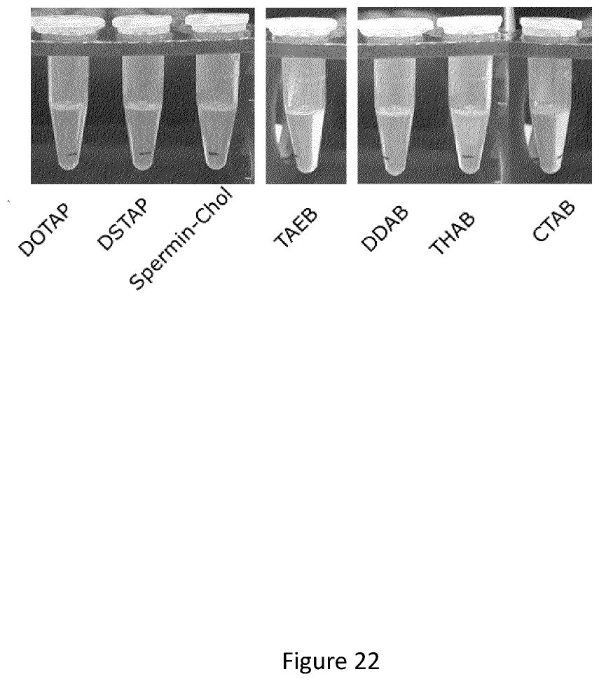

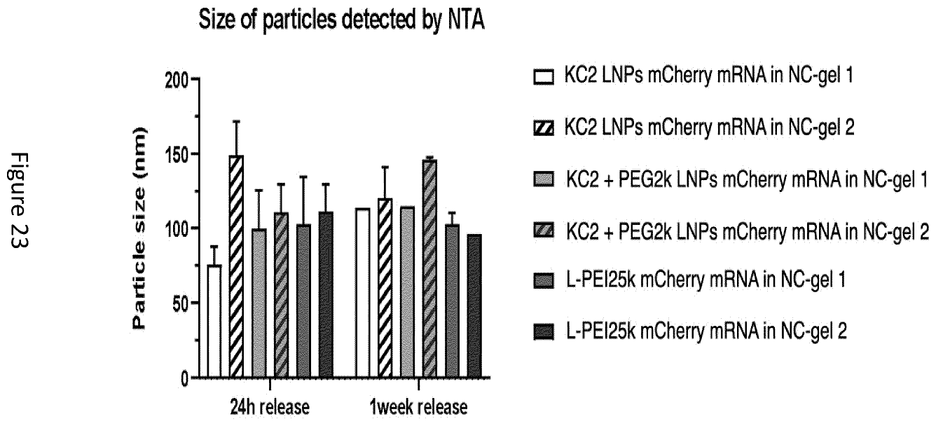

Transfection systems based on hydrophobic ion pairing of mCherry mRNA at NP2 with various cationic counterions were prepared in water and following transferred to DMSO. The HIP: mRNA complexes in DMSO were embedded in NCCell with composition SuBen: GTH: EtOH: DMSO ((55:20:5:20)+0.25% cholesterol) and were visually inspected. Examples of NCCells embedding HIP: mRNA complexes with different counterions are shown in FIG. 22. Similar homogeneous solutions are also obtained with different NCCell compositions.

The results presented in FIG. 22 display transparent NCCell formulations indicating homogeneous distribution of the complexes in the samples.

### Conclusion

Various cationic counterions used to complex mRNA at NP2 were successfully embedded in NCCell with composition SuBen: GTH: EtOH: DMSO ((55:20:5:20)+0.25% cholesterol). Identical results are observed when embedding into NCCells of different compositions.

### Example 16: NCCells Release Particles

Aim: Demonstrate controlled release of transfection particles from NCCells in vitro using NTA for particle sizing and quantification.

### Methods:

NCCell preparation: NCCell formulation 1 and 2 with composition SuBen: GTH: EtOH: DMSO 55:20:5:20 and SuBen: GTH: EtOH: DMSO 55:20:5:20+0.25% Cholesterol respectively, were prepared according to example 1.

KC2 and KC2-PEG2k mCherry mRNA LNPs preparation:

Transfection particles with composition KC2: Chol: DSPC (50:40:10) with/without 1.5% DMG-PEG2k with mCherry mRNA at NP5 were prepared according to example 4.

PEI-pDNA and PEI-mRNA preparation: transfection complexes with composition L-PEI25K NP25 was prepared according to example 2.

Transfection complexes in DMSO: 50 μL of polyplexes or lipoplexes were mixed with 50 μL DMSO and were transferred to DMSO using a vacuum oven as described in example 8.

Embedding transfection complexes in NCCell: 50 μL transfection complex in DMSO and 450 μL NCCell were mixed in an eppendorf tube, and the sample was stirred at room temperature for 5 min.

In vitro release of particles: 100 μL NCCell compositions embedding transfection particles was injected into glass vials containing 1 mL PBS. The release samples were heated to 37° C. during the period of the experiment. 1 mL PBS was sampled at the 24 h and 1 week timepoint and particle size and number were quantified by NTA as describes in example 6.

### Results and Discussion

Transfection systems with composition KC2: Chol: DSPC (50:40:10) with/without 1.5% DMG-PEG2k with mCherry mRNA at NP5, and LPEI25k with mCherry pDNA or mRNA at NP25 were prepared in water and following transferred to DMSO. The particles in

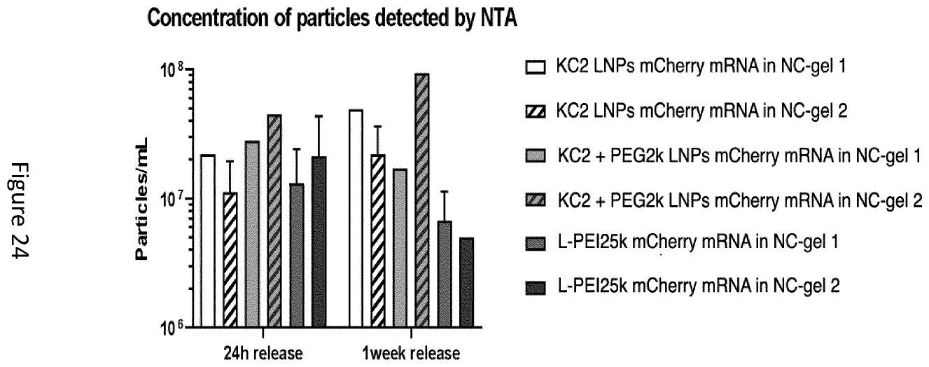

DMSO were embedded in NCCell compositions (SuBen: GTH: EtOH: DMSO 55:20:5:20 with/without +0.25% Cholesterol) and injected into buffer for in vitro release testing. Examples of release data are show in FIGS. 23 and 24.

The results presented in FIGS. 23 and 24 show that KC2 LNPs and PEI particles can be release from NCCells and that particle size is preserved for up to one week. The particle concentrations determined after 24 h and 1 week release is similar, which demonstrate that release of transfection systems continue for up to one week.

### Conclusion

PEI and KC2 transfection particles were embedded in NCCells and in vitro release was demonstrated. The particle size and concentration (particle number) were shown to be equivalent at the 24 h and 1 week timepoint, which demonstrate particle stability and steady release from NCCells.

### Example 17: Cryo-TEM Imaging of Polyplex Particles in Water and DMSO

Aim: Demonstrate particle integrity of polyplexes in water, DMSO and gels. Methods:

Prep of particles: L-PEI25K: L-PEI25K-PEG550 (50:50) pDNA (IL-12, 0.2 μg/mL) NP25 particles were prepared as described in example 2 and transferred into DMSO as described in example 8.

Cryo-TEM analysis was performed on free particles in water and upon transfer to DMSO as previously described in reference J. R. Henriksen and T. L. Andresen, Thermodynamic Profiling of Peptide Membrane Interactions by Isothermal Titration calorimetry: A Search for Pores and Micelles, Biophys. J., 2011, 101, 100-109.

### Results and Discussion

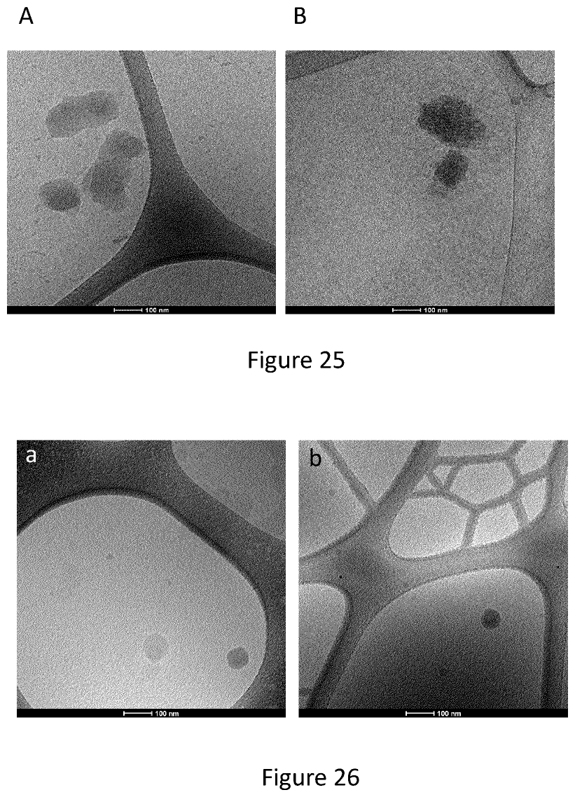

Polyplexes with composition L-PEI25K: L-PEI25K-PEG550 (50:50) pDNA NP25 were prepared in water and transferred to DMSO. Particles in water and in DMSO were subjected to Cryo-TEM analysis, FIG. 25. The images showed particles with sizes in the range of 100-200 nm and appeared similar in water and DMSO.

### Conclusion

Polyplexes with composition L-PEI25K: L-PEI25K-PEG550 (50:50) pDNA NP25 can be prepared in water and transferred into the organic solvent DMSO with preserved particle integrity.

### Example 18: Cryo-TEM Imaging of Lipoplexes in Water and DMSO

Aim: Demonstrate particle integrity of lipoplexes in water and DMSO.

### Methods:

Particle preparation: DOTAP: Cholesterol (50:50 liposomal formulation) pDNA NP5 lipoplex particles (IL-12, 0.2 μg/mL) were prepared as described in example 3 and transferred into DMSO as described in example 8.

Cryo-TEM analysis was performed on free particles in water and DMSO as described in example 17.

### Results and Discussion

Lipoplexes with composition DOTAP: Cholesterol (50:50 liposomal formulation) pDNA NP5 were prepared in water and transferred to DMSO. Particles in water and in DMSO were subjected to Cryo-TEM analysis and representative images are shown in FIG. 26. The images showed particles with sizes in the range of 100-150 nm and appeared similar in water and DMSO.

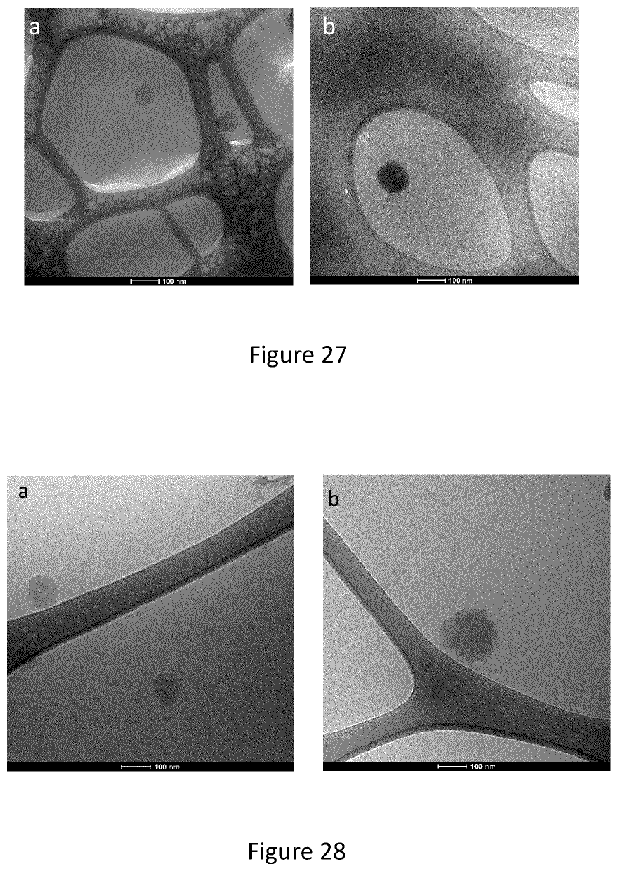

Solid lipid nanoparticles with the composition DLin-KC2: Cholesterol: DSPC (50:40:10) pDNA NP5 were analysed by Cryo-TEM in water or DMSO and representative images are shown in FIG. 27. The images showed particles with sizes in the range of 100-150 nm and appeared similar in water and DMSO.

### Conclusion

Lipoplexes prepared from liposomes mixing with pDNA in water or lipoplexes prepared as solid lipid nanoparticles prepared by ethanol injection into pH 4 buffer can be transferred into the organic solvent DMSO with preserved particle integrity.

### Example 19: Cryo-TEM Imaging of Polyplex Particles in Water Released from NCCells

Aim: Demonstrate particle integrity of polyplexes upon release from NCCells into PBS. Methods:

NCCell preparation: NCCell formulation with composition (SuBen: GTH: EtOH: DMSO 55:20:5:20) were prepared according to example 1.

Particle preparation and formulation: L-PEI25K pDNA NP25 particles (IL-12, 0.2 μg/mL) were prepared as described in example 2 and transferred into DMSO as described in example 8. The particles in DMSO were solubilized in the NCCell formulation as described in example 12.

In vitro release of transfection particles: 100 μl of the NCCell formulation was injected into 500 μl of PBS in a glass vial. After 24 hours the PBS was harvested.

Cryo-TEM analysis was performed on free particles directly prepared in water and on particles released from NCCells into PBS as described in example 17.

### Results and Discussion

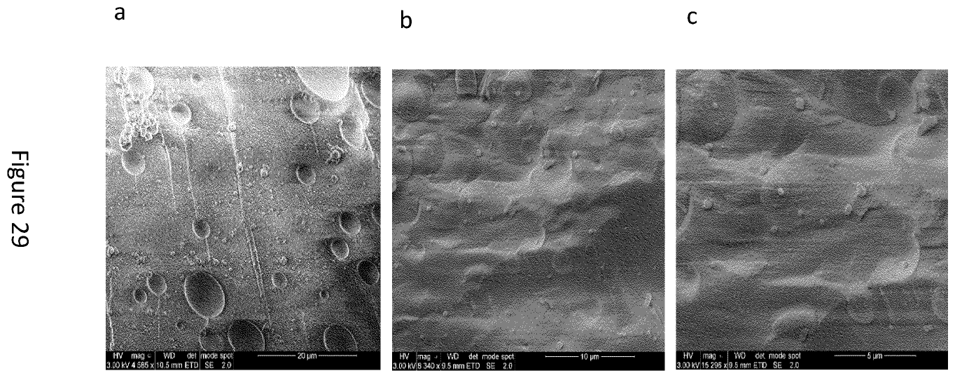

Polyplexes with composition L-PEI25K pDNA NP25 prepared in water or collected from NCCell release medium was subjected to Cryo-TEM analysis. (FIG. 28). The images showed particles with sizes in the rage of 100-200 nm and appeared similar in water and in release medium demonstrating that polyplexes with the formulation L-PEI25K pDNA NP25 can be transferred into organic solvents such as DMSO and incorporated into NCCells and remain intact after release into PBS.

### Conclusion

Polyplexes with composition L-PEI25K pDNA NP25 can be prepared in water and transferred into the organic solvent DMSO followed by solubilization in NCCell composition and released into buffer with preserved particle integrity.

### Example 20: Cryo-SEM Imaging of NCCells Containing Polyplex Particles

Aim: Demonstrate presence of intact polyplex particles in and on the surface of NCCells

### Methods:

L-PEI25K: pDNA (IL-12, 0.2 μg/ml) NP25 particles were prepared as described in example 2 and transferred into DMSO as described in example 8.

100 μl of NCCells with embedded particles were injected into 6 ml PBS buffer and incubated at 37° C. for 24 hours. During this time the NCCell will initiate curing and start releasing particles.

The NCCell samples were adhered to SEM stubs with a 50:50 mixture of colloidal graphite powder (agar scientific) and Tissue-Tek OCT compound (Ted Pella) and immediately frozen in liquid nitrogen. NCCell samples were then loaded onto a Leica EM VCT 100 Cryo Transfer Shuttle and transferred to a Leica EM MED020 freeze fracture and high vacuum coating system. Samples were then fractured and sublimated for 1 minute at −90° C. and sputter coated with 6 nm of carbon/platinum. Following coating, the samples were moved via the VCT 100 Cryo Transfer Shuttle under vacuum and at −140° C. to the Thermo Scientific Quanta 3D FEG FIB/SEM for subsequent SEM imaging. Cryo-SEM analysis was performed at high vacuum at −140° C. with an accelerating voltage of 2 kV.

### Results and Discussion

Polyplexes with composition L-PEI25K: pDNA NP25 embedded into NCCells were imaged using Cryo-SEM. On the surface of the NCCells there were presence of water channels and nanoparticles showing that the polyplexes were intact (FIG. 29).

### Conclusion

Cryo-SEM analysis of NCCells containing polyplexes show surface-associated intact nanoparticles.

### Example 21: Controlled Release of Transfection Particles from NCCells Evaluated by Fluorescence

Aim: Demonstrate controlled release of transfection particles from NCCells in vitro using Cy5 labelled pDNA (mCherry) and fluorescence for quantification.

### Methods:

mCherry pDNA was labelled with Cy5 at 025:1 (v: w) ratio using Label IT® Tracker™ Intracellular Nucleic Acid Localization Kits according to manufactures protocol NCCell preparation: NCCell compositions LOIB: GTH: EtOH: DMSO (70:10:5:15), LOIB: GTO: EtOH: DMSO (70:10:5:15), SuBen: EtOH: DMSO (60:15:25), SuBen: GTH: EtOH: DMSO (60:5:15:20), SuBen: GTH: EtOH: DMSO (55:20:5:20), LOIB: GTO: EtOH: DMSO (65:7.5:5:22.5) were prepared according to example 1. PEI-pDNA particles: transfection complexes with composition L-PEI25K NP25 mCherry pDNA, L-PEI25K-PEG550 NP25 mCherry pDNA,-PEI25K-PEG1000 NP25 mCherry pDNA and -PEI25K-PEG2000 NP25 mCherry pDNA were prepared according to example 2. mCherry pDNA was labelled with Cy5 and mixed with unlabeled mCherry pDNA at the ratio 1:3. Polyplexes were prepared at 0.2 pDNA μg/mL.

Lipid based pDNA transfection particles: Transfection particles with composition DC-chol: Chol (50:50), DC-Chol: Chol: DOPE (33:33:33), DC-Chol: Chol: DOPE (30:65:5), KC2: Chol: DSPC (50:40:10) with mCherry pDNA at NP5, were prepared according to example 4. mCherry pDNA was labelled with Cy5 and mixed with unlabeled mCherry pDNA at the ratio 1:3. Lipoplexes were prepared at 0.2 pDNA μg/ml.

Transfection complexes in DMSO: 50 μL of polyplexes or lipoplexes were mixed with 50 μL DMSO and were transferred to DMSO using a vacuum oven as described in example 8.

Embedding transfection complexes in NCCell: 50 μL transfection complex in DMSO and 450 μL NCCell were mixed in an Eppendorf tube, and the sample was stirred at room temperature for 5 min.

In vitro release of particles: 100 μL NCCell compositions embedding transfection particles were injected into glass vials containing 2 mL PBS. The release samples were heated to 37° C. during the period of the experiment. 1 mL PBS was sampled at various timepoints and replaced with 1 ml PBS. Released particles were quantified by fluorescence spectroscopy (Tecan Spark, Ex/Em. 600/670 nm). Standard curves with concentrations between 0.08-5 μg/ml were prepared and the results were reported as cumulative release.

### Results and Discussion

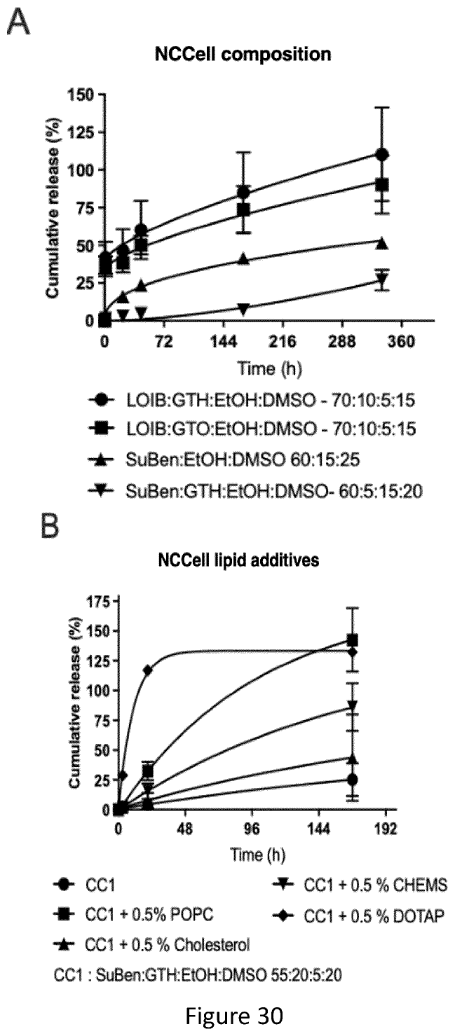

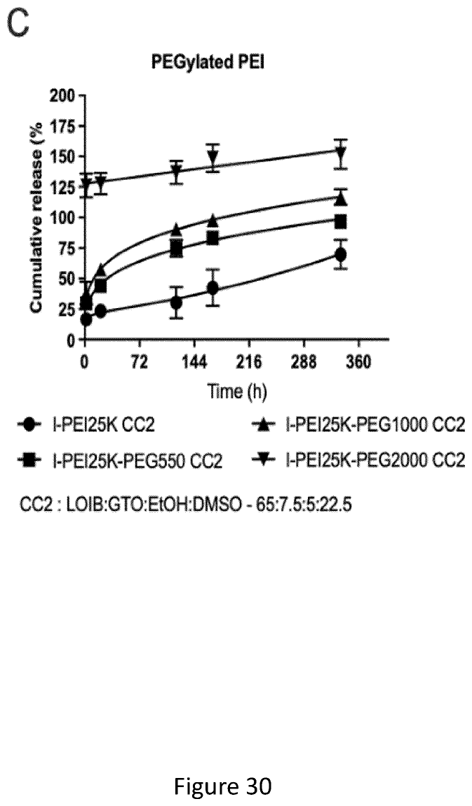

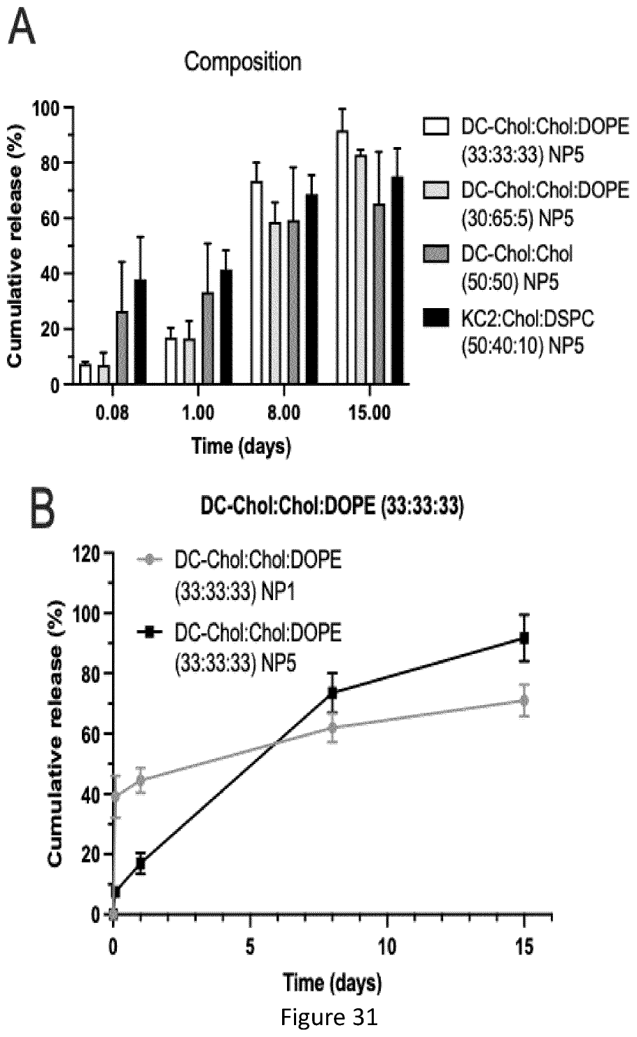

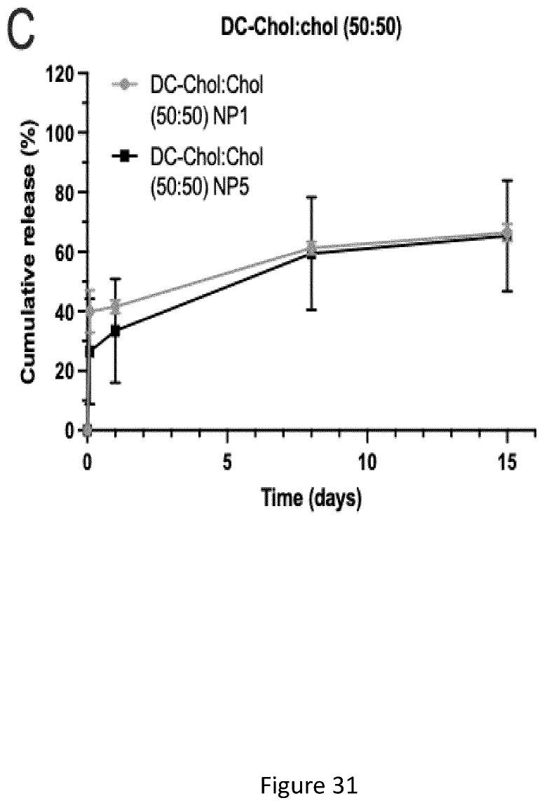

Transfection particles were prepared using Cy5 labelled mCherry pDNA and transferred to NCCells. In vitro release studies were performed in PBS and the cumulative release was quantified using fluorescence spectroscopy. The results are compiled in FIGS. 30 and 31.

The cumulative release of PEI-based particles could be modulated by the composition of the NCCell (FIG. 30A). Addition of lipids to the NCCell may further be used to modulate the release rate (FIG. 30B). PEG functionalization of the PEI polymers used for preparation of the PEI-pDNA particles can further be used for tuning of the release rate (FIG. 30C).

Lipid based pDNA transfection particles were released from NCCells (FIG. 31A), where lower NP ratio were found to stimulate a higher initial release (FIG. 31B-C).

### Conclusion

Controlled release of both PEI and lipid-based transfection systems was demonstrated using fluorescent labelled pDNA. Composition of the NCCell as well as the transfection system was shown to impact the release rate.

### Example 22: Release of PS Oligos from NCCells Evaluated by Fluorescence

Aim: Demonstrate solubility and controlled release of silencing particles made of PS oligos (AUM BioTech) from NCCells in vitro using Cy5-labelled PS and fluorescence for quantification.

### Methods:

NCCell_preparation: NCCell compositions SuBen: GTH: EtOH: DMSO (55:20:5:20+0.25% w/w Cholesterol), were prepared according to example 1.

PEI-PS transfection particles: Complexes with composition L-PEI40K PS oligo were prepared according to example 2 at NP25. Polyplexes were prepared at 0.1 PS μg/μl.

Liposome-based PS transfection particles: DOTAP: Chol: DOPE (50:45:5) NP5, were prepared according to example 3. Lipoplexes were prepared at 0.1 PS μg/μL.

LNP-based_PS_transfection particles: KC2: DC-Chol: Chol: DSPC (25:25:40:10) NP5, were prepared according to example 4 and 7. LNPs were prepared at 0.1 PS μg/μL. PS: PS was dissolved in H₂O at 0.1 PS μg/μL.

Transfection complexes in DMSO: 25 μL of polyplexes, lipoplexes or free PS (2.5 μg) were mixed with 50 μL DMSO and were transferred to DMSO using a vacuum oven as described in example 8.

Embedding transfection complexes in NCCell: 50 μL transfection complex in DMSO and 450 μL NCCell were mixed in an Eppendorf tube, and the sample was stirred at room temperature for 5 min.

In vitro release of particles: 100 μL NCCell compositions embedding transfection particles were injected into glass vials containing 2 mL PBS. The release samples were incubated at 37° C. during the period of the experiment. 1 mL PBS was sampled at various timepoints and replaced with 1 ml PBS. Released particles were quantified by fluorescence spectroscopy (Tecan Spark, Ex/Em. 600/670 nm). Standard curves with concentrations between 0.06-2 μg/ml were prepared and the results were reported as cumulative release.

### Results and Discussion

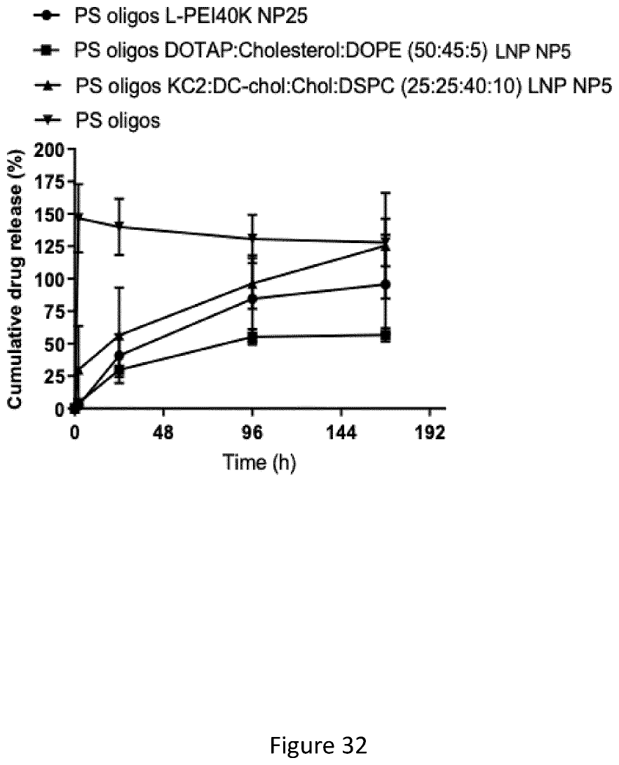

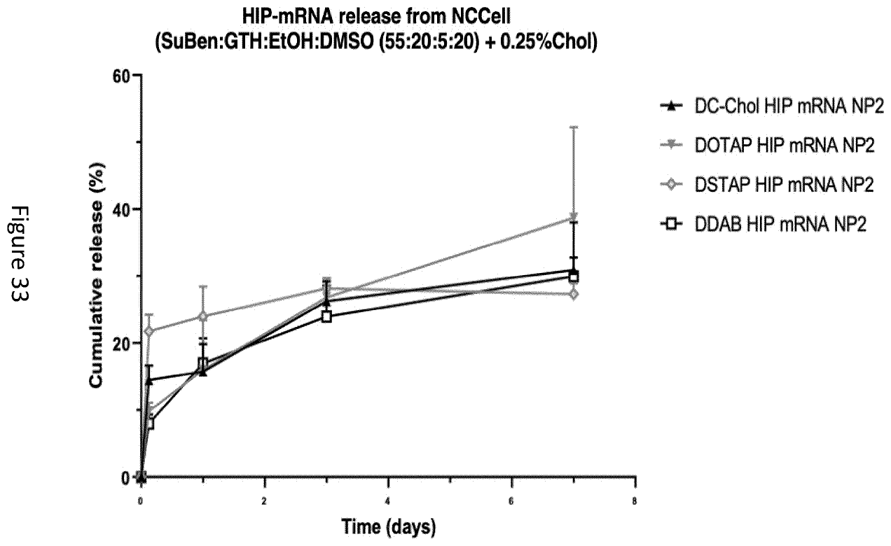

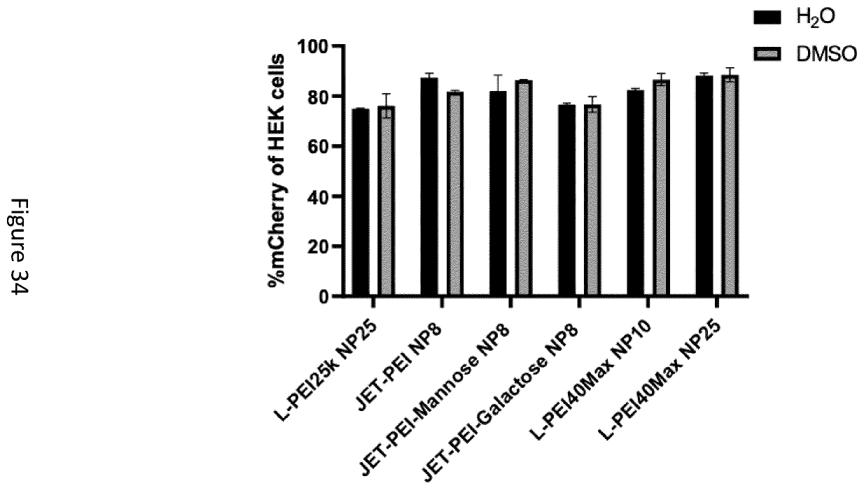

Transfection particles were prepared using Cy5-labelled PS oligos and transferred to NCCells. In vitro release studies were performed in PBS and the cumulative release was quantified using fluorescence spectroscopy. PS oligos complexed as particles displayed controlled release over a week. Free PS oligos dissolved in NCCells released completely within two hours suggesting that complexation into particles is necessary for long term release from NCCells. The results are compiled in FIG. 32.

### Conclusion

Controlled release of both PEI and lipid-based transfection particles embedded in NCCells was demonstrated using fluorescently labelled PS-oligos. All free PS was released within two hours showing that complexation is necessary for controlled release.

### Example 23: Controlled Release of HIP: MRNA Complexes from NCCells Evaluated by Ribogreen

Aim: Demonstrate controlled release of transfection HIP complexes from NCCells in vitro using Ribogreen assay for quantification.

### Methods:

NCCell preparation: NCCell composition SuBen: GTH: EtOH: DMSO (55:20:5:20)+0.25% Cholesterol was prepared according to example 1.

Preparation of ion-paired mRNA transfection complexes: Transfection complexes consisting of the HIPs DC-chol, DOTAP, DSTAP, DDAB were prepared with mCherry mRNA at NP2 according to example 5. The HIP: mRNA complexes were prepared at 0.1 μg/μL mRNA in water.

Transfer of HIP: mRNA complexes to DMSO: 50 μL of the HIP: mRNA complexes were mixed with 25 L DMSO and transferred to DMSO using a vacuum oven as described in example 8. The final mRNA concentration is then up-concentrated to 0.2 μg/μL Embedding transfection complexes in NCCell: 25 μL HIP: mRNA complex in DMSO were mixed with 475 μL NCCell in an Eppendorf tube, resulting in 10 μg mRNA per gram of NCCell. The sample was stirred at room temperature for 5 min.

In vitro release of HIP: mRNA complexes: 100 μL NCCell embedding the HIP: mRNA transfection complexes (1 μg mRNA) were injected into glass vials containing 2 mL PBS. The release samples were heated to 37° C. during the period of the experiment. 1 mL PBS was sampled at various timepoints and replaced with 1 ml of fresh PBS. Released HIP: mRNA complexes in the collected PBS fractions were diluted 1:1 with 0.5% TritonX and incubated for 30 minutes at 60° C. Then 100 μL of each sample were placed in a black 96-well plate and the mRNA was quantified following the Ribogreen assay procedure as described in example 6 using a Tecan Spark fluorescent plate reader (Ex/Em. 485/535 nm). Standard curve with concentrations between 100-2.5 ng/ml were prepared and the results were reported as cumulative release.

### Results and Discussion

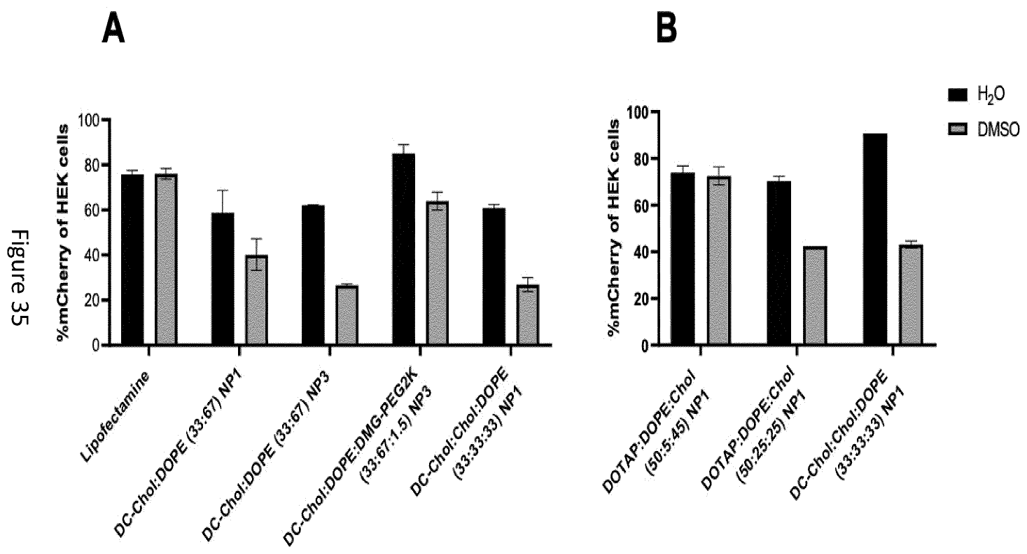

Transfection complexes using hydrophobic ion pairs (HIPs) were prepared with mCherry mRNA, transferred to DMSO and embedded in the NCCells. In vitro release studies were performed in PBS and the cumulative release was quantified using the Ribogreen assay kit. The results, compiled in FIG. 33, show the different mRNA release profile when using large molecules as HIPs.

The cumulative release of HIP: mRNA complexes can be modulated by the molecule that acts as an HIP. Complexes made with larger HIP molecules result in a small burst followed by sustained release phase where 30% of the mRNA is released in a week (FIG. 33).

### Conclusion

Controlled release of mRNA transfection complexes was demonstrated using different HIP molecules to complex the mRNA before embedment in the NCCell. Type of HIP used to complex the mRNA was shown to impact the release rate of mRNA from the NCCells.

### Example 24: Transfection Capabilities of pDNA, mRNA, and siRNA Particles are Maintained in Organic Media

Aim: Demonstrate in vitro transfection capabilities of a variety of polyplexes, liposomes, and solid lipid nanoparticles particles complexed with pDNA, mRNA, or SiRNA in DMSO

### Method:

Preparation of mCherry pDNA polyplexes: Transfection particles with composition L-PEI25k NP25, JET-PEI NP8, JET-PEI-Mannose NP8, JET-PEI-Galactose NP8, and L-PEI40Max NP10, and NP25 with mCherry pDNA were prepared at 0.4 μg/μl according to example 2

Preparation of pDNA and mRNA liposome based SLNPs: Transfection particles with composition Lipofectamine, DC-Chol: DOPE (33:67) NP1 and NP3, DC-Chol: DOPE: DMG-PEG2K (33:67:1.5) NP3 and DC-Chol: Chol: DOPE (33:33:33) NP1 with mCherry pDNA were prepared at 0.2 μg/μl according to example 3.

Transfection particles with composition DOTAP: DOPE: Chol (50:5:45) NP1, DOTAP: DOPE: Chol (50:25:25) NP1 and DC-Chol: Chol: DOPE (33:33:33) NP1 with mCherry mRNA were prepared at 0.2 μg/μl according to example 3.

SLNP Variants with mRNA and pDNA:

Transfection particles with composition KC2-Chol: DSPC (50:40:10) NP5 and KC2: DC-Chol: Chol: DSPC (25:25:40:10) NP5 with mCherry pDNA were prepared at 0.4 μg/μl according to example 4.

Transfection particles with composition KC2-Chol: DSPC (50:40:10) NP5, KC2-Chol: DSPC (50:40:10)+1.5% PEG NP5, KC2: DC-Chol: Chol: DSPC (25:25:40:10) NP5, DOTAP: Chol (50:50) NP3 and DOTAP: Chol: DOPE (50:45:5) NP3 with mCherry mRNA were prepared at 0.4 μg/μl according to example 4.

Luciferase siRNA Particles Variant Preparations:

Transfection particles with composition KC2: chol: DSPC (50:40:10) NP5 (0.4 siRNA μg/μl), MC3: chol: DSPC (50:40:10) NP5 (0.4 siRNA μg/μl) and DOTAP: Chol: DOPE (50:45:5) NP2 (0.2 siRNA μg/μl) with luciferase siRNA were prepared according to example 3 and 4.

Transfection complexes in DMSO: 50 μl of lipid particles were mixed with 50 μl DMSO and were transferred to DMSO using a vacuum oven as described in example 8.

In vitro transfection of HEK cells with pDNA and mRNA particles: 20K HEK293 cells (Human embryonic kidney 293 cells) were seeded per well in a 96 well plate 24 h prior to transfection in 100 μl DMEM high glucose, GlutaMAX™ Supplement 10% FBS, 1% Sodium Pyruvate (100 mM), and 1% ml Penicillin-Streptomycin (10,000 U/mL)). Treatment was 0.4 μg of pDNA or mRNA per well in 100 μl transfection volume and incubated for 24 h in a humidified tissue culture incubator at 37° C. with 5% CO₂. Transfection efficiency was tested for both particles in water and DMSO. The percent of mCherry positive HEK cells of the live fraction was quantified by flow cytometry. Cells were spun down to remove media and then resuspended in 200 μl 1×PBS and analyzed by flow cytometry. The viability of the cells is assayed based on morphology using SSC-A and FSC-A parameters. Transfection is measured using SSC-A against mCherry (YL2-A channel on Attune NxT) parameters.

### In Vitro Silencing of MDA-MB-231-Luc Cell Line:

20K MDA-MB-231-luc cells (Human epithelial, breast cancer cell line genetically modified to express luciferase) were seeded per well in a 96 well flat clear bottom, white, tissue culture treated plate 24 h prior to transfection in 100 μl RPMI 16400 medium, GlutaMAX™ Supplement 10% FBS, 1% Sodium Pyruvate (100 mM), and 1% ml Penicillin-Streptomycin (10,000 U/mL)). Treatment was 0.2 μg of pDNA or mRNA per well in 100 μl transfection volume and incubated for 48 h in a humidified tissue culture incubator at 37° C. with 5% CO₂. Transfection efficiency was tested for both particles in water and DMSO.

For analysis, the supernatant is removed, and 100 μl (150 μl/ml) D-luciferin (stock: 30 mg/ml) is added to the cells and incubated for 5 min. The plate is placed in SpectraMax® iD5 Multi-Mode Microplate Reader, where bioluminescence is measured for all wavelengths. Lower bioluminescence compared to untreated cells indicates a stronger silencing of the luciferase gene by the luc siRNA. A scrambled siRNA is included as a control.

### Results:

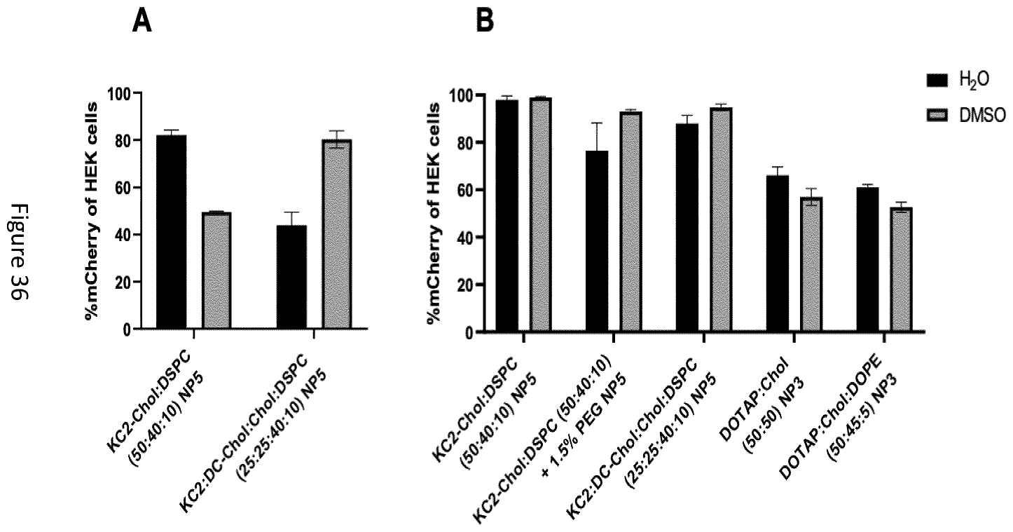

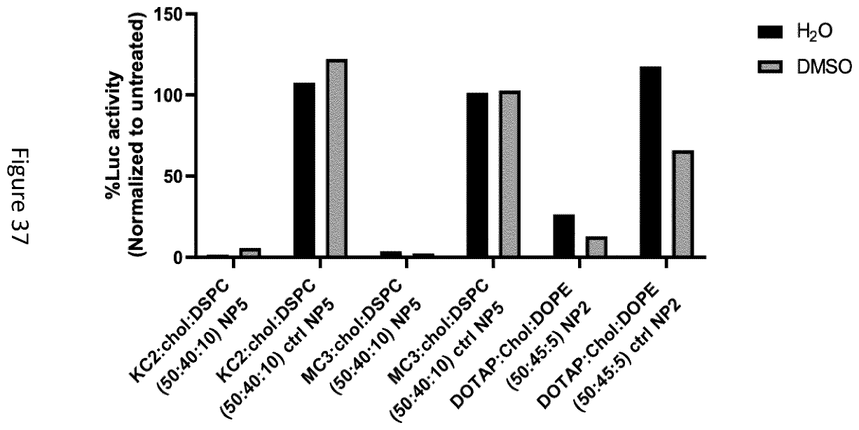

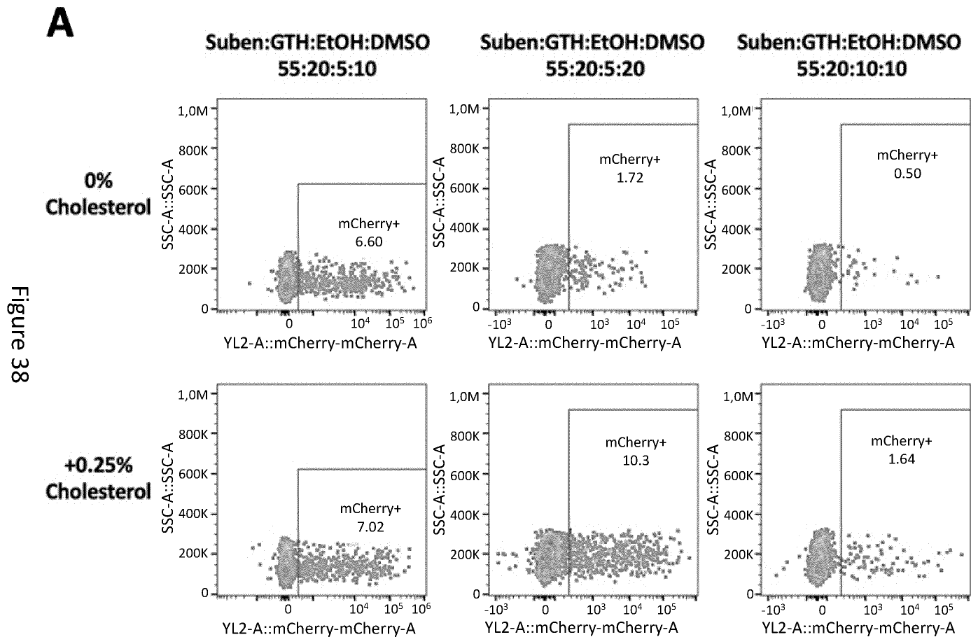

All mCherry pDNA and mRNA formulations maintained transfection efficiency in HEK cells upon transfer to DMSO (FIG. 34-36).

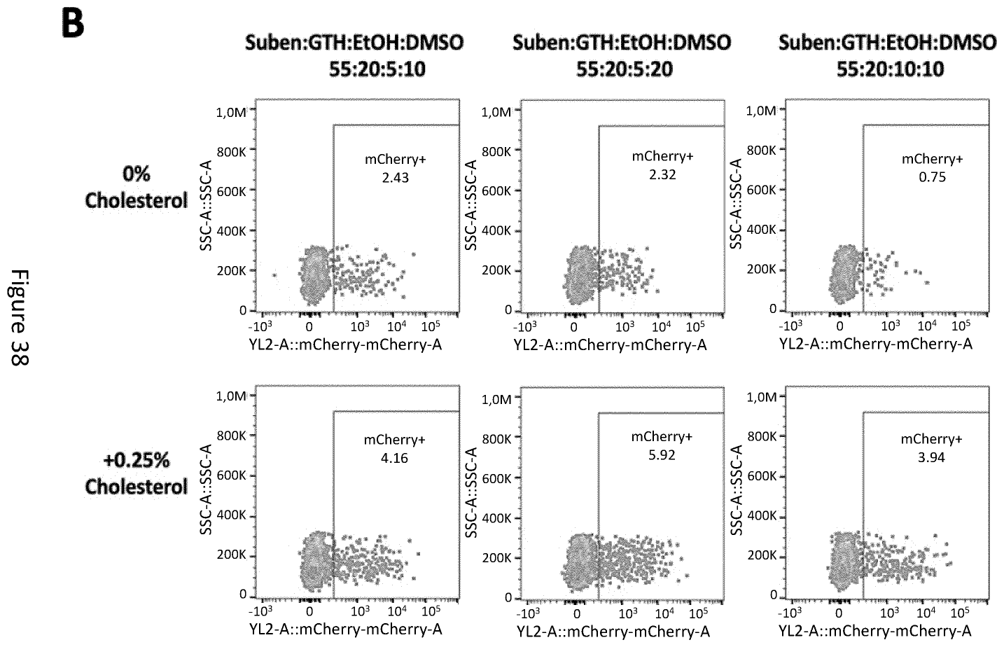

SLNPs and liposomes complexed with luciferase siRNA maintain the ability to silence MDA-MB-231-luc cells after transferring to DMSO (FIG. 37) However, silencing is best at 48 hrs post transfection compared to cells transfected with particles complexed with scrambled siRNA.

### Conclusion:

A variety of formulations of polymers, liposomes, and SLNPs complexed with mCherry pDNA, mCherry mRNA, or luc siRNA maintain transfection efficiency after being transferred to DMSO. This is demonstrated as transfection and silencing in different cell lines.

### Example 25: NCCells Provide Sustained Release of Effective Transfection Particles

Aim: Demonstration of sustained transfection capabilities of mRNA mCherry transfection particles released from a NCCell formulation

Method: Transfections particles DC-Chol-Chol-DOPE (30:65:5) liposomes mRNA (mCherry, 40 μg/ml) NP5 or L-PEI40K mRNA (mCherry, 40 μg/ml) particles were formulated in three different NCCell compositions; SuBen: GTH: EtOH: DMSO (55:20:5:10), SuBen: GTH: EtOH: DMSO (55:20:5:20) or SuBen: GTH: EtOH: DMSO (55:20:10:10) with or without the addition of 0.25% Cholesterol. Transfections were performed 24 transwell (5 μm pore) culture plates with HEK293 (Human embryonic kidney 293 cells) in 2 ml culture volume (culture media: 500 ml DMEM high glucose, GlutaMAX™ Supplement 50 ml FBS, 5 ml Sodium Pyruvate (100 mM) and 5 ml Penicillin-Streptomycin (10,000 U/mL)). NCCells (100 μl) were injected into the transwell insert using a 2-component 1 ml syringe and 23G needle and allowed to release transfection particles for 48 h (50.000 HEK cells in 2 ml media) and the inserts were then transferred to a new well (25.000 HEK cells in 2 ml media) and allowed to transfect for an additional 72 h (time point 120 h). For insert transfer the NCCell injected insert is removed from the well, all remaining supernatant is removed and discarded before placing in the next well where particles are to be released for intended period. For transfection analysis the cells are resuspended in 250 μL of PBS and transferred to a round bottom 96 well plate. Once in the 96 well plate, the cells were centrifuged for 3 min at 600G, after which the supernatant is discarded, and the cells are resuspended in 200 μl of PBS. This process is repeated twice.

After washing, the cells are resuspended in 200 μl PBS and analyzed by flow cytometry. The viability of the cells is assayed based on morphology using SSC-A and FSC-A parameters. Transfection is measured using SSC-A and mCherry (YL2-A channel on Attune NxT) parameters.

### Results:

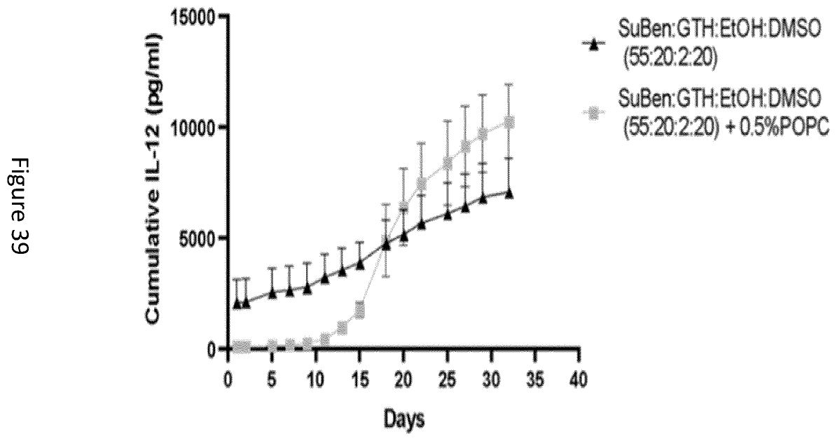

All NCCell mCherry formulations transfected of HEK cells at both the 48 h (FIG. 38A) and 120 h time-points (FIG. 38B) demonstrating that released DC-Chol: Chol: DOPE mCherry mRNA particles retain transfection capability upon release from NCCells.

### Conclusion:

The NCCell technology can retain and provide a sustained release of mRNA transfection particles.

### Example 26: NCCells can Provide Sustained Release of Transfection Particles Yielding a Continuous Production of IL-12 at Levels Capable of Activation T Lymphocytes

Aim: Demonstration of sustained transfection capabilities of transfection particles released from NCCell formulation and T cell activation mediated by continuously produced IL-12 from transfected cells.

### Method:

Transfections particles (L-PEI/pDNA (IL-12) NP 25, 40 μg pDNA/ml) was formulated and prepared in a NCCell (SuBen: GTH: EtOH: DMSO (55:20:2:20) or SuBen: GTH: EtOH: DMSO (55:20:2:20)+0.5% POPC) as described in example 1, 2, 8 and 12.

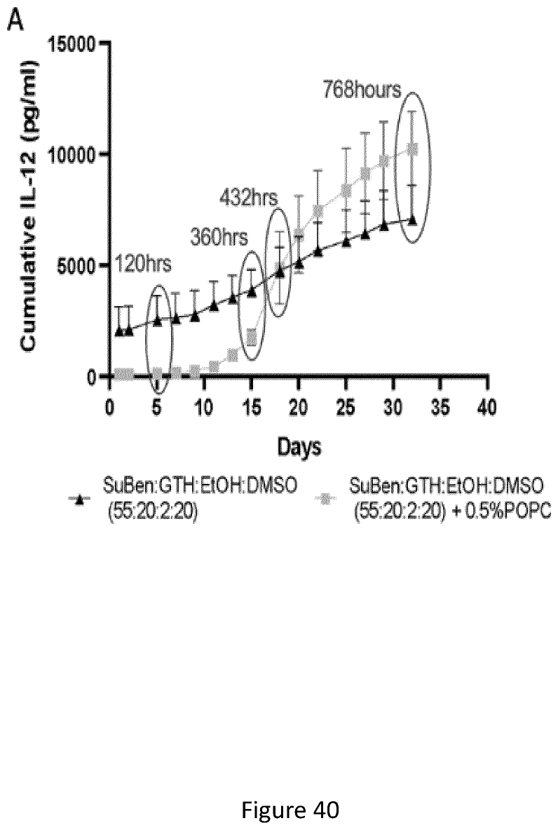

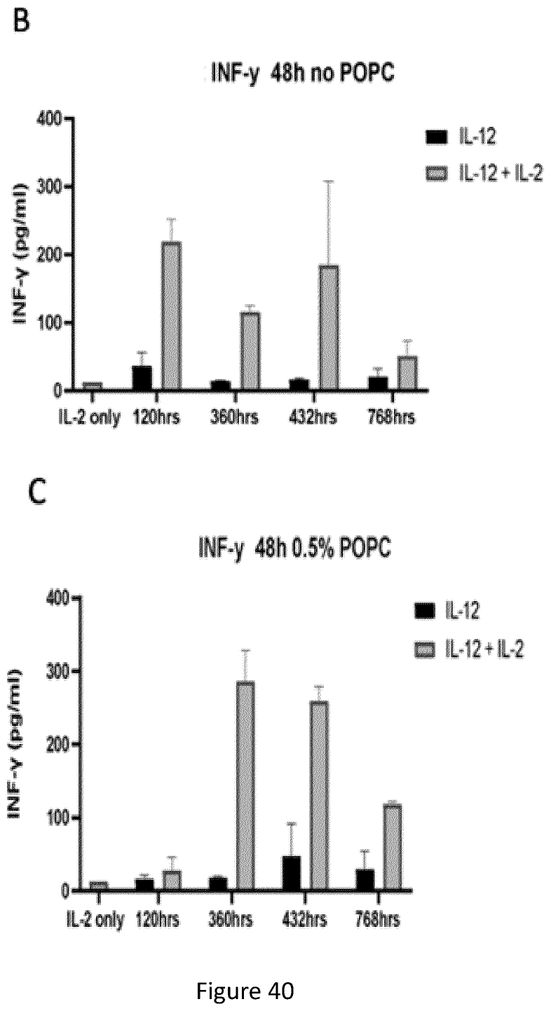

Transfections were performed 24 transwell (5 μm pore) culture plates with 100.000 HEK293 in 2 ml culture volume. NCCells were injected into the transwell insert and the insert were transferred to a new well after 1 hr, 24 and left for another 24 hrs (48 hrs), then transferred to a new well and left for another 72 hours (120 hrs). The transfer of the insert was continued as indicated in FIG. 16. Every time inserts were transferred culture media (supernatant) was collected and stored at −20° C. until analysis. Transfection efficiency evaluated by Mouse IL-12 p70 DuoSet ELISA (Sandwich) according to manufactures protocol (Manufacturer). Analysis were performed using a SpectraMax iD5 Multi-Mode Microplate Reader (450 nm+570 nm for reference) and Graphpad Prism was used to generate standard curve. IL-12 concentrations produced were plotted as cumulative protein amount as a function of time (FIG. 39-40).

Immune activating capabilities of the IL-12 produced by the HEK cells in the transwell system was investigated by adding 100 μl of the supernatant collected at the 120 hrs, 360 hrs, 432 hrs and 768 hrs timepoints. Supernatant was added to purified naïve pan T cell harvested from the spleen of a female treatment naïve balb/C mouse. T cells were activated by aCD3 (0.5 μg/ml) and aCD28 (5 μg/ml) for 24 hours in the T cells media (complete RPMI1640+1% ITS). Prior to seeding, T cells were stained with CFSE for proliferation assay. 100K T cells were seeded in a 96-well plate (round) in 100 μl media. Cells were stimulated for 24 and 48 hours with IL-2 0.2 pl/ml (stock: 100 μg/ml) before addition of the supernatant containing IL-12.

T cell were characterised by flow cytometry (Attune, ThermoFisher) and analysed for INF-y production after stimulation with IL-12 transfection supernatants by ELISA. Flow Stain: CFSE-FITC, CD8-APC and VD (eFlour780)-APC-Cy7 channel. Results are presented in FIG. 40.

### Results:

NCCell formulations with L-PEI/pDNA (IL-12) NP 25 transfection particles were, in the transwell system, capable of providing sustained release of pDNA particles that induced production of IL-12 by HEK cells. The addition of POPC delayed the onset of IL-12 but yielded higher cumulative IL-12 protein levels over the period investigated (FIG. 39).

The IL-12 levels produced at multiple intervals were demonstrated to activate T cell production of INF-y supporting that the IL-12 protein is therapeutically active and production levels sufficient to yield cellular activity (FIG. 40).

### Conclusion:

The NCCell compositions (SuBen: GTH: EtOH: DMSO (55:20:2:20) and SuBen: GTH: EtOH: DMSO (55:20:2:20)+0.5% POPC) formulated with transfections particles (L-PEI/pDNA (IL-12) NP 25, 40 μg pDNA/ml) were able to continuously transfect HEK cell in culture and stimulate the production of IL-12. This finding validate that transfection particles are stable and functional even when released over long periods. The levels and function of the produced IL-12 was furthermore validated to be capable of activating T cells in vitro.

### Example 27: NCCells Increase T Lymphocyte Infiltration and Cytotoxic T Cell to Regulatory T Cell Ratio in Syngeneic Murine Cancer Models

Aim: Demonstrate the improved T cell recruitment and polarization of tumors injected with NCCells, providing a sustained release of IL-12 transfection particles compared to high dose repeated or a single injection of free transfection particles.

### Method:

Study part 1-Comparison of NCCell sustained release of 2 μg IL-12 pDNA to high dose single or repeated injection of free transfection particles 50 μg IL-12 pDNA per injection in the CT26 cancer model.

Transfection particles (L-PEI25K NP25/pDNA (IL-12), particles for a total conc. of 90 μg/ml in 50 μl NCCell) were formulated and prepared in a NCCell (SuBen: GTH: EtOH: DMSO (55:20:5:20)+0.25% chol) as described in Example 1, 2, 8 and 12. For free particle comparison, L-PEI25K NP25/pDNA (IL-12) was included at a concentration of 2 μg/μl and injected for a total volume of 25 μl, equalling a dose of 50 μg per injection.

Murine CT26 tumors were established in female Balb/C mice. CT26 syngeneic murine colorectal cancer cells were cultured in RPMI 1640 medium (Gibco™, Thermo Fisher Scientific, or Sigma-Aldrich, Merck) as previously described. Media was supplemented with 10% fetal bovine serum (Gibco™, Thermo Fisher Scientific or Biowest, VWR) and 1% penicillin-streptomycin (Gibco™, Thermo Fisher Scientific or Sigma-Aldrich, Merck). Cells were kept in a humidified tissue culture incubator at 37° C. with 5% CO2. Cells were subcultured when confluent, and Trypsin-EDTA (0.25%) (Thermo Fisher Scientific) was used to remove adherent cells. Mice were injected under general anesthesia (Isoflurane 2%, 95% 02) with 300.000 cells in 100 μl PBS. Tumors were allowed to establish for 14 days.

Day 0, mice were, under general anaesthesia, injected in tumors with 50 μl of NCCell formulation (total dose 4.5 μg pDNA) or 25 μl of free transfection particles (total dose 50 μg pDNA), mice with tumor injected with empty NCCell were included as controls. NCCell injections were performed using a 1 ml syringe and a 23 Ga. hypodermic needle, and free particles were injected using a 29 Ga. needle. Two days after the first injection, tumor infiltration was investigated by flow cytometry (described below) of tumors removed from sacrificed mice (cervical dislocation). To demonstrate the benefit of NCCell sustained transfection technology, remaining mice in the group injected with free transfection particles on day 0 had an additional intratumoral injection of free transfection particle performed (25 μl) on day 3. On day six, after the first injection of NCCell or two injections of free transfection particles, remaining mice were sacrificed, and tumor T cell immune infiltration was evaluated.

For tumor analyses, tumors were carefully dissected and placed in MACS tissue storage solution (Miltenyi Biotec). Weighed tumors were minced and enzymatically digested (Tumor Dissociation Kit, mouse, Miltenyi Biotec) (37° C. water bath, 40 min. with shaking. Digested tumors were passed through 70 μm strainers and diluted in PBS. Cell counts were determined (MUSER Cell Count and Viability Assay, MUSE® Cell Analyzer, Merck Millipore). 1-10×106 cells were Fc-blocked (5-10 min. on ice) with murine anti CD16/32 monoclonal antibodies (BD Biosciences) followed by dead cell labeling with fixable viability dye (VD)-efluor780 (eBioscience) and surface staining (30 min. on ice) with specific antibodies. The flow cytometric surface staining panel included CD45, Ter119, CD8α, CD25, PD-1, CD3, CD4.

For intracellular staining of IFN-γ and FoxP3 cells were fixed (30 min. at RT) and permeabilized using the eBioscience™ Foxp3/Transcription Factor Staining Buffer Set (Thermo Fisher Scientific).

The samples were acquired on an LSRfortessa X20 Flow Cytometer using FACSDiva software (BD Biosciences) and data analyzed using FlowJo software (Treestar). Spectral spillover compensation controls were included using UltraComp eBeads™ Plus Compensation Beads (Invitrogen, Thermo Fisher Scientific) and ArC™ Amine Reactive Compensation Bead Kit (Invitrogen, Thermo Fisher Scientific). Debris and doublets were excluded based on forward, and side scatter. Fluorescence minus one (FMO) samples were used for the identification of positive and negative populations.

Study part 2—Comparison of NCCell sustained release of 2 μg IL-12 pDNA to high dose single or repeated injection of free transfection particles 50 μg IL-12 pDNA per injection in the MC38 cancer model.

Transfection particles (L-PEI25K/pDNA (IL-12), particles for a total conc. of 2 μg in 50 μl NCCell were formulated and prepared in a NCCell (SuBen: GTH: EtOH: DMSO (55:20:5:20)) as described in Example 1, 2, 8 and 12. For free particle comparison, L-PEI25K/pDNA (IL-12) was included at a pDNA concentration of 0.1 μg/μl and injected as a volume of 50 μl, total pDNA dose 5 μg.

Murine MC38 tumors were established in female C57BL/6 mice. MC38 cancer cells were cultured (RPMI) and injected as described for the CT26 model above (300.000 cells injected in PBS). Tumors were allowed to establish for 15 days before injection of transfection particles or NCCell transfection technology (day 0); untreated controls were included for comparison. All injections and handling of mice were performed as described above in the CT26 study. After six days (day 6), tumors were dissected and prepared for flow cytometric analyses as described above.

### Results:

Study 1—CT26 cancer model

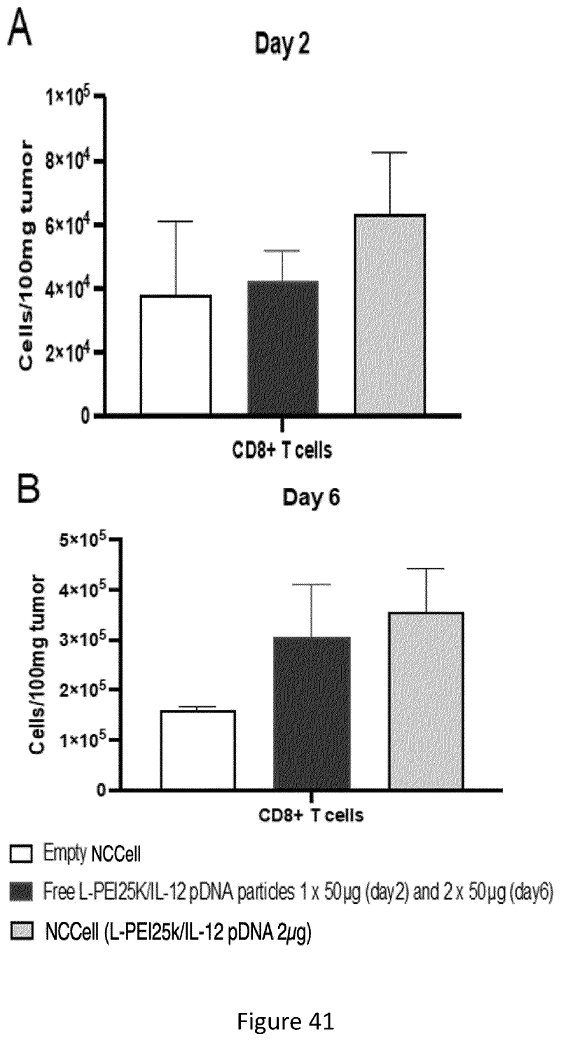

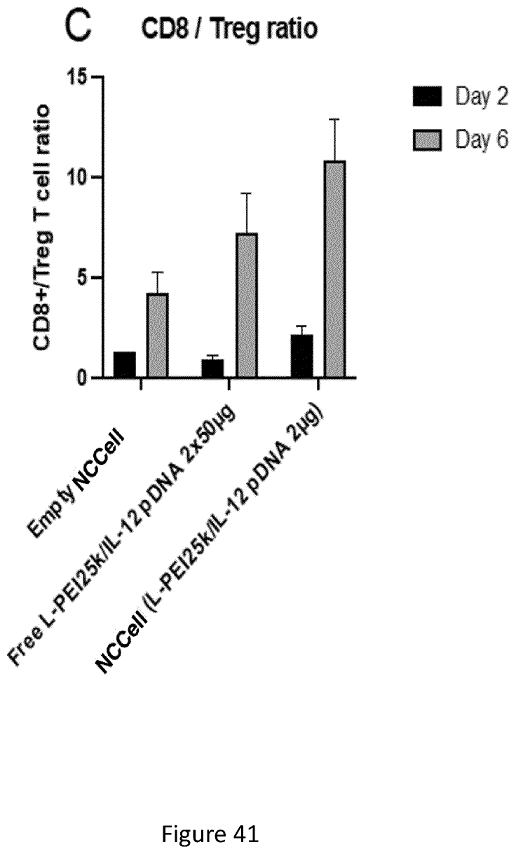

Despite injecting 25 times higher doses of intratumoral free IL-12 pDNA transfection particles compared to the NCCell sustained release technology, the NCCell injected tumors displayed improved T cell activation. In the NCCell injected tumor, a higher number (cells/100 mg of tumor) of cytotoxic (CD8+) T cells was observed compared to tumors injected with free particles and controls (FIG. 41). This pattern was also present at the day six analyses were NCCell injected (single injection, total dose 2 μg IL-12 pDNA) again displayed higher intratumoral concentrations of cytotoxic T cells compared to free particles injected (two injections, total dose 100 μg IL-12 pDNA) and controls (FIG. 41). Equally important, the NCCell treated tumors displayed the highest cytotoxic to regulatory T cells ratio at both the day two and day six time points (FIG. 41)

Study part 2—MC38 cancer model:

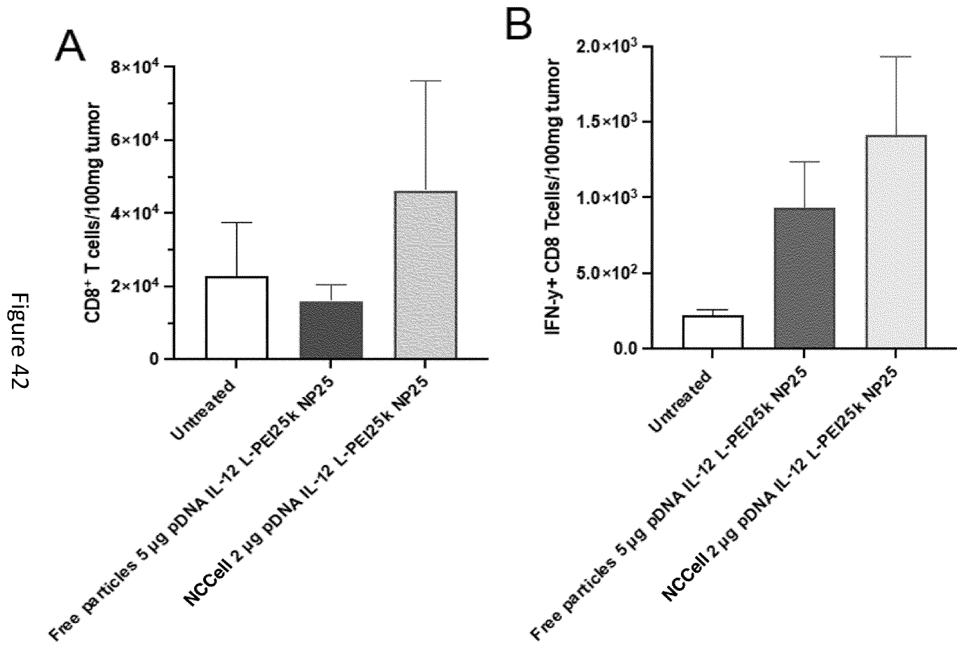

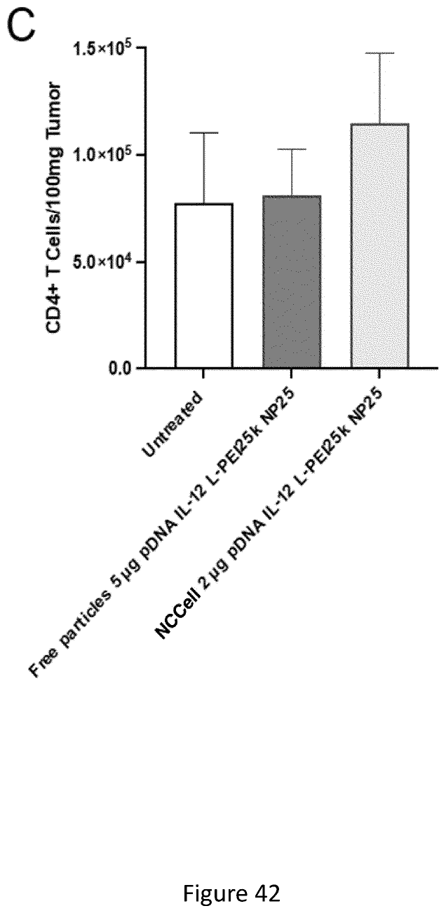

As for the CT26 tumors, the NCCell IL-12 pDNA injected tumors displayed a higher concentration of cytotoxic (CD8) T cells compared to free IL-12 pDNA particles and controls. This was again observed despite injecting a 2.5 times lower amount of pDNA. The benefit of the NCCell delivery was also demonstrated by an increased number of activated IFN-γ positive CD8 T cells in tumors and increased numbers of CD4 T cells (FIG. 42).

### Conclusion:

The NCCell sustained-release IL-12 transfection technology increase intratumoral cytotoxic T cell concentration and activation compared to free particles and empty NCCell. Most importantly, the benefit of sustained-release NCCells was demonstrated as even repeated injection of much higher pDNA free transfection particles did not increase T cell infiltration compared to a single injection of NCCell IL-12 transfection system. The studies demonstrate the therapeutic potential of the sustained release NCCell technology compared to rapid wash-out and loss of free transfection particles activity.

### Example 28: NCCells Sustained IL-12 Transfection Reduces Tumor Growth of a Murine Syngeneic Colorectal Cancer Models

Aim: To demonstrate the therapeutic efficacy of the sustained release NCCell IL-12 transfection technology

### Method:

Study part 1—Comparison of NCCell sustained release of 4 μg L-PEI25k NP25 and 8 μg DC-Chol: Chol: DOPE (30:65:5) IL-12 pDNA particles in a MC38 murine cancer model.

Transfection particles; formulation 1: L-PEI25K NP25/IL-12 pDNA 4 μg (80 μg/ml) particles were formulated in a 50 μl NCCell composition (SuBen: GTH: EtOH: DMSO 55:20:5:20+0.25% Chol), Formulation 2: DC-Chol: Chol: DOPE (30:65:5) NP5/IL-12 pDNA 8 μg (160 μg/ml) particles were formulated in a 50 μl NCCell composition (SuBen: GTH: EtOH: DMSO 55:20:5:20+0.25% Chol).

Murine MC38 tumors were established in female C57BL/6 mice, as described in example 27. Tumors were allowed to establish for 15 days. Tumors larger than 50 mm3 were included in the study. Mice were blindly randomized to treatment groups. Under general anesthesia (isoflurane 1.5-2% in 95% 02), mice were injected centrally in the tumor using a 23 Ga needle and 1 ml syringe twice a week. Tumors were measured 2-3 times a week.

Study part 2—Demonstrating tumor control from NCCell sustained IL-12 mRNA transfection of 4 μg (80 μg/g) DC-Chol: Chol: DOPE (30:65:5) NP5 particle in the CT26 murine cancer model.

Transfection particle 80 μg/g DC-Chol: Chol: DOPE (30:65:5) IL-12 mRNA liposomes were formulated and prepared in a NCCell (SuBen: GTH: EtOH: DMSO 55:20:5:20+0.25% Chol) as described in example 1, 3, 8 and 12-14.

Murine CT26 tumors were established in female Balb/c mice, as described in example 27.

Tumors were allowed to establish for 15 days. Mice were blindly randomized to treatment groups. Under general anaesthesia (isoflurane 1.5-2% in 95% 02), mice were injected centrally in the tumor with 50 μl NCCell using a 23 Ga needle and 1 ml. The mice were treated twice with one week interval. Tumors were measured 2-3 times a week. Mice with a tumor size >1500 mm3 were euthanized and recorded as the day of death for survival analysis.

### Results:

Study 1—IL-12 pDNA efficacy in MC38 cancer model

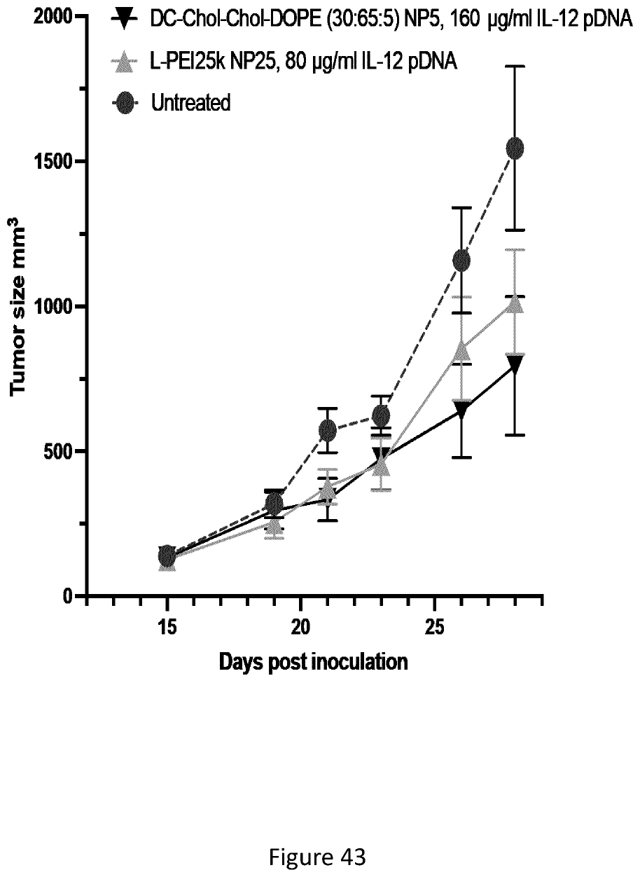

Tumor growth curves demonstrated that NCCell formulation 1 and formulation 2 delayed tumor growth (FIG. 43). None of the treated mice displayed failure to thrive or weight loss in comparison to untreated controls. The injected volume of injected NCCell formulation has not been deducted from the tumor volume on growth curves.

Study 2—IL-12 mRNA efficacy in CT26 cancer model

The tumor growth curves show that DC-Chol: Chol: DOPE (30:65:5) NP5 liposomes with IL-12 mRNA released from NCCell delay tumor growth and increase median survival time (FIG. 44). None of the treated mice displayed failure to thrive or weight loss in comparison to untreated controls. The injected volume of injected NCCell formulation has not been deducted from the tumor volume on the growth curves.

### Conclusion:

The NCCell sustained-release of IL-12 pDNA or mRNA transfection technology is therapeutically active and able to reduce tumor growth of two different murine syngeneic tumors.

### Example 29: Stability of SLNP Transfection Particles in Water and DMSO

### Aim:

Compare transfection capacity of freshly made and old transfection particles stored in water (at 4° C.) and in DMSO (at room temperature) for 1 week.

### Methods:

KC2 LNP Variants with mCherry mRNA and pDNA Preparation:

Transfection particles with composition KC2: Chol: DSPC (50:40:10) NP5, KC2: DC-Chol: Chol: DSPC (25:25:40:10) NP5 and DC-Chol: Chol: DOPE (30:65:5) with mCherry pDNA and mRNA at NP5 were prepared at 0.4 μg/μL according to example 4.

Transfection complexes in DMSO: 50 μL of lipid particles were mixed with 50 μL DMSO and were transferred to DMSO using a vacuum oven as described in example 6.

In vitro transfection of HEK cells: 20K HEK293 cells (Human embryonic kidney 293 cells) were seeded per well in a 96 well plate 24 h prior to transfection in 100pl DMEM high glucose, GlutaMAX™ Supplement 10% FBS, 1% Sodium Pyruvate (100 mM) and 1% ml Penicillin-Streptomycin (10,000 U/mL)). Treatment was 0.4 μg of pDNA or mRNA per well in 100 μl transfection volume and incubated for 24 h in a humidified tissue culture incubator at 37° C. with 5% CO2. The particles were either tested freshly made or tested after being stored for one week at 4° C. for the particles in water and at room temp for the ones in DMSO. The percent of mCherry positive HEK cells of the live fraction was quantified by flow cytometry. The ratio of percent of mCherry positive HEK293 for the old and fresh particles was calculated and is presented in FIG. 15. A ratio of 1 corresponds to unchanged performance of the particles upon storage.

### Results and Discussion

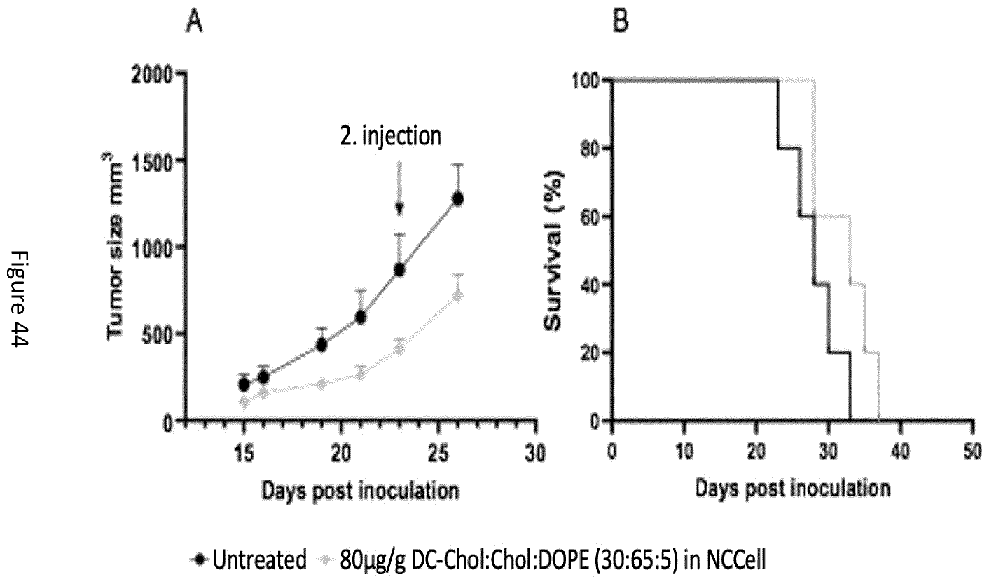

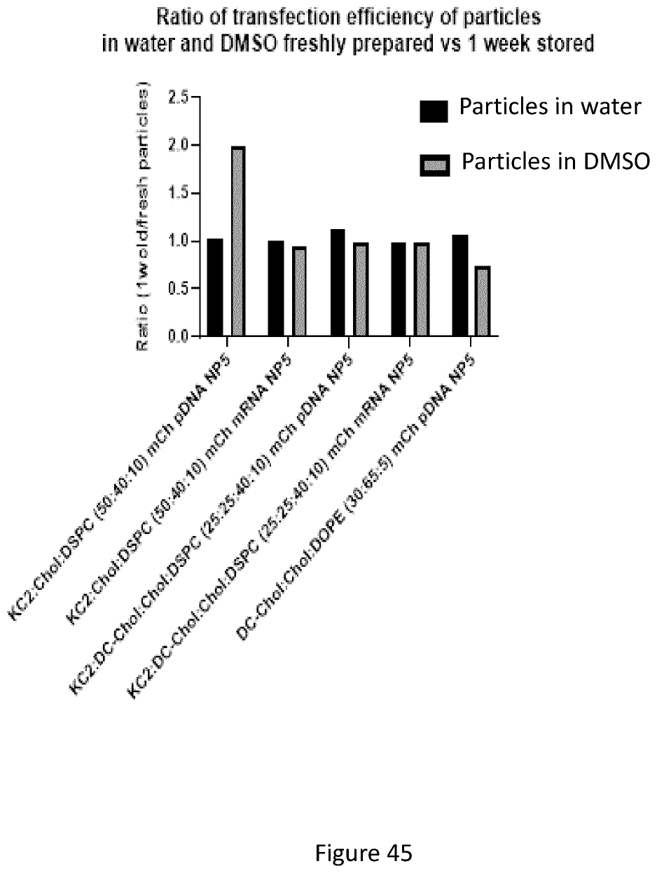

The capacity of the transfection particles prepared and stored in water to transfect HEK cells with mCherry pDNA and mRNA in vitro is presented in FIG. 45. Here freshly prepared transfection particles are compared to particles that have been stored 1 week.

The results show that a range of KC2 and DC-chol based particles in water maintain their transfection capacity at least 1 week at 4C, as seen by the maintained transfection efficiency between freshly prepared particles and 1 week stored particles.

### Conclusion

The transfection efficacy of the particles in water and DMSO is preserved for at least 1 week stored at either 4° C. or room temperature.

### Example 30: NCCell Display Gene Silencing Capability

Aim: To demonstrate gene silencing capability of LNP particles released from an NCCell.

Methods: siRNA particles KC2: Chol: DSPC (50:40:10) LNP were prepared as described in example 1, 8, 14 and 24 in an NCCell (SuBen: GTH: EtOH: DMSO (55:20:5:20)) at NP5 ratio, at a final concentration of 40 μg/ml Luc siRNA or scrambled control siRNA.

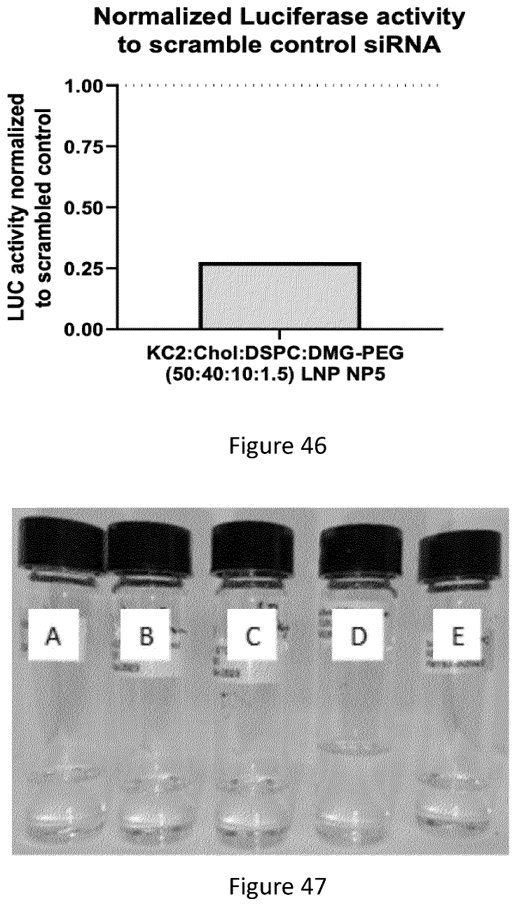

In a 24 transwell plate (5 μm pore) 50.000 MDA-MB-231-Luc cells were seeded, 24 h prior to injecting gene silencing NCCell in the transwell insert, in 2 mL RPMI 1640 medium, GlutaMAX™ Supplement 10% FBS, 1% Sodium Pyruvate (100 mM), and 1% ml Penicillin-Streptomycin (10,000 U/mL). NCCells (100 μl) were injected into the transwell insert using a 2-component 1 ml syringe and 23G needle and allowed to release transfection particles for 48 h. After 48 h cells were washed in PBS and resuspended in 200 μL freshly prepared PBS suspension containing Luciferin (150 μg/ml) and bioluminescence recorded for 30 minutes with 5 min steps between image acquisitions as described in example 24. Difference in luminescence signal between scrambled control siRNA and Luc siRNA was determined (FIG. 46).

### Results and Discussion

The NCCell composition with KC2: Chol: DSPC siRNA particles display gene silencing upon release of siRNA particles in the transwell system. The observed luminescence difference (FIG. 46) indicates that Luc expression in cells is silenced by the Luc siRNA released from the NCCell depot in the transwell insert.

### Example 31: Preparation of NCCell with Various Solvents

Aim: Demonstrate the preparation of NCCell using various class 3 solvents.

Methods: NCCell with the following compositions SuBen: GTH: EtOH: DMSO 55:20:2:15, SuBen: GTO: 1-propanol 60:20:20, SuBen: GTO: 1-butanol 60:20:20, SuBen: GTO: anisole 60:20:20, SuBen: GTO: propylene carbonate 60:20:20 were prepared as described in example 1.

Results and discussion: NCCell can be prepared using several Class 3 solvents including 1-propanol, 1-butanol and anisole using both GTH and GTO as co-solvents. All NCCell were clear and had similar fluidity when judged by observing the flow of the compositions upon tilting of the vials. FIG. 47 illustrates the following NCCell formulations using GTO or GTH as co-solvents:
A. SuBen: GTH: EtOH: DMSO 55:20:2:15
B. SuBen: GTO: 1-propanol 60:20:20
C. SuBen: GTO: 1-butanol 60:20:20
D. SuBen: GTO: anisole 60:20:20
E. SuBen: GTO: propylene carbonate 60:20:20

Conclusion: NCCell with suitable fluidity for injection can be prepared using Class 3 solvents including 1-propanol, 1-butanol, anisole, and these variants can help modify the release profiles, solubility and toxicity of embedded compounds, complexes, and particles.

### Example 32: Synthesis of Mannose Functionalized PEI

Aim: Describe the preparation of mannose functionalized polyethyleneimine (PEI).

Methods: Three molar ratios of α-D-mannopyranosylphenyl isothiocyanate (MPITC) were reacted to L-PEI (40 kDa) to achieve 1 mol %, 5 mol %, or 10 mol % coverage of PEI (mol % of reactive amines) with MPITC. MPITC (11.6, 58.1 or 116.1 μmol respectively) was dissolved in dry methanol (950 μL) and added to an Eppendorf tube (2 mL) with L-PEI (50 mg, 40 kDa) dissolved in dry methanol (50 μL) in a drop-wise manner. The reaction was then agitated on a Hulamixer™ at room temperature until no MPITC could be detected by thin layer chromatography (TLC) analysis (Dichloromethane:methanol, 1:1 v/v, UV256 detection). The crude reaction was diluted with mQ water to lower the methanol concentration to 10% and it was then loaded into a dialysis cassette (Slide-A-Lyzer 3.5k MWCO). The three PEI-MPITC compounds were dialyzed overnight in mQ water at room temperature and the mQ water frequently exchanged, yielding the wanted compound.

Conclusion: Mannose-functionalized PEI polymers were successfully isolated with different ratios of mannose, namely 1 mol %, 5 mol %, and 10 mol %.

### Example 33: Preparation of Mannose-PEI Particles and In Vitro Transfection Using Free Mannose-PEI Particles, and Particles Embedded in NCCell

Aim: Prepare mannose-PEI transfection particles and demonstrate mRNA transfection ability from free particles and particles embedded in NCCell in the DC2.4 cell line.

### Methods:

### Study Part 1: Mannose-PEI Free Particles

Mannose-functionalized PEI 25 kDa polymers were synthesized as described in example 32 with different PEI/mannose ratios (1 mol %, 5 mol %, or 10 mol % of mannose). Polyplexes of mCherry mRNA were prepared from the synthesized mannose-PEI as described in example 2. Specifically, mCherry mRNA in ultrapure water (20 μL) was mixed with 20 μL mannose-PEI in water at different PEI/Mannose ratios (1 mol %, 5 mol %, and 10 mol %). The polyplexes were prepared to have a final mRNA concentration of 0.1 μg/μL and at N/P ratio of 12. Polyplexes of mCherry mRNA were further prepared with commercially available JetPEI-Mannose (Polyplus) in a similar manner at N/P ratio of 8 and a final mRNA concentration of 0.1 μg/μL. All transfection particles were transferred from water to DMSO using standard vacuum transfer as described in example 8, yielding particles in DMSO at a concentration of 0.2 μg/μL.

20K DC2.4 cells were seeded per well in a 96 well culture plate in complete RPMI (RPMI containing 10% Fetal bovine serum, 1% Penicillin-streptomycin (10,000 U/mL)) in 100 μL volume of media. 24 hours later, cells were treated with either 200 ng or 400 ng of transfection particles in a transfection volume of 100 μL and the cells incubated for 24 hours in a humidified tissue culture incubator at 37° C. with 5% CO2.

24 hours after transfection, the proportion of mCherry transfected cells as a frequency of live cells was determined by flow cytometry by analysing the frequency of cells that expressed mCherry. For flow cytometry staining, cells were harvested from the 24 well plates in flow cytometry staining buffer (FACS buffer) and washed once with FACS buffer. Fc receptors were blocked by adding 10 μL of 10% Fc block solution (murine-anti CD16/32 (BD Biosciences)) for 5 minutes. Next, cells were stained with viability dye (VD)-efluor780 (eBioscience, for 30 minutes on ice). Finally, cells were washed twice with FACS buffer and samples acquired with a LSRfortessa X20 Flow Cytometer using a FACSDiva software (BD Biosciences). Single cells were gated based on forward scatter area (FSC-A) and forward scatter height (FSC-H), cell debris excluded by side scatter area (SSC-A) and forward scatter area (FSC-A), cell viability was determined by excluding cells that were positive for the viability dye, and finally, frequency of cells positive for mCherry were determined. Data was analysed using FlowJo software (Treestar).

### Study Part 2: Mannose-PEI NCCell

Mannose-PEI (1 mol %) polyplexes of mRNA mCherry were prepared as described in example 2 at N/P ratio of 20 and transferred to DMSO by vacuum oven transfer as described in example 8, yielding transfection particles dissolved in DMSO at a concentration of 0.4 μg/μL. The particles were embedded into NCCell as described in example 12 (SuBen: GTH: EtOH: DMSO 55:20:2:15 with 0.5% POPC and additional L-PEI 25 kDa at N/P 20). The final concentration of mCherry mRNA in the NCCell was 0.04 μg/μL.

75K DC2.4 cells were seeded per well in a 24 transwell (8 μm pore) culture plate from Corning (Cat: 10167000) in 1 mL of complete RPMI media (RPMI containing 10% Fetal bovine serum, 1% Penicillin-streptomycin (10,000 U/mL)). 24 hours later, 100 μL of NCCell containing a total of 4 μg of mCherry mRNA was injected into the transwell insert, the NCCell material was drawn up using a 1 ml syringe and 18G needle, and then injected using a 1 ml syringe and 23G needle. 1 mL of complete media was added to the transwells and the cells incubated for 48 hours in a humidified tissue culture incubator at 37° C. with 5% CO₂. 48 hours after transfection, the proportion of mCherry transfected cells as a frequency of live cells was determined by flow cytometry as explained in study part 1: Mannose-PEI free particles.

### Results and Discussion:

### Study Part 1: Mannose-PEI Free Particles

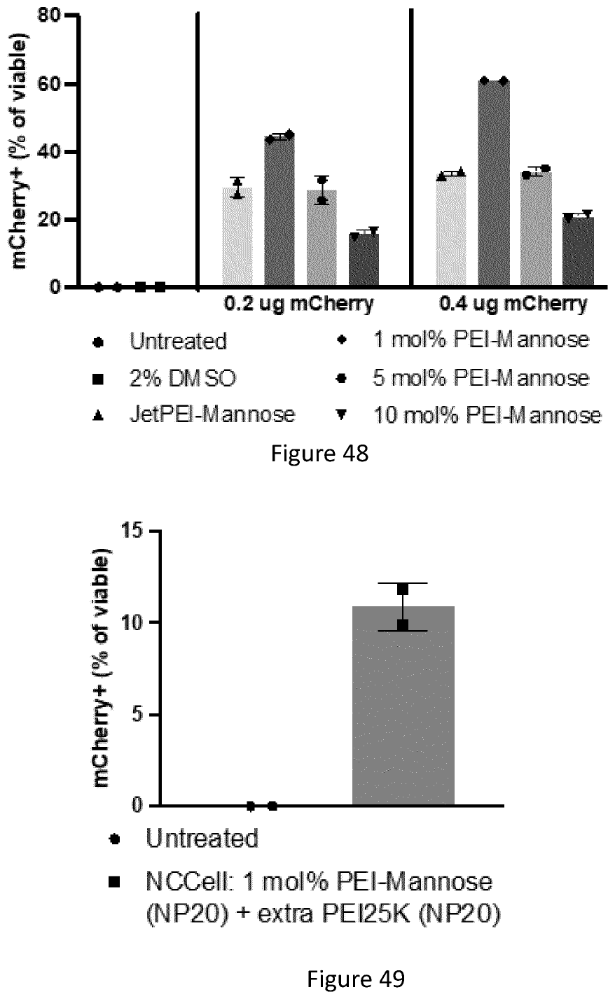

All transfection particles (JetPEI-Mannose, 1 mol % PEI-Mannose, 5 mol % PEI-Mannose and 10 mol % PEI-Mannose) successfully transfected DC2.4 cells with mCherry mRNA from DMSO, both at 0.2 μg and 0.4 μg doses as evidenced by the transcription of mCherry. Notably, 1 mol % PEI-Mannose achieved highest percentage of mCherry positive cells, as can be observed in FIG. 48.

### Study Part 2: Mannose-PEI NCCell

NCCell loaded with 1 mol % PEI-Mannose (N/P ratio of 20) and additional free 25K PEI (N/P at 20) were, in a transwell system able to transfect DC2.4 cells with mCherry mRNA at a dose of 4 μg/well, as can be observed in FIG. 49.

Conclusion: mCherry mRNA PEI-Mannose transfection particles with various ratios of PEI to Mannose successfully transfected DC2.4 cells both as free particles in DMSO and upon release from NCCell.

### Example 34: Stability of mRNA and pDNA Transfection Particles Stored for 35 Days at Various Conditions

Aim: Demonstrate how transfection particles of mRNA or pDNA can be stored in water or DMSO for at least 35 days and still demonstrate in vitro transfection.

Methods: Transfection particles of pDNA and mRNA were formed according to example 2. Specifically, 20 μL of L-PEI 40 kDa in water was mixed with 20 μL of a solution of either mCherry pDNA or mCherry mRNA at a N/P ratio of 25. The final mRNA or pDNA concentration was 0.2 mg/mL. The particles were either kept in water during storage or transferred to DMSO as described in example 8. The transfection ability of the particles, at the preparation day and 35 days later, was investigated when stored in DMSO or water at different temperatures.

The mCherry transfection experiment was performed in HEK cells and evaluated by flow cytometry as described in example 24. In a 96-well plate, the cells were treated with 400 ng of pDNA or mRNA transfection particles in a final volume of 100 μL.

### Results and Discussion:

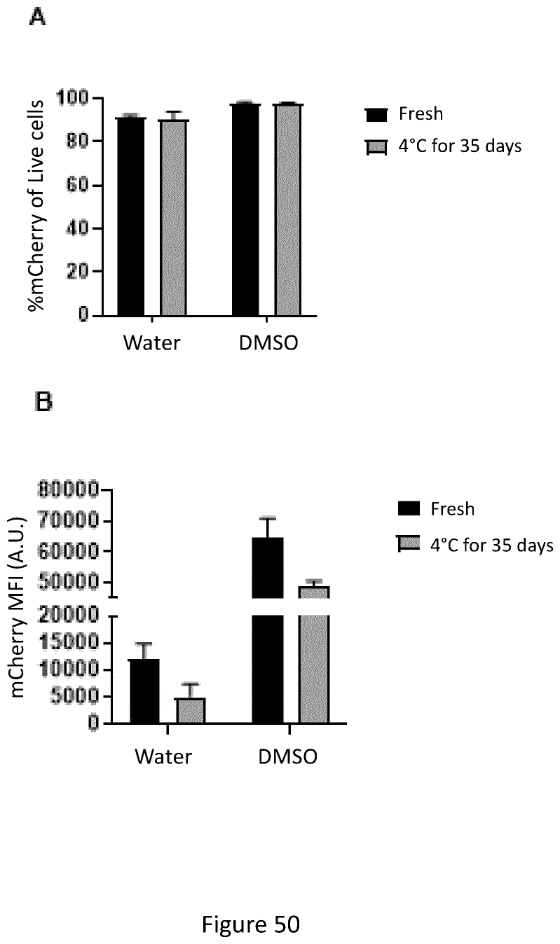

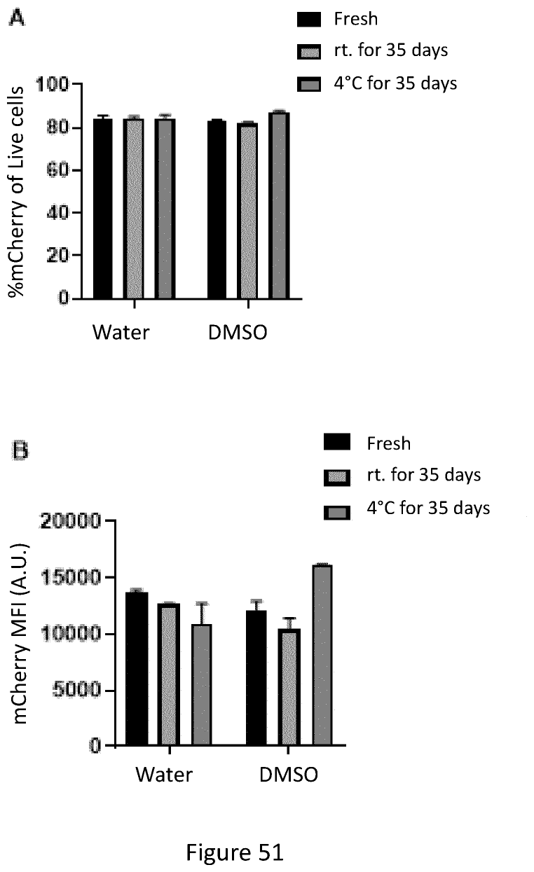

mRNA mCherry transfection particle stability: mCherry mRNA transfection particles stored at 4° C. for 35 days in DMSO and water show a similar number of mCherry positive cells compared to freshly prepared transfections particles (FIG. 50). These results demonstrate that the mRNA L-PEI 40 kDa transfection particles were stable during storage at 4° C. for at least 35 days. The mCherry MFI was also similar between freshly prepared particles and particles stored for 35 days, however, transfection from DMSO increased the MFI for both fresh and stored particles (FIG. 51).

pDNA mCherry transfection particle stability: Transfection with pDNA mCherry transfection particles stored for 35 days at room temperature (rt) or 4° C. stored in water or DMSO yield comparable percentage of mCherry positive cells as freshly prepared transfections particles (FIG. 50). The mCherry MFI was similar between fresh particle preparations or stored particles at both conditions (FIG. 51). The results indicate good stability of the pDNA L-PEI 40 kDa transfection particles when stored at various temperatures for at least 35 days.

Conclusion: L-PEI 40 kDa transfection particles prepared from mCherry mRNA or mCherry pDNA display a maintained transfection capability after 35 days of storage compared to freshly prepared particles. Transfection capability was maintained both for particles in water and in DMSO.

### Example 35: Method for Up-Concentrating pDNA Transfection Particles in NCCell

Aim: Demonstrate how a combination of freeze-drying and vacuum oven transfer can increase the concentration of transfection particles that can be embedded into NCCell compositions.

Principle: To achieve higher concentrations of transfection particles, these can be concentrated in two steps. First, the solvent volume can be reduced by freeze-drying, and secondly, with standard vacuum transfer (vacuum distillation) of the particles from water to DMSO as described in example 8. By initial reduction of the water content by freeze-drying, it was possible to reach higher concentrations of transfection particles in organic media such as DMSO, which can be embedded into NCCell.

### Methods:

Preparation of L-PEI40 kDa/pDNA transfection particles: Transfection particles were prepared as previously described in example 2. Specifically, 250 μL of L-PEI 40 kDa in water was mixed with 250 μL of mCherry pDNA in water to yield a N/P ratio of 4. The final pDNA concentration was 0.5 mg/mL in a volume of 0.5 mL. The mixture was left to complex for 30 minutes to yield the polyplexes followed by mixing with 50 μL of DMSO.

Preparation of DOTAP/pDNA hydrophobic ion-pairs: Hydrophobic ion pairs of pDNA and DOTAP were prepared according to example 9. Specifically, 375 μL of an aqueous solution of DOTAP was mixed with 125 μL of a solution of mCherry pDNA. The mixture was prepared at an N/P ratio of 2 and the final pDNA concentration was 0.5 mg/mL. The mixture was left for 30 minutes to form the HIPs followed by mixing with 50 μL of DMSO.

Up-concentrating pDNA complexes by freeze-drying: The samples were snap-frozen and added to the freeze-dryer and the volume was reduced for approximately 1-2 hours until around 0.1-0.2 mL was remaining. The remaining water was removed by vacuum transfer as described in example 8, yielding transfection particles dissolved in DMSO at a concentration of 5 mg/mL. The particles were embedded into the NCCell (SuBen: GTH: EtOH: DMSO 55:20:5:20) as described in example 12 to have a final transfection particle concentration of 0.5 mg/mL.

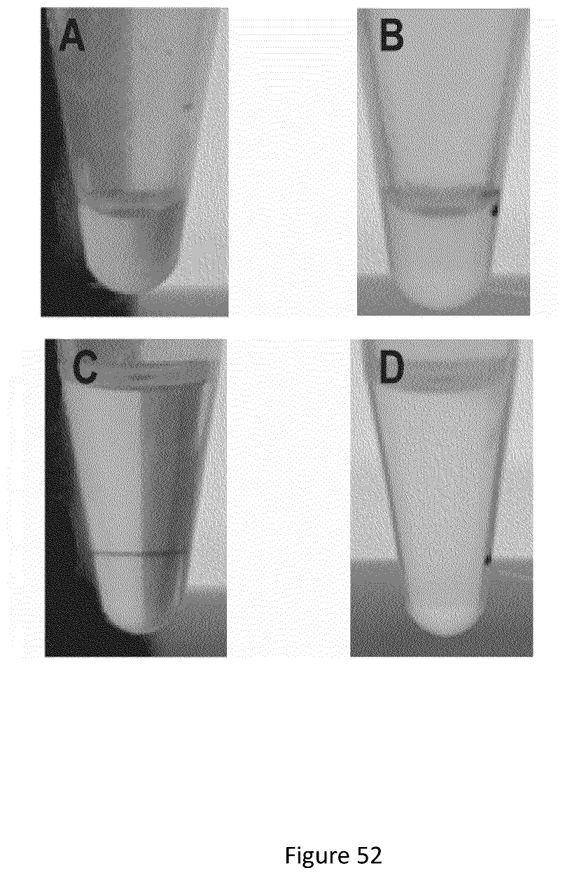

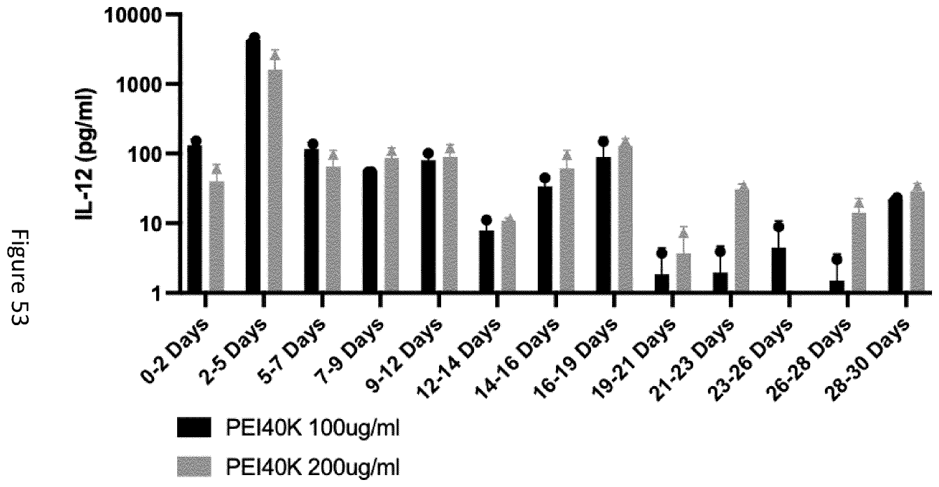

Results and discussion: After up-concentration by freeze-drying and vacuum transfer, the transfection particles remained soluble in DMSO at a concentration of 5 mg/mL as shown for both L-PEI 40 kDa polyplex of pDNA and DOTAP HIP of pDNA in FIGS. 52A+B. Furthermore, both DOTAP HIP and L-PEI polyplex displayed good solubility when mixed with NCCell to a final pDNA concentration of 0.5 mg/ml (10 times dilution), as demonstrated by the clear solutions in FIG. 52C+D.

Conclusion: pDNA lipoplexes and polyplexes can be concentrated using a two-step method of freeze-drying followed by standard vacuum transfer reaching concentrations of 0.5 mg/mL in the NCCell.

### Example 36: In Vitro Sustained Transfection from NCCell with OX40L/IL-12 Dual Plasmid

Aim: To demonstrate that NCCell loaded with a pDNA construct encoding for two genes (dual plasmid) can produce sustained transfection of both encoded targets.

Method: Plasmid DNA encoding IL-12 and OX40L (dual plasmid) was formulated as L-PEI 40 kDa pDNA (IL-12/OX40L dual plasmid) transfection particles, according to example 2. The particles were transferred to DMSO as shown in example 8 and mixed with NCCell (SuBen: GTH: EtOH: DMSO 55:20:2:15+0.5% POPC) according to example 12. The final pDNA concentration in the NCCell was 100 μg/mL and 200 μg/mL.

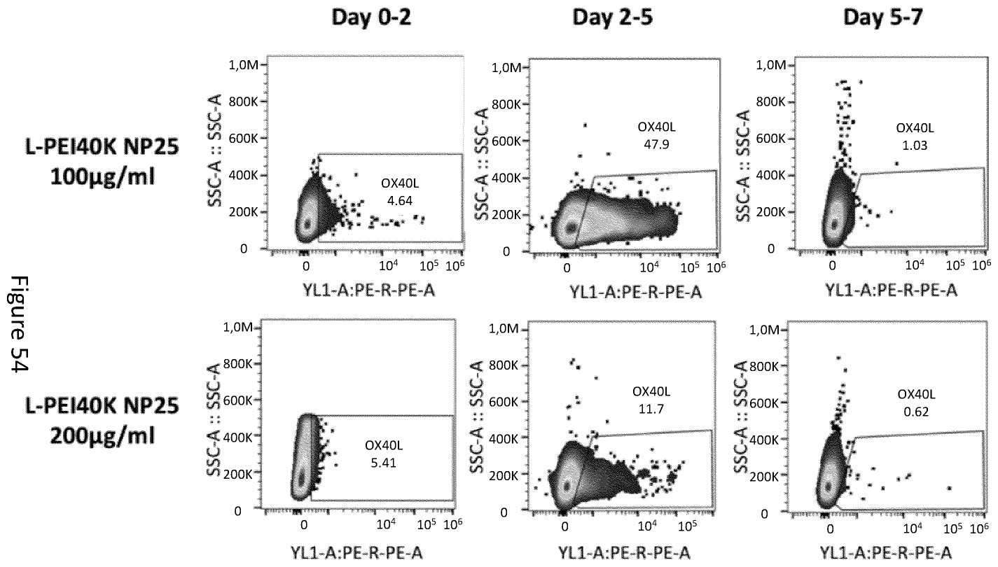

Transfections were performed in 24 transwell (5 μm pore) culture plates with HEK293 (Human embryonic kidney 293) cells in 2 mL of culture volume (culture media: 500 mL DMEM high glucose, GlutaMAX™ Supplement 10% FBS, 1% Sodium Pyruvate (100 mM) and 1% Penicillin-Streptomycin (10,000 U/mL)). NCCell (100 μL) was injected into the transwell insert using a 2-component 1 mL syringe and 23G needle. The NCCell was allowed to release transfection particles for 48 hours (50.000 HEK cells in 2 mL media) before the inserts were transferred to a new well (25.000 HEK cells in 2 mL media) and allowed to transfect for additional 48-72 hours. The specific timepoints are detailed in FIG. 53. Culture media (supernatant) was collected every time inserts were transferred and stored at −20° C. until IL-12 transcription analysis, and cells harvested for quantification of OX-40L expression by flow cytometric analysis.

IL-12 transcription efficiency was evaluated by Mouse IL-12 p70 DuoSet ELISA (Sandwich) according to manufacturer's protocol. Analysis was performed using a SpectraMax iD5 Multi-Mode Microplate Reader (450 nm+570 nm for reference) and Graphpad Prism was used to generate standard curve. For OX-40L transcription analysis, the cells were resuspended in 250 μL of PBS and transferred to a round bottom 96 well plate. Once in the 96 well plate, the cells were centrifuged for 3 minutes at 600G, after which the supernatant was discarded, and the cells were resuspended in 200 μL of PBS. This process was repeated twice. After washing, the cells were resuspended in 200 μL PBS and analyzed by flow cytometry on the Attune NxT flow cytometer. The viability of the cells was evaluated by gating out cells negative for the viability dye VD-efluor780 (RL3-A channel), and OX-40L expression was quantified by OX-40L PE-antibody (YL1-A channel). Data was analysed using FlowJo software (Treestar).

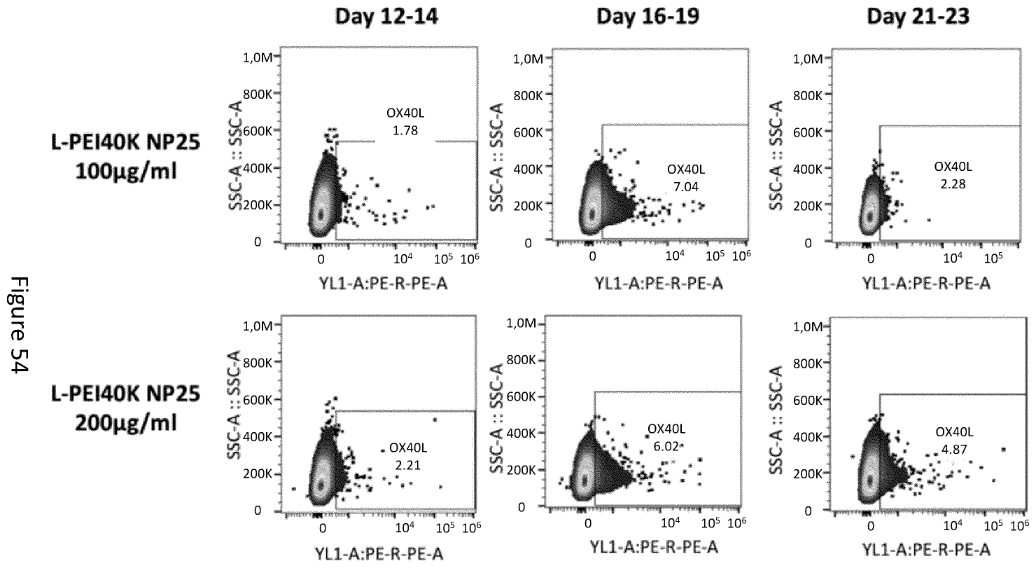

Results and discussion: Transfection particles L-PEI40K pDNA (IL-12/0X40L dual plasmid) formulated in NCCell composition SuBen: GTH: EtOH: DMSO 55:20:2:15 (+0.5% POPC), were able to provide sustained release of transfection particles. This was confirmed by the sustained transcription of IL-12 (FIG. 53) and OX40L (FIG. 54) upon repeated transfer of the NCCell (injected in transwell insert) to new HEK cells. The ability to provide sustained release of pDNA particles from NCCell compositions was demonstrated for both plasmid concentrations.

Conclusion: NCCell can release pDNA transfection particles that encodes for two genes, in this case IL-12 and OX-40L, and induce sustained transcription for up to 30 days.

### Example 37: Sustained In Vitro Release of siRNA Particles from NCCell Evaluated by Fluorescence

Aim: To demonstrate that the NCCell technology can provide sustained in vitro release of multiple siRNA transfection particles.

### Methods:

Preparation of NCCell: NCCell was prepared as described in example 1. The NCCell compositions used for this experiment were SuBen: GTH: EtOH: DMSO 55:20:2:15+0.25% chol, SuBen: GTH: EtOH: DMSO 55:15:2:15+0.25% chol, SuBen: GTH: EtOH: DMSO 55:20:2:15+0.25% chol+1% POPC.

Preparation of lipoplexes: Lipoplexes of Cy5-siRNA were prepared according to example 3. Lipoplexes of following composition (DOTAP: DC-Chol: Chol: DOPE 25:25:25:25) were prepared at a N/P ratio of 4. The final siRNA concentration was 0.1 μg/μL in 200 μL water.

Preparation of polyplexes: Polyplexes of Cy5-siRNA were prepared according to example 2. Specifically, particles of L-PEI 40 kDa at N/P ratio of 25 were prepared at a concentration of 0.1 μg/μL in 200 μL water.

Transfer to DMSO and embedding siRNA particles into NCCell: The various siRNA particles in water were mixed with 50 μL of DMSO and transferred to DMSO by vacuum oven transfer as described in example 8. The particles in 50 μL DMSO were mixed with 450 μL of NCCell and stirred for 5 minutes, yielding NCCell embedded with siRNA particles at a concentration of 0.04 μg/μL.

In vitro release of particles: The in vitro release of particles was performed as described in example 21. 100 μL of NCCell was injected into 2 mL of PBS and release fractions of 1 mL were taken out and replaced with fresh PBS at multiple timepoints. The released Cy5 labelled siRNA was quantified by fluorescent spectroscopy (Tecan Spark, Ex/Em. 600/670 nm) and compared to a standard of particles in PBS. The data was plotted as cumulative release.

### Results and Discussion:

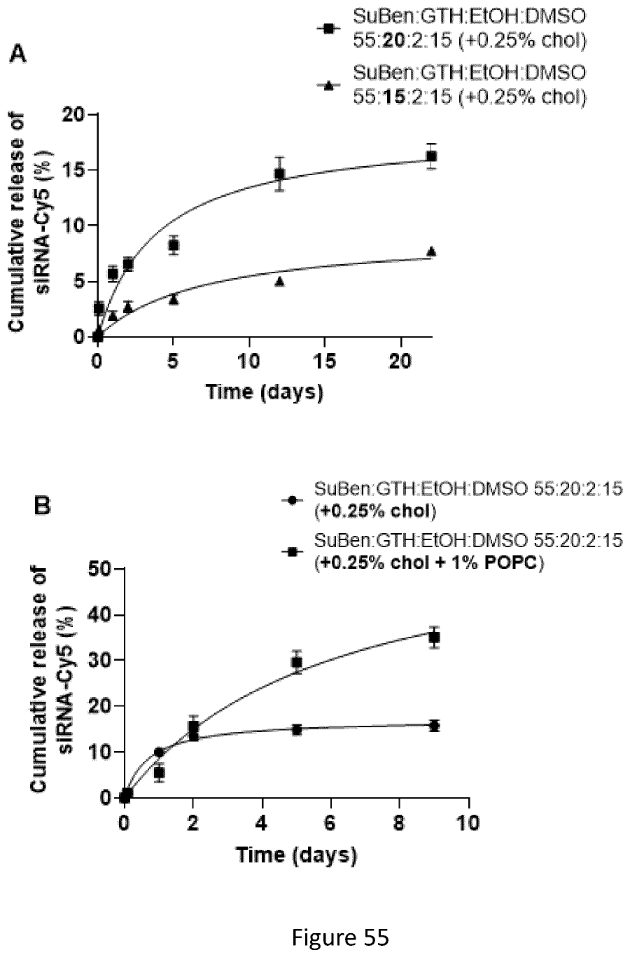

Controlled release by varying the GTH content: Lipoplex of siRNA with composition DOTAP: DC-Chol: Chol: DOPE 25:25:25:25 was released from NCCell containing two different levels of GTH. FIG. 55A demonstrates that the rate of the particle release was decreased when lowering the GTH content of the NCCell. This correlates with the NCCell material having higher viscosity upon decrease in GTH content, which enhances the diffusion barrier and slows down the release. Furthermore, lipoplexes prepared with siRNA demonstrated sustained release from both NCCell compositions for up to 22 days in vitro.

Controlled release by lipid additives: L-PEI 40 kDa polyplex of siRNA was released from NCCell at different rates depending on the addition of lipids to the NCCell formulation. FIG. 55B shows that the addition of 1% of the lipid POPC approximately doubled the release rate when compared to NCCell without POPC. PEI-polyplexes of siRNA further demonstrated successful sustained release for at least 9 days in vitro.

Conclusion: It was possible to obtain sustained release from NCCell compositions of particles loaded with siRNA in a similar manner as seen for particles of mRNA and pDNA as described in example 21. The release rate was tuned by varying GTH content and by addition of lipids to the NCCell.

### Example 38: In Vitro Silencing by siRNA-Complexes

Aim: Demonstrates that siRNA-complexes in water and DMSO can successfully silence target genes in various cell lines in vitro.

### Method:

### GFP Silencing in HEK-GFP Cell Line:

Polyplexes of siRNA were prepared according to example 2. Specifically, L-PEI 40 kDa polyplex of GFP siRNA was prepared at N/P ratio of 25 at a concentration of 0.1 μg/μL. Mannose-JetPEI (Polyplus) polyplexes of GFP siRNA were prepared at N/P ratio of 8 (with and without extra added L-PEI 25 kDa at N/P ratio 25) in water at a concentration of 0.1 μg/μL. Lipoplexes of GFP siRNA were prepared as described in example 3. Specifically, lipoplex of composition DOTAP: DC-Chol: Chol: DOPE 25:25:25:25 was prepared at N/P ratio of 5. The final siRNA concentration in water was 0.1 μg/μL. Polyplexes and lipoplexes of GFP siRNA were transferred to DMSO according to example 8 to have transfection particles at 0.2 μg/μL in DMSO. Scrambled siRNA polyplexes (PEI40K/siRNA (scrambled), 0.1 μg/μL siRNA in water and 0.2 μg/μL DMSO) were prepared in the same manner and included as unspecific siRNA controls.

20K HEK293 (Human embryonic kidney 293) cells genetically modified to express GFP were seeded per well in a 96 well plate (20.000 cells/well) 24 hours prior to transfection in 100 μL DMEM high glucose, GlutaMAX™ Supplement 10% FBS, 1% Sodium Pyruvate (100 mM), and 1% Penicillin-Streptomycin (10,000 U/mL)). Treatment was performed with 100 ng of siRNA per well in 100 μL transfection volume and incubated for 24 hours in a humidified tissue culture incubator at 37° C. with 5% CO₂. The MFI of GFP positive HEK-GFP cells of the live fraction was quantified by flow cytometry. Cells were spun down to remove media and then resuspended in 200 μL 1×PBS and analysed by flow cytometry using the Attune NxT flow cytometer. The viability of the cells was assessed based on morphology using SSC-A and FSC-A parameters. Silencing was quantified by GFP parameters (BL3-A channel). The MFI of treated cells was normalized to that of the untreated cells, or to that of scrambled controls to determine silencing levels.

hKRAS Silencing in MiaPaca2 Cell Line:

PAMAM generation 0.0 polyplex of hKRAS siRNA-cholesterol was prepared as described in example 2 at N/P ratio of 10. The final siRNA-cholesterol concentration in water was 25 μM. Lipoplexes of the composition DOTAP: DC-Chol: Chol: DOPE were prepared according to example 3 with either siRNA or siRNA-cholesterol (KRAS) at N/P ratio of 4. The siRNA/siRNA-cholesterol concentration was 25 UM in water.

Transfections were performed in 24 well culture plates with 80.000 MiaPaca2 Cells in 500 μL culture volume (RPMI 10% FBS, 1% Sodium Pyruvate (100 mM), and 1% Penicillin-Streptomycin (10,000 U/mL)). For cells treated with siRNA-cholesterol PAMAM polyplexes, the culture media was serum-free. 5 μL of polyplex or lipoplex suspensions were pipetted in each well, for final concentrations of 250 nM per well. The cells were incubated for 48 hours, after which the cells were lysed and stored at −20° C. until RNA extraction using silica spin columns (RNeasy Mini Kit, (Qiagen 74106) performed according to manufacturer's protocol). Silencing efficiency was evaluated by RTqPCR using a Biorad CFX96 Touch Real-Time PCR Detection System and Biorad CFX Manager program. RTqPCR was performed on the target gene (KRAS) and two housekeeping genes (GAPDH and HPRT). Results were analysed using the delta-delta Ct method (also referred to as the 2-\Ct method) on Microsoft Excel, and Graphpad Prism was used to generate figures. KRAS expression was normalized to both housekeeping genes, data are shown for normalization to HPRT.

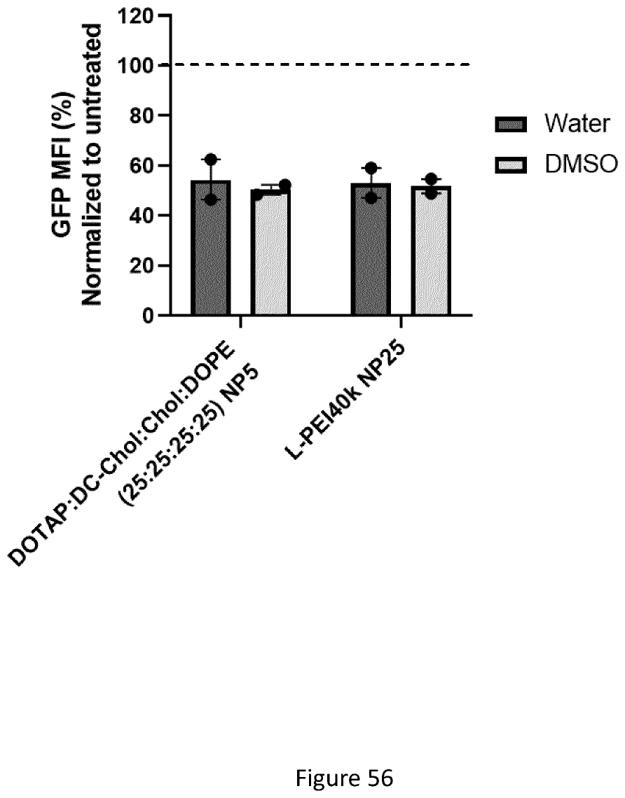

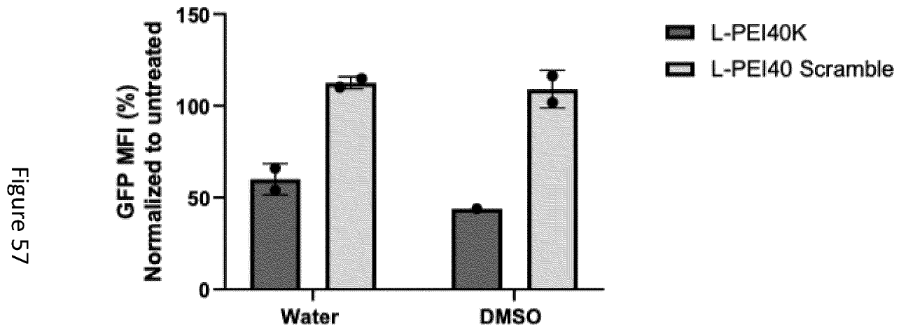

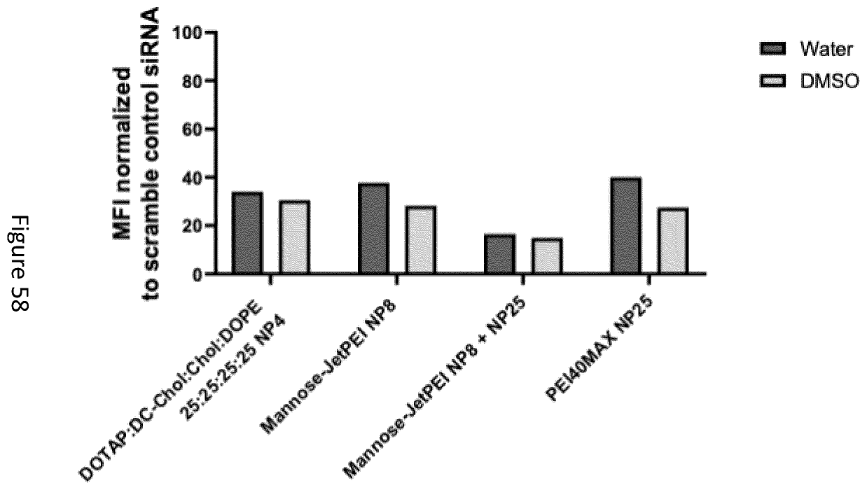

Results and discussion: Polyplexes and lipoplexes of siRNA GFP demonstrated successful silencing of GFP expression in HEK-GFP cells as shown in FIG. 56, FIG. 57 and FIG. 58.

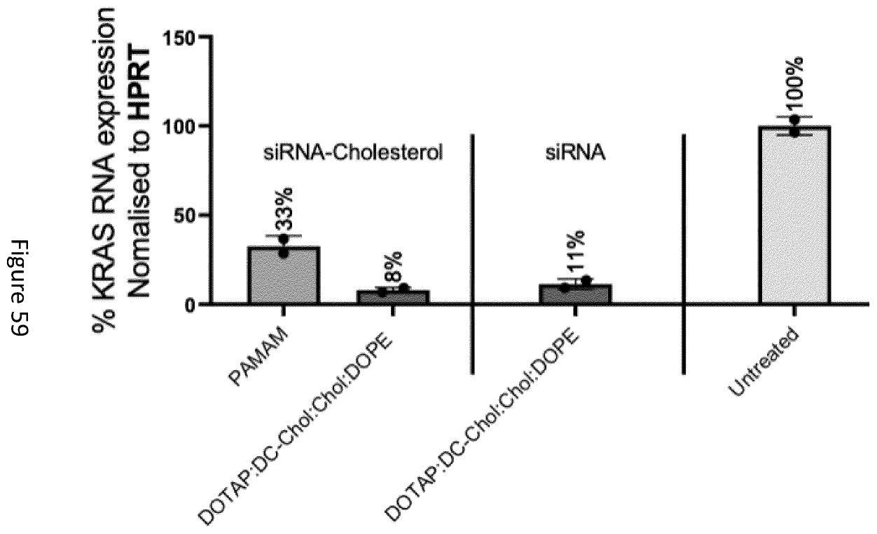

PAMAM polyplex of hKRAS siRNA-cholesterol as well as lipoplexes of siRNA and siRNA-cholesterol (KRAS) were capable of silencing KRAS expression in MiaPaca2 cells as demonstrated in FIG. 59.

Conclusion: Polyplexes and lipoplexes prepared with siRNA/siRNA-cholesterol successfully silence gene expression of GFP and KRAS from both water and DMSO.

### Example 39: Sustained In Vitro Silencing by siRNA-Complexes Released from NCCell

Aim: To demonstrate sustained silencing of GFP from siRNA transfection lipoplexes and polyplexes released from NCCell in vitro.

Method: Lipoplexes of siRNA were prepared according to example 3. Liposome of composition DOTAP: DC-Chol: Chol: DOPE (25:25:25:25) was mixed with GFP siRNA at N/P ratio of 5 to form a lipoplex. The final siRNA concentration in water was 0.1 μg/μL. Polyplex was prepared as described in example 2, and specifically prepared with L-PEI 40 kDa and GFP siRNA at N/P ratio of 25. The final siRNA concentration was 0.1 μg/μL. Lipoplexes and polyplexes of GFP siRNA were transferred to DMSO using vacuum oven transfer as described in example 8, yielding transfection particles in DMSO at a concentration of 0.2 μg/μL. Finally, the particles were embedded into NCCell (SuBen: GTH: EtOH: DMSO 55:20:2:15 (+0.5% POPC)) at concentrations of 0.02 μg/μL according to example 12 and 13.

Transfections were performed in 24 transwell (5 μm pore) culture plates with HEK293 cells (Human embryonic kidney 293) genetically modified to express GFP, in 2 mL culture volume (culture media: 500 mL DMEM high glucose, GlutaMAX™ Supplement 10% FBS, 1% Sodium Pyruvate (100 mM) and 1% Penicillin-Streptomycin (10,000 U/mL)). NCCell (100 μL) was injected into the transwell insert using a 2-component 1 ml syringe and 23G needle and allowed to release transfection particles for periods of 48-72 hours. This process was continued for 14 days. For transfection periods of 48 hours, a cell concentration of 50.000 HEK-GFP cells in 2 mL media was used, and for 72 hours transfection period, a concentration of 25.000 HEK-GFP cells in 2 mL media was used. After transfection periods ended, inserts were moved to a new well containing freshly seeded cells. For insert transfer, the insert containing NCCell was removed from the well, and all remaining supernatant was removed and discarded before placing in the next well where particles were to be released for intended period.

For silencing analysis, the cells were resuspended in 250 μL of PBS and transferred to a round bottom 96 well plate. Once in the 96 well plate, the cells were centrifuged for 3 minutes at 600G, after which the supernatant was discarded, and the cells were resuspended in 200 μL of PBS. This process was repeated twice. After washing, the cells were resuspended in 200 μL PBS and analyzed by flow cytometry using the Attune NxT flow cytometer. The viability of the cells was determined based on morphology using SSC-A and FSC-A parameters, GFP silencing was quantified using GFP parameters (BL3-A channel). The MFI of treated cells was normalized to that of the untreated cells to determine silencing efficiency.

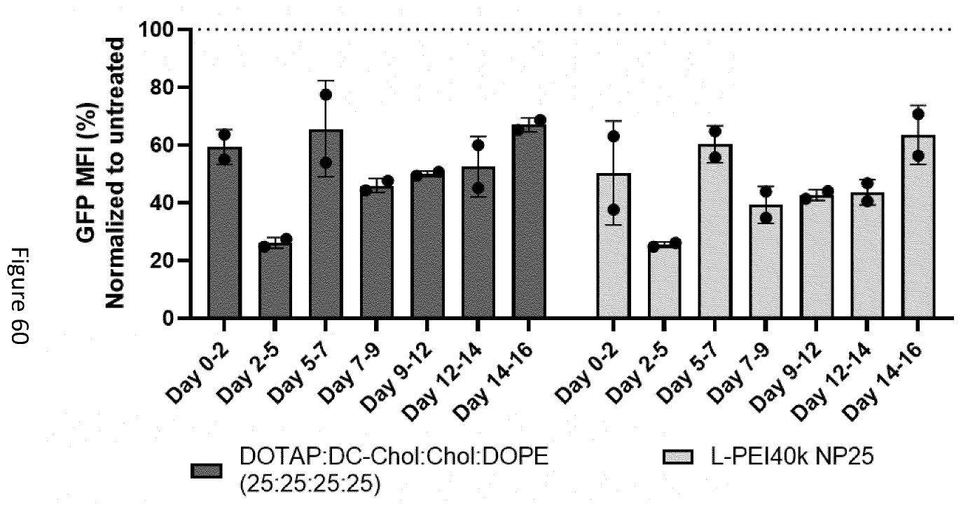

Results and discussion: Lipoplexes and polyplexes of GFP siRNA formulated in NCCell provided sustained silencing of GFP in HEK-GFP cells as evidenced by the observed reduction in GFP MFI on flow cytometry detailed in FIG. 60. The observed sustained silencing supports that effective siRNA transfections particles are continuously released from the NCCell formulations.

Conclusion: The NCCell formulations can provide sustained release of GFP silencing siRNA lipoplexes and polyplexes.

### Example 40: Stability of siRNA L-PEI 40 kDa Polyplexes Stored for 35 Days in DMSO

Aim: To demonstrate that siRNA L-PEI 40 kDa particles can silence GFP after 35 days of storage in DMSO to a similar extent as freshly prepared particles.

Methods: The siRNA particles were prepared according to example 2. Specifically, 125 μL of an aqueous solution of L-PEI 40 kDa was mixed with 125 L of GFP siRNA in water at a N/P ratio of 25. The mixture was vortexed for 5 seconds and left for 30 minutes. The resulting siRNA concentration was 0.1 mg/mL. The samples were transferred to DMSO by vacuum transfer as described in example 8. The samples were stored in DMSO for 35 days at either −20° C. or 4° C. The GFP silencing capacity of the particles was evaluated in HEK293 cells and compared to the silencing capacity of freshly prepared particles according to example 38.

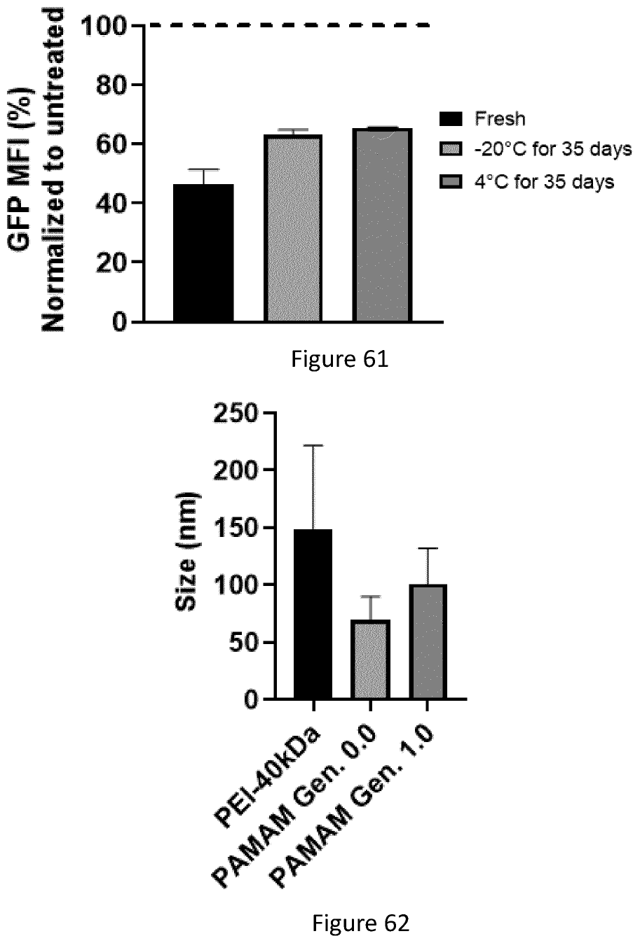

Results and discussion: The results demonstrate that when transfection particles of siRNA were stored for 35 days at either 4° C. or −20° C., the silencing capacity was comparable to freshly prepared particles. This is shown in FIG. 61, where both freshly prepared particles and stored particles yield a 40-50% knockdown of GFP.

Conclusion: The silencing capacity of L-PEI 40 kDa siRNA transfection particles was maintained for at least 35 days upon storage in DMSO at −20° C. and at 4° C.

### Example 41: Chemical Modifications of ASOs

Aim: Demonstrate the synthesis of chemical modifications of ASOs as Cy7 and DOTA labelling.

Principle: Chemical modifications of ASOs are particularly interesting for biodistribution (BD) studies. This can be relevant with either fluorescent labelling (Cy7) or DOTA-Gd labelling for analysis of biodistribution studies using fluorescent spectroscopy or ICP-MS, respectively. ASOs can be produced with an amine linker, which can be used for coupling reactions to the various modifications.

### Method:

Synthesis of sulfo-cyanine7-/5AmMC6/+G*+T*+C*A*C*A*A*T*G*C*A*T*T*+C*+T*+A: The oligonucleotide (1.1 mg, 209 nmol) was dissolved in sodium borate saline (900 μL, 20 mM, 150 mM NaCl, pH 8.5) in a DNA LoBind® 1.5 ml tube. Sulfo-Cyanine7 NHS (3.5 mg, 4.2 μmol) was dissolved in DMSO (100 μL) and added to the tube with oligonucleotide. The reaction was left to react overnight at room temperature on a Hulamixer™. The reaction was analyzed by analytical UHPLC (Agilent AdvanceBio Oligonucleotides using method 1). Full conversion was confirmed. The crude compound was purified by a PD-10 column (Cytiva) equilibrated with PBS (Dulbecco's). The pure fractions were pooled (540 μg, 43% yield) and analyzed by SDS PAGE and MALDI TOF MS. Nanodrop UV-Vis determined DOL to 1.12. MALDI TOF MS (m/z) (ZipTip with TEEA, HCCA matrix): Calc. mass: 6146; Found mass: 6151 [M+H]⁺. None of the native oligonucleotide was seen in the product by MALDI TOF MS. SDS PAGE (NuPAGE 10% Bis-Tris with MES running buffer) confirmed a purity of >85% by SYBRsafe stain.

Synthesis of DOTA-/5AmMC6/+G*+T*+C*A*C*A*A*T*G*C*A*T*T*+C*+T*+A: The oligonucleotide (3.0 mg, 550 nmol) was dissolved in sodium borate saline (1020 μL, 20 mM, 150 mM NaCl, pH 8.5) in a DNA LoBind® 1.5 mL tube. DOTA-NHS (8.4 mg, 11.0 μmol) was dissolved in DMSO (84 μL) and added to the tube with oligonucleotide.

The reaction was left to react overnight at room temperature on a Hulamixer™. The reaction was analyzed by analytical UHPLC (Agilent AdvanceBio Oligonucleotides using method 1). Full conversion was confirmed. The crude compound was purified by a PD-10 column (Cytiva) equilibrated with NaOAc saline (20 mM, 150 mM NaCl, pH 5.5). The pure fractions were pooled (1.85 mg, 58% yield) and analyzed by MALDI TOF MS (m/z) (ZipTip with TEEA, HCCA matrix): Calc. mass: 5843; Found mass: 5846 [M+H]⁺

Gadolinium loading into DOTA: DOTA-/5AmMC6/+G*+T*+C*A*C*A*A*T*G*C*A*T*T*+C*+T*+A (1.85 mg, 317 nmol) was dissolved in NaOAc saline (2911 μL, 20 mM, 150 mM NaCl, pH 5.5) in a 15 mL falcon tube. GdCl₃ (31.7 μmol, 100 mM) was dissolved in HCl (100 mM) and NaOAc saline (20 mM, 150 mM NaCl pH 5.5) in a 10:90 v/v ratio and 317 μL was added to the DOTA-conjugated oligonucleotide. The chelation was left to react for 48 hours at 2-8° C. The reaction was analyzed by analytical UHPLC (Agilent AdvanceBio Oligonucleotides using method 1). Full conversion was confirmed. The crude compound was purified by a PD-10 column (Cytiva) equilibrated with Tris saline (20 mM, 150 mM NaCl pH 7.5). The pure fractions were pooled (1.61 mg, 85% yield) and analyzed by ICP MS (Fredrik has the data).

Conclusion: The various modified ASOs were successfully isolated and can be used for in vitro and in vivo studies.

### Example 42: Preparation of Polyplexes of ASOs

Aim: To describe the preparation of polyplexes of antisense oligonucleotides (ASOs) using amine-containing polymers like PEI and PAMAM.

Principle: The polyplexes were formed by complexing the cations of the polymer, usually from amine groups, with the anionic phoshorothioate groups of the ASO. Cationic polymers were among others polyethyleneimine (PEI) and polyamidoamine (PAMAM).

Methods: The polyplexes were prepared by simple mixing similarly to example 2 and examples of the preparation of PEI-40 kDa and PAMAM generation 0.0 polyplexes of ASOs are described below.

PEI-40 kDa: A 250 μg/mL solution of the ASO was mixed 1:1 (volume ratio) with a PEI-40 kDa solution (N/P ratio of 25) yielding a final ASO concentration of 125 g/mL in 80 μL. The mixture was vortexed for 5 seconds and left complexing for 30 minutes, which yielded the wanted polyplex in water.

PAMAM generation 0.0:20 μL of an aqueous ASO solution at 500 μg/mL was mixed with 60 μL of a PAMAM generation 0.0 solution (N/P ratio of 10) yielding a final ASO concentration of 125 μg/mL. The mixture was vortexed for 5 seconds and left complexing for 30 minutes, which yielded the polyplex in water. Polyplex of PAMAM generation 1.0 was prepared in the same manner.

NTA measurements: The polyplexes were evaluated by measuring particle size by NTA. The NTA measurements were performed according to the method described in example 6.

Results and discussion: FIG. 62 shows the particle sizes of the polyplexes measured by NTA, demonstrating that both PEI-40 kDa and PAMAM formed particles when complexed with ASOs. The generation of PAMAM (Gen 0.0 or 1.0) seemed to impact the size of the particles that were formed, meaning that a higher generation resulted in larger sized particles.

Conclusion: Polyplexes of ASO were formed similarly as polyplexes of other oligonucleotides like mRNA, pDNA, and siRNA as demonstrated in example 2. The polyplexes of PEI-40 kDa and PAMAM forms particles upon complexation with ASOs.

### Example 43: Hydrophobic Ion Pairs of ASOs Form Particles when Using Large, Hydrophobic Counterions

Aim: To describe the preparation of hydrophobic ion pairs (HIPs) of ASOs using cationic counterions with various hydrophobicity.

Principle: HIPs were formed between the anionic phosphorothioate groups of ASOs and the cationic hydrophobic counterions by mixing a solution of ASO with a solution of the hydrophobic counterion. This created complexes with improved organic solvent solubility and can thereby be incorporated into the NCCell. Suitable cationic hydrophobic counterion were described in example 9.

### Methods:

General method for preparation of ASO-HIPs: Stock solutions of the hydrophobic cationic counterions were prepared by dissolving the compounds in ultrapure water at concentrations of 1-10 mg/mL depending on the solubility of the compound. The cationic stock solution was diluted further to match the wanted N/P ratio and was then mixed with an aqueous solution of the ASO by pipetting up and down followed by vortexing for 5 seconds. Final ASO concentrations of 100-500 μg/mL were suitable. The mixture was left at room temperature for 15-30 minutes to form the HIPs. Afterward, the HIPs of ASOs can be used for silencing experiments or further processed. The NTA measurements were performed according to example 6.

Preparation of HIPs of spermine-cholesterol, DOTAP, BTMAC, and TEAB: The ASO HIP complexes of each counterion were prepared at N/P ratios of 2 and at final ASO concentrations of 250 μg/mL in 20 μL.

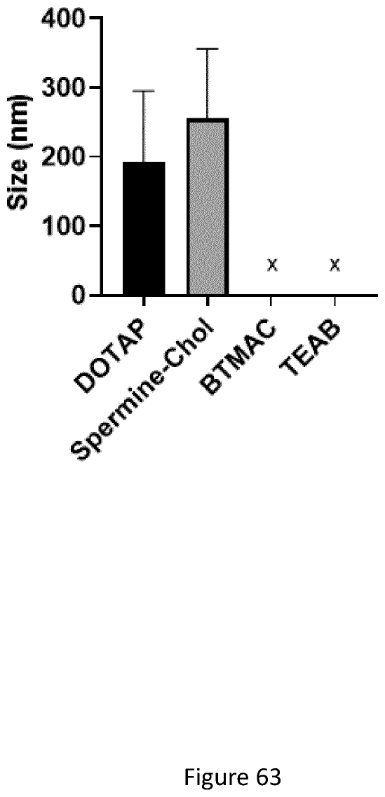

Results and discussion: FIG. 63 shows size measurements of the formed HIPs measured by NTA. The results suggest that hydrophobic and large counterions (Spermine-chol, DOTAP) form HIPs with a particle-like structures, while less hydrophobic counterions do not form particles (BTMAC, TEAB).

Conclusion: HIPs prepared by mixing solutions of ASOs and hydrophobic counterions can be obtained. Depending on the hydrophobicity and size of the counterion, the complexes can form particles.

### Example 44: Hydrophobic Ion Pairs of ASOs Prepared by Bligh-Dyer Method

Aim: To demonstrate the preparation of HIPs of ASOs with various cationic counterions by a Bligh-Dyer phase-separation method.

Principle: The Bligh-Dyer method uses a methanol-water-chloroform monophase to prepare the HIP complexes. Upon addition of more water and chloroform, the mixture separates into an organic and aqueous phase where the HIPs are expected to transfer to the organic phase. Hydrophobic cations like spermine-cholesterol, DOTAP, CTAB and others can be used to prepare HIPs with this method.

### Methods:

General method for preparation of HIPs with Bligh-Dyer method: Stock solutions of ASOs were prepared in nuclease-free water, while the counterion stock solutions were prepared in methanol. The stock solutions were diluted with water or methanol to match the desired N/P ratio. 150 μL of water (containing the ASO), 300 μL of methanol (containing the counterion), and 150 μL of chloroform were mixed to obtain a volume ratio of 1:2:1 (water: methanol: chloroform), which resulted in the formation of the Bligh-Dyer monophase. The sample was gently mixed and left incubating at room temperature for 30 minutes. Afterwards, water and chloroform were added to the mixture to reach a 1:1:1 volume ratio of water: methanol: chloroform leading to the formation of a biphasic system. The organic phase containing the HIP complex was isolated by centrifugation and the chloroform was easily removed by evaporation to yield the HIPs as precipitate. The precipitate was dissolved at the wanted concentration in water or DMSO and used for silencing experiments or further processed.

Preparation of HIPs using the counterions CTAB, THAB, and spermine-chol: The preparation of ASO-HIPs using the counterions CTAB, THAB, and spermine-cholesterol were performed by the Bligh-Dyer method. The HIPs were prepared at N/P ratio of 2, and the resulting precipitates were dissolved in DMSO to yield ASO concentrations of 1 mg/mL. The DLS size measurements were performed according to example 6.

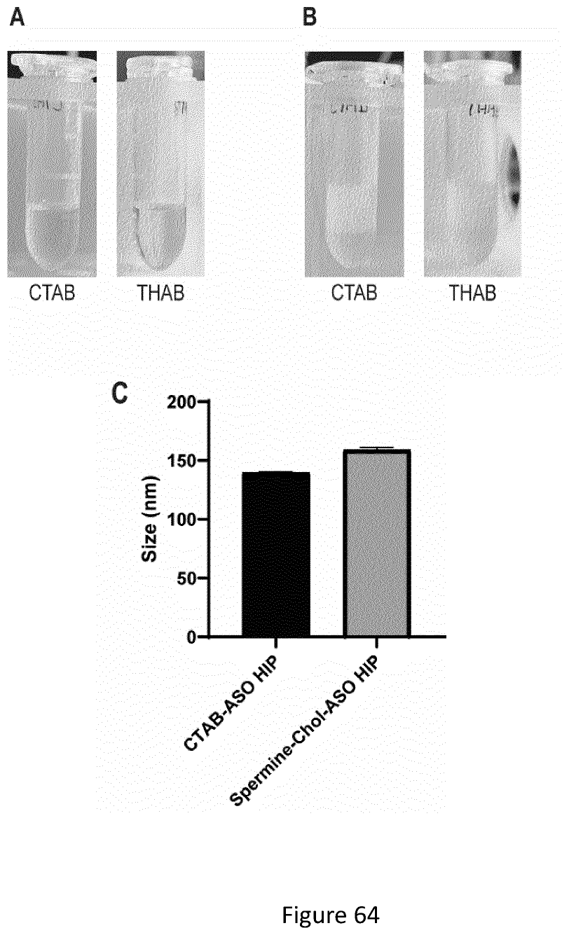

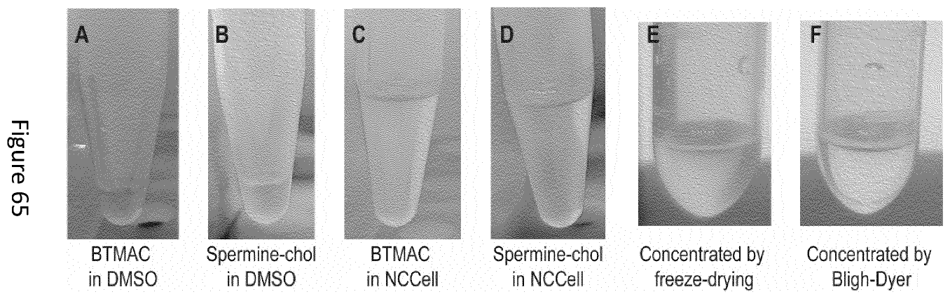

Results and discussion: The hydrophobicity of the counterion was important for preparation of the HIPs and transfer to the organic phase. Only the HIPs prepared by more hydrophobic counterions were transferred to the organic phase and thereby successfully isolated by this method. Specifically, hydrophobic counterions like CTAB, THAB, and spermine-cholesterol were successfully used. The Bligh-Dyer monophase was first prepared (FIG. 64A), and by adjusting the solvent ratio the biphasic system was formed (FIG. 64B). The ASO was labelled with the fluorophore Cy5, and it was therefore possible to follow how the ASO ended up in the organic phase by visual inspection, indicating the formation of hydrophobic ion pairs. Furthermore, size measurements of the resulting HIPs were performed by DLS, which is shown in FIG. 64C for the counterions CTAB and spermine-cholesterol. This data demonstrates that particles were formed.

Conclusion: Hydrophobic ion pairs of ASOs can be formed using the Bligh-Dyer method. Hydrophobic counterions made it possible to transfer ASO HIPs to the organic phase. The counterions CTAB and spermine-cholesterol furthermore formed particles when complexed with ASOs.

### Example 45: Methods for Loading High Concentrations of ASO Complexes into NCCell

Aim: Demonstrate the opportunities for up-concentrating ASO complexes by freeze-drying or phase-separation to increase the ASO pay-load of the NCCell compositions.

### Methods:

Freeze-drying: Polyplexes and HIPs of ASOs were prepared as described in example 42 and 43, respectively. The resulting complexes were in water at a concentration of 0.1-0.5 mg/mL. Freeze-drying was performed by mixing the solution of the ASO complexes with 5% DMSO, snap-freezing the samples in liquid nitrogen, and freeze-drying until fully dried into a powder. The ASO complexes were redissolved in DMSO at ASO concentrations of up to 50 mg/mL. The ASO complexes dissolved in DMSO were further embedded into the NCCell (SuBen: GTH: EtOH: DMSO 55:20:2:15) as described in example 15 and thereby diluted 10 times to have a final ASO concentration in the NCCell of up to 5 mg/mL.

Bligh-Dyer phase separation method: The HIPs of ASOs were prepared by the Bligh-Dyer method according to example 44. The prepared complexes were collected as powder after removal of chloroform. The powder was easily dissolved in DMSO at an ASO concentration of up to 50 mg/mL. The mixture was afterwards mixed with NCCell (SuBen: GTH: EtOH: DMSO 55:20:2:15) according to example 15 yielding a final ASO concentration in the NCCell of up to 5 mg/mL.

Results and discussion: After freeze-drying, the ASO complexes were easily dissolved in organic solvent as demonstrated by dissolving in DMSO using BTMAC or spermine-cholesterol as counterions as seen in FIG. 65A+B. BTMAC and spermine-cholesterol were highly different in structure and hydrophobicity, however, ASOs complexed with both counterions led to easily organic soluble complexes. Likewise, mixing with the NCCell resulted in homogenous solution using both counterions as seen in FIG. 65C+D. The polyplex of ASO prepared with PAMAM Gen 0.0 also resulted in a homogeneous solution in the NCCell at a concentration of 5 mg/mL. The same homogeneous solution was obtained when the up-concentration of the PAMAM-ASO polyplex was performed by the Bligh-Dyer phase separation method. This is demonstrated in FIG. 65E+F, where the PAMAM-ASO polyplex was fully dissolved in NCCell at a concentration of 5 mg/mL.

Conclusion: Both freeze-drying and Bligh-Dyer phase separation were suitable methods for up-concentrating the ASO complexes yielding a loading of up to 5 mg/ml of ASO in the NCCell. These methods were therefore useful for reaching in vitro and in vivo relevant ASO concentrations in the NCCell.

### Example 46: Tailored In Vitro Release of ASOs from NCCell Analyzed by Fluorescence

Aim: Demonstrate sustained in vitro release of ASO complexes from NCCell using fluorescent read-out.

### Methods:

NCCell preparation: NCCell of following compositions SuBen: GTH: EtOH: DMSO (55:20:2:15), (55:17.5:2:12.5) (55:15:2:10) were prepared according to example 1.

Preparation of ASO complexes and embedding into NCCell: Spermine-cholesterol HIP of ASO-Cy5 was prepared according to example 43 at N/P ratio of 2. PAMAM Gen 0.0 polyplex of ASO-Cy5 was prepared at N/P ratio of 10 according to example 42. ASO complexes were prepared at a concentration of 0.2 μg/μL in water. The complexes were freeze-dried, dissolved in DMSO (50 μL) and mixed with 450 μL of NCCell as described in example 45. The final ASO-Cy5 concentration in the NCCell was 0.04 μg/μL.

In vitro release of particles: 100 μL NCCell compositions embedding complexes were injected into glass vials containing 2 mL PBS. The release samples were incubated at 37° C. during the period of the experiment. 1 mL PBS was sampled at various timepoints and replaced with 1 mL of PBS. Released complexes were quantified by fluorescence spectroscopy (Tecan Spark, Ex/Em. 600/670 nm). Standard curves with concentrations between 0.06-2 μg/ml were prepared and the results were reported as cumulative release.

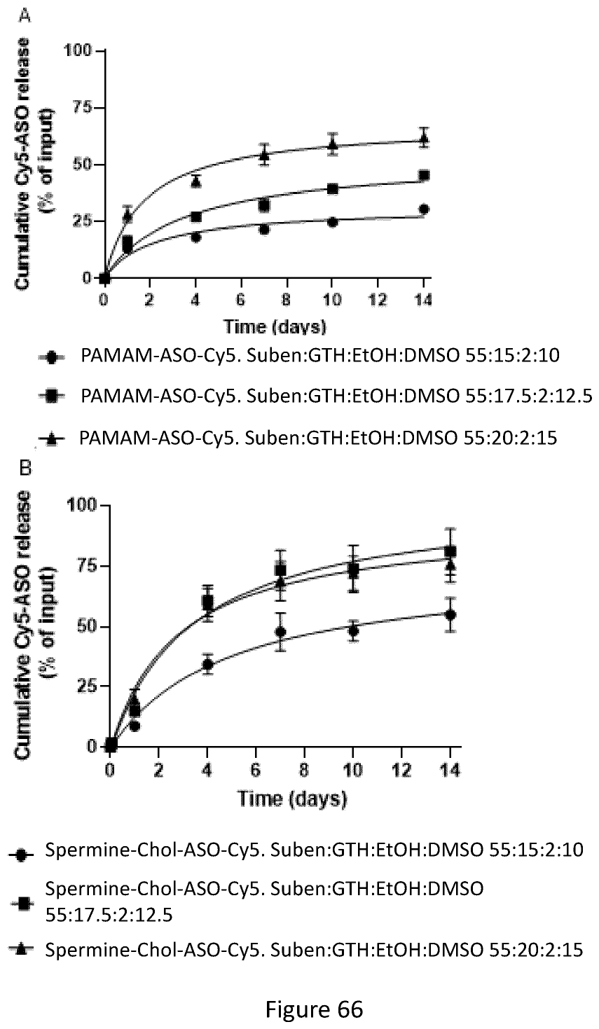

Results and discussion: Cy5-labeled ASOs were complexed to PAMAM or Spermine-Cholesterol and embedded into three different NCCells. Release kinetics was monitored over 14 days by measuring ASO-Cy5 in release media. FIG. 66 demonstrates that the rate of particle release was decreased when lowering the solvent and co-solvent content of the NCCells. The effect of lowering the solvent and co-solvent content from SuBen: GTH: EtOH: DMSO (55:20:2:15) to SuBen: GTH: EtOH: DMSO (55:15:2:10) were significant for both PAMAM (FIG. 66A) and Spermine-cholesterol (FIG. 66B) Cy5-ASO complexes. Both PAMAM polyplex and spermine-chol HIP demonstrated sustained ASO release for 2 weeks.

Conclusion: Release kinetics of ASOs complexed to PAMAM and Spermine-Cholesterol and embedded in NCCells can be modified for long and short-term sustained release by adjusting the co-solvent and solvent content of the NCCell.

### Example 47: NCCell Composition Controls In Vivo Release Kinetics of ASO HIPs and Polyplexes

Aim: Demonstrate how NCCell composition determines release kinetics of ASO formulated as HIP complexes or polyplexes

Principle: By loading a fluorescently labelled ASO (ASO-Cy5) into the NCCell formulated as different complexes, it was possible to analyze the remaining amount of the ASO in the gel at different timepoint. This was done by recovering the NCCell from the mice at the indicated timepoint, dissolving in organic media, and analyzing the amount of Cy5-ASO in the NCCell.

### Methods:

Preparation of polyplexes and HIPs: Spermine-cholesterol HIP of ASO was prepared at N/P ratio of 2 according to example 43. The HIP was concentrated by freeze-drying, dissolved in DMSO, and embedded into NCCell (SuBen: GTH: EtOH: DMSO 55:20:2:15) as described in example 45. The final ASO concentration in the NCCell was 1 mg/mL with 2% of the ASO labelled with Cy5. PAMAM-ASO polyplex was prepared as described in example 42 at N/P ratio of 10. The polyplex was concentrated by freeze-drying, dissolved in DMSO, and mixed with NCCell (SuBen: GTH: EtOH: DMSO 55:20:2:15 or SuBen: GTH: EtOH: DMSO 55:15:2:10) as described in example 45. The final ASO concentration in the NCCell was 0.5 mg/mL with 4% ASO-Cy5.

In vivo setup: On day 0, non-tumor bearing Balb/CJrJ mice were under anesthesia (isoflurane) injected in the flank with 50 μL of NCCell containing the Cy-5 labelled ASO, or 50 μL of NCCell without ASO, as a control. At different timepoints (day 1, 3, 7, and 14 or day 3), mice were euthanized by cervical dislocation, and the injected NCCell recovered from each mouse. Each treatment group contained 3-4 mice.

Analysis of remaining ASO in NCCell: The 50 μL of NCCell, recovered from the individual mice at different timepoints, was mixed with 450 μL of DMSO in a 4 mL glass vial. The mixture was heated to 50° C. for 2 hours and vortexed regularly. After 2 hours of heating, the NCCell was dissolved. 100 μL of the DMSO mixture with dissolved NCCell was transferred to a black 96-well plate. This process was performed for all the recovered NCCell injections, resulting in 3-4 samples per group at each timepoint. A standard curve was prepared by dissolving particles of the ASOs in DMSO at different concentrations, which were also transferred to the black 96-well plate. The fluorescence was measured by fluorescent spectroscopy (Tecan Spark, Ex/Em. 600/670 nm) and the remaining concentration of the ASO in the NCCell was calculated from the standard curve. The data were plotted as percentage of released ASO based on the known starting concentration of the ASO before injection.

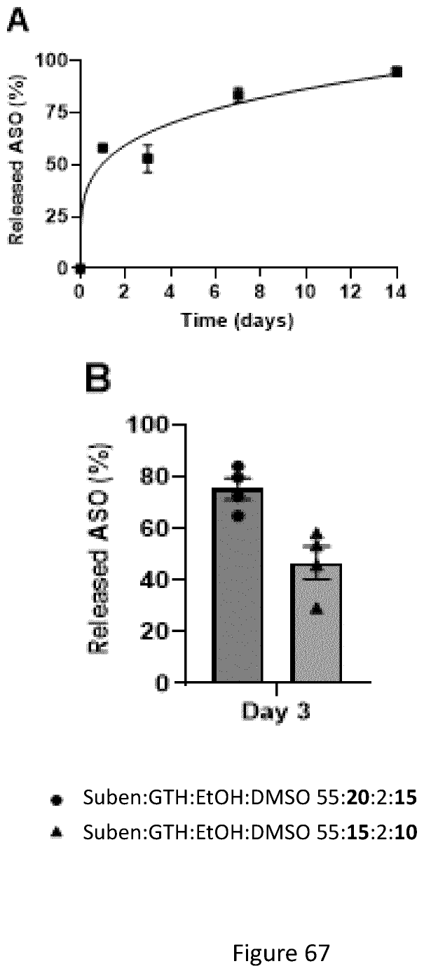

Results and discussion: The ASO release results demonstrate sustained release for up to 14 days in vivo using spermine-cholesterol ASO HIP as shown in FIG. 67A. Within the first 3 days around 50% of the ASO was released from the NCCell, whereafter the release slowed down and was sustained for around 14 days, reaching full release. FIG. 67B demonstrates that by lowering the co-solvent (GTH) and solvent (DMSO) content of the NCCell, the initial burst release can be reduced. This was specifically shown for PAMAM polyplex of ASO by measuring the amount of ASO released on day 3 after injection. The data illustrates the potential of tuning the ASO release in vivo to match the desired release profile.

Conclusion: It was possible to measure in vivo release by analyzing the remaining Cy5-labelled ASO in the NCCell recovered from mice. The results demonstrate that a sustained ASO release for 14 days was achieved in vivo using the specific NCCell ASO formulation. Furthermore, adjusting the co-solvent and solvent ratios of the NCCell components made it possible to control the release.

### Example 48: Pharmacokinetics and Biodistribution of Gd-DOTA Labelled ASO Released from NCCell Analyzed with ICP-MS

Aim: Investigate the biodistribution of Gd-labelled-ASOs after intratumoral injection of an NCCell composition releasing spermine-chol HIP complexed ASOs or free ASOs.

Methods: Gadolinium-labeled ASOs were synthesized as described in example 41. Spermine-Chol Gd-ASO complexes were prepared using the hydrophobic ionpairing method as described in example 43. The ASO complex was concentrated by freeze-drying, dissolved i in DMSO and embedded into NCCell (SuBen: GTH: EtOH: DMSO 55:20:2:15) according to example 45. The final Gd-ASO concentration in the NCCell was 0.8 μg/g. Free Gd-ASO was prepared in PBS buffer at a concentration of 0.95 μg/μL.

On Day 0, 4T1 tumor bearing Balb/CJRj mice were, under anesthesia (isoflurane), injected intratumorally with 50 μL of NCCell or 42 μL of free Gd-ASO in PBS, with a total concentration of 40 μg Gd-ASO per injection. NCCell was drawn up using a 1 mL syringe and 18G needle, and 50 μL injected i.t. using a 1 ml syringe and 23G needle. Free ASO was injected using a 23G needle.

Blood was collected from different groups 10 minutes, 1 hour, 4 hours, 24 hours, 72 hours and 5 days after treatment. Organs and other fluids (liver, spleen, lungs, kidneys) were harvested 24 hours and 5 days after treatment. For sample processing, full organ weight was recorded, as well as the weight of the tissue used, and was further processed for ICP-MS measurements. To prepare tissue for ICP-MS analysis, 50-100 mg of organs or NCCell were digested in 500 μL HNO₃, (69%) 50 μL HCl (37%) and 300 μL H₂O₂ (35%) at 65° C. overnight followed by further dilutions in water and 2% HCl and subjected to ICP-MS analysis using Ir (0.5 ppb) as internal standard.

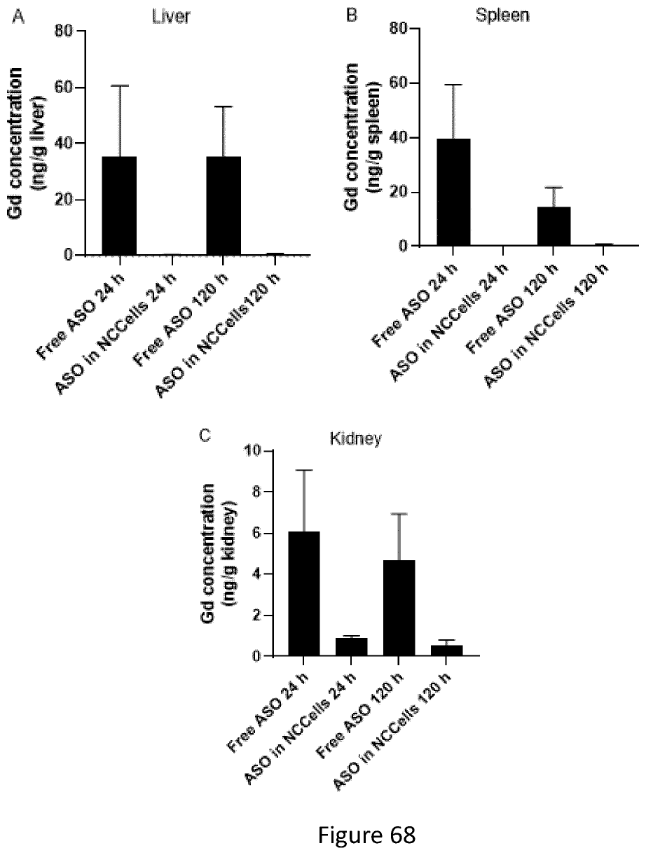

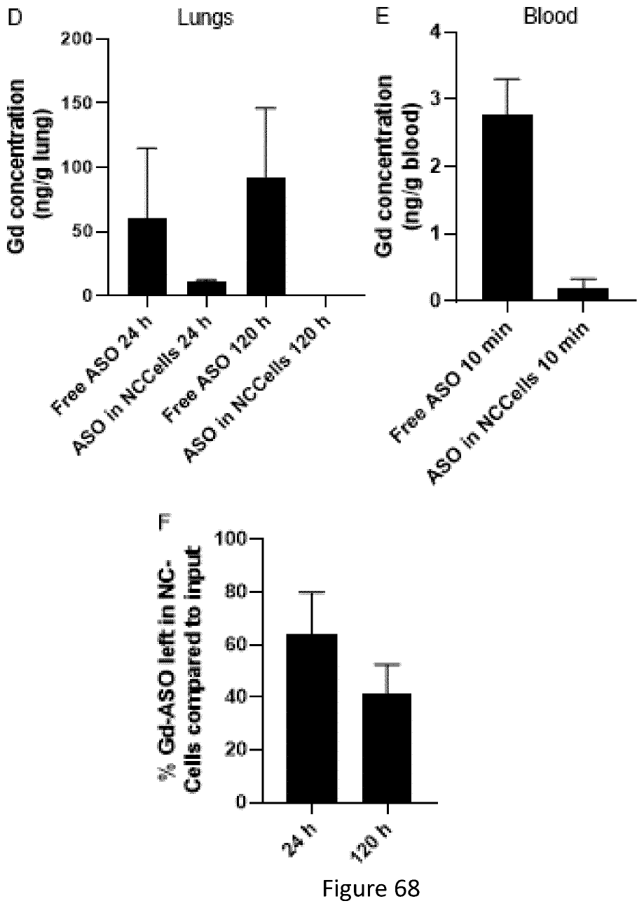

Results and discussion: The highest concentration of free ASO was observed in the lungs, followed by the liver and spleen. Low concentrations could also be detected in kidney tissue FIG. 68A+D. There were no major differences after 24 hours and 120 hours. ASOs complexed with spermine-cholesterol and released from NCCell showed very low distribution into the evaluated organs with only low but detectable amounts in the lungs after 24 hours (FIG. 68A-D).

There was very low signal in all blood samples. Blood activity of the free ASOs could be detected 10 minutes after injection but there were no detectable Gd signal in the blood at later timepoints (FIG. 68E).

The amount of Gd-ASO-complexed to Spermine-Cholesterol left in the NCCell relative to the initially embedded concentration was 65% and 40% after 24 and 120 hours, respectively (FIG. 68F).

The data showed that ASOs complexed with spermine-cholesterol and embedded into NCCell display a different biodistribution pattern than free ASO when injected intratumorally. Free ASO could be readily detected in liver, spleen and lung tissues whereas complexed ASO released from NCCell could not.

After 120 hours there was still ASO-complex present in the NCCell, supporting that a slow release of ASOs in the injection area was provided. The sustained release supports the ability to have continuous and long-lasting gene silencing in the target area. The reduced systemic spillover of ASOs may be beneficial for reducing ASO-induced systemic toxicity. The NCCell technology may allow for silencing of genes that are not tolerated at the systemic level or in organs where distribution of ASOs after free injections is seen.

Conclusion: Free ASOs injected intratumorally distribute to liver, kidney and spleen and could also be detected early in blood, 10 minutes after injections. ASOs embedded into NCCell as complexes could not be detected in blood or tissues, demonstrating a local distribution of ASOs in the surrounding area. After approximately five days 40% of Gd-labeled-ASO was still present in the NCCell, showing that a sustained release was achieved.

### Example 49: In Vivo Cellular Uptake of ASO-Complexes Released from NCCell

Aim: Determine the intratumoral uptake of ASO complexes released from NCCell formulations using Cy5-labelled ASOs and flow cytometric analysis of cell populations.

### Method:

Formulation of ASOs in NCCell: Spermine-cholesterol HIP of ASO was prepared according to example 43 at N/P ratio of 2. PAMAM generation 0.0 polyplex of ASO was prepared as described in example 42 at N/P ratio of 10. Both samples were prepared at an ASO concentration of 0.5 mg/mL (20% was Cy5-labelled ASO) in 1.7 mL of water. The samples were concentrated by freeze-drying, dissolved in DMSO, and embedded into NCCell (SuBen: GTH: EtOH: DMSO 55:20:2:15) as described in example 45. The final ASO concentration in the NCCell was 200 M (20% Cy5.ASO).

Development of murine CT26 tumors: Murine CT26 tumors were established in female Balb/C mice. CT26 syngeneic murine colorectal cancer cells were cultured in RPMI 1640 medium (Gibco™, Thermo Fisher Scientific, or Sigma-Aldrich, Merck) as previously described. Media was supplemented with 10% fetal bovine serum (Gibco™, Thermo Fisher Scientific or Biowest, VWR) and 1% penicillin-streptomycin (Gibco™, Thermo Fisher Scientific or Sigma-Aldrich, Merck). Cells were kept in a humidified tissue culture incubator at 37° C. with 5% CO₂. Cells were subcultured when confluent, and Trypsin-EDTA (0.25%) (Thermo Fisher Scientific) was used to remove adherent cells. Mice were injected under general anesthesia (Isoflurane 2%, 95% O₂) with 300.000 cells in 100 μL PBS using a 29 Ga. insulin syringe. Tumors were allowed to establish for 2 weeks until reaching a mean volume of 140 mm3. A total of 11 mice were included in the study. The mice were randomized according to tumor size and divided into treatment groups.

Treatment of tumors: On day 0, mice included in treatment groups were injected i.t. with 50 μL of NCCell (Sp-chol HIP or PAMAM polyplex, both at 200 UM ASO concentration in NCCell) under general anesthesia (Isoflurane 2%, 95% O2). NCCell injections were performed using a 1 ml syringe and a 25 Ga. needle. Untreated mice were included as controls. On day 1 and day 3 after the treatment with Cy5-ASO formulated in NCCell, uptake of the Cy5-labeled ASOs in different cell subsets in the tumor and tumor-draining lymph node was investigated by flow cytometry.

Cell-uptake analysis by flow cytometry: Tumors and tumor-draining lymph nodes were dissected and fragments of NCCell material were removed from the tumor samples. Tumor samples were mechanically disrupted with scissors and placed directly in enzyme mixture (Tumor Dissociation Kit, mouse, Miltenyi Biotec). Samples were enzymatically digested by placing the tubes in a 37° C. water bath for 40 min. with shaking.

Digested tumors were passed through 70 μm strainers and diluted in PBS for 2 washing steps. 10-20×10⁶ cells were transferred to 96-well plates for staining. Cell pellets were Fc-blocked (5-10 min. on ice) with murine anti CD16/32 monoclonal antibodies (BD Biosciences) followed by dead cell labelling with fixable viability dye (VD)-efluor780 (eBioscience) and surface staining (30 min. on ice) with specific antibodies. The flow cytometric surface staining panel included CD45, Ter119, Ly6g, CD4, CD8, CD31, CD3 and CD64.

The samples were acquired on an LSRfortessa X20 Flow Cytometer using FACSDiva software (BD Biosciences) and data analyzed using FlowJo software (Treestar). Spectral spillover compensation controls were included for all stains using UltraComp eBeads™ Plus Compensation Beads (Invitrogen, Thermo Fisher Scientific). The Cy5 signal was compensated using CT26 cells pulsed with Cy5-labelled ASO for 24 hours in vitro.

Debris and doublets were excluded based on forward and side scatter. Fluorescence minus one (FMO) samples were used for the identification of positive and negative populations.

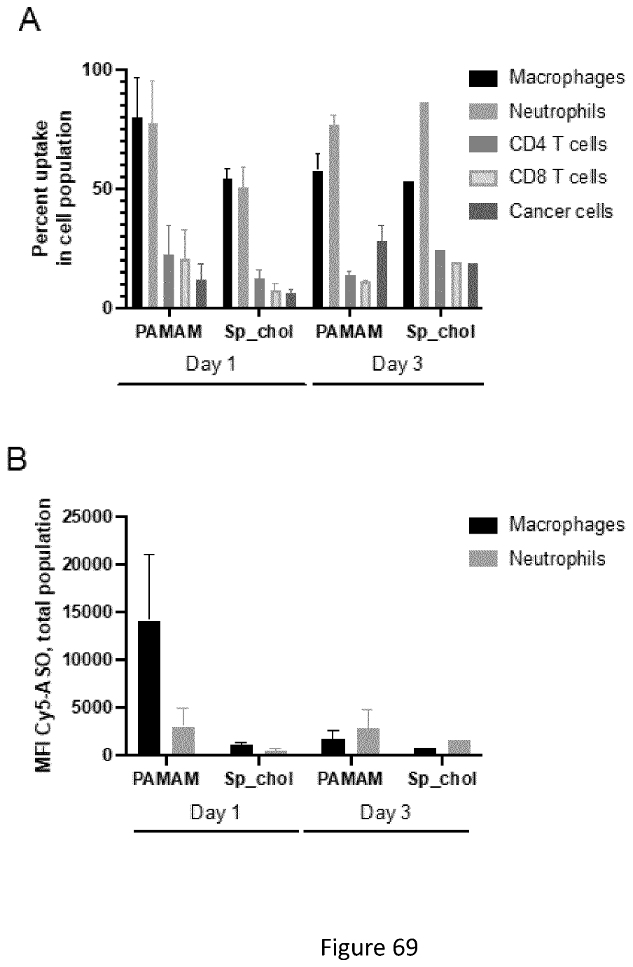

Results and discussion: At both timepoints examined (day 1 and day 3) intracellular uptake of Cy5-labeled ASO was clearly detectable by flow cytometry in cell populations from tumors. Specifically, ASOs were identified in macrophage and neutrophil populations with a very high uptake (50-99% positive cells within population), and in T cells as well as cancer cells with a moderate uptake (10-20% positive cells) (FIG. 69A). No positive cells were observed in the tumor-draining lymph node for any of the treatments, suggesting that cellular uptake takes place close to the site of injection.

The median fluorescent intensity (MFI) was used as a semi-quantitative measure for Cy5-labeled ASO in each cell population. A marked increase in MFI was observed on day 1 in the macrophage population in tumors treated with PAMAM polyplex of ASO, but on day 3 the MFI values were comparable between treatments (FIG. 69B). The tumor retention supports that the NCCell technology may allow for silencing of gene targets not tolerated at the system level.

Conclusion: High cell uptake of ASO formulations released from NCCell in both macrophages and neutrophils was observed, while moderate cell uptake was observed for cancer cells and immune cells. Finally, no cell uptake was found in the tumor-draining lymph node indicating that uptake was focused at the injection site.

### Example 50: In Vitro MALAT1 and KRAS Silencing from ASO-Complexes

Aim: To demonstrate silencing in vitro from ASO-complexes of MALAT1 and KRAS in the 4T1 and MiaPaca2 cell lines.

### Method:

### MALAT1 Silencing in 4T1 Cell Line:

PAMAM Gen 0.0 polyplex of ASO MALAT1 was prepared as described in example 42 at N/P ratio of 10. Spermine-cholesterol HIP of ASO MALAT1 was prepared according to example 43 at N/P ratio of 2. The complexes were freeze-dried and dissolved in DMSO as described in example 45, yielding ASO complexes at 25 M in DMSO. Free Malat1 ASO was prepared at 25 UM in DMSO.

Transfections were performed in 24 well culture plates with 40.000 4T1 Cells in 500 μL culture volume (RPMI 10% FBS, 1% Sodium Pyruvate (100 mM), and 1% Penicillin-Streptomycin (10,000 U/mL)). 5 μL of ASO complexes or free ASO were injected in each well, for a final concentration of 250 nM. The cells were incubated for 48 hours, after which the cells were lysed and stored at −20° C. until RNA extraction using silica spin columns (RNeasy Mini Kit, (Qiagen 74106) performed according to manufacturer's protocol). Silencing efficiency evaluated by RTqPCR using a Biorad CFX96 Touch Real-Time PCR Detection System and Biorad CFX Manager program. RTqPCR was performed on the target gene (MALAT1) and two housekeeping genes (GAPDH and HPRT). Results were analysed using the delta-delta Ct method (also referred to as the 2-1ict method). MALAT1 expression was normalized to the housekeeping genes and silencing reported as a reduction in expression compared to untreated controls.

### KRAS Silencing in MiaPaca2 Cell Line:

PAMAM generation 0.0 polyplexes of KRAS ASO was prepared similarly to example 42 at N/P ratio of 10. Furthermore, HIP of spermine-cholesterol and KRAS ASO was prepared at N/P ratio of 2 according to example 43. The complexes were freeze-dried and dissolved in DMSO at a final concentration of 200 UM as described in example 45. Free KRAS ASO was also prepared at 200 UM in DMSO.

Transfections were performed in 24 well culture plates with 80.000 MiaPaca2 Cells in 500 μL culture volume (RPMI 10% FBS, 1% Sodium Pyruvate (100 mM), and 1% Penicillin-Streptomycin (10,000 U/mL)). 1.25 μL, 2.5 μL or 5 μL of ASO complexes or free ASO was injected in each well, for final ASO concentrations of 500 nM, 1 UM and 2 μM, respectively. The cells were incubated for 48 hours, after which the cells were lysed and stored at −20° C. until RNA extraction using silica spin columns (RNeasy Mini Kit, (Qiagen 74106) performed according to manufacturer's protocol). Silencing efficiency evaluated by RTqPCR using a Biorad CFX96 Touch Real-Time PCR Detection System and Biorad CFX Manager program. RTqPCR was performed on the target gene (KRAS) and two housekeeping genes (GAPDH and HPRT). Results were analysed using the delta-delta Ct method (also referred to as the 2-1ict method). KRAS expression was normalized to the housekeeping genes and silencing reported as reduction in comparison to expression levels in untreated controls.

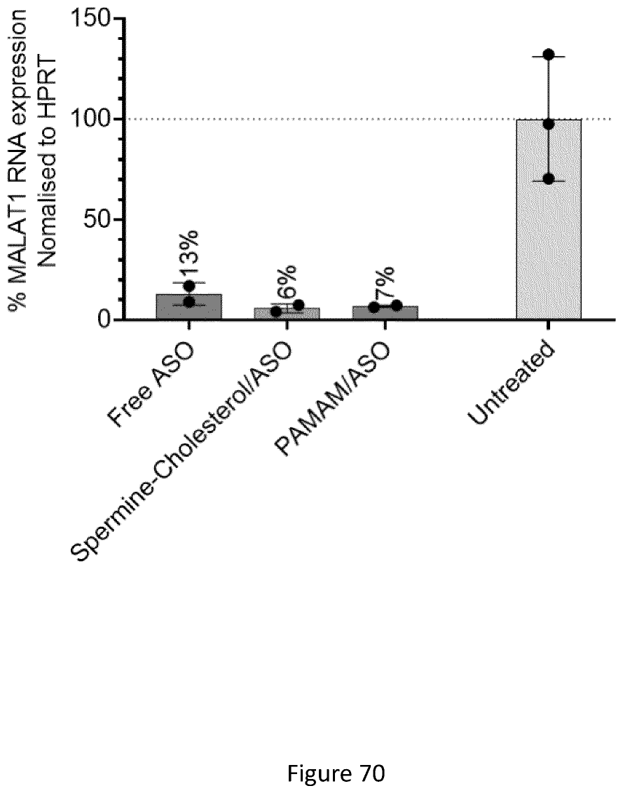

Results and discussion: PAMAM polyplexes and spermine-cholesterol HIP complexes of MALAT1 ASO silenced MALAT1 expression in 4T1 cells as demonstrated in FIG. 70. Silencing achieved by polyplexes and HIP complexes was superior or equal to that of free ASOs.

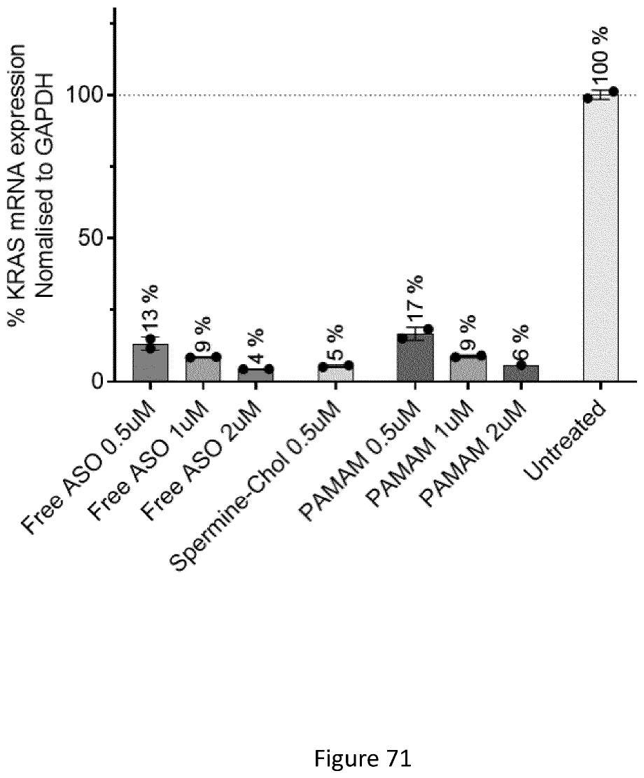

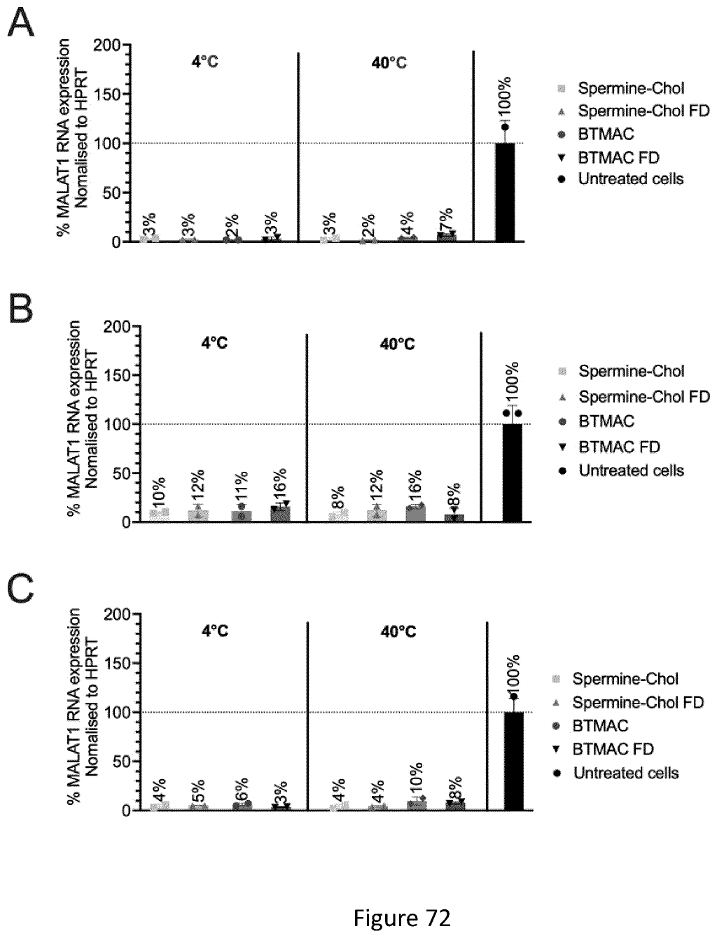

PAMAM polyplexes and spermine-cholesterol HIP complexes of KRAS ASO successfully silenced KRAS expression in MIA PaCa-2 cells as shown in FIG. 71. The silencing capacity demonstrated to be dose dependent. Silencing achieved by the complexes was similar to free ASOs at the same concentrations.

Conclusion: Polyplexes and HIP complexes of ASOs successfully silenced the gene expression of target genes (MALAT1 and KRAS) in 4T1 and MIA PaCa-2 cell lines.

### Example 51: Stability of HIP Complexes of ASO when Stored Over Time

Aim: Demonstrate that ASO-HIP complexes can be stored freeze dried or dissolved in DMSO without affecting silencing capacity over time.

Method: HIP complexes (Spermine-cholesterol/ASO (MALAT1), N/P ratio 2, 50 M; and BTMAC/ASO (MALAT1), N/P ratio 4, 50 μM) were prepared as described in example 43. The HIPs were freeze-dried and dissolved in DMSO according to example 45. The HIP complexes were stored either dissolved in DMSO or as freeze-dried powders, at 4° C. or 40° C. for various length of time. After storage, freeze dried samples were resuspended in DMSO for treating cells.

For each timepoint, transfections were performed in 24 well culture plates with 40.000 4T1 Cells in 500 μL culture volume (RPMI 10% FBS, 1% Sodium Pyruvate (100 mM), and 1% Penicillin-Streptomycin (10,000 U/mL)). 5 μL of HIP complexes in DMSO was injected in each well, for a final ASO concentration of 500 nM. The cells were incubated for 48 hours, after which the cells were lysed and stored at −20° C. until RNA extraction using silica spin columns (RNeasy Mini Kit, (Qiagen 74106) performed according to manufacturer's protocol). Silencing efficiency evaluated by RTqPCR using a Biorad CFX96 Touch Real-Time PCR Detection System and Biorad CFX Manager program. RTqPCR was performed on the target gene (MALAT1) and two housekeeping genes (GAPDH and HPRT). Results were analysed using the delta-delta Ct method (also referred to as the 2-1ict method). MALAT1 expression was normalized to the housekeeping genes and silencing reported as reduction normalized to expression levels in untreated controls.

Results and discussion: The HIP complexes Spermine-cholesterol/ASO (MALAT1) and BTMAC/ASO (MALAT1), stored either dissolved in DMSO or as freeze-dried powders, at both 4° C. and 40° C. were capable of silencing MALAT1 expression. This was the case for all timepoints; 14 days (FIG. 71A), 6 weeks (FIG. 71B), and 3 months (FIG. 71C) without loss of efficacy over time.

Conclusion: HIP complexes prepared from MALAT1 ASO maintained gene silencing capabilities for at least 3 months when stored either dissolved in DMSO or as freeze-dried powders, at both 4° C. and 40° C.

### Example 52: Sustained In Vitro Silencing of MALAT1 by ASO Released from NCCell

Aim: To demonstrate sustained silencing of MALAT1 from ASO-HIP complexes released from NCCell in vitro.

Method: PAMAM gen 0.0 polyplex of MALAT1 ASO was prepared according to example 42 at N/P ratio of 10. The polyplex was concentrated by freeze-drying, dissolved in DMSO, and embedded into NCCell (SuBen: GTH: EtOH: DMSO 55:20:2:15) as described in example 45, yielding a final ASO concentration of 500 μg/mL in the NCCell.

Transfections were performed in 24 transwell (8 μm pore) culture plates with 40.000 4T1 cells in 2 mL culture volume (RPMI 10% FBS, 1% Sodium Pyruvate (100 mM), and 1% Penicillin-Streptomycin (10,000 U/mL)).

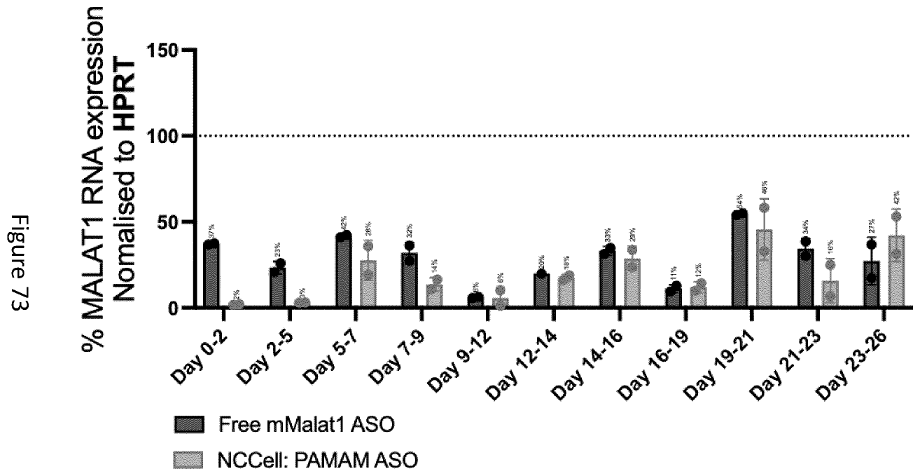

NCCell (100 μL) were injected into the transwell insert (8 μm pore) using a 2-component 1 ml syringe and 23G needle and allowed to release transfection particles for 48 hours, then transferred to a new well and left for another 72 hours (time points indicated in FIG. 73). Free ASOs were also added freshly at each timepoint at a concentration of 25 nM. The transfer of the insert was continued for more than 3 weeks. Every time inserts were transferred, cells were lysed and stored at −20° C. until RNA extraction using silica spin columns (RNeasy Mini Kit, (Qiagen 74106) performed according to manufacturer's protocol). Silencing efficiency was evaluated by RTqPCR using a Biorad CFX96 Touch Real-Time PCR Detection System and Biorad CFX Manager program. RTqPCR was performed on the target gene (MALAT1) and two housekeeping genes (GAPDH and HPRT). Results were analysed using the delta-delta Ct method (also referred as the 2-1\Ct method). MALAT1 expression was normalized to the housekeeping genes.

Results and discussion: NCCell embedded with PAMAM polyplexes of MALAT1 ASO demonstrated sustained silencing of MALAT1 in 4T1 cells for 26 days as shown in FIG. 73. The sustained silencing was evaluated by repeated transfer of the same NCCell inserts to fresh cells. Silencing was superior or equal to treatment with 25 nM free ASO at each timepoint. This finding validates that ASO polyplexes embedded into NCCell were stable, even when released over long periods of time.

Conclusion: The NCCell composition (SuBen: GTH: EtOH: DMSO 55:20:2:15) containing PAMAM polyplexes provided sustained release of MALAT1 silencing ASOs as evident by the observed silencing of MALAT1 expression for 26 days.

### Example 53: KRAS Efficacy In Vivo

Aim: To determine the therapeutic effect of KRAS knockdown by ASO released from intratumoral NCCell in the MIA-PaCa2 tumor model by measuring tumor growth control. Determine the effect of treatment on tumor cell proliferation.

### Method:

Formulation of ASOs in NCCell: PAMAM generation 0.0 polyplex of hKRAS ASO was prepared as described in example 42 at N/P ratio of 10. The polyplex was concentrated by freeze-drying, dissolved in DMSO and embedded into NCCell (SuBen: GTH: EtOH: DMSO 55:20:2:15) as shown in example 45. The final hKRAS ASO concentration in the NCCell was 4 mg/mL.

Development of human MiaPaca xenograft tumors: Human MiaPaca2 xenograft tumors were established in female 55 Balb/C-nu (BALB/cAnN-Foxn1nu/nu/Rj) “nude” mice. The cells were cultured in RPMI 1640 medium (Gibco™, Thermo Fisher Scientific, or Sigma-Aldrich, Merck). Media was supplemented with 10% fetal bovine serum (Gibco™, Thermo Fisher Scientific or Biowest, VWR) and 1% penicillin-streptomycin (Gibco™, Thermo Fisher Scientific or Sigma-Aldrich, Merck). Cells were kept in a humidified tissue culture incubator at 37° C. with 5% CO2. Cells were subcultured when confluent, and Trypsin-EDTA (0.25%) (Thermo Fisher Scientific) was used to remove adherent cells. Mice were injected under general anesthesia (Isoflurane 2%, 95% O₂) with 5,000,000 cells in 100 μL PBS using a 29 Ga. insulin syringe. Tumors were allowed to establish for 35 days until reaching a mean volume of 78 mm3. A total of 16 mice were included in the study. The mice were randomized according to tumor size and divided into either untreated controls (n=8) or NCCell treated mice (n=8).

Treatment of tumors: One day after inclusion, mice were injected in tumors with 50 μL of NCCell formulation (200 μg of hKRAS ASO pr injection) under general anaesthesia (Isoflurane 2%, 95% O₂). NCCell injections were performed using a 1 mL syringe and a 25 Ga. hypodermic needle. For the duration of the study, the mice were hereafter treated at different times according to the description above (a total of 5 treatments). Mice were excluded from the study if wounding of the tumor prevented retention of the treatment (a total of 2 mice were excluded after 3 and 4 treatments, respectively). Tumor sizes were determined by measurement with electronic caliber and tumor volumes were calculated according to the formula: V=1/2 (Length×Width²).

Tumor analysis: On day 1 after receiving the 5th treatment, mice were sacrificed, and the tumors were dissected out. The NCCell fragments were removed, and the tumor mass was determined by weighing the remaining tissue. After weighing, tumor samples were mechanically disrupted with scalpels and placed directly in enzyme mixture (Tumor Dissociation Kit, mouse, Miltenyi Biotec). Samples were enzymatically digested by placing the tubes in a 37° C. water bath for 40 minutes with shaking.

Digested tumors were passed through 70 μm strainers and diluted in PBS for 2 washing steps. 10-20×10⁶ cells were transferred to 96-well plates for staining. Cell pellets were Fc-blocked (5-10 min. on ice) with human anti CD16/32 monoclonal antibodies (BD Biosciences) followed by dead cell labelling with fixable viability dye (VD)-efluor780 (eBioscience). For intracellular staining of Ki67, cells were fixed (30 min. at RT) and permeabilized using the eBioscience™ Foxp3/Transcription Factor Staining Buffer Set (Thermo Fisher Scientific).

The samples were acquired on an LSRfortessa X20 Flow Cytometer using FACSDiva software (BD Biosciences) and data analyzed using FlowJo software (Treestar). Spectral spillover compensation controls were included for all stains using UltraComp eBeads™ Plus Compensation Beads (Invitrogen, Thermo Fisher Scientific). Debris and doublets were excluded based on forward, and side scatter. Fluorescence minus one (FMO) control was used for the identification of the Ki67 positive population.

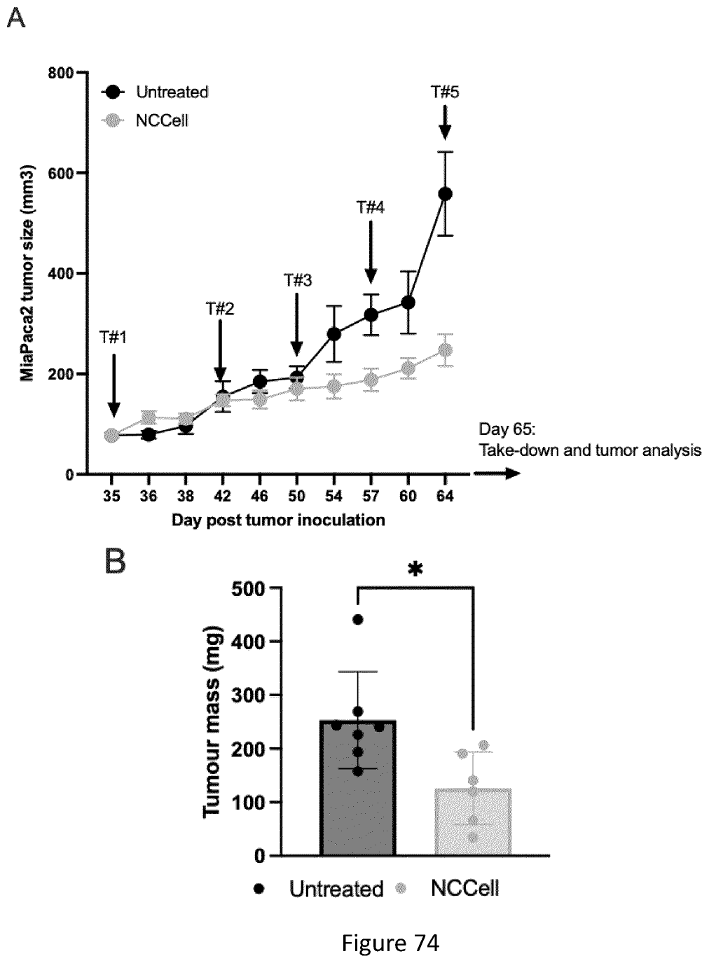

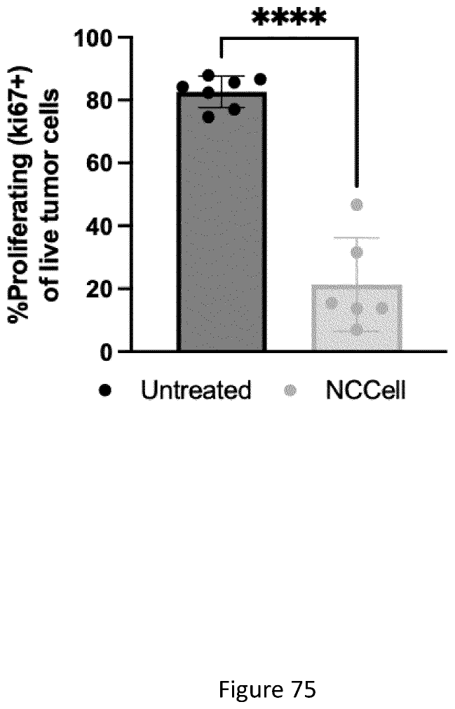

Results and discussion: Treatment with hKRAS ASO loaded NCCell intratumorally reduced the tumor growth rate, as well as the tumor burden at the end of the study (FIG. 74). When examining the proliferation rate of the tumor cells, a significantly reduced proliferation was observed in the hKRAS ASO groups, as expected when KRAS expression is lowered (FIG. 75).

Conclusion: Treatment of the MiaPaca tumors with NCCell loaded with hKRAS ASO, demonstrated reduced tumor growth, and tumor cell proliferation compared to the untreated group

1. A composition comprising:
a. a nucleic acid component; and
b. a hydrophobic component;
wherein:
(i) the hydrophobic component comprises a hydrophobic carbohydrate, a lipid, a hydrophobic polymer, or mixture thereof;
(ii) the hydrophobic component comprises a hydrophobic or amphiphilic molecule that contains at least one primary, secondary, tertiary or quaternary amine;
(iii) the nucleic acid component and the hydrophobic component are dispersed or dissolved in an organic solvent; and
(iv) the composition has a higher viscosity in an aqueous environment as compared to its viscosity in a non-aqueous environment.

2. The composition according to claim 1, wherein the organic solvent diffuses from the composition when the composition is in an aqueous environment.

3. The composition according to claim 1, wherein the composition has a viscosity that is at least 10,000 centipoise (cP) higher in an aqueous environment than in its viscosity in a non-aqueous environment.

4. The composition according to claim 1, wherein the composition is a liquid when in a non-aqueous environment.

5. The composition according to claim 1, wherein the composition transforms to a gel-like state when transferred from a non-aqueous environment to an aqueous environment.

6. The composition according to claim 1, wherein the composition transforms to a solid when transferred from a non-aqueous environment to an aqueous environment, optionally wherein the solid comprises a crystalline solid or an amorphous solid.

7. The composition according to claim 1, wherein the organic solvent is selected from DMSO, benzyl alcohol, benzyl benzoate, propylene carbonate, NMP, and polyethylene glycol.

8. The composition according to claim 1, wherein the aqueous environment is within the body of a subject.

9. The composition according to claim 8, wherein the aqueous environment is within a tissue of the subject, such as a muscle, cancer tissue or lymph node.

10. The composition according to claim 7, wherein the carbohydrate, lipid, polymer or mixture thereof contains the at least one primary, secondary, tertiary or quaternary amine.

11. The composition according to claim 1, wherein the nucleic acid component comprises a free nucleic acid.

12. The composition according to claim 1, wherein the nucleic acid component comprises a hydrophobic ion-pairing (HIP) complex.

13. The composition according to claim 1, wherein the nucleic acid component comprises a nanoparticle.

14. The composition according to claim 13, wherein the nanoparticle is a lipid nanoparticle or a polymer nanoparticle.

15. The composition according to claim 13, wherein the nanoparticle is 20-500 nm in size.

16. A composition according to claim 1 wherein the hydrophobic carbohydrate comprises a hydrophobic derivative of a disaccharide or a trisaccharide or a mixture thereof.

17. The composition according to claim 16, wherein the hydrophobic carbohydrate comprises a derivative of sucrose, lactose, maltose, trehalose or raffinose.

18. The composition according to claim 1, wherein the composition comprises at least 30% (w/w) of hydrophobic carbohydrate.

19. The composition according to claim 1, wherein the composition further comprises a co-solvent.

20. The composition according to claim 19 wherein the co-solvent is a lipid, a phospholipid, a pegylated lipid, a monoglyceride, a diglyceride or a triglyceride.

21. The composition according to claim 1, wherein the composition forms a depot when in an aqueous environment.

22. The composition according to claim 1, wherein the composition forms an NCCell when in an aqueous environment.

23. The composition according to claim 1, wherein the nucleic acid component comprises a targeting ligand that targets a receptor on a cell surface.

24. The composition according to claim 1, wherein the nucleic acid component comprises an oligonucleotide and/or a polynucleotide.

25. The composition according to claim 1, wherein the nucleic acid component comprises DNA or RNA.

26. The composition according to claim 1, wherein the nucleic acid component comprises a therapeutic nucleic acid.

27. The composition according to claim 26, wherein the therapeutic nucleic acid is selected from siRNA, ASO, mRNA, and DNA.

28. The composition according to claim 1, wherein the composition is formulated as an injectable.

29. The composition according to claim 1, wherein the composition is for use in transfection.

30. The composition according to claim 1, wherein the composition further comprises an imaging agent.

31. The composition according to claim 1, for use in medicine.

32. The composition according to claim 1, for use in therapy.

33. The composition according to claim 1, for use as a controlled release system for a nucleic acid-based component in a subject.

34. The composition according to claim 1, for use in treating a disease treatable by gene engineering.

35. The composition according to claim 1, for use in treating a disease treatable by a nucleic acid-based therapy.

36. The composition for use according to claim 35, wherein the nucleic acid-based therapy is selected from DNA, pDNA, ssDNA, dsDNA, antisense DNA, eecDNA, microDNA, spcDNA, episomal DNA, linear DNA, RNA (messenger RNA (mRNA), self-replicating mRNA, transfer RNA (tRNA), ribosomal RNA (rRNA)), small single or double stranded RNA (dsRNA, RNA interference (RNAi) including microRNA (miRNA), small interfering RNA (siRNA or ASO, with modifications including phosphorotioate (PS), PS morpholino, 2′-O-methyl, 2′-O-methoxyethyl, 2′fluoro, 5′methylcystine, G-clamp), splice switching antisense oligonucleotide (SSO), CRISPR-Cas9 sgRNAs, piwi-interacting RNA (piRNA) and/or repeat associated small interfering RNA (rasiRNA).

37. The composition for use according to claim 31, for use in treating cancer, an inflammatory disease, an immune system disorder, a genetic disease, a regenerative disorder, a non-healing tissue disorder, myelodysplastic syndrome, an autoimmune disorder, rheumatoid disease, a deficiency diseases, a hereditary disease, a storage disease, a degenerative disorder, anaemia, an endocrine disorder, a hormone imbalances, hormone inactivation or a psychological disorder.

38. The composition for use according to claim 31, wherein the use comprises administration by injection or catheterization.

39. A method of treating a subject by nucleic acid-based therapy, wherein the method comprises administering to the subject a composition according to claim 1.

40. The method of claim 39, wherein the nucleic acid therapy comprises administration of DNA, pDNA, ssDNA, dsDNA, antisense DNA, eecDNA, microDNA, spcDNA, episomal DNA, linear DNA, RNA (messenger RNA (mRNA), self-replicating mRNA, transfer RNA (tRNA), ribosomal RNA (rRNA)), small single or double stranded RNA (dsRNA, RNA interference (RNAi) including microRNA (miRNA), small interfering RNA (siRNA or ASO, with modifications including phosphorotioate (PS), PS morpholino, 2′-O-methyl, 2′-O-methoxyethyl, 2′fluoro, 5′methylcystine, G-clamp), splice switching antisense oligonucleotide (SSO), CRISPR-Cas9 sgRNAs, piwi-interacting RNA (piRNA) and/or repeat associated small interfering RNA (rasiRNA).

41. The method of claim 39, wherein the method comprises treating a disease selected from cancer, an inflammatory disease, an immune system disorder, a genetic disease, a regenerative disorder, a non-healing tissue disorder, myelodysplastic syndrome, an autoimmune disorder, rheumatoid disease, a deficiency diseases, a hereditary disease, a storage disease, a degenerative disorder, anaemia, an endocrine disorder, a hormone imbalances, hormone inactivation or a psychological disorder.

42. The method according to claim 39, wherein the method comprises administration by injection or catheterization.

43. Use of a composition according to claim 1 in an in vitro method for transfecting one or more cells.

44. A method of producing a composition according to claim 1, wherein the method comprises dissolving or dispersing in an organic solvent, (a) a nucleic acid component; (b) a hydrophobic carbohydrate, a lipid, a hydrophobic polymer, or mixture thereof; and (c) a hydrophobic component, wherein the hydrophobic or amphiphilic molecule contains at least one primary, secondary, tertiary or quaternary amine.
